(19) United States
(12) Patent Application Publication
(10) Pub. No.: US 2024/0199750 A1
(43) Pub. Date: Jun. 20, 2024

(54) MULTISPECIFIC PROTEINS COMPRISING AN NKP46-BINDING SITE, A CANCER ANTGIENGE BINDING SITE FUSED TO A CYTOKINE FOR NK CELL ENGAGING

(71) Applicant: INNATE PHARMA, Marseille (FR)

(72) Inventors: Stephanie CORNEN, Marseille (FR); Laurent GAUTHIER, Marseille (FR); Yannis MOREL, Marseille (FR); Olivier DEMARIA, Marseille (FR)

(21) Appl. No.: 18/283,385

(22) PCT Filed: Mar. 24, 2022
(86) PCT No.: PCT/EP2022/057824

§ 371 Date: Sep. 21, 2023

Related U.S. Application Data
(60) Provisional application No. 63/166,374, filed on Mar. 26, 2021.

(51) Int. Cl. C07K 16/28; A61K 39/00; A61P 35/00; C07K 14/54; C07K 14/55; C07K 14/56; C07K 14/565; C12N 5/0783

(52) U.S. Cl. CPC C07K 16/2866; A61P 35/00; C07K 14/54; C07K 14/5418; C07K 14/5434; C07K 14/5443; C07K 14/55; C07K 14/56; C07K 14/565; C07K 16/2803; C07K 16/283; C12N 5/0646; A61K 39/00; A61K 2039/505; C07K 2317/31; C07K 2317/92; C07K 2319/30

(57) ABSTRACT

Multi-specific proteins that bind to NKp46 and a cytokine receptor on NK cells, and optionally that further bind CD16A on NK cells, and that also bind to an antigen of interest (e.g. a cancer antigen) on a target cell (e.g. a cancer cell). The multi-specific proteins are capable of increasing NK cell cytotoxicity toward a target cell that expresses the antigen of interest (e.g., a cell that contributes to disease, a cancer cell).

### CROSS-REFERENCE TO RELATED APPLICATIONS

This application claims the benefit of U.S. Provisional Application No. 63/166,374 filed 26 Mar. 2021, the disclosure of which is incorporated herein by reference in its entirety; including any drawings and sequence listings.

### REFERENCE TO THE SEQUENCE LISTING

The present application is being filed along with a Sequence Listing in electronic format. The Sequence Listing is provided as a file entitled “NKp46-12 PCT_ST25 txt”, created Mar. 22, 2022, which is 820 KB in size. The information in the electronic format of the Sequence Listing is incorporated herein by reference in its entirety.

### FIELD OF THE INVENTION

Multispecific proteins that bind and specifically redirect effector cells to lyse a target cell of interest via interaction with multiple receptors are provided. The proteins have utility in the treatment of disease, notably cancer or infectious disease.

### BACKGROUND

Interleukin 2 (IL2 or IL-2) is one example of a pluripotent cytokine that acts on a cytokine receptor expressed by NK cells. IL-2 is mainly produced by activated T cells, especially CD4+ T helper cells, and functions in aiding the proliferation and differentiation of B cells, T cells and NK cells. IL-2 is also essential for Treg function and survival. In eukaryotic cells, human IL-2 (uniprot: P60568) is synthesized as a precursor peptide of 153 amino acids with a 20 residue signal sequence, that gives rise to a mature secreted IL-2 having the amino acid sequence of SEQ ID NO: 404. Interleukin 2 has four antiparallel, amphiphilic alpha helices. These four alpha helices form a quaternary structure that is essential for its function. In most cases, IL-2 works through three different receptors: interleukin 2 receptor alpha (IL-2Rα; CD25), interleukin 2 receptor beta (IL-2Rβ; CD122), and interleukin 2 receptor gamma (IL-2Rγ; CD132). IL-2Rβ and IL-2Rγ are essential for IL-2 signaling, while IL-2Rα (CD25) is not necessary for signaling, but can confer high affinity binding of IL-2 to receptors. The trimer receptor (IL-2αβγ) formed by the combination of IL-2Rα, β, and γ is the IL-2 high affinity receptor (KD about 10 pM), and the dimer receptor (IL-2βγ) is an intermediate affinity receptor (KD about 1 nM).

Immune cells express dimer or trimer IL-2 receptors. Dimer receptors are expressed on cytotoxic CD8+ T cells and natural killer cells (NK), while trimer receptors are mainly expressed on activated lymphocytes and CD4⁺CD25+FoxP3+inhibitory regulatory T cells (Treg). Because resting effector T cells and NK cells do not have CD25 on the cell surface, they are relatively insensitive to IL-2. Treg cells consistently express the highest level of CD25 in the body. Due to the low concentrations of IL-2 that typically exists in tissues, IL-2 preferentially activates cells that express the high affinity receptor complex (CD25:CD122:CD132), and therefore under normal circumstances, IL-2 will preferentially stimulate Treg cell proliferation.

IL-15, IL-12, IL-7, IL-27, IL-18, IL-21, and IFN-α share many aspects of receptor binding, complex assembly and signaling with IL-2. For example IL-15, IL-21 and IL-7 like IL-2 both act on NK cells via the common-γ chain receptor (CD132). IL-15 binds to the IL-15 receptor (IL-15R) which is composed of three subunits: IL-15Rα, CD122, and CD132. Two of these subunits, CD122 and CD132, are shared with the receptor for IL-2, but IL-2 receptor has an additional subunit (CD25). IL-15Rα (CD215) specifically binds IL15 with very high affinity, and is capable of binding IL-15 independently of other subunits. IL-21 is another example of a type I cytokine, and its IL-21 receptor (IL-21R) has been shown to form a heterodimeric receptor complex with the IL-2/IL-15 receptor common gamma chain (CD132).

NK cells have the potential to mediate anti-tumor immunity. However, NK cells have been shown to cause toxicity in mice through their hyper-activation and secretion of multiple inflammatory cytokines when IL-2 was administered together with IFN-a (Rothschilds et al, Oncoimmunology. 2019; 8(5):). In addition, NK cells were also shown to cause toxicity of the cytokine IL-15 that also signals through IL-2Rβγ (see WO2020247843 citing Guo et al, J Immunol. 2015; 195(5):2353-64).

One potential solution to the immune toxicity mediated by cytokines such as IL-2 was to fuse it to or associate it with a tumor-specific antibody. However, it was found that that while IL-2 indeed synergized with antitumor antibody in anti-tumor effect in vivo, the inclusion of IL-2 and anti-tumor antigen antibody in the same molecule presented no efficacy or toxicity advantage. The IL-2 moiety entirely governed biodistribution, explaining the observation that immunocytokines recognizing irrelevant antigen performed equivalently to tumor-specific immunocytokines when combined with antibody (Tzeng et al. Proc Natl Acad Sci USA. 2015 Mar. 17; 112(11): 3320-332).

Studies focusing on the effect of cytokines on NK cells have generally focused on single cytokines or simple combinations. More recently, it has been reported that IL-15, IL-18, IL-21, and IFN-α, alone and in combination, and their potential to synergize with IL-2, and that very low concentrations of both innate and adaptive common γ chain cytokines synergize with equally low concentrations of IL-18 to drive rapid and potent NK cell CD25 and IFN-γ expression (Nielsen et al. Front Immunol. 2016; 7: 101). However, administration of cytokines to humans has involved toxicity, which makes combination treatment with cytokines challenging. Furthermore, little remains known on potential synergies or interaction between cytokine receptor signaling pathways and other activating receptors in NK cells. There is therefore a need for new ways to mobilize NK cells in the treatment of disease, particularly cancer.

### SUMMARY OF THE INVENTION

The present invention arises from the discovery of functional multi-specific proteins that bind to NKp46 and a cytokine receptor on NK cells, and optionally that further bind CD16A on NK cells, and that also bind to an antigen of interest (e.g. a cancer antigen) on a target cell (e.g. a cancer cell). The multi-specific proteins are capable of increasing NK cell cytotoxicity toward a target cell that expresses the antigen of interest (e.g., a cell that contributes to disease, a cancer cell).

The multi-specific protein's ability to bind, in cis, to NKp46 and to the cytokine receptor (and optionally further through CD16A) at the surface of an NK cell is believed to lead to a particularly advantageous cell surface receptor signalling, in turn resulting a potent anti-tumor response by NK cells.

IL2-mediated immune toxicity is known to be driven by NK cells. However, the multi-specific proteins of the disclosure bearing an IL-2 moiety displayed a strong NK-cell mediated anti-tumor activity without immune toxicity. The incorporation of a cytokine in the multi-specific proteins increased the potency of cytokine-receptor-mediated activity in NK cells by two orders of magnitude, compared to the cytokine alone. In parallel, when binding both NKp46 and CD16A and comprising a cytokine (IL-2 variant) that substantially retained the affinity for its receptor on NK cells of the wild-type IL-2, the multispecific protein displayed an EC50 for induction of cytotoxicity toward tumor cells that was well below its EC50 for induction of NK cell proliferation. The multispecific proteins will therefore be highly effective in activating NK cell-mediated cytotoxicity at the concentrations where they begin to induce significant cytokine-receptor signalling and/or NK cell proliferation, even when the cytokine is attenuated. This may explain the observed lack of toxicity, as the concentration (e.g., dose) of protein that is used (e.g., administered) can be highly efficacious yet remain below the level that causes NK-cell mediated immune toxicity. The proteins therefore represent a versatile platform that can readily integrate different cytokines and different levels of attenuation for cytokines.

When the protein was made to incorporate a CD122 ABD embodied as a IL2 variant with loss of binding to CD25, the multispecific protein displayed a 4-log decrease in ability to induce signalling in and/or activation of Treg cells compared to native IL-2. When combined with the aforementioned 2-log increase in the ability to induce signaling in NK cells compared to the native IL-2, the multispecific proteins have a 1,000,000-fold increase in potency for induction of cytokine receptor signaling in NK cells compared to in Tregs. Accordingly, the CD122-binding multi-specific proteins can be used at concentrations where they mediated NK cell proliferation and/or infiltration while minimizing Treg proliferation that may negatively affect the anti-tumor response.

The proteins employed displayed highly favorable in vivo efficacy despite being designed to bind their targets on tumor cells only in monovalent manner. By avoiding the increase in affinity caused by multivalent binding to a target antigen on tumor cells, the monovalent binding to the tumor antigen, NKp46 (and CD16A) and cytokine receptor permitted the multispecific protein to be readily tuned. It was observed that each ABD (i.e. the tumor antigen ABD, CD16A ABD, NKp46 ABD and cytokine receptor ABD) provided a distinct contribution to the potentiation of NK cell-mediated anti-tumor activity in vivo. The exemplary proteins with monovalent tumor antigen binding incorporated a cytokine with an affinity that confers binding to its receptor on NK cells that is not higher than the affinity conferred by the NKp46 ABD for NKp46. Anti-tumor cell activity was higher than comparable conventional antibodies even though conventional antibodies bind bivalently and therefore typically have considerably higher binding affinity for their target.

In vivo, the multispecific protein dramatically increased NK cell infiltration in tumors; the multispecific protein caused a 9-fold increase in NKp46-expressing NK cells in tumors, compared to 1.3-fold increase by gold standard ADCC-inducing antibody obinutuzumab that shared the same anti-tumor VH/VL pair, and compared to a 1.6-fold increase by the same multispecific protein lacking the CD122 ABD.

The examples made use of a variant IL-2 cytokine (IL-2v) which was modified to reduce affinity for its receptor(s) on T cells (CD25) but retained substantially full affinity (comparable to wild-type IL-2) for its receptor on NK cells (CD122 and/or CD132). The NKp46 binding domain (exemplified as a VH/VL pair comprised in a Fab or scFv), the CD16-binding Fc domain and the cytokine were placed adjacent to one another in series within the protein, each separated from the adjacent element (i.e. NKp46 ABD, Fc domain or cytokine) solely by a short flexible peptide linker. These configurations of multispecific proteins were designed to present the respective antigen binding domains so as to permit co-engagement of NKp46 and cytokine receptor on the same cell surface plane (i.e. in NKp46, cytokine receptor (and further CD16A) are bound in cis). Further, the examples used a Fc domain that binds CD16A, showing that binding to CD16A does not negatively affect the tumor and NK-targeted biodistribution and instead led to triple co-engagement of NKp46, CD16A and cytokine receptor, and in turn permitted the incorporation of a cytokine that retained binding affinity for its receptor on NK cells. By incorporating into the multispecific protein anti-NKp46 VH/VL domains that conferred a binding affinity for NKp46 in the low nanomolar range for the KD (KD of about 15 nM), cytokines could be used that retained good affinity, optionally substantially full binding affinity, for their receptor on NK cells sufficient to mediate potent signaling in NK cells. Typically, cytokines such as the ones described herein generally have an affinity for binding to their receptor on NK cells that is no stronger than that of the affinity of the multispecific protein for NKp46 (affinity can be determined as the KD).

It is believed, in view of these results, that targeting the cytokine, e.g. a type 1 cytokine such as an IL-2, IL-15, IL-21, IL-7, IL-27 or IL-12 cytokine, an IL-18 cytokine or a type 1 interferon (e.g. IFN-α, IFN-β), to an NKp46-bearing NK cell surface promotes cis-presentation to the cytokine's receptor (e.g. IL2/15βγ, IL-21R, IL-7Ra, IL-27Ra, IL-12R, IL-18R, IFNAR), as shown in FIG. 1 for the cytokine IL2 and cytokine receptor complex IL2βγ). As shown herein, IL2v placed immediately adjacent to (and on the C-terminal side of) either a CD16A-binding ABD or NKp46-binding ABD permitted the triple receptor cis-presentation to occur (the IL2v was connected to the adjacent domain by a linker peptide of as little as five amino acid residues). Advantageously, use of a dimeric Fc domain nas the CD16A-binding ABD provides FcRn, in turn conferring a half life sufficiently long to induce tumor infiltration and proliferation of NK cells in vivo.

The multispecific proteins directed to NKp46 on NK cells have the advantage that they permit a range of cytokines to be used and/or tested without a requirement for reduced binding affinity for their receptor on NK cells (e.g. CD122). The cytokine therefore may or may not be modified to attenuate or decrease binding affinity for its receptor. The multispecific proteins directed to NKp46 on NK cells can thus make use of any one of several cytokines in their wild-type form, particularly where the cytokine does not have substantially reduced activity at its receptor on NK cells, and/or where the cytokine's affinity for its receptor is no stronger than the affinity of the NKp46 ABD for NKp46. Accordingly, in any embodiment, the cytokine ABD (e.g. cytokine moiety within the multispecific protein) can be specified as having a binding affinity and/or an activity (e.g. induction of signalling) on its receptor on NK cells that is not substantially reduced compared to the wild-type form of the cytokine. Optionally, the cytokine moiety induces signalling at its receptor on NK cells (e.g. CD122) that is at least 70% or 80% of that observed with the wild-type form of the cytokine. Accordingly, in any embodiment, the cytokine ABD (e.g. cytokine moiety within the multispecific protein) can be specified as having an affinity for its receptor on NK cells that is not substantially reduced compared to the wild-type form of the cytokine. In some embodiments, the cytokine moiety has a binding affinity for its receptor on NK cells (e.g. CD122) that is within 3-log, 2-log or 1-log of that of the wild-type form of the cytokine (e.g. the cytokine moiety has a KD for binding to the cytokine receptor that is not more than 3-, 2- or 1-log higher than that observed for the wild-type form of the cytokine). Affinity can be KD for binding to recombinant receptor protein, as determined using SPR. Signaling or receptor binding affinity of cytokines can be specified as being when incorporated into an otherwise equivalent multispecific protein.

The high efficacy combined with low immune toxicity is therefore a particular advantage of a therapeutic molecule that combines the ability to bind each of NKp46 and cytokine receptor (e.g. CD122), and further CD16A, on an individual NK cell, particularly for a therapeutic agent having a long in vivo half-life. In particular, the incorporation of an Fc domain that binds FcRn permitted a half-life sufficiently long to permit NK cells to proliferate and accumulate at the site of the tumor, in vivo.

The multispecific proteins are particularly advantageous due to high potency in enhancing NK cell activity (e.g. NK cell proliferation, activation, cytotoxicity and/or cytokine release, including by tumor-infiltrating NK cells), yet with low immune toxicity, as evidenced by low systemic increase or release of cytokines IL-6 and TNF-α. The present disclosure provides examples using protein formats that permit sufficient distance between NKp46 and cytokine receptor (e.g. CD122) and CD16A binding domains to permit all three receptors to be bound by a single NK cell, thereby providing combined NK cell receptor activation. Importantly, the combined binding on a single cell may account for the minimal off-target immune toxicity and lack of fratricidal killing of NKp46-expressing and/or CD16-expressing cells (e.g., NK cells) because the multispecific protein is bound by at least one activating receptor in addition to cytokine receptor (e.g. CD122) at the surface of the NKp46 and/or CD16+ effector cell.

The multispecific proteins are further advantageous due to their ability to potentiate the activity and/or proliferation of both NKp46⁺CD16⁺ and NKp46⁺CD16A⁻ NK cells. As shown herein, combined dual binding to NKp46 and CD122, in the absence of binding to CD16A, demonstrates strong potentiation of NK cell activity. In healthy individuals, the CD16⁻ population represents 5-15% of the total NK cell population, while in some cancer patients the proportion of CD16⁻ NK cells is greatly increased, making up as much as 50% of the total NK cell population. Further, the tumor micro-environment has been shown to affect the phenotype of CD16A⁺ NK cells by either inducing shedding of CD16A from the surface of the cells or promoting conversion from CD16A⁺ to CD16⁻ NK cells. In addition, due to CD16A polymorphism, some individuals have mutations in CD16A (e.g. at residue 158 of CD16A) that result in reduced ability to mediate ADCC. Overcoming CD16A deficiencies, for example as may occur in the tumor environment, while increasing both the number of and activation of NKp46+ NK cells in the tumor, is particularly advantageous. Yet further, multispecific proteins do not require binding or signaling via NKG2D and can be used to potentiate NK cell activity in patients having NK and/or T cells characterized by relatively low levels of surface expression of the activating receptor NKG2D, for example as is known to be a general or common feature in gastric and prostate cancer.

Provided, inter alia, is a multispecific protein comprising: (a) a NKp46-binding domain that binds to a human NKp46 polypeptide, (b) a binding domain that binds an antigen of interest (e.g. a tumor-associated or cancer antigen; an antigen of interest present expressed by a target cell), (c) an optional CD16A-binding domain (e.g. an Fc dimer) that binds to a human CD16A polypeptide, and (c) an antigen binding domain that binds to a human cytokine receptor polypeptide expressed on NK cells (e.g. a receptor such as CD122 (IL2/15Rβ), IL-21R, IL-7Ra, IL-27Ra, IL-12R, IL-18R, IFNAR (IFNAR1 and/or IFNAR2). Provided also, is a multispecific protein comprising a NKp46-binding domain that binds to a human NKp46 polypeptide, a binding domain that binds an antigen of interest (e.g. a tumor-associated or cancer antigen; an antigen of interest present expressed by a target cell), an Fc domain (e.g. an Fc domain dimer) that is bound by human FcRn (and optionally that is further bound by a human CD16A polypeptide), and an antigen binding domain that binds to a human cytokine receptor polypeptide (e.g. CD122 (IL2/15Rβ), IL-21R, IL-7Ra, IL-27Ra, IL-12R, IL-18R, IFNAR (IFNAR1 and/or IFNAR2). The antigen binding domain that binds a cytokine receptor can be a variant cytokine having a modification that reduces binding to a receptor counterpart found on non-NK cells (e.g. T cells, Treg cells) compared to its wild-type form.

In any embodiment, the ABD that binds to a human NKp46 polypeptide and the ABD that binds a human cytokine receptor can be specified as being configured to be capable of adopting a membrane planar binding confirmation.

In any embodiment, the multispecific protein can be specified as being capable of interacting with, binding to or co-engaging NKp46 and the cytokine receptor, and optionally further CD16A, on the surface of an NK cell.

In any embodiment, optionally the ABD that binds to a human NKp46 polypeptide and the ABD that binds a human cytokine receptor, and optionally further the Fc domain, are specified as being positioned or connected within the multispecific protein in series (e.g. with respect to the N- and C-termini of the multispecific protein).

The ABD that binds NKp46 (e.g. a Fab, single variable domain or scFv) can optionally be specified as being connected to the CD16A− binding domain (e.g. an Fc domain) by an Ig-derived (e.g. a peptide from a hinge domain or heavy or light chain constant domain) or non-Ig-derived domain linker, optionally wherein the domain linker is a flexible polypeptide linker. The ABD that binds a cytokine receptor can optionally be specified as comprising a wild-type or variant cytokine connected to the rest of the multispecific protein or to the NKp46 ABD by a domain linker, optionally a flexible polypeptide linker. The cytokine can be specified as being positioned C-terminal to both the NKp46− and CD16A− binding domains on the multispecific protein, and optionally further the cytokine is connected to the rest of the multispecific protein (or e.g., a domain thereof, the NKp46 ABD) via a peptide linker of 15, 10 or 5 residues or less. The NKp46− and CD16A− binding domains can optionally be specified as being placed adjacent to one another on the multispecific protein and optionally connected to one another by a peptide linker (e.g. an immunoglobulin-derived linker such as a hinge-derived linker, a non-immunoglobulin-derived linker, a flexible linker) having a length of 15, 10 or 5 residues or less.

In one embodiment, the ABD that binds to a human NKp46 polypeptide is positioned adjacent to the Fc domain within the protein (or on a polypeptide chain thereof), and wherein one of the ABD that binds to a human NKp46 polypeptide and the Fc domain are positioned adjacent to the ABD that binds a human cytokine receptor, optionally further wherein the ABD that binds a human cytokine is connected to the ABD that binds to a human NKp46 polypeptide or the Fc domain by a linker peptide having 20 or less than 20 amino acid residues, optionally less than 15 amino acid residues, optionally less than 10 amino acid residues, optionally between 5 and 15 residues, optionally between 5 and 10 residues, optionally between 3 and 5 residues.

In any aspect, the ABD that binds a cytokine receptor can be a human cytokine polypeptide, for example CD122 (IL2/15Rβ), IL-21R, IL-7Ra, IL-27Ra, IL-12R, IL-18R, IFNAR (IFNAR1 and/or IFNAR2). The ABD that binds a cytokine receptor can optionally be a human cytokine polypeptide (e.g. IL-2, IL-15, IL-21) that is modified (e.g. by introducing amino acid modifications) to reduce the binding affinity for a cytokine receptor to which it binds, optionally wherein binding affinity is selectively reduced for a receptor not expressed at the surface of NK cells or a receptor also expressed at the surface of non-NK cells (e.g. T cells, Treg cells). For example, the ABD that binds a human cytokine receptor can be a variant cytokine that displays reduced binding affinity for cytokine receptor present on T cells compared to the non-modified or wild-type cytokine polypeptide.

As further described herein, where a cytokine has more than one receptor as its natural binding partner and one of the receptors is expressed on non-NK cells, the cytokine polypeptide can be modified to reduce binding to such receptor that is expressed on non-NK cells (e.g. Treg cells, T cells) compared to its wild-type cytokine counterpart.

In one embodiment, exemplified by the protein incorporating the CDRs of the NKp46-1 VH/VL pair, the NKp46-binding domain binds to the D1/D2 junction of the NKp46 polypeptide. Based on x-ray crystallo studies of NKp46 complexed with the NKp46-binding domain, it is believed that the junction of the NKp46-binding polypeptide and the IL-2 moiety is positioned at about 70 Angstroms from the cell surface, which corresponds to the predicted distance from the cell surface for the cytokine binding site of CD122. As shown in FIG. 1C, binding to the D1/D2 junction of the NKp46 polypeptide and/or to the region or epitope bound by NKp46-1 can provide a positioning of the NKp46 ABD at a distance from the NK cell surface that permits optimal engagement of a cytokine receptor such as CD122. In turn, domain linkers of reduced length (e.g. between 2 and 5 residues, between 2 and 10 residues; 3, 4, 5, 6, 7, 8, 9 or 10 residues) can be used between the cytokine and the NKp46 ABD or rest of the multispecific protein without any decrease in potency. When other domains on NKp46 are bound, longer domain linkers can be used, e.g. between 5 and 15 residues, between 10 and 15 residues, or more. The proteins described herein with a limited number of very short linkers therefore have the advantage of having minimal non-natural (or non-immunoglobulin derived) amino acid sequences.

In one embodiment, the multispecific protein comprises an NKp46-binding domain or portion thereof fused, optionally via a domain linker, to a cytokine receptor-binding domain, e.g. a cytokine that binds a receptor expressed at the surface of an NK cell.

In one embodiment, the multispecific protein comprises an NKp46-binding domain or portion thereof fused, optionally via a domain linker, to a cytokine receptor-binding domain, e.g. a cytokine that binds a receptor expressed at the surface of an NK cell. In one embodiment, the portion of the NKp46-binding domain comprises a single variable domain (e.g. a first variable domain fused to a first constant domain) that, together with a complementary variable domain (e.g. a second variable domain fused to a second constant region), forms an ABD (e.g., a Fab) that binds NKp46.

In one embodiment, the multispecific protein comprises: (i) a first polypeptide chain comprising, from N- to C-terminal, an NKp46-binding domain or portion thereof comprising a variable domain, a human CH1 or CL constant domain, optionally a domain linker, and a wild-type or variant IL-2, IL-15, IL-21, IL-7, IL-27, IL-12, IL-18, IFN-α or IFN-β polypeptide, and
(ii) a second polypeptide chain comprising, from N- to C-terminal, a variable domain that associates with the variable domain of (i) to form a NKp46-binding domain, and a human CH1 or CL constant domain;
wherein one of the constant domains of (i) and (ii) is a CH1 and the other is a CL such that the constants domains of (i) and (ii) associate by CH1-CL dimerization. In one embodiment, the protein further comprises a dimeric Fc domain and an ABD that binds an antigen of interest.

In one embodiment, provided is a protein comprising a NKp46 ABD-cytokine unit. In one embodiment, the protein is a multispecific protein comprising a V_{H} and a V_{L} that associate to form an ABD that binds a cancer antigen or other antigen of interest, and a NKp46 ABD-cytokine unit (and optionally further a CD16A ABD (e.g. a dimeric Fc domain)). The NKp46 ABD-cytokine unit comprises an NKp46 ABD fused, optionally via a domain linker, to a cytokine that binds a receptor expressed at the surface of an NK cell.

In any embodiment herein, a multispecific protein can thus comprise a NKp46 ABD-cytokine unit that is formed from the association of two polypeptide chains and has one of the following structures:

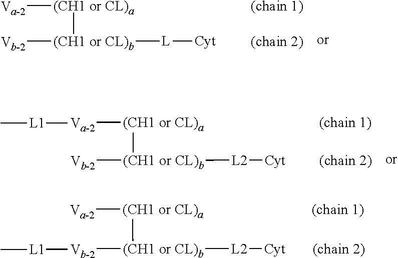

wherein:
Vₐ₋₂ and V_{b-2} are each a V_{H} domain or a V_{L} domain, wherein one of Vₐ₋₂ and V_{b-2} is a V_{H} and the other is a V_{L} and wherein Vₐ₋₂ and V_{b-2} form an ABD that binds NKp46; CH1 is a human immunoglobulin CH1 domain and CL is a human light chain constant domain;
one of (CH1 or CL)ₐ and (CH1 or CL)_{b} is a CH1 and the other is a CL such that a (CH1/CL) pair is formed;
L, L1 and L2 are each an amino acid domain linker, wherein L, L1 and L2 can be different or the same, wherein L1 is a linker connecting the NKp46 ABD-cytokine unit to the rest of the multispecific protein (e.g., a protein comprising a V_{H} and a V_{L} and that associate to form an ABD that binds a cancer antigen); and Cyt is a cytokine polypeptide or portion thereof that binds to a cytokine receptor present on NK cells, optionally wherein Cyt is a wild-type or variant human IL-2, IL-15, IL-21, IL-7, IL-27, IL-12, IL-18, IFN-α or IFN-β polypeptide. The Cyt can be specified as having a free C-terminus (no further domains or amino acid sequences fused to the Cyt at its free terminus). Optionally, chain 1 can be specified as having a free C-terminus. “-” can be specified to indicate connection via covalent bond (e.g. peptide bond) to other amino acid residues.

In one embodiment, the NKp46 ABD-cytokine unit comprises: (i) a first polypeptide chain comprising, from N- to C-terminal, an NKp46-binding domain or portion thereof comprising a variable domain comprising an amino acid sequence at least 80%, 90%, 95%, 98% or 99% identical to an amino acid sequence of any of SEQ ID NOS: 3, 5, 7, 9, 11, 13, 112, 113, 115, 116, 117, 119, 120, 121, 123, 124, 125, 127, 128, 129 or 236-313, a human CH1 or CL constant domain, optionally a domain linker, and a wild-type or variant IL-2, IL-15, IL-21, IL-7, IL-27, IL-12, IL-18, IFN-α or IFN-β polypeptide comprising an amino acid sequence at least 80%, 90%, 95%, 98% or 99% identical to an amino acid sequence of any of SEQ ID NOS: 404-439 or to a fragment thereof of at least 40, 50, 60, 80 or 100 contiguous amino acids thereof; and
(ii) a second polypeptide chain comprising, from N- to C-terminal, a variable domain that associates with the variable domain of (i) to form a NKp46-binding domain wherein the variable domain comprises an amino acid sequence at least 80%, 90%, 95%, 98% or 99% identical to an amino acid sequence of any of SEQ ID NOS: 4, 6, 8, 10, 12, 14, 114, 118, 122, 126, 130 or 314-403, and a human CH1 or CL constant domain.

In one embodiment, the NKp46 ABD-cytokine unit comprises: (i) a first polypeptide chain comprising, from N- to C-terminal, an NKp46-binding domain or portion thereof comprising a variable domain comprising an amino acid sequence at least 80%, 90%, 95%, 98% or 99% identical to an amino acid sequence of any of SEQ ID NOS: 4, 6, 8, 10, 12, 14, 114, 118, 122, 126, 130 or 314-403, a human CH1 or CL constant domain, optionally a domain linker, and a wild-type or variant IL-2, IL-15, IL-21, IL-7, IL-27, IL-12, IL-18, IFN-α or IFN-β polypeptide comprising an amino acid sequence at least 80%, 90%, 95%, 98% or 99% identical to an amino acid sequence of any of SEQ ID NOS: 404-439 or to a fragment thereof of at least 40, 50, 60, 80 or 100 contiguous amino acids thereof; and
(ii) a second polypeptide chain comprising, from N- to C-terminal, a variable domain that associates with the variable domain of (i) to form a NKp46-binding domain wherein the variable domain comprises an amino acid sequence at least 80%, 90%, 95%, 98% or 99% identical to an amino acid sequence of any of SEQ ID NOS: 3, 5, 7, 9, 11, 13, 112, 113, 115, 116, 117, 119, 120, 121, 123, 124, 125, 127, 128, 129 or 236-313, and a human CH1 or CL constant domain.

In another embodiment where the NKp46 ABD-cytokine unit is placed on one polypeptide chain of a protein, the NKp46 ABD-cytokine unit can have the structure:

L1-Vₐ₋₂-L2-V_{b-2}-L3-Cyt
wherein:
Vₐ₋₂ and V_{b-2} are each a V_{H} domain or a V_{L} domain, wherein one of Vₐ₋₂ and V_{b-2} is a V_{H} and the other is a V_{L} and wherein Vₐ₋₂ and V_{b-2} form an ABD that binds NKp46;
L1, L2 and L3 are each an amino acid domain linker, wherein L1, L2 and L3 can be different or the same, wherein L1 is a linker connecting the NKp46 ABD-cytokine unit to the rest of the multispecific protein (e.g., a protein comprising a V_{H} and a V_{L} and that associate to form an ABD that binds a cancer antigen); and Cyt is a cytokine polypeptide or portion thereof that binds to a cytokine receptor present on NK cells, optionally wherein Cyt is a wild-type or variant human IL-2, IL-15, IL-21, IL-7, IL-27, IL-12, IL-18, IFN-α or IFN-β polypeptide. L1, L2 and L3 can each be independently specified as having a length of 15, 10 or 5 residues or less.

In one aspect of any embodiment herein, the cytokine or cytokine receptor ABD (either as a free cytokine or as incorporated into a multispecific protein) binds its receptor, as determined by SPR, with a binding affinity (KD) of 1 μM or lower, 200 nM or lower, 100 nM or lower, 50 nM or lower, or 25 nM or lower. In one embodiment, the cytokine or cytokine receptor ABD binds its receptor, as determined by SPR, with a binding affinity (KD) that is 1 nM or higher than 1 nM, optionally that is higher than 10 nM optionally that is higher than 15 nM. In one embodiment, the cytokine or cytokine receptor ABD binds its receptor, as determined by SPR, with a binding affinity (KD) between about 1 nm and about 200 nm, optionally between about 1 nm and about 100 nm, optionally between about 10 nM and about 1 μM, optionally between about 10 nM and about 200 μM, optionally between about 10 nM and about 100 nM, optionally between about 15 nM and about 1 μM, or optionally between about 15 nM and about 200 nM.

In one embodiment, the cytokine is a wild-type cytokine or a fragment or variant thereof that has at least 80% of the ability of a wild-type cytokine counterpart to induce signaling in NK cells, optionally wherein signaling is assessed by bringing the isolated cytokine moiety into contact with an NK cell and measuring STAT phosphorylation in the NK cells. In one embodiment, the cytokine is a wild-type cytokine or a fragment thereof that retains at least 70%, 80% or 90% of the affinity for its cytokine receptor present on NK cells, compared to the wild-type cytokine counterpart. In one embodiment, the cytokine is a variant cytokine, wherein the cytokine retains at least 70%, 80% or 90% of the affinity for its cytokine receptor present on NK cells, compared to the wild-type cytokine counterpart. In one embodiment, the cytokine does not comprise mutations that substantially reduce the affinity of the cytokine for the cytokine receptor present on NK cells. In one embodiment, the multispecific protein (or the cytokine when included in the multispecific protein) exhibits an EC₅₀ for cytokine pathway signaling in NK cells that is lower than that observed with its wild-type cytokine counterpart alone. In one embodiment, the multispecific protein (or the cytokine when included in the multispecific protein) exhibits an EC₅₀ for cytokine pathway signaling in NK cells that is lower than that observed with the cytokine alone or in a protein of comparable structure but lacking a NKp46 ABD and/or a CD16 ABD. Optionally the EC₅₀ for cytokine pathway signaling in NK cells is at least 10-fold or 100-fold lower, optionally wherein cytokine pathway signaling is assessed by bringing the respective cytokine or multispecific protein into contact with an NK cell and measuring STAT phosphorylation in the NK cells.

In one embodiment, the multispecific protein is configured such that an Fc domain (or CD16-binding domain), the NKp46-binding domain and the cytokine receptor-binding domain are each capable of binding to their respective NKp46, CD16A or cytokine receptor binding partner when such binding partners are present together at the surface of a cell (e.g. an NK cell). In some embodiments, the multispecific protein can be characterized by monovalent binding to NKp46 (e.g. the multispecific protein comprises only one NKp46 ABD), monovalent (or optionally bivalent) binding to antigen of interest, monovalent binding to CD16A (e.g. the multispecific protein comprises only one Fc domain dimer), and monovalent binding to cytokine receptor (e.g., the multispecific protein comprises only one cytokine receptor ABD).

In one embodiment, the multispecific protein is configured e.g., through placement or configuration of the domain within a multispecific protein, optionally through use of one or more using domain linkers having a maximal potential length of 18 Angstroms (5 amino acid residues), 36 Angstroms (10 residues) or 54 Angstroms (15 residues) when in a stretched configuration such that the NKp46-binding domain and the cytokine receptor-binding domain, and the CD16-binding domain when present and capable of binding CD16, can assume a membrane planar binding conformation such that each of NKp46, CD16A and cytokine receptor are bound at the surface of an NK cell.

The multispecific protein can thus be configured such that the cytokine receptor-binding domain is placed, topologically within the multimeric protein, terminally (e.g. C-terminal) to both the NKp46-binding domain and the CD16A-binding domain within the multispecific protein. For example the cytokine receptor ABD can be placed C-terminally on a polypeptide chain of the multispecific protein such that the positioning allows the cytokine receptor ABD to be topologically C-terminal within the multimeric protein). The NKp46 ABD and the CD16A ABD (e.g. a dimeric Fc domain) can be positioned adjacent to one another topologically in the protein, optionally connected to one another via a short domain linker. The NKp46 ABD, CD16A ABD and cytokine (or parts thereof) can thus be connected or positioned in series in the protein (or on polypeptide chain(s) thereof. Advantageously, the protein comprises a dimeric Fc domain (the Fc domain for example specified as consisting of two Fc monomers placed on separate polypeptide chains). Thus, in a multispecific protein the NKp46 ABD, the dimeric Fc domain and the cytokine are advantageously positioned adjacent to one another topologically (within the topology of the multispecific protein). In one embodiment, an NKp46-binding domain (or a part thereof, e.g. a VH or VL) and an Fc domain monomer (or CD16A-binding domain) are placed adjacent to one another on a same polypeptide chain(s), e.g. the adjacent NKp46-binding domain and the CD16A-binding domain can be separated by a domain linker but without any intervening protein domain (e.g. without an intervening domain that binds an antigen, and the cytokine moiety can be placed C-terminally thereto.

For example, in preferred multispecific proteins having particularly advantageous NK cell potentiation activity, the multispecific protein comprises a Fc domain dimer comprised of a first and second Fc domain monomer positioned on different polypeptide chains (that dimerize via CH3-CH3 association). The first Fc domain monomer can be fused at its C-terminus to an anti-NKp46 ABD (or portion thereof), and the anti-NKp46 ABD (or portion thereof) is in turn fused at its C-terminus to a cytokine. The portion of an anti-NKp46 ABD can be for example a ((VH or VL)-CH1) unit or ((VH or VL)-CL) unit where the ABD is a Fab. FIG. 2A to 2C, 2E, 2G to 2J, 2L, 2M show exemplary domain configurations where the anti-NKp46 ABD and the cytokine are topologically adjacent to one another with respect to N- and C-termini and are fused via one Fc domain monomer. FIGS. 2D, 2F, 2K and 2N show domain configurations where anti-NKp46 ABD and cytokine are each fused to the C-terminus of a different Fc domain monomer.

In any embodiment, the cytokine receptor-binding domain (cytokine receptorABD), the NKp46-binding domain (NKp46 ABD) and the CD16-binding domain (CD16 ABD) can be specified as being placed within the one or more polypeptide chains that make up the multispecific protein so that the domains are oriented in a configuration in which they are adjacent to one another or in series, from N to C terminal, on the multimeric (e.g. heteromultimeric) protein. Domains can be optionally separated by a domain linker e.g. a linking peptide of 5-20 residues that does not itself bind to a predetermined antigen.

In any embodiment, the multispecific protein can be specified as being configured e.g., through placement or configuration of the domain within a multispecific protein, such that the NKp46 ABD and the cytokine receptor ABD (e.g. the cytokine moiety) have the ability to assume a position where they are on the same side or face of the Fc domain dimer within the multispecific protein molecule, so as to enhance the ability to bind NKp46, CD16A and cytokine receptor in a membrane planar binding conformation. This configuration can be readily implemented in any in any of the heterodimeric, heterotrimeric or heterotetrameric proteins of the disclosure, for example by positioning the NKp46 ABD (or a part thereof, if the ABD is formed from association of two polypeptide chains) and the cytokine receptor ABD (or a part thereof, if the ABD is formed from association of two polypeptide chains) on the same polypeptide chain together with one of the Fc domain monomers.

The multispecific protein can have the formula (X₁)-L₁-(X₂)-L₂-(X₃), where one of X, and X₂ is an NKp46 ABD (e.g. a Fab, an scFv, a VHH) or a part thereof (e.g. the part may be an a VH or VL, a VH-CH1, VH-CL, VL-CL, VL-CH1) and the other is a Fc dimer or part thereof (e.g. an Fc monomer), and X₃ is a cytokine, wherein L₁ and L₂ are each an optional domain linker. The cytokine is positioned at the C-terminus of the polypeptide chain on which it (or a part thereof) is placed. L₁ connects X₁ and X₂ via a covalent bond (e.g. peptide bond). L₂ connects X₂ and X₃ via a covalent bond (e.g. peptide bond). X₁ to X₃ can optionally be specified as being arranged from the topological N- to C-terminus of the protein. The multispecific protein can further comprise an ABD that binds an antigen of interest, e.g. connected at the N-terminus of X, or positioned N-terminally within the topology of the protein.

As demonstrated herein, it will be appreciated that the different elements X₁, X₂ and X₃ (and further the ABD that binds an antigen of interest) can each be readily distributed onto two or more different polypeptide chains within the protein. In one embodiment, the multispecific protein can optionally be characterized as comprising a first polypeptide chain comprising the formula (X₁)-L₁-(X₂)-L₂-(X₃), where one of X₁ and X₂ is an NKp46 ABD or part thereof and the other is a Fc monomer, and X₃ is a cytokine or a part thereof, wherein L, and L₂ are each an optional domain linker. The cytokine can thus be positioned at the C-terminus of the polypeptide chain on which it (or a part thereof) is placed. The multispecific protein can further comprise an ABD that binds an antigen of interest, wherein the ABD (or a part thereof) is placed on the first polypeptide chain or on a separate polypeptide chain that associates (e.g. dimerizes) with first polypeptide chain (or with any other chain of the protein). The protein can then comprise one, two or more additional polypeptide chains that provide the complementary domains for the NKp46 ABD (when the NKp46 ABD is a part of an ABD,) the Fc monomer (so as to form an Fc dimer), the cytokine (where the cytokine is a part of a cytokine) and/or the ABD that binds an antigen of interest. Such additional polypeptide chains can thus associate (e.g. dimerize) with first or other polypeptide chain of the protein via non-covalent interactions and optionally further covalent interactions.

In another embodiment, the multispecific protein can optionally be characterized as comprising (i) a first polypeptide chain comprising the formula (X₁)-L₁-(X₂a), where X₁ is a first Fc monomer and X₂a is a first part of a NKp46 ABD, and (ii) a second polypeptide chain comprising the formula (X_{2b})-L₂-(X₃), wherein X_{2b} is a second part of a NKp46 ABD (e.g. a VH or VL, a VH-CH1, VH-CL, VL-CL, VL-CH1) that associates with X₂a to form an NKp46 ABD, and X₃ is a cytokine or a part thereof. L₁ and L₂ are each a domain linker. The multispecific protein can further comprise an ABD that binds an antigen of interest, wherein the ABD (or a part thereof) is placed on the first polypeptide chain (e.g. N-terminal to X₁) or on a separate polypeptide chain that associates (e.g. dimerizes) with first polypeptide chain (or with any other chain of the protein). The multispecific protein can further comprise an ABD that binds CD16A, optionally the ABD is a dimeric Fc domain; the ABD that binds CD16A (or a part thereof) ca be placed on the first polypeptide chain (e.g. N-terminal to X₁), and when the ABD is a dimeric Fc domain one of the Fc monomers can be placed on the first polypeptide chain and the second Fc monomer can be placed on a separate polypeptide chain that associates (e.g. dimerizes) with first polypeptide chain.

In one embodiment, the NKp46-binding domain binds NKp46 such that the NKp46 binding domain of the multispecific protein, when bound to NKp46 at the surface of a cell, is about 70 Angstroms from the cell membrane. In one such embodiment, exemplified by the protein incorporating the CDRs of the NKp46-1 VH/VL pair, the NKp46-binding domain binds to the D1/D2 junction of the NKp46 polypeptide. Optionality, the NKp46-binding domain exhibits decreased binding to the NKp46 mutant 2 (having a mutation at residues K41, E42 and E119) and mutant Supp7 (having a mutation at residues Y121 and Y194) compared to the wild-type NKp46 polypeptide. In one embodiment, an NKp46 antigen binding domain can be characterized as displaying decreased binding to a NKp46 mutant polypeptide having one, two, three, four or five of the mutations: K41, E42, E119, Y121 and Y194 compared to a wild-type NKp46 polypeptide.

In one embodiment, the NKp46-binding domain binds NKp46 such that the NKp46 binding domain of the multispecific protein, when bound to NKp46 at the surface of a cell, is less than about 70 Angstroms, optionally less than about 50 Angstroms, from the cell membrane. In one such embodiment, exemplified by the protein incorporating the CDRs of the NKp46-4 VH/VL pair, the NKp46-binding domain binds to the D1 domain of the NKp46 polypeptide which is positioned more proximal to the NK cell membrane compared to the D1/D2 junction. Optionality, the NKp46-binding domain exhibits decreased binding to the NKp46 mutant 6 (having a mutation at residues R101 and V102) and mutant Supp6 (having a mutation at residues E104 and L₁₀₅) compared to the wild-type NKp46 polypeptide. In one embodiment, an NKp46 antigen binding domain can be characterized as displaying decreased binding to a NKp46 mutant polypeptide having one, two, three or four of the mutations: R101, V102, E104 and L₁₀₅ compared to a wild-type NKp46 polypeptide. In one embodiment, the multispecific protein comprises a domain linker of at least 10 amino acid residues between an NKp46 binding domain that binds within the D1 domain and the cytokine.

In another embodiment, exemplified by the protein incorporating the CDRs of the NKp46-3 VH/VL pair, the NKp46-binding domain binds to the D2 domain of the NKp46 polypeptide which is positioned more proximal to the NK cell membrane compared to the D1/D2 junction. Optionality, the NKp46-binding domain exhibits decreased binding to the NKp46 mutant 19 (having a mutation at residues 1135, and S136) and mutant Supp8 (having a mutation at residues P132 and E133) compared to the wild-type NKp46 polypeptide. In one embodiment, an NKp46 antigen binding domain can be characterized as displaying decreased binding to a NKp46 mutant polypeptide having one, two, three or four of the mutations: 1135, S136, P132 and E133 compared to a wild-type NKp46 polypeptide. In one embodiment, the multispecific protein comprises a domain linker of at least 10 amino acid residues between an NKp46 binding domain that binds within the D2 domain and the cytokine.

In any embodiment herein, the multispecific protein can be characterized by having only one cytokine receptor binding domain.

In any embodiment herein, the multispecific protein can be characterized by having only one NKp46 binding domain.

Certain exemplary heteromultimeric proteins can comprise the following general domain organization of structure 1a or 1b, where the CD16 ADC (e.g. Fc domain) and NKp46 ABD are immediately adjacent to other another within the protein, and NKp46 ABD is immediately adjacent to the cytokine receptor ABD (embodied as a cytokine (Cyt)), and wherein the NKp46 ABD is interposed between the CD16 ABD and the Cyt:

(CD16ABD)(NKp46ABD)(Cyt) (Structure 1a)

or

(Fc domain dimer)(NKp46ABD)(Cyt) (Structure 1b)
wherein the NKp46 ABD and the CD16ABD (e.g. Fc domain dimer) are connected by a domain linker and the NKp46 ABD and Cyt are connected by a domain linker.

The NKp46 ABD can conveniently be a Fab, a single domain antibody or an scFv. The CD16 ABD can be an Fc domain, a Fc domain dimer, an Fc domain of human IgG1 subtype. The Cyt can be for example an IL-2, IL-15, IL-21, IL-7, IL-27, IL-12, IL-18, IFN-α or IFN-β) polypeptide, optionally wherein the polypeptide is a variant cytokine that differs by at least one residue from the wild-type human cytokine counterpart.

The protein of structure 1a can comprise an ABD that binds an antigen of interest on a target cell (Antigen ABD) placed terminal to (e.g. N-terminal to) the CD16 ABD (e.g. Fc domain dimer), as in a heteromultimeric protein having the structure 1c or 1d below:

(Antigen ABD)ₙ(CD16ABD)(NKp46ABD)(Cyt) (Structure 1c)

or

(Antigen ABD)ₙ(Fc domain dimer)(NKp46ABD)(Cyt) (Structure 1d)
wherein “n” is 1 or 2, and the Antigen ABD and the CD16 ABD (e.g. Fc domain dimer) are connected by a linker, optionally wherein the linker is an immunoglobulin hinge polypeptide, wherein the CD16 ABD and the NKp46 ABD are connected by a linker and the NKp46 ABD and Cyt are connected by a linker. Where “n” is 2, Structure 1d can also be represented as Structure 1e:

(Antigen ABD)

(Fc domain dimer)(NKp46ABD)(Cyt) (Structure 1e)

(Antigen ABD)

In any embodiment, the multispecific protein can be characterized as binding monovalently to each of the NKp46 polypeptide and the cytokine receptor, and being capable of directing an NKp46-expressing NK cell to lyse a target cell expressing the antigen of interest. Advantageously, in on embodiment, the presence of NK cells and target cells, the multi-specific protein can bind (i) to antigen of interest on target cells, (ii) to NKp46 on NK cells, (iii) to CD16A on NK cells and (iv) to the cytokine receptor on NK cells (e.g. CD122, IL-21R, IL-7Rα, IL-27Rα, IL-12R, IL-18R, IFNAR), and when bound to such proteins on the target cell and NK cells, can induce signaling in and/or activation of the NK cells through NKp46 (the protein acts as an NKp46 agonist) and the cytokine receptor (the protein acts as a cytokine receptor agonist), thereby promoting activation of NK cells and/or lysis of target cells, notably via the activating signal transmitted by NKp46.

In one embodiment, in the presence of NK cells and target cells, the multi-specific protein can induce the cytotoxicity of, cytokine receptor pathway signaling in (as assessed by STAT signaling) and/or activation of the NK cells, wherein such cytotoxicity, activation and/or signaling is greater (e.g. at least 100-fold or 1000-fold lower EC₅₀ value) than that observed when the multi-specific protein is contacted with NK cells in the absence of target cells.

Optionally, the multi-specific protein can bind NKp46 and CD122 on NK cells (e.g. the protein comprises an IL2 or IL15 moiety, optionally an modified or variant IL2 or IL15 with decreased binding to CD25), and, when bound to both NKp46 and CD122, can induce signaling in the NK cells through both NKp46 and CD122. Optionally, the multi-specific protein can bind NKp46, CD16A and CD122 on NK cells, and, when bound to NKp46, CD16 and CD122, can induce signaling in the NK cells through NKp46, CD16A and CD122. Cytokine receptor signaling can be assessed by measuring STAT5, optionally wherein the observed signaling is greater than that observed with a comparator protein in which the NKp46 binding domain is replaced with a control ABD (e.g. that does not bind to any protein present in the assay system).

Optionally, the multi-specific protein can bind NKp46 and IL-21R on NK cells (e.g. the protein comprises an IL21 moiety), and, when bound to both NKp46 and IL-21R, can induce signaling in the NK cells through both NKp46 and IL-21R. Optionally, the multi-specific protein can bind NKp46, CD16A and IL-21R on NK cells, and, when bound to NKp46, CD16A and IL-21R, can induce signaling in the NK cells through NKp46, CD16A and IL-21R. Optionally, cytokine signaling is assessed by measuring STAT3, wherein the observed signaling is greater than that observed with a comparator protein in which the NKp46 binding domain is replaced with a control ABD (e.g. that does not bind to any protein present in the assay system).

Optionally, the multi-specific protein can bind NKp46 and IL-18R on NK cells (e.g. the protein comprises an IL18 moiety), and, when bound to both NKp46 and IL-18R (IL-18Rα and/or IL-18RP), can induce signaling in the NK cells through both NKp46 and IL-18R. Optionally, the multi-specific protein can bind NKp46, CD16A and IL-18R on NK cells, and, when bound to NKp46, CD16A and IL-18R, can induce signaling in the NK cells through NKp46, CD16A and IL-18R. Optionally, cytokine signaling signaling is assessed by measuring STAT3, wherein the observed signaling is greater than that observed with a comparator protein in which the NKp46 binding domain is replaced with a control ABD (e.g. that does not bind to any protein present in the assay system).

Optionally, the multi-specific protein can bind NKp46 and IL-7R (e.g. IL-7Rα (CD127) and/or CD132) on NK cells (e.g. the protein comprises an IL-7 moiety), and, when bound to both NKp46 and IL-7R, can induce signaling in the NK cells through both NKp46 and IL-7Rα. Optionally, the multi-specific protein can bind NKp46, CD16A and IL-7R on NK cells, and, when bound to NKp46, CD16A and IL-7R, can induce signaling in the NK cells through NKp46, CD16A and IL-7R. Optionally, cytokine signaling signaling is assessed by measuring STAT5, wherein the observed signaling is greater than that observed with a comparator protein in which the NKp46 binding domain is replaced with a control ABD (e.g. that does not bind to any protein present in the assay system).

Optionally, the multi-specific protein can bind NKp46 and IL-27R (e.g. IL-27Rα and/or GP130) on NK cells (e.g. the protein comprises an IL-27 moiety), and, when bound to both NKp46 and IL-27R, can induce signaling in the NK cells through both NKp46 and IL-27R. Optionally, the multi-specific protein can bind NKp46, CD16A and IL-27R on NK cells, and, when bound to NKp46, CD16A and IL-27R, can induce signaling in the NK cells through NKp46, CD16A and IL-27R. Signalling via NKp46 and/or CD16A can be assessed by a marker of NK cell activation (e.g. a marker used in the Examples, CD69 expression, etc.). Optionally, cytokine signaling signaling is assessed by measuring STAT1, wherein the observed signaling is greater than that observed with a comparator protein in which the NKp46 binding domain is replaced with a control ABD (e.g. that does not bind to any protein present in the assay system).

Optionally, the multi-specific protein can bind NKp46 and IL-12R (e.g., IL-12Rβ1 and/or IL-12Rβ2) on NK cells (e.g. the protein comprises an IL-27 moiety), and, when bound to both NKp46 and IL-12R, can induce signaling in the NK cells through both NKp46 and IL-12R. Optionally, the multi-specific protein can bind NKp46, CD16A and IL-12R on NK cells, and, when bound to NKp46, CD16A and IL-12R, can induce signaling in the NK cells through NKp46, CD16A and IL-12R. Optionally, cytokine signaling signaling is assessed by measuring STAT4, wherein the observed signaling is greater than that observed with a comparator protein in which the NKp46 binding domain is replaced with a control ABD (e.g. that does not bind to any protein present in the assay system).

Optionally, the multi-specific protein can bind NKp46 and IFNAR on NK cells, and, when bound to both NKp46 and IFNAR (IFNAR1 and/or IFNAR2), can induce signaling in the NK cells through both NKp46 and IFNAR. For example, the multi-specific protein can comprise an IFN-α or IFN-β moiety) Optionally, the multi-specific protein can bind NKp46, CD16A and IFNAR on NK cells, and, when bound to both NKp46, CD16A and IFNAR, can induce signaling in the NK cells through NKp46, CD16A and IFNAR. Optionally, cytokine signaling signaling is assessed by measuring STAT (e.g., STAT1, STAT2 or IFN regulatory factor (IRF)-9), wherein the observed signaling is greater than that observed with a comparator protein in which the NKp46 binding domain is replaced with a control ABD (e.g. that does not bind to any protein present in the assay system).

Signalling via NKp46 and/or CD16A can be assessed by a marker of NK cell activation (e.g. a marker used in the Examples, CD69 expression, etc.).

In some embodiment, the multispecific protein comprises at least a portion of a human Fc domain, e.g. a portion sufficient such that the Fc domain is bound by a human FcRn polypeptide, optionally wherein said FcRn binding affinity as assessed by SPR is within 1-log of that of a conventional human IgG1 antibody.

The multispecific proteins advantageously are able to potently mobilize both CD16+ and CD16− NK cells (all NK cells are NKp46+).

In one aspect, the multispecific protein comprises two or more polypeptide chains, i.e. it comprises a multi-chain protein (also referred as a multimeric protein). For example, the multispecific protein or multi-chain protein can be a hetero-dimer, hetero-trimer or hetero-tetramer or may comprise more than four polypeptide chains.

Any antigen binding domain (e.g. the ABD that binds the antigen of interest (e.g. tumor antigen), NKp46, or cytokine receptor) can be contained entirely on a single polypeptide chain, for example as an scFv or single antigen binding domain such as a sdAb or nanobody, a V_{NAR} or VHH domain or a DARPin® protein module). Alternatively, an antigen binding domain can be made of two or more protein domains placed on separate polypeptide chains, such that the antigen binding domain binds its target when two or more complementary protein domains (e.g. as VH/VL pairs) are associated in the multimeric protein.

The multispecific protein can bind to the antigen of interest (e.g. cancer antigen) in monovalent or multivalent manner. Where the multispecific protein binds the antigen of interest monovalently, binds NK46 monovalently and binds the cytokine receptor monovalently, the multispecific protein can be indicated as having a 1:1:1 configuration. Where the multispecific protein binds the antigen of interest bivalently, binds NK46 monovalently and binds the cytokine receptor monovalently, the multispecific protein can be indicated as having a 2:1:1 configuration. Representative examples of different 1:1:1 and 2:1:1 configurations are shown in FIG. 2.

In any aspect, the multispecific protein can be characterized as having a structure in which the freedom of motion (intrachain domain motion) or flexibility of one or more antigen binding domains (ABDs) is increased, e.g. compared to the ABDs of a conventional human IgG antibody. In one embodiment, provided is a multispecific protein comprising a structure that permits the antigen binding site of the first antigen binding domain and the antigen binding site of the second antigen binding domain to be separated by a distance that results in enhanced function, e.g., the ability of the multispecific protein to induce NKp46 signaling and lysis of target cells, e.g., optionally a distance of less than 80 ångström (Å). Multispecific proteins wherein the ABDs possess greater flexibility and/or are separated by an optimized distance may enhance the formation of a lytic NKp46-target synapse, thereby potentiating NKp46-mediated signaling. Such flexibility and/or domain of motion can be readily achieved through the use of linkers (e.g. flexible amino acid based linkers) that separate the NKp46 binding domain from the Fc domain (e.g. the Fc domain dimer, or more generally the rest of the multispecific protein).

In any aspect, the multispecific protein can be characterized as having increased freedom of motion of the antigen binding domains (e.g. compared to the ABDs of a conventional human IgG antibody, e.g., a human IgG1 antibody). One example of such a protein is a multimeric Fc domain-containing protein (e.g. a heterodimer or heterotrimer) in which an antigen binding domain (e.g., the ABD that binds NKp46) is linked or fused to an Fc domain via a flexible linker. The linker can provide flexibility or freedom of motion of one or more ABDs by conferring the ability to bend thereby potentially decreasing the angle between the ABD and the Fc domain (or between the two ABDs) at the linker. Optionally, both antigen binding domains (and optionally more if additional ABDs are present in the multispecific protein) are linked or fused to the Fc domain via a linker, typically a flexible peptide linker. Optionally, the protein with increased freedom of motion permits the protein to adopt a conformation in which the distance between the NKp46 binding site and the antigen of interest binding site is less that than observed in proteins in which both binding domains were Fabs, or less than in full length antibodies.

An ABD can be connected to an Fc domain monomer (or CH2 or CH3 domain thereof) via a domain linker. The linker can be a polypeptide linker, for example peptide linkers comprising a length of at least 5 residues, at least 10 residues, at least 15 residues, at least 20 residues, or more. In other embodiments, the linkers comprises a length of between 2-4 residues, between 2-5 residues, between 2-6 residues, between 2-8 residues, between 5-10 residues, between 2-15 residues, between 4-15 residues, between 3-15 residues, between 5-15 residues, between 10-15 residues, between 4-20 residues, between 5-20 residues, between 2-20 residues, between 10-30 residues, or between 10-50 residues. Optionally a linker comprises an amino acid sequence derived from an antibody constant region, e.g., an amino acid sequence from a CH1 or CK domain (e.g an an N-terminal sequence from a CH1 or CK domain) or from a hinge. Optionally a linker comprises the amino acid sequence RTVA. Optionally a linker is a flexible linker predominantly or exclusively comprised of glycine and/or serine residues, e.g., comprising an amino acid sequence (G×S)ₙ where G is 1, 2, 3 or 4 and n is an integer from 1-10, from 1-6 or from 1-4. Optionally the linker comprises 1-20 or 1-10 further amino acid residues.

In one embodiment, provided is a heterotrimer having a polypeptide chain 1, 2 and 3:

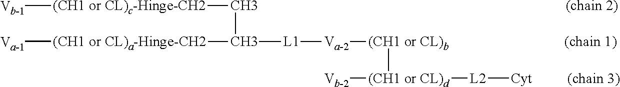

wherein:
Vₐ₋₁, V_{b-1}, Vₐ₋₂ and V_{b-2} are each a V_{H} domain or a VL domain, wherein Vₐ₋₁ and V_{b-1} bind an antigen of interest (optionally wherein one of Vₐ₋₁ and V_{b-1} is a V_{H} and the other is a V_{L} and wherein Vₐ₋₁ and V_{b-1} form a first antigen binding domain (ABD) that binds an antigen of interest), wherein one of Vₐ₋₂ and V_{b-2} is a V_{H} and the other is a V_{L} and wherein Vₐ₋₂ and V_{b-2} form a second ABD that binds NKp46;
CH1 is a human immunoglobulin CH1 domain and CL is a human light chain constant domain;
one of (CH1 or CL)ₐ and (CH1 or CL)_{c} is a CH1 and the other is a CL such that a (CH1/CL) pair is formed;
one of (CH1 or CL)_{b} and (CH1 or CL)_{d} is a CH1 and the other is a CL such that a (CH1/CL) pair is formed;
Hinge is an immunoglobulin hinge region or portion thereof;
L₁ and L₂ are each an amino acid domain linker, wherein L₁ and L₂ can be different or the same;
CH2 and CH3 are human immunoglobulin CH2 and CH3 domains, respectively; and
Cyt is a cytokine polypeptide or portion thereof that binds to a cytokine receptor present on NK cells, optionally wherein Cyt is a wild-type or variant human IL-2, IL-15, IL-21, IL-7, IL-27, IL-12, IL-18, IFN-α or IFN-β polypeptide. In another embodiment that can be made using the same domains and domain linkers, provided is a heterotrimer having a polypeptide chain 1, 2 and 3:

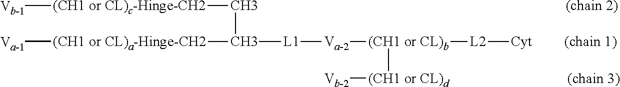

In one embodiment, provided is a heterodimer having a polypeptide chain 1 and 2:

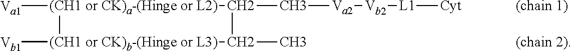

wherein:
Vₐ₋₁, V_{b-1}, Vₐ₋₂ and V_{b-2} are each a V_{H} domain or a V_{L} domain, wherein Vₐ₋₁ and V_{b-1} bind an antigen of interest (optionally wherein one of Vₐ₋₁ and V_{b-1} is a V_{H} and the other is a V_{L} and wherein Vₐ₋₁ and V_{b-1} form a first antigen binding domain (ABD) that binds an antigen of interest), wherein one of Vₐ₋₂ and V_{b-2} is a V_{H} and the other is a V_{L} and wherein Vₐ₋₂ and V_{b-2} form a second ABD that binds NKp46;
CH1 is a human immunoglobulin CH1 domain and CL is a light chain constant domain; one of (CH1 or CL)ₐ and (CH1 or CL)_{b} is a CH1 and the other is a CL such that a (CH1/CL) pair is formed;
Hinge is an immunoglobulin hinge region or portion thereof;
L₁, L₂ and L₃ are each an amino acid domain linker, wherein L₁, L₂ and L₃ can be different or the same;
CH2 and CH3 are human immunoglobulin CH2 and CH3 domains, respectively; and
Cyt is a cytokine polypeptide or portion thereof that binds to a cytokine receptor present on NK cells, optionally wherein Cyt is a wild-type or variant human IL-2, IL-15, IL-21, IL-7, IL-27, IL-12, IL-18, IFN-α or IFN-β polypeptide.

The disclosure further provides further heterodimer, heterotrimer and heterotetramer multispecific molecules and domain arrangements as further described herein. In one aspect, a multispecific protein is a heteromultimer, heterodimer, heterotrimer, heterotetramer having a structure or domain arrangement as shown in any of FIGS. 2A to 2N.

In one aspect of any of the embodiments described herein, an ABD (e.g., the anti-NKp46 ABD, the ABD that binds the antigen of interest or tumor antigen) can be specified as comprising an immunoglobulin heavy chain variable domain (VH) and an immunoglobulin light chain variable domain (VL), wherein each VH and VL comprises three complementary determining regions (CDR-1 to CDR-3). In one aspect of any of the embodiments described herein, a VH can be specified as having the amino acid sequence of a human VH domain. In one aspect of any of the embodiments described herein, a VL can be specified as having the amino acid sequence of a human VL domain.

In one aspect of any of the embodiments, a VH region comprises an amino acid sequence having at least about 80%, 85%, 90%, 95%, 97%, 98% or 99% identity, to the amino acid sequence encoded by a gene of a human V gene group selected from the group consisting of IGHV1-18, IGHV1-2, IGHV1-24, IGHV1-3, IGHV1-45, IGHV1-46, IGHV1-58, IGHV1-69, IGHV1-69-2, IGHV1-69D, IGHV1-8, IGHV2-26, IGHV2-5, IGHV2-70, IGHV2-70D, IGHV3-11, IGHV3-13, IGHV3-15, IGHV3-20, IGHV3-21, IGHV3-23, IGHV3-23D, IGHV3-30, IGHV3-30-3, IGHV3-30-5, IGHV3-33, IGHV3-43, IGHV3-43D, IGHV3-48, IGHV3-49, IGHV3-53, IGHV3-62, IGHV3-64, IGHV3-64D, IGHV3-66, IGHV3-7, IGHV3-72, IGHV3-73, IGHV3-74, IGHV3-9, IGHV3-NL1, IGHV4-28, IGHV4-30-2, IGHV4-30-4, IGHV4-31, IGHV4-34, IGHV4-38-2, IGHV4-39, IGHV4-4, IGHV4-59, IGHV4-61, IGHV5-10-1, IGHV5-51, IGHV6-1, IGHV7-4-1, IGHV1-38-4, IGHV1/OR15-1, IGHV1/OR15-5, IGHV1/OR15-9, IGHV1/OR21-1, IGHV2-70, IGHV2/OR16-5, IGHV3-16, IGHV3-20, IGHV3-25, IGHV3-35, IGHV3-38, IGHV3-38-3, IGHV3/OR15-7, IGHV3/OR16-10, IGHV3/OR16-12, IGHV3/OR16-13, IGHV3/OR16-17, IGHV3/OR16-20, IGHV3/OR16-6, IGHV3/OR16-8, IGHV3/OR16-9, IGHV4-61, IGHV4/OR15-8, IGHV7-81, and IGHV8-51-1. Optionally, a VH region comprises a VH comprising an amino acid sequence (e.g. CDR(s) and/or a human framework region(s), for example according to Kabat numbering) from said gene. In one aspect of any of the embodiments, a VH region comprises an amino acid sequence having at least about 80%, 85%, 90%, 95%, 97%, 98% or 99% identity, to the amino acid sequence of SEQ ID NOS: 236-313.

In one aspect of any of the embodiments, a VL region comprises an amino acid sequence having at least about 80%, 85%, 90%, 95%, 97%, 98% or 99% identity, to the amino acid sequence encoded by a gene of a human V gene group selected from the group consisting of IGKV1-12, IGKV1-13, IGKV1-16, IGKV1-17, IGKV1-27, IGKV1-33, IGKV1-39, IGKV1-5, IGKV1-6, IGKV1-8, IGKV1-9, IGKV1-NL1, IGKV1D-12, IGKV1D-13, IGKV1D-16, IGKV1D-17, IGKV1D-33, IGKV1D-43, IGKV1D-8, IGKV2-24, IGKV2-28, IGKV2-29, IGKV2-30, IGKV2-40, IGKV2D-26, IGKV2D-28, IGKV2D-29, IGKV2D-30, IGKV2D-40, IGKV3-11, IGKV3-15, IGKV3-20, IGKV3D-11, IGKV3D-15, IGKV3D-20, IGKV3D-7, IGKV4-1, IGKV5-2, IGKV6-21, IGKV6D-21, IGKV1-37, IGKV1/OR2-0, IGKV1/OR2-108, IGKV1D-37, IGKV1D-42, IGKV2D-24, IGKV3-7, IGKV3/OR2-268, IGKV3D-20, IGKV6D-41, IGLV1-36, IGLV1-40, IGLV1-44, IGLV1-47, IGLV1-51, IGLV10-54, IGLV2-11, IGLV2-14, IGLV2-18, IGLV2-23, IGLV2-8, IGLV3-1, IGLV3-10, IGLV3-12, IGLV3-16, IGLV3-19, IGLV3-21, IGLV3-22, IGLV3-25, IGLV3-27, IGLV3-9, IGLV4-3, IGLV4-60, IGLV4-69, IGLV5-37, IGLV5-39, IGLV5-45, IGLV5-52, IGLV6-57, IGLV7-43, IGLV7-46, IGLV8-61, IGLV9-49, IGLV1-41, IGLV1-50, IGLV11-55, IGLV2-33, IGLV3-32, IGLV5-48 and IGLV8/OR8-1. Optionally, a VL region comprises a VL comprising an amino acid sequence (e.g. CDR(s) and/or a human framework region(s), for example according to Kabat numbering) from said gene. In one aspect of any of the embodiments, a VL region comprises an amino acid sequence having at least about 80%, 85%, 90%, 95%, 97%, 98% or 99% identity, to the amino acid sequence of SEQ ID NOS: 314-403.

In one aspect of any of the embodiments described herein, an ABD comprises an scFv or Fab, wherein the scFv comprises a VH comprising an amino acid sequence at least 90% identical to a sequence selected from SEQ ID NOS: 3, 5, 7, 9, 11, 13, 112, 113, 115, 116, 117, 119, 120, 121, 123, 124, 125, 127, 128, 129, 132, 134, 136, 138, 140, 142, 144, 146, 148, 150, 152, 154 and any of 236-313, a domain linker, and a VL comprising an amino acid sequence at least 90% identical to a sequence selected from SEQ ID NOS: 4, 6, 8, 10, 12, 14, 114, 118, 122, 126, 130, 133, 135, 137, 139, 141, 143, 145, 147, 149, 151, 153, 155 and any of 314-403; and wherein the Fab comprises one VH comprising an amino acid sequence at least 90% identical to a selected from SEQ ID NOS: 3, 5, 7, 9, 11, 13, 112, 113, 115, 116, 117, 119, 120, 121, 123, 124, 125, 127, 128, 129, 132, 134, 136, 138, 140, 142, 144, 146, 148, 150, 152, 154 and any of 236-313, one VL comprising an amino acid sequence at least 90% identical to a sequence selected from SEQ ID NOS: 4, 6, 8, 10, 12, 14, 114, 118, 122, 126, 130, 133, 135, 137,139, 141, 143, 145, 147,149, 151, 153, 155 and any of 314-403, one human CH1 domain comprising an amino acid sequence at least 90% identical to SEQ ID NO: 156 and one human CL domain comprising an amino acid sequence at least 90% identical to SEQ ID NO: 159, wherein the VH is fused to one of the CH1 or CL domains, and the VL is fused to the other of the CH1 or CL domains.

In one aspect of any of the embodiments described herein, an IL2 comprises an amino acid sequence having at least about 80%, 85%, 90%, 95%, 97%, 98% or 99% identity to the IL-2 polypeptide of any of SEQ ID NOS: 404-417, or to a contiguous sequence of at least 40, 50, 60, 70, 80 or 100 amino acid residues thereof. Optionally the IL2 further comprises 2, 3, 4, 5 or more amino acid substitutions that reduce binding to CD25, e.g. substitutions at any of the residues disclosed herein.

In one aspect of any of the embodiments described herein, an IL15 comprises an amino acid sequence having at least about 80%, 85%, 90%, 95%, 97%, 98% or 99% identity to the IL-15 polypeptide of any of SEQ ID NO: 418, or to a contiguous sequence of at least 40, 50, 60, 70, 80 or 100 amino acid residues thereof.

In one aspect of any of the embodiments described herein, an IL12 comprises an amino acid sequence having at least about 80%, 85%, 90%, 95%, 97%, 98% or 99% identity to the IL-12 polypeptide of any of SEQ ID NOS: 438 and/or 439, or to a contiguous sequence of at least 40, 50, 60, 70, 80 or 100 amino acid residues thereof.

In one aspect of any of the embodiments described herein, an IL7 comprises an amino acid sequence having at least about 80%, 85%, 90%, 95%, 97%, 98% or 99% identity to the IL-7 polypeptide of any of SEQ ID NO: 435, or to a contiguous sequence of at least 40, 50, 60, 70, 80 or 100 amino acid residues thereof.

In one aspect of any of the embodiments described herein, an IL27 comprises an amino acid sequence having at least about 80%, 85%, 90%, 95%, 97%, 98% or 99% identity to the IL-21 polypeptide of any of SEQ ID NOS: 436 and/or 437, or to a contiguous sequence of at least 40, 50, 60, 70, 80 or 100 amino acid residues thereof.

In one aspect of any of the embodiments described herein, an IL21 comprises an amino acid sequence having at least about 80%, 85%, 90%, 95%, 97%, 98% or 99% identity to the IL-27 polypeptide of any of SEQ ID NOS: 420 or 421, or to a contiguous sequence of at least 40, 50, 60, 70, 80 or 100 amino acid residues thereof.

In one aspect of any of the embodiments described herein, an IL18 comprises an amino acid sequence having at least about 80%, 85%, 90%, 95%, 97%, 98% or 99% identity to the IL-18 polypeptide of any of SEQ ID NO: 422, or to a contiguous sequence of at least 40, 50, 60, 70, 80 or 100 amino acid residues thereof.

In one aspect of any of the embodiments described herein, an IFN-α comprises an amino acid sequence having at least about 80%, 85%, 90%, 95%, 97%, 98% or 99% identity to the IFN-α polypeptide of any of SEQ ID NOS: 423-433, or to a contiguous sequence of at least 40, 50, 60, 70, 80 or 100 amino acid residues thereof.

In one aspect of any of the embodiments described herein, an IFN-β comprises an amino acid sequence having at least about 80%, 85%, 90%, 95%, 97%, 98% or 99% identity to the IFN-α polypeptide of any of SEQ ID NOS: 434, or to a contiguous sequence of at least 40, 50, 60, 70, 80 or 100 amino acid residues thereof.

In one aspect of any of the embodiments described herein, an Fc domain comprises an amino acid sequence having at least about 80%, 85%, 90%, 95%, 97%, 98% or 99% identity to the Fc polypeptide of any of SEQ ID NOS: 160-165, or to a contiguous sequence of at least 40, 50, 60, 70, 80 or 100 amino acid residues thereof.

In one aspect of any of the embodiments described herein, a CH1, CH2 and CH3 domain respectively comprise an amino acid sequence having at least about 80%, 85%, 90%, 95%, 97%, 98% or 99% identity to the CH1 polypeptide of SEQ ID NO: 156, 157 or 158, or to a contiguous sequence of at least 40, 50, 60, 70, 80 or 100 amino acid residues thereof.

In one aspect of any of the embodiments described herein, a CK or CL domain comprises an amino acid sequence having at least about 80%, 85%, 90%, 95%, 97%, 98% or 99% identity to the CK polypeptide of any of SEQ ID NO: 159, or to a contiguous sequence of at least 40, 50, 60, 70, 80 or 100 amino acid residues thereof.

In one aspect of any of the embodiments described herein, a hinge domain comprises an amino acid sequence having at least about 80%, 85%, 90%, 95%, 97%, 98% or 99% identity to the CK polypeptide of any of SEQ ID NO: 166-170.

In one aspect of any of the embodiments described herein, a multispecific protein comprises:
(a) an ABD that binds to the antigen of interest, wherein the ABD comprises a VH comprising an amino acid sequence at least 70%, 80% or 90% identical to a sequence selected from SEQ ID NOS: 132, 134, 136, 138, 140, 142, 144, 146, 148, 150, 152, 154 and any of 236-313, a domain linker, and a VL comprising an amino acid sequence at least 90% identical to a sequence selected from SEQ ID NOS: 133, 135, 137, 139, 141, 143, 145, 147, 149, 151, 153, 155 and any of 314-403; and
(b) an ABD that binds to a human NKp46 polypeptide, wherein the ABD comprises a VH comprising an amino acid sequence at least 70%, 80% or 90% identical to a sequence selected from SEQ ID NOS: 3, 5, 7, 9, 11, 13, 112, 113, 115, 116, 117, 119, 120, 121, 123, 124, 125, 127, 128, 129 and any of 236-313, a domain linker, and a VL comprising an amino acid sequence at least 90% identical to a sequence selected from SEQ ID NOS: 4, 6, 8, 10, 12, 14, 114, 118, 122, 126, 130 and any of 314-403;
(c) a Fc domain dimer comprising two Fc domain monomer polypeptides, wherein each Fc domain monomer polypeptide comprises an amino acid sequence at least 70%, 80% or 90% identical to a sequence selected from SEQ ID NOS: 160-165; and
(d) a cytokine polypeptide comprising an amino acid sequence at least 70%, 80% or 90% identical to a sequence selected from SEQ ID NOS: 404-436, or to a contiguous sequence of at least 40, 50, 60, 70, 80 or 100 amino acid residues thereof, fused, via a domain linker, to the C-terminus of one of the polypeptide chains of the multispecific protein.

In one aspect of any of the embodiments described herein, a hinge domain comprises an amino acid sequence having at least about 80%, 85%, 90%, 95%, 97%, 98% or 99% identity to the CK polypeptide of any of SEQ ID NO: 166-170.

In one aspect of any of the embodiments described herein, a multispecific protein comprises:
(a) an ABD that binds to the antigen of interest, wherein the ABD comprises an scFv or Fab,
   a. wherein the scFv comprises a VH comprising an amino acid sequence at least 90% identical to a sequence selected from SEQ ID NOS: 132, 134, 136, 138, 140, 142, 144, 146, 148, 150, 152, 154 and any of 236-313, a domain linker, and a VL comprising an amino acid sequence at least 90% identical to a sequence selected from SEQ ID NOS: 133, 135, 137, 139, 141, 143, 145, 147, 149, 151, 153, 155 and any of 314-403; and
   b. wherein the Fab comprises one VH comprising an amino acid sequence at least 90% identical to a selected from SEQ ID NOS: 132, 134, 136, 138, 140, 142, 144, 146, 148, 150, 152, 154 and any of 236-313, one VL comprising an amino acid sequence at least 90% identical to a sequence selected from SEQ ID NOS: 133, 135, 137, 139, 141, 143, 145, 147, 149, 151, 153, 155 and any of 314-403, one human CH1 domain comprising an amino acid sequence at least 90% identical to SEQ ID NO: 156 and one human CL domain comprising an amino acid sequence at least 90% identical to SEQ ID NO: 159, wherein the VH is fused to one of the CH1 or CL domains, and the VL is fused to the other of the CH1 or CL domains,
(b) an ABD that binds to a human NKp46 polypeptide, wherein the ABD comprises an scFv or Fab,
   a. wherein the scFv comprises a VH comprising an amino acid sequence at least 90% identical to a sequence selected from SEQ ID NOS: 3, 5, 7, 9, 11, 13, 112, 113, 115, 116, 117, 119, 120, 121, 123, 124, 125, 127, 128, 129 and any of 236-313, a domain linker, and a VL comprising an amino acid sequence at least 90% identical to a sequence selected from SEQ ID NOS: 4, 6, 8, 10, 12, 14, 114, 118, 122, 126, 130 and any of 314-403; and
   b. wherein the Fab comprises one VH comprising an amino acid sequence at least 90% identical to a selected from SEQ ID NOS: 3, 5, 7, 9, 11, 13, 112, 113, 115, 116, 117, 119, 120, 121, 123, 124, 125, 127, 128, 129 and any of 236-313, one VL comprising an amino acid sequence at least 90% identical to a sequence selected from SEQ ID NOS: 4, 6, 8, 10, 12, 14, 114, 118, 122, 126, 130 and any of 314-403, one human CH1 domain comprising an amino acid sequence at least 90% identical to SEQ ID NO: 156 and one human CL domain comprising an amino acid sequence at least 90% identical to SEQ ID NO: 159, wherein the VH is fused to one of the CH1 or CL domains, and the VL is fused to the other of the CH1 or CL domains,
(c) a Fc domain dimer comprising two Fc domain monomer polypeptides, wherein each Fc domain monomer polypeptide comprises an amino acid sequence at least 90% identical to a sequence selected from SEQ ID NOS: 160-165; and
(d) a cytokine polypeptide comprising an amino acid sequence at least 90% identical to a sequence selected from SEQ ID NOS: 404-436, or to a contiguous sequence of at least 40, 50, 60, 70, 80 or 100 amino acid residues thereof, fused, via a domain linker, to the C-terminus of one of the polypeptide chains of the multispecific protein.

In one embodiment, a multispecific protein comprises: a polypeptide comprising an amino acid sequence having at least 80%, 90% or 95% sequence identity to the amino acid sequence of a first chain of a heterotrimeric protein described herein, a polypeptide comprising an amino acid sequence having at least 80%, 90% or 95% sequence identity to the amino acid sequence of a second chain of a heterotrimeric protein described herein and a polypeptide comprising an amino acid sequence having at least 80%, 90% or 95% sequence identity to the amino acid sequence of a third chain of a heterotrimeric protein described herein. In one embodiment, a multispecific protein comprises: a polypeptide comprising an amino acid sequence having at least 80%, 90% or 95% sequence identity to the amino acid sequence of a first chain of a heterodimeric protein described herein, and a polypeptide comprising an amino acid sequence having at least 80%, 90% or 95% sequence identity to the amino acid sequence of a second chain of a heterodimeric protein described herein.

In one embodiment, a multispecific protein comprises: a polypeptide comprising an amino acid sequence of a first chain of a heterotrimeric protein described herein, a polypeptide comprising an amino acid sequence of a heterotrimeric protein described herein and a polypeptide comprising an amino acid sequence of a third chain of a heterotrimeric protein described herein. In one embodiment, a multispecific protein comprises: a polypeptide comprising an amino acid sequence of a first chain of a heterodimeric protein described herein, and a polypeptide comprising an amino acid sequence of a second chain of a heterodimeric protein described herein.

In one embodiment, a multispecific protein comprises: a polypeptide comprising an amino acid sequence having at least 80%, 90% or 95% sequence identity to the amino acid sequence of SEQ ID NO: 175, a polypeptide comprising an amino acid sequence having at least 80%, 90% or 95% sequence identity to the amino acid sequence of SEQ ID NO: 176 and a polypeptide comprising an amino acid sequence having at least 80%, 90% or 95% sequence identity to the amino acid sequence of SEQ ID NO: 177.

In one embodiment, a multispecific protein comprises: a polypeptide comprising an amino acid sequence having at least 80%, 90% or 95% sequence identity to the amino acid sequence of SEQ ID NO: 193, a polypeptide comprising an amino acid sequence having at least 80%, 90% or 95% sequence identity to the amino acid sequence of SEQ ID NO: 185; and a polypeptide comprising an amino acid sequence having at least 80%, 90% or 95% sequence identity to the amino acid sequence of SEQ ID NO: 194.

In one embodiment, a multispecific protein comprises: a polypeptide comprising an amino acid sequence having at least 80%, 90% or 95% sequence identity to the amino acid sequence of SEQ ID NO: 199, a polypeptide comprising an amino acid sequence having at least 80%, 90% or 95% sequence identity to the amino acid sequence of SEQ ID NO: 200; and a polypeptide comprising an amino acid sequence having at least 80%, 90% or 95% sequence identity to the amino acid sequence of SEQ ID NO: 201.

In one embodiment, a multispecific protein comprises: a polypeptide comprising an amino acid sequence having at least 80%, 90% or 95% sequence identity to the amino acid sequence of SEQ ID NO: 209, a polypeptide comprising an amino acid sequence having at least 80%, 90% or 95% sequence identity to the amino acid sequence of SEQ ID NO: 210; and a polypeptide comprising an amino acid sequence having at least 80%, 90% or 95% sequence identity to the amino acid sequence of SEQ ID NO: 211.

In one embodiment, a multispecific protein comprises: a polypeptide comprising an amino acid sequence having at least 80%, 90% or 95% sequence identity to the amino acid sequence of SEQ ID NO: 212, a polypeptide comprising an amino acid sequence having at least 80%, 90% or 95% sequence identity to the amino acid sequence of SEQ ID NO: 213; and a polypeptide comprising an amino acid sequence having at least 80%, 90% or 95% sequence identity to the amino acid sequence of SEQ ID NO: 214.

In one embodiment, a multispecific protein comprises: a polypeptide comprising an amino acid sequence having at least 80%, 90% or 95% sequence identity to the amino acid sequence of SEQ ID NO: 215, a polypeptide comprising an amino acid sequence having at least 80%, 90% or 95% sequence identity to the amino acid sequence of SEQ ID NO: 216; and a polypeptide comprising an amino acid sequence having at least 80%, 90% or 95% sequence identity to the amino acid sequence of SEQ ID NO: 217.

In one embodiment, a multispecific protein comprises: a polypeptide comprising an amino acid sequence having at least 80%, 90% or 95% sequence identity to the amino acid sequence of SEQ ID NO: 218, a polypeptide comprising an amino acid sequence having at least 80%, 90% or 95% sequence identity to the amino acid sequence of SEQ ID NO: 219; and a polypeptide comprising an amino acid sequence having at least 80%, 90% or 95% sequence identity to the amino acid sequence of SEQ ID NO: 220.

In one aspect the invention provides an isolated multispecific heterotrimeric protein comprising a first polypeptide chain comprising an amino acid sequence which is at least 50%, 60%, 70%, 80%, 85%, 90%, 95%, 98 or 99% identical to the sequence of a first polypeptide chain of a T5, T6, T25 or T26 protein disclosed herein; a second polypeptide chain comprising an amino acid sequence which is at least 50%, 60%, 70%, 80%, 85%, 90%, 95%, 98 or 99% identical to the sequence of a second polypeptide chain of the respective T5, T6, T25 or T26 protein disclosed herein; and optionally a third polypeptide chain comprising an amino acid sequence which is at least 50%, 60%, 70%, 80%, 85%, 90%, 95%, 98 or 99% identical to the sequence of a third polypeptide chain of a T5, T6, T25 or T26 protein disclosed herein. In one embodiment, CDRs are excluded from the sequences that are considered for computing sequence identity. In one embodiment, VH and/or VL variable regions are excluded from the sequences that are considered for computing sequence identity of a polypeptide chain. Optionally each VH region comprises an amino acid sequence having at least about 80%, 85%, 90%, 95%, 97%, 98% or 99% identity, to the amino acid sequence of SEQ ID NOS: 3, 5, 7, 9, 11, 13, 112, 113, 115, 116, 117, 119, 120, 121, 123, 124, 125, 127, 128, 129, 132, 134, 136, 138, 140, 142, 144, 146, 148, 150, 152, 154 or and any of 236-313. Optionally each VL region comprises an amino acid sequence having at least about 80%, 85%, 90%, 95%, 97%, 98% or 99% identity, to the amino acid sequence of SEQ ID NOS: 4, 6, 8, 10, 12, 14, 114, 118, 122, 126, 130, 133, 135, 137, 139, 141, 143, 145, 147, 149, 151, 153, 155 or any of 314-403.

In one aspect of any of the embodiments described herein, provided is a recombinant nucleic acid encoding a first polypeptide chain, and/or a second polypeptide chain, and/or a third polypeptide chain and/or a fourth polypeptide. In one aspect of any of the embodiments described herein, the invention provides a recombinant host cell comprising a nucleic acid encoding a first polypeptide chain, and/or a second polypeptide chain and/or a third polypeptide chain, optionally wherein the host cell produces a multimeric or other protein according to the invention with a yield (final productivity or concentration before or after purification) of at least 1, 2, 3 or 4 mg/L. Also provided is a kit or set of nucleic acids comprising a recombinant nucleic acid encoding a first polypeptide chain of the according to the invention, a recombinant nucleic acid encoding a second polypeptide chain according to the invention, and, optionally, a recombinant nucleic acid encoding a third polypeptide chain according to the invention. Also provided are methods of making dimeric, trimeric and tetrameric proteins according to the invention.

In another embodiment, the disclosure provides novel variant IL-2 polypeptides that are particularly suited for use in an antigen binding protein, particularly an antigen binding protein that has an Fc domain, yet further wherein the Fc domain is modified to decrease or lack the ability to bind CD16A and/or other Fc gamma receptors. Provided herein is a modified or variant IL-2 polypeptide comprising the amino acid substitution T41A, wherein numbering of residues is with respect to the IL-2 polypeptide of SEQ ID NO: 404. In one embodiment, provided herein is a modified or variant IL-2 polypeptide comprising at least two, three, four, five, six or more amino acid substitutions, wherein the polypeptide comprises the substitutions T41A. In one embodiment, provided herein is a modified or variant IL-2 polypeptide comprising at least three amino acid substitutions, wherein the polypeptide comprises substitutions at residues R38, F42 and T41 compared to a human wild type IL-2 polypeptide, wherein number of residues is with respect to the IL-2 polypeptide of SEQ ID NO: 404. In one aspect, a modified or variant IL-2 polypeptide comprises at least three amino acid substitutions compared to a human wild type IL-2 polypeptide, wherein the polypeptide comprises the substitutions: R38A, F42K and T41X, wherein X is any amino acid residue. In one aspect, a modified or variant IL-2 polypeptide comprises at least three (e.g. 3, 4, 5, 6 or more) amino acid substitutions compared to a human wild type IL-2 polypeptide, wherein the polypeptide comprises the substitutions: R38A, F42K and T41A. In one aspect, the modified or variant IL2 comprises the amino acid sequence of SEQ ID NO: 408. In one aspect, the modified or variant IL2 comprises an amino acid sequence having at least about 80%, 85%, 90%, 95%, 97%, 98% or 99% identity to the IL-2 polypeptide of SEQ ID NO: 408, or to a contiguous sequence of at least 40, 50, 60, 70, 80 or 100 amino acid residues thereof. Provided also are proteins or polypeptides comprising the aforementioned modified or variant IL-2 polypeptide. In one embodiment, provided is the modified or variant IL-2 polypeptide fused (e.g., at its N-terminus) to a heterologous amino acid sequence or polypeptide. In one embodiment, provided is a polypeptide or protein (e.g. an Fc domain-containing protein, antigen binding protein, multispecific protein, or antibody) that binds an antigen of interest (e.g. cancer antigen or a receptor on an immune cell, optionally an NK cell) comprising the modified or variant IL-2 polypeptide, optionally wherein the IL-2 polypeptide is fused via a domain linker to the polypeptide or protein, optionally wherein the IL-2 polypeptide is fused via a domain linker to the C-terminus of the protein or polypeptide. In one embodiment, the protein or polypeptide that comprises the variant IL-2 comprises a human Fc domain, yet further wherein the Fc domain is modified to decrease or lack the ability to bind CD16A and/or other Fc gamma receptors. In some embodiments, the modified IL-2 has reduced binding affinity for CD25 relative to wild type IL-2. In some embodiments, the modified IL-2 has reduced activity on resting or activated T cells relative to wild type IL-2.

Any of the methods can further be characterized as comprising any step described in the application, including notably in the “Detailed Description of the Invention”). The invention further relates to methods of identifying, testing and/or making proteins described herein. The invention further relates to a multispecific protein obtainable by any of present methods. The disclosure further relates to pharmaceutical or diagnostic formulations containing at least one of the multispecific proteins disclosed herein. The disclosure further relates to methods of using the subject multispecific proteins in methods of treatment or diagnosis.

These and additional advantageous aspects and features of the invention may be further described elsewhere herein.

### BRIEF DESCRIPTION OF THE FIGURES

FIGS. 1A, 1B, 1C and 1D show structure/function relationships for multispecific NK cell engager (NKCE) protein comprising a variant IL2 (IL2v) binding on one face to a tumor antigen on a tumor cell, and on another face to an NK cell via a triple receptor cis-presentation of IL2βγ complex, NKp46 and CD16A. IL2v capture on NK cells may improve binding to CD122 and mimic CD25-mediated IL-2 presentation.

FIG. 2A shows an exemplary multispecific protein in T5 format that binds to NKp46, CD16A and CD122 on an NK cell, and to CD20 on a tumor cell. FIGS. 2B to 2N show different configurations of multispecific proteins that differ in the number of antigen of interest (e.g. cancer antigen) binding sites (1 or 2 sites), and in the configuration of the domains around an Fc domain dimer. In FIGS. 2G, H, J, K, L, M and N, the star in the CH3 domain indicates mutations H435R and Y436F (Kabat EU numbering).

FIG. 3 activation of TReg cells by T6 format proteins that contained either a wild-type IL-2 or a variant IL2, and that lacked binding to NKp46, CD16A and antigen of interest. The T6 protein containing the variant IL2 showed a strongly decreased ability to activate Treg cells compared to the T6 protein containing the wild-type IL-2.

FIG. 4 shows % of pSTAT5 cells among NK cells. CD20-T5-NKp46-IL2v and IC-T6-IC-IL2v displayed comparable activation of Treg cells, CD4 T cells and CD8 T cells. However, the CD20-T5-NKp46-IL2v resulted in an approximately 2-log increase in percent of pSTAT5+ cells among the NK cells, compared to IC-T6-IC-IL2 that did not bind NKp46 or CD16A. The CD20-T5-NKp46-IL2v protein permitted a selective activation of NK cells over Treg cells, CD4 T cells and CD8 T cells.

FIG. 5 shows % of pSTAT5 cells among NK cells. CD20-T5-NKp46-IL2v which bound both CD16 and NKp46 (in addition to the IL2v moiety) resulted in strong increase in potency (an approximately 1-log increase) in percent of pSTAT5+ cells among the NK cells, compared to CD20-T6-NKp46-IL2v. IL-2R signaling in NK cells was therefore enhanced by each of NKp46 and CD16, and with a particularly strong enhancement when both NKp46 and CD16 were bound in addition to IL-2R.

FIG. 6 shows % of CD69-expressing NK cells, in the absence of tumor cells. The CD20-T5-NKp46 that bound CD20, NKp46 and CD16 but lacked the IL2v moiety did not activate NK cells in the absence of tumor cells, while all the proteins containing the IL2v moiety resulted in strong NK cell activation, with an additional benefit seen for the proteins that had a NKp46 binding domain and a wild-type Fc domain compared to the IC-T6-IC-IL2v protein that lacked CD16 and NKp46 binding.

FIG. 7 shows % of proliferating NK cells in the absence of tumor cells on the y-axis and concentration of test protein on the x-axis. The CD20-T5-NKp46 that bound CD20, NKp46 and CD16 but lacked the IL2v moiety did not induce proliferation of NK cells, while all the proteins containing the IL2v moiety resulted in strong NK cell proliferation, although with differences in potency. All NK cell engager proteins with NKp46 binding domain and/or wild-type Fc domain (in addition to IL2v) were more potent in inducing NK cell proliferation compared to the IC-T6-IC-IL2v protein that lacked CD16 and NKp46 binding.

FIGS. 8A and 8B show % specific lysis induced by NK cells in a cytotoxicity assay at ET ratio 10:1. FIGS. 8C and 8D show % specific lysis induced by NK cells in a cytotoxicity assay at ET ratio 2:1. All of the NK cell engagers that retained the ability to bind both CD16 and NKp46 (in addition to CD20) displayed similarly high potency in terms of EC50 values in induction of NK cell cytotoxicity toward the tumor cells. The nature of the IL-2 polypeptide (either wild-type of the mutated IL2v) did not appear to differentially affect NK cell cytotoxicity, and furthermore the presence of IL2, whether as wild-type or IL2v, did not result in improved EC50 values in induction of cytotoxicity.

FIG. 9 shows cytokine production by NK cells in the presence of NK cell engagers and tumor cells. The NK cell engager proteins with NKp46 binding domain and wild-type Fc domain were more potent in inducing MIP1b and IFNγ production by NK cells. In absence of tumor targeting, as exemplified by the IC-T5-NKp46-IL2v construct that did not bind CD20, the NK cell engager did not elicit cytokine production by the NK cells.

FIG. 10 shows that administration to mice of CD20-T5-NKp46-IL2v NK cell engager protein that bound CD20, NKp46, CD16A and CD122 (right panel) showed strong anti-tumor efficacy as a single injection, compared to obinutuzumab (left panel).

FIG. 11 shows administration of the 25 μg dose of CD20-T5-NKp46-IL2v resulted in very strong and long anti-tumor activity in large volume tumors in mice, permitting tumors to generally stay below 300 mm³ in volume for the duration of the study.

FIG. 12 shows that CD20-T5-NKp46-IL2v NK cell engager protein that binds CD20, NKp46, CD16A and CD122 showed strong efficacy in a mouse tumor model when administered as two injections separated by a one week, starting at day 9 post tumor engraftment when tumors had grown to 60 mm³ in volume. In comparison, in the CD20-T5-IC-IL2v group and in the CD20-F5-NKp46, tumor were somewhat initially controlled during week after treatment but then rapidly grew past 300 mm³ in volume thereafter. NKp46 and CD122 binding are important to efficient control tumor growth and their simultaneous targeting drives the strong antitumor efficacy of the CD20-T5-NKp46-IL2v molecule.

FIG. 13 left hand panel shows that tumors harvested from mice treated by the CD20-T5-NKp46-IL2v NK cell engager protein that bound CD20, NKp46, CD16A and CD122 showed high expression of the ncr1 transcript (encoding for NKp46 protein and highly specific for NK cells), demonstrating an increase of NK cell infiltration in tumor. In comparison, tumors harvested in mice treated by the CD20-F5-NKp46 protein or obinutuzumab showed only minor increase of ncr1 transcripts revealing a much lower NK cell infiltrate in tumors. The right hand panel shows that tumors harvested in mice treated by the CD20-T5-NKp46-IL2v NK cell engager protein showed higher expression of the interferon gamma (ifng) transcript compared to other treated conditions, showing that NK cells are activated.

FIG. 14 shows in the upper right hand panel shows that treatment with the CD20-T5-NKp46-IL2v NK cell engager controlled tumor growth (days of treatment shown with arrows). However, NK cell depletion resulted in a loss of control of tumor growth by about day 30 (lower right hand panel) while tumor are still controlled in mice not depleted for NK cells.

FIG. 15 shows % of pSTAT5 cells among PBMC. All the GA101-T5-NKp46-IL2v NK cell engager proteins were comparable in their ability to induce IL2R signaling preferentially in NK cells over TReg cells, CD8 T cells and CD4 T cells whatever the length of the linker between the IL2v and the C-terminus of the NKp46-binding Fab.

FIG. 16 shows cytotoxicity potentiated by the NKCE proteins in the “T5” and “T6” formats in which the NKp46-binding domain based on the NKp46-1 VH/VL pair is positioned between the Fc domain dimer and the C-terminal IL2v, with 10 amino linker, short (5 aa) linker or long (15 aa) linker; the proteins were all comparable in their ability to potentiated NK cell cytotoxicity towards tumor cells.

FIG. 17 shows cytotoxicity potentiated by the NKCE proteins in the T5” and “T6” formats in which the NKp46-binding domain based on the NKp46-4 VH/VL pair is positioned between the Fc domain dimer and the C-terminal IL2v, with 10 amino linker, short (5 aa) linker or long (15 aa) linker; the proteins were all comparable in their ability to potentiated NK cell cytotoxicity towards tumor cells.

FIG. 18 shows the structure of the proteins tested in FIGS. 15, 16 and 17.

FIG. 19 shows % of pSTAT5 cells among PBMC cells. The CD20-T5-NKp46-IL2v, CD20-T5-NKp46-IL2v2 and CD20-T5-NKp46-IL2v3 having different IL-2 moieties were comparable and each resulted in an approximately 2-log increase in percent of pSTAT5+ cells among the NK cells, compared to IC-T6-IC-IL2 (IL2pWT) that contained wild-type IL-2 and did not bind NKp46 or CD16A. The CD20-T5-NKp46-IL2v protein therefore permitted a selective activation of NK cells over Treg cells, CD4 T cells and CD8 T cells. Substitution of different “not-alpha” cytokine variants does not impact ability of the NKCE-IL2v protein to selectively activate NK cells over Treg cells, CD4 T cells and CD8 T cells.

FIG. 20 shows IL-6 production and the right panel showing TNFα production over time following treatment with a 25 or 70 μg dose of CD20-T5-NKp46-IL2v NK cell engager; the left bar shows plasma concentration of cytokine for the 70 μg dose and the right bar shows plasma concentration of cytokine for the 25 μg dose.

FIGS. 21A and 21B show the domain structure of different multispecific proteins lacking cytokine moieties used to study the mechanism of action of multispecific proteins, including Format 2 (F2), Format 5 (F5), Format 7 (F7), Format 13 (F13) and Format 14 (F14) proteins.

FIG. 22 (22A: CD107 and 22B: CD69) shows activation of NK cells by multispecific proteins that bind CD19, CD16A and NKp46, in the presence of target-antigen expressing cells. Each of the CD19×NKp46 binding proteins (respectively including NKp46-1, NKp46-2, NKp46-3, NKp46-4 or NKp46-9 variable regions) activated NK cells in the presence of Daudi cells.

FIG. 23 shows ability to direct purified NK cells to lyse CD19-positive Daudi tumor target cells by CD19×NKp46 binding proteins. CD19-F6-NKp46 protein whose Fc domain does not bind CD16A due to a N297 substitution was as potent in mediating NK cell lysis of Daudi target cells as the bivalent CD19-binding full-length IgG1 anti-CD19 antibody, and the CD19-F5-NKp46 (F5 format protein) whose Fc domain binds CD16A was even more potent.

FIG. 24 shows that the NKp46×EGFR NKCE protein, whose Fc domain binds CD16, is highly potent in mediating A549 target cell lysis.

FIG. 25 shows that the NKp46×ROR1 NKCE protein, whose Fc domain binds CD16, is highly potent in mediating Mino tumor target cell lysis.

FIG. 26 shows that the NKp46×KIR3DL2 NKCE protein, whose Fc domain binds CD16, is highly potent in mediating HUT78 tumor target cell lysis.

FIG. 27 shows % of pSTAT5 cells among NK cells on the y-axis and concentration of test protein on the x-axis upon, where incubation with CD20-T5A-NKp46-IL15 induced a decrease in the EC50 for STAT5 phosphorylation among the NK cells, compared to CD20-T6AB3-ICb-IL15 that did not bind NKp46 or CD16A.

FIG. 28 shows % of proliferating NK, CD4 T or CD8 T cells on the y-axis and concentration of test protein on the x-axis, where incubation with CD20-T5A-NKp46-IL15 resulted in strong NK cell proliferation, with an increase in potency compared to the CD20-T6AB3-IC-IL15 protein that did not bind NKp46 or CD16A. The increase in potency was selective for NK cells, as there was no increase potency by CD20-T5A-NKp46-IL15 for induction of CD4 and CD8 T cell proliferation.

FIG. 29 shows % specific lysis of tumor cells by NK cells on the y-axis and concentration of test protein on the x-axis. The IC-T5A-NKp46-IL15 that lacked binding to CD20 on targeted cells did not induce significant cytotoxicity toward the tumor cells, while the CD20-T5A-NKp46-IL15 displayed high potency.

FIG. 30, left hand panel, shows % of CD69 expressing NK cells on the y-axis and concentration of test protein on the x-axis, and right hand panel shows median fluorescence intensity (medFI) of CD69 expression in NK cells. CD20-T5A-NKp46-IL18v induced a decrease in the range of two orders of magnitude in the EC₅₀ for activation of NK cells, compared to GA101-T6AB3-IC-IL18v that did not bind CD16A or NKp46 on NK cells.

FIG. 31 shows % of IFN-γ expressing NK cells on the y-axis and concentration of test protein on the x-axis upon incubation with CD20-T5A-NKp46-IL18v which induced a decrease in the range of two orders of magnitude in the EC₅₀ for activation of NK cells, compared to CD20-T6AB3-IC-IL18v that did not bind CD16A or NKp46 on NK cells.

FIG. 32 shows % of proliferating NK, CD4 T or CD8 T cells on the y-axis and concentration of test protein on the x-axis upon incubation with the CD20-T5A-NKp46-IL18v protein which resulted in strong NK cell proliferation that was selective over CD4 and CD8 T cells. The CD20-T6AB3-IC-IL18v protein that did not bind CD16A or NKp46 did not show significant activation of NK cells.

FIG. 33 shows % specific lysis of tumor cells by NK cells on the y-axis and concentration of test protein on the x-axis, upon incubation with CD20-T5A-NKp46-IL18v protein. The IC-T5A-NKp46-IL18v that lacked binding to CD20 on targeted cells did not induce significant cytotoxicity.

FIG. 34 shows % of pSTAT3 cells among NK cells, CD4 T or CD8 T cells on the y-axis and concentration of test protein on the x-axis, upon incubation with CD20-T5A-NKp46-IFNαv which induced potent STAT3 phosphorylation selectively among the NK cells over CD4 or CD8 T cells, compared to CD20-T6AB3-IC-IFNαv that did not induce any significant NK cell activation.

FIG. 35 shows % specific lysis of tumor cells by NK cells on the y-axis and concentration of test protein on the x-axis upon incubation with CD20-T5A-NKp46-IFNαv which displayed high potency in terms of EC₅₀ values in induction of NK cell cytotoxicity.

FIG. 36 shows the difference from baseline of the B cell count (cells/μL) over the 14 days before and 30 days following treatment of non-human primates with the NKCE proteins, showing that the NKCE proteins induced B cell depletion, while control (vehicle) did not FIG. 37 shows production of different cytokines over the course of 24 hours following administration of the NKCE proteins to non-human primates.

FIG. 38 shows mean values for red blood cells, platelets, hemoglobin, hematocrit, mean corpuscular volume, mean corpuscular hemoglobin and mean corpuscular hemoglobin concentration for non-human primates treated with the NKCEs or vehicle over a period from 14 days before treatment through 30 days following treatment

FIG. 39 shows levels of white blood cells, lymphocytes, monocytes, neutrophils, eosinophils and basophils cells in non-human primates over a period from 14 days before treatment through 30 days following treatment

FIG. 40 shows levels of NK cells, CD8+ T cells, FoxP3-CD4+ T cells and FoxP3⁺CD4+ T cells in non-human primates over a period from 14 days before treatment through 30 days following treatment.

FIG. 41 shows results of flow cytometry for staining for expression of NK1.1 and CD3 in cells from tumors in mice following treatment with CD20-T5-NKp46-IL2v, showing that CD20-T5-NKp46-IL2v stimulated accumulation of NK cells (NK1.1⁺CD3−) at the tumor site.

FIG. 42 shows number of NK cell/spleen, % of CD69-expressing NK cells in spleen and Ki67-expressing NK cells in spleen, following treatment of mice. The CD20-F5-NKp46 protein and obinutuzumab did not increase the number of NK cells, while treatment with CD20-T5-NKp46-IL2v caused a strong increase in NK cells in the spleen. Additionally, CD20-T5-NKp46-IL2v caused a strong increase in activated or proliferating NK cells among total NK cells.

FIG. 43 shows number of NK cell/μL of blood, % of CD69-expressing NK cells in blood, % of CD69-expressing NK cells in spleen and Ki67-expressing NK cells in spleen, following treatment of mice. Treatment with CD20-T5-NKp46-IL2v caused a strong increase in NK cell number per μl of blood and also in the spleen, as well as strong increase in activated (CD69-expressing) NK cells among NK cells in blood and spleen.

### DETAILED DESCRIPTION OF THE INVENTION

### Definitions

As used in the specification, “a” or “an” may mean one or more. As used in the claim(s), when used in conjunction with the word “comprising”, the words “a” or “an” may mean one or more than one.

Where “comprising” is used, this can optionally be replaced by “consisting essentially of”, or optionally by “consisting of”.

As used herein, the term “antigen binding domain” or “ABD” refers to a domain comprising a three-dimensional structure capable of immunospecifically binding to an epitope. Thus, in one embodiment, said domain can comprise a hypervariable region, optionally a V_{H} and/or V_{L} domain of an antibody chain, optionally at least a V_{H} domain. In another embodiment, the binding domain may comprise at least one complementarity determining region (CDR) of an antibody chain. In another embodiment, the binding domain may comprise a polypeptide domain from a non-immunoglobulin scaffold.

The term “antibody” herein is used in the broadest sense and specifically includes full-length monoclonal antibodies, polyclonal antibodies, multispecific antibodies (e.g., bispecific antibodies), and antibody fragments and derivatives, so long as they exhibit the desired biological activity. Various techniques relevant to the production of antibodies are provided in, e.g., Harlow, et al., ANTIBODIES: A LABORATORY MANUAL, Cold Spring Harbor Laboratory Press, Cold Spring Harbor, N.Y., (1988). An “antibody fragment” comprises a portion of a full-length antibody, e.g. antigen-binding or variable regions thereof. Examples of antibody fragments include Fab, Fab′, F(ab)₂, F(ab′)₂, F(ab)₃, Fv (typically the V_{L} and V_{H} domains of a single arm of an antibody), single-chain Fv (scFv), dsFv, Fd fragments (typically the V_{H} and CH1 domain), and dAb (typically a V_{H} domain) fragments; V_{H}, V_{L}, VhH, and V-NAR domains; minibodies, diabodies, triabodies, tetrabodies, and kappa bodies (see, e.g., Ill et al., Protein Eng 1997; 10: 949-57); camel IgG; IgNAR; and multispecific antibody fragments formed from antibody fragments, and one or more isolated CDRs or a functional paratope, where isolated CDRs or antigen-binding residues or polypeptides can be associated or linked together so as to form a functional antibody fragment. Various types of antibody fragments have been described or reviewed in, e.g., Holliger and Hudson, Nat Biotechnol 2005; 23, 1126-1136; WO2005040219, and published U.S. Patent Applications 20050238646 and 20020161201.

The term “hypervariable region” when used herein refers to the amino acid residues of an antibody that are responsible for antigen binding. The hypervariable region generally comprises amino acid residues from a “complementarity-determining region” or “CDR” (e.g. residues 24-34 (L1), 50-56 (L2) and 89-97 (L3) in the light-chain variable domain and 31-35 (H1), 50-65 (H2) and 95-102 (H3) in the heavy-chain variable domain; Kabat et al. 1991) and/or those residues from a “hypervariable loop” (e.g. residues 26-32 (L1), 50-52 (L2) and 91-96 (L3) in the light-chain variable domain and 26-32 (H1), 53-55 (H2) and 96-101 (H3) in the heavy-chain variable domain; Chothia and Lesk, J. Mol. Biol 1987; 196:901-917). Typically, the numbering of amino acid residues in this region is performed by the method described in Kabat et al., supra. Phrases such as “Kabat position”, “variable domain residue numbering as in Kabat” and “according to Kabat” herein refer to this numbering system for heavy chain variable domains or light chain variable domains. Using the Kabat numbering system, the actual linear amino acid sequence of a peptide may contain fewer or additional amino acids corresponding to a shortening of, or insertion into, a FR or CDR of the variable domain. For example, a heavy chain variable domain may include a single amino acid insert (residue 52a according to Kabat) after residue 52 of CDR H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

By “framework” or “FR” residues as used herein is meant the region of an antibody variable domain exclusive of those regions defined as CDRs. Each antibody variable domain framework can be further subdivided into the contiguous regions separated by the CDRs (FR1, FR2, FR3 and FR4).

By “constant region” as defined herein is meant an antibody-derived constant region that is encoded by one of the light or heavy chain immunoglobulin constant region genes.

By “constant light chain” or “light chain constant region” or “CL” as used herein is meant the region of an antibody encoded by the kappa (Cκ) or lambda (Cλ) light chains. The constant light chain typically comprises a single domain, and as defined herein refers to positions 108-214 of Cκ, or Cλ, wherein numbering is according to the EU index (Kabat et al., 1991, Sequences of Proteins of Immunological Interest, 5th Ed., United States Public Health Service, National Institutes of Health, Bethesda).

By “constant heavy chain” or “heavy chain constant region” as used herein is meant the region of an antibody encoded by the mu, delta, gamma, alpha, or epsilon genes to define the antibody's isotype as IgM, IgD, IgG, IgA, or IgE, respectively. For full length IgG antibodies, the constant heavy chain, as defined herein, refers to the N-terminus of the CH1 domain to the C-terminus of the CH3 domain, thus comprising positions 118-447, wherein numbering is according to the EU index.

By “Fab” or “Fab region” as used herein is meant a unit that comprises the V_{H}, CH1, V_{L}, and CL immunoglobulin domains. The term Fab includes a unit that comprises a V_{H}-CH1 moiety that associates with a V_{L}-CL moiety, as well as crossover Fab structures in which there is crossing over or interchange between light- and heavy-chain domains. For example a Fab may have a V_{H}-CL unit that associates with a V_{L}-CH1 unit. Fab may refer to this region in isolation, or this region in the context of a protein, multispecific protein or ABD, or any other embodiments as outlined herein.

By “single-chain Fv” or “scFv” as used herein are meant antibody fragments comprising the V_{H} and V_{L} domains of an antibody, wherein these domains are present in a single polypeptide chain. Generally, the Fv polypeptide further comprises a polypeptide linker between the V_{H} and V_{L} domains which enables the scFv to form the desired structure for antigen binding. Methods for producing scFvs are well known in the art. For a review of methods for producing scFvs see Pluckthun in The Pharmacology of Monoclonal Antibodies, vol. 113, Rosenburg and Moore eds. Springer-Verlag, New York, pp. 269-315 (1994).

By “Fv” or “Fv fragment” or “Fv region” as used herein is meant a polypeptide that comprises the V_{L} and V_{H} domains of a single antibody.

By “Fc” or “Fc region”, as used herein is meant the polypeptide comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM, Fc may include the J chain. For IgG, Fc comprises immunoglobulin domains Cγ2 (CH2) and Cγ3 (CH3) and optionally the hinge between Cγ1 and Cγ2. Although the boundaries of the Fc region may vary, the human IgG heavy chain Fc region is usually defined to comprise residues C226, P230 or A231 to its carboxyl-terminus, wherein the numbering is according to the EU index. Fc may refer to this region in isolation, or this region in the context of an Fc polypeptide, as described below. By “Fc polypeptide” or “Fc-derived polypeptide” as used herein is meant a polypeptide that comprises all or part of an Fc region. Fc polypeptides herein include but are not limited to antibodies, Fc fusions and Fc fragments. Also, Fc regions according to the invention include variants containing at least one modification that alters (enhances or diminishes) an Fc associated effector function. Also, Fc regions according to the invention include chimeric Fc regions comprising different portions or domains of different Fc regions, e.g., derived from antibodies of different isotype or species.

By “variable region” as used herein is meant the region of an antibody that comprises one or more Ig domains substantially encoded by any of the V_{L} (including Vκ (Vκ) and Vλ) and/or V_{H} genes that make up the light chain (including κ and λ) and heavy chain immunoglobulin genetic loci respectively. A light or heavy chain variable region (V_{L} or V_{H}) consists of a “framework” or “FR” region interrupted by three hypervariable regions referred to as “complementarity determining regions” or “CDRs”. The extent of the framework region and CDRs have been precisely defined, for example as in Kabat (see “Sequences of Proteins of Immunological Interest,” E. Kabat et al., U.S. Department of Health and Human Services, (1983)), and as in Chothia. The framework regions of an antibody, that is the combined framework regions of the constituent light and heavy chains, serves to position and align the CDRs, which are primarily responsible for binding to an antigen.

The term “specifically binds to” means that an antibody or polypeptide can bind preferably in a competitive binding assay to the binding partner, e.g. NKp46, as assessed using either recombinant forms of the proteins, epitopes therein, or native proteins present on the surface of isolated target cells. Competitive binding assays and other methods for determining specific binding are further described below and are well known in the art.

When an antibody or polypeptide is said to “compete with” a particular multispecific protein or a particular monoclonal antibody (e.g. NKp46-1, -2, -4, -6 or -9 in the context of an anti-NKp46 mono-specific antibody or a multi-specific protein), it means that the antibody or polypeptide competes with the particular multispecific protein or monoclonal antibody in a binding assay using either recombinant target (e.g. NKp46) molecules or surface expressed target (e.g. NKp46) molecules. For example, if a test antibody reduces the binding of NKp46-1, -2, -4, -6 or -9 to a NKp46 polypeptide or NKp46-expressing cell in a binding assay, the antibody is said to “compete” respectively with NKp46-1, -2, -4, -6 or -9.

The term “affinity”, as used herein, means the strength of the binding of an antibody or protein to an epitope. The affinity of an antibody is given by the dissociation constant K_{D}, defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant K_{A} is defined by 1/K_{D}. Preferred methods for determining the affinity of proteins can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of proteins is the use of surface plasmon resonance (SPR) screening (such as by analysis with a BIAcore™ SPR analytical device).

Within the context of this invention a “determinant” designates a site of interaction or binding on a polypeptide.

The term “epitope” refers to an antigenic determinant, and is the area or region on an antigen to which an antibody or protein binds. A protein epitope may comprise amino acid residues directly involved in the binding as well as amino acid residues which are effectively blocked by the specific antigen binding antibody or peptide, i.e., amino acid residues within the “footprint” of the antibody. It is the simplest form or smallest structural area on a complex antigen molecule that can combine with e.g., an antibody or a receptor. Epitopes can be linear or conformational/structural. The term “linear epitope” is defined as an epitope composed of amino acid residues that are contiguous on the linear sequence of amino acids (primary structure). The term “conformational or structural epitope” is defined as an epitope composed of amino acid residues that are not all contiguous and thus represent separated parts of the linear sequence of amino acids that are brought into proximity to one another by folding of the molecule (secondary, tertiary and/or quaternary structures). A conformational epitope is dependent on the 3-dimensional structure. The term ‘conformational’ is therefore often used interchangeably with ‘structural’. Epitopes may be identified by different methods known in the art including but not limited to alanine scanning, phage display, X-ray crystallography, array-based oligo-peptide scanning or pepscan analysis, site-directed mutagenesis, high throughput mutagenesis mapping, H/D-Ex Mass Spectroscopy, homology modeling, docking, hydrogen-deuterium exchange, among others. (See e.g., Tong et al., Methods and Protocols for prediction of immunogenic epitopes”, Briefings in Bioinformatics 8(2):96-108; Gershoni, Jonathan M; Roitburd-Berman, Anna; Siman-Tov, Dror D; Tarnovitski Freund, Natalia; Weiss, Yael (2007). “Epitope Mapping”. BioDrugs 21 (3): 145-56; and Flanagan, Nina (May 15, 2011); “Mapping Epitopes with H/D-Ex Mass Spec: ExSAR Expands Repertoire of Technology Platform Beyond Protein Characterization”, Genetic Engineering & Biotechnology News 31 (10).

“Valent” or “valency” denotes the presence of a determined number of antigen-binding moieties in the antigen-binding protein. A natural IgG has two antigen-binding moieties and is bivalent. A molecule having one binding moiety for a particular antigen is monovalent for that antigen.

By “amino acid modification” herein is meant an amino acid substitution, insertion, and/or deletion in a polypeptide sequence. An example of amino acid modification herein is a substitution. By “amino acid modification” herein is meant an amino acid substitution, insertion, and/or deletion in a polypeptide sequence. By “amino acid substitution” or “substitution” herein is meant the replacement of an amino acid at a given position in a protein sequence with another amino acid. For example, the substitution Y50W refers to a variant of a parent polypeptide, in which the tyrosine at position 50 is replaced with tryptophan. Amino acid substitutions are indicated by listing the residue present in wild-type protein/position of residue/residue present in mutant protein. A “variant” of a polypeptide refers to a polypeptide having an amino acid sequence that is substantially identical to a reference polypeptide, typically a native or “parent” polypeptide. The polypeptide variant may possess one or more amino acid substitutions, deletions, and/or insertions at certain positions within the native amino acid sequence.

“Conservative” amino acid substitutions are those in which an amino acid residue is replaced with an amino acid residue having a side chain with similar physicochemical properties. Families of amino acid residues having similar side chains are known in the art, and include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine).

The term “identity” or “identical”, when used in a relationship between the sequences of two or more polypeptides, refers to the degree of sequence relatedness between polypeptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

Preferred methods for determining identity are designed to give the largest match between the sequences tested. Methods of determining identity are described in publicly available computer programs. Preferred computer program methods for determining identity between two sequences include the GCG program package, including GAP (Devereux et al., Nucl. Acid. Res. 12, 387 (1984); Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well-known Smith Waterman algorithm may also be used to determine identity.

An “isolated” molecule is a molecule that is the predominant species in the composition wherein it is found with respect to the class of molecules to which it belongs (i.e., it makes up at least about 50% of the type of molecule in the composition and typically will make up at least about 70%, at least about 80%, at least about 85%, at least about 90%, at least about 95%, or more of the species of molecule, e.g., peptide, in the composition). Commonly, a composition of a polypeptide will exhibit 98%, 98%, or 99% homogeneity for polypeptides in the context of all present peptide species in the composition or at least with respect to substantially active peptide species in the context of proposed use.

In the context herein, “treatment” or “treating” refers to preventing, alleviating, managing, curing or reducing one or more symptoms or clinically relevant manifestations of a disease or disorder, unless contradicted by context. For example, “treatment” of a patient in whom no symptoms or clinically relevant manifestations of a disease or disorder have been identified is preventive or prophylactic therapy, whereas “treatment” of a patient in whom symptoms or clinically relevant manifestations of a disease or disorder have been identified generally does not constitute preventive or prophylactic therapy.

As used herein, the phrase “NK cells” refers to a sub-population of lymphocytes that is involved in non-conventional immunity. NK cells can be identified by virtue of certain characteristics and biological properties, such as the expression of specific surface antigens including CD56 and/or NKp46 for human NK cells, the absence of the alpha/beta or gamma/delta TCR complex on the cell surface, the ability to bind to and kill cells that fail to express “self” MHC/HLA antigens by the activation of specific cytolytic machinery, the ability to kill tumor cells or other diseased cells that express a ligand for NK activating receptors, and the ability to release protein molecules called cytokines that stimulate or inhibit the immune response. Any of these characteristics and activities can be used to identify NK cells, using methods well known in the art. Any subpopulation of NK cells will also be encompassed by the term NK cells. Within the context herein “active” NK cells designate biologically active NK cells, including NK cells having the capacity of lysing target cells or enhancing the immune function of other cells. NK cells can be obtained by various techniques known in the art, such as isolation from blood samples, cytapheresis, tissue or cell collections, etc. Useful protocols for assays involving NK cells can be found in Natural Killer Cells Protocols (edited by Campbell K S and Colonna M). Humana Press. pp. 219-238 (2000).

As used herein, an agent that has “agonist” activity at NKp46 is an agent that can cause or increase “NKp46 signaling”. “NKp46 signaling” refers to an ability of an NKp46 polypeptide to activate or transduce an intracellular signaling pathway. Changes in NKp46 signaling activity can be measured, for example, by assays designed to measure changes in NKp46 signaling pathways, e.g. by monitoring phosphorylation of signal transduction components, assays to measure the association of certain signal transduction components with other proteins or intracellular structures, or in the biochemical activity of components such as kinases, or assays designed to measure expression of reporter genes under control of NKp46-sensitive promoters and enhancers, or indirectly by a downstream effect mediated by the NKp46 polypeptide (e.g. activation of specific cytolytic machinery in NK cells). Reporter genes can be naturally occurring genes (e.g. monitoring cytokine production) or they can be genes artificially introduced into a cell. Other genes can be placed under the control of such regulatory elements and thus serve to report the level of NKp46 signaling.

“NKp46” refers to a protein or polypeptide encoded by the Ncr1 gene or by a cDNA prepared from such a gene. Any naturally occurring isoform, allele, ortholog or variant is encompassed by the term NKp46 polypeptide (e.g., an NKp46 polypeptide 90%, 95%, 98% or 99% identical to SEQ ID NO 1, or a contiguous sequence of at least 20, 30, 50, 100 or 200 amino acid residues thereof). The 304 amino acid residue sequence of human NKp46 (isoform a) is shown below:

(SEQ ID NO: 1)
MSSTLPALLC VGLCLSQRIS AQQQTLPKPF IWAEPHFMVP
KEKQVTICCQ GNYGAVEYQL HFEGSLFAVD RPKPPERINK
VKFYIPDMNS RMAGQYSCIY RVGELWSEPS NLLDLVVTEM
YDTPTLSVHP GPEVISGEKV TFYCRLDTAT SMFLLLKEGR
SSHVQRGYGK VQAEFPLGPV TTAHRGTYRC FGSYNNHAWS
FPSEPVKLLV TGDIENTSLA PEDPTFPADT WGTYLLTTET
GLQKDHALWD HTAQNLLRMG LAFLVLVALV WFLVEDWLSR
KRTRERASRA STWEGRRRLN TQTL.

SEQ ID NO: 1 corresponds to NCBI accession number NP_004820, the disclosure of which is incorporated herein by reference. The human NKp46 mRNA sequence is described in NCBI accession number NM_004829, the disclosure of which is incorporated herein by reference.

### Producing Polypeptides

The proteins described herein can be conveniently configured and produced using well known immunoglobulin-derived domains, notably heavy and light chain variable domains, hinge regions, CH1, CL, CH2 and CH3 constant domains, and wild-type or variant cytokine polypeptides. Domains placed on a common polypeptide chain can be fused to one another either directly or connected via linkers, depending on the particular domains concerned. The immunoglobulin-derived domains will preferably be humanized or of human origin, thereby providing decreased risk of immunogenicity when administered to humans. As shown herein, advantageous protein formats are described that use minimal non-immunoglobulin linking amino acid sequences (e.g. not more than 4 or 5 domain linkers, in some cases as few as 1 or 2 domain linkers, and use of domains linkers of short length), thereby further reducing risk of immunogenicity.

Immunoglobulin variable domains are commonly derived from antibodies (immunoglobulin chains), for example in the form of associated V_{L} and V_{H} domains found on two polypeptide chains, or a single chain antigen binding domain such as a scFv, a V_{H} domain, a V_{L} domain, a dAb, a V-NAR domain or a V_{H}H domain. In certain advantageous proteins formats disclosed herein that directly enable the use of a wide range of variable regions from Fab or scFv without substantial further requirements for pairing and/or folding, the an antigen binding domain (e.g., ABD₁ and ABD₂) can also be readily derived from antibodies as a Fab or scFv.

The term “antigen-binding protein” can be used to refer to an immunoglobulin derivative with antigen binding properties. The binding protein comprises an immunologically functional immunoglobulin portion capable of binding to a target antigen. The immunologically functional immunoglobulin portion may comprise immunoglobulins, or portions thereof, fusion peptides derived from immunoglobulin portions or conjugates combining immunoglobulin portions that form an antigen binding site. Each antigen binding moiety comprises at least the necessarily one, two or three CDRs of the immunoglobulin heavy and/or light chains from which the antigen binding moiety was derived. In some aspects, an antigen-binding protein can consist of a single polypeptide chain (a monomer). In other embodiments the antigen-binding protein comprises at least two polypeptide chains. Such an antigen-binding protein is a multimer, e.g., dimer, trimer or tetramer. Examples of antigen binding proteins includes antibody fragments, antibody derivatives or antibody-like binding proteins that retain specificity and affinity for their antigen.

Typically, antibodies are initially obtained by immunization of a non-human animal, e.g., a mouse, rat, guinea pig or rabbit, with an immunogen comprising a polypeptide, or a fragment or derivative thereof, typically an immunogenic fragment, for which it is desired to obtain antibodies (e.g. a human polypeptide). The step of immunizing a non-human mammal with an antigen may be carried out in any manner well known in the art for stimulating the production of antibodies in a mouse (see, for example, E. Harlow and D. Lane, Antibodies: A Laboratory Manual, Cold Spring Harbor Laboratory Press, Cold Spring Harbor, NY (1988), the entire disclosure of which is herein incorporated by reference). Human antibodies may also be produced by using, for immunization, transgenic animals that have been engineered to express a human antibody repertoire (Jakobovitz et al. Nature 362 (1993) 255), or by selection of antibody repertoires using phage display methods. For example, a XenoMouse (Abgenix, Fremont, CA) can be used for immunization. A XenoMouse is a murine host that has had its immunoglobulin genes replaced by functional human immunoglobulin genes. Thus, antibodies produced by this mouse or in hybridomas made from the B cells of this mouse, are already humanized. The XenoMouse is described in U.S. Pat. No. 6,162,963, which is herein incorporated in its entirety by reference. Antibodies may also be produced by selection of combinatorial libraries of immunoglobulins, as disclosed for instance in (Ward et al. Nature, 341 (1989) p. 544, the entire disclosure of which is herein incorporated by reference). Phage display technology (McCafferty et al (1990) Nature 348:552-553) can be used to produce antibodies from immunoglobulin variable (V) domain gene repertoires from unimmunized donors. See, e.g., Griffith et al (1993) EMBO J. 12:725-734; U.S. Pat. Nos. 5,565,332; 5,573,905; 5,567,610; and 5,229,275). When combinatorial libraries comprise variable (V) domain gene repertoires of human origin, selection from combinatorial libraries will yield human antibodies.

In any embodiment, an antigen binding domain can be obtained from a humanized antibody in which residues from a complementary-determining region (CDR) of a human antibody are replaced by residues from a CDR of the original antibody (the parent or donor antibody, e.g. a murine or rat antibody) while maintaining the desired specificity, affinity, and capacity of the original antibody. The CDRs of the parent antibody, some or all of which are encoded by nucleic acids originating in a non-human organism, are grafted in whole or in part into the beta-sheet framework of a human antibody variable region to create an antibody, the specificity of which is determined by the engrafted CDRs. The creation of such antibodies is described in, e.g., WO 92/11018, Jones, 1986, Nature 321:522-525, Verhoeyen et al., 1988, Science 239:1534-1536. An antigen binding domain can thus have non-human hypervariable regions or CDRs and human frameworks region sequences (optionally with back mutations).

Additionally, a wide range of antibodies are available in the scientific and patent literature, including DNA and/or amino acid sequences, or from commercial suppliers. Antibodies will typically be directed to a pre-determined antigen. Examples of antibodies include antibodies that recognize an antigen expressed by a target cell that is to be eliminated, for example a proliferating cell or a cell contributing to a disease pathology. Examples include antibodies that recognize tumor antigens, microbial (e.g. bacterial or parasite) antigens or viral antigens.

Alternatively, antigen binding domains used in the proteins described herein can be readily derived from any of a variety of non-immunoglobulin scaffolds, for example affibodies based on the Z-domain of staphylococcal protein A, engineered Kunitz domains, monobodies or adnectins based on the 10th extracellular domain of human fibronectin III, anticalins derived from lipocalins, DARPins® (designed ankyrin repeat domains, multimerized LDLR-A module, avimers or cysteine-rich knottin peptides. See, e.g., Gebauer and Skerra (2009) Current Opinion in Chemical Biology 13:245-255, the disclosure of which is incorporated herein by reference.

As further exemplified herein, an antigen binding domain can conveniently comprise a VH and a VL (a VH/VL pair). In some embodiments, the VH/VL pair can be integrated in a Fab structure further comprising a CH1 and CL domain (a CH1/CL pair). A VH/VL pair refers to one VH and one VL domain that associate with one another to form an antigen binding domain. A CH1/CL pair refers to one CH1 and one CL domain bound to one another by covalent or non-covalent interactions, preferably non-covalent interactions, thus forming a heterodimer (e.g., within a protein such as a heterotrimer, heterotetramer, heteropentamer that can comprise one or more further polypeptide chains). The constant chain domains forming the pair can be present on the same or on different polypeptide chain, in any suitable combination.

Exemplary CDRs or VH and VL domains that bind NKp46 can be derived from the anti-NKp46 antibodies provided herein (see section “NKp46 variable region and CDR sequences”), or can be selected from any of the CDRs, VH and VL domains of PCT publication nos. WO2016/207278 and WO2017/114694, the disclosure of which are incorporated herein by reference. Variable regions can be used directly, or can be modified by selecting hypervariable or CDR regions from the NKp46 antibodies and placing them into the desired V_{L} or V_{H} framework, for example human frameworks. Antigen binding domains that bind NKp46 can also be derived de novo using methods for generating antibodies. Antibodies can be tested for binding to NKp46 polypeptides. In one aspect of any embodiment herein, a polypeptide (e.g. multispecific protein) that binds to NKp46 will be capable of binding NKp46 expressed on the surface of a cell, e.g. native NKp46 expressed by a NK cell.

Antigen binding domains (ABDs) that bind an antigen of interest can be selected based on the desired predetermined antigen of interest (e.g. an antigen other than NKp46), and may include for example cancer antigens such as antigens present on tumor cells and/or on immune cells capable of mediating a pro-tumoral effect, e.g. a monocyte or a macrophage, optionally a suppressor T cell, regulatory T cell, or myeloid-derived suppressor cell (for the treatment of cancer); bacterial or viral antigens (for the treatment of infectious disease); or antigens present on pro-inflammatory immune cells, e.g. T cells, neutrophils, macrophages, etc. (for the treatment of inflammatory and/or autoimmune disorder).

As used herein, the term “bacterial antigen” includes, but is not limited to, intact, attenuated or killed bacteria, any structural or functional bacterial protein or carbohydrate, or any peptide portion of a bacterial protein of sufficient length (typically about 8 amino acids or longer) to be antigenic. Examples include gram-positive bacterial antigens and gram-negative bacterial antigens. In some embodiments the bacterial antigen is derived from a bacterium selected from the group consisting of Helicobacter species, in particular Helicobacter pyloris; Borrelia species, in particular Borrelia burgdorferi; Legionella species, in particular Legionella pneumophilia; Mycobacteria s species, in particular M. tuberculosis, M. avium, M. intracellulare, M. kansasii, M. gordonae; Staphylococcus species, in particular Staphylococcus aureus; Neisseria species, in particular N. gonorrhoeae, N. meningitidis; Listeria species, in particular Listeria monocytogenes; Streptococcus species, in particular S. pyogenes, S. agalactiae; S. faecalis; S. bovis, S. pneumoniae; anaerobic Streptococcus species; pathogenic Campylobacter species; Enterococcus species; Haemophilus species, in particular Haemophilus influenzae; Bacillus species, in particular Bacillus anthracis; Corynebacterium species, in particular Corynebacterium diphtheriae; Erysipelothrix species, in particular Erysipelothrix rhusiopathiae; Clostridium species, in particular C. perfringens, C. tetani; Enterobacter species, in particular Enterobacter aerogenes, Klebsiella species, in particular Klebsiella 1S pneumoniae, Pasteurella species, in particular Pasteurella multocida, Bacteroides species; Fusobacterium species, in particular Fusobacterium nucleatum; Streptobacillus species, in particular Streptobacillus moniliformis; Treponema species, in particular Treponema pertenue; Leptospira; pathogenic Escherichia species; and Actinomyces species, in particular Actinomyces israeli.

As used herein, the term “viral antigen” includes, but is not limited to, intact, attenuated or killed whole virus, any structural or functional viral protein, or any peptide portion of a viral protein of sufficient length (typically about 8 amino acids or longer) to be antigenic. Sources of a viral antigen include, but are not limited to viruses from the families: Retroviridae (e.g., human immunodeficiency viruses, such as HIV-1 (also referred to as HTLV-III, LAV or HTLV-III/LAV, or HIV-III; and other isolates, such as HIV-LP; Picornaviridae (e.g., polio viruses, hepatitis A virus; enteroviruses, human Coxsackie viruses, rhinoviruses, echoviruses); Calciviridae (e.g., strains that cause gastroenteritis); Togaviridae (e.g., equine encephalitis viruses, rubella viruses); Flaviviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., Ebola viruses); Paramyxoviridae (e.g., parainfluenza viruses, mumps virus, measles virus, respiratory syncytial virus); Orthomyxoviridae (e.g., influenza viruses); Bunyaviridae (e.g., Hantaan viruses, bunya viruses, phleboviruses and Nairo viruses); Arenaviridae (hemorrhagic fever viruses); Reoviridae (e.g., reoviruses, orbiviruses and rotaviruses); Bornaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g., African swine fever virus); and unclassified viruses (e.g., the agent of delta hepatitis (thought to be a defective satellite of hepatitis B virus), Hepatitis C; Norwalk and related viruses, and astroviruses). Alternatively, a viral antigen may be produced recombinantly.

As used herein, the terms “cancer antigen” and “tumor antigen” are used interchangeably and refer to antigens (other than the cytokine receptor expressed on NK cells, NKp46, and CD16) that are differentially expressed by cancer cells or are expressed by non-tumoral cells (e.g. immune cells) having a pro-tumoral effect (e.g. an immunosuppressive effect), and can thereby be exploited in order to target cancer cells. Cancer antigens can be antigens which can potentially stimulate apparently tumor-specific immune responses. Some of these antigens are encoded, although not necessarily expressed, or expressed at lower levels or less frequently, by normal cells. These antigens can be characterized as those which are normally silent (i.e., not expressed) in normal cells, those that are expressed only at certain stages of differentiation and those that are temporally expressed such as embryonic and fetal antigens. Other cancer antigens are encoded by mutant cellular genes, such as oncogenes (e.g., activated ras oncogene), suppressor genes (e.g., mutant p53), fusion proteins resulting from internal deletions or chromosomal translocations. Still other cancer antigens can be encoded by viral genes such as those carried on RNA and DNA tumor viruses. Still other cancer antigens can be expressed on immune cells capable of contributing to or mediating a pro-tumoral effect, e.g. cell that contributes to immune evasion, a monocyte or a macrophage, optionally a suppressor T cell, regulatory T cell, or myeloid-derived suppressor cell.

The cancer antigens are usually normal cell surface antigens which are either over-expressed or expressed at abnormal times, or are expressed by a targeted population of cells. Ideally the target antigen is expressed only on proliferative cells (e.g., tumor cells) or pro-tumoral cells (e.g. immune cells having an immunosuppressive effect), however this is rarely observed in practice. As a result, target antigens are in many cases selected on the basis of differential expression between proliferative/disease tissue and healthy tissue. Example of cancer antigens include: Receptor Tyrosine Kinase-like Orphan Receptor 1 (ROR1), Crypto, CD4, CD19, CD20, CD30, CD38, CD47, Glycoprotein NMB, CanAg, Her2 (ErbB2/Neu), a Siglec family member, for example CD22 (Siglec2) or CD33 (Siglec3), CD79, CD123, CD138, CD171, PSCA, L1-CAM, PSMA (prostate specific membrane antigen), BCMA, CD52, CD56, CD80, CD70, E-selectin, EphB2, Melanotransferrin, Mud 6 and TMEFF2. Examples of cancer antigens also include Immunoglobulin superfamily (IgSF) such as cytokine receptors, Killer-Ig Like Receptor, CD28 family proteins, for example, Killer-Ig Like Receptor 3DL2 (KIR3DL2), B7-H3, B7-H4, B7-H6, PD-L1. Examples also include MAGE, MART-1/Melan-A, gp100, major histocompatibility complex class I-related chain A and B polypeptides (MICA and MICB), HLA-G, adenosine deaminase-binding protein (ADAbp), cyclophilin b, colorectal associated antigen (CRC)-C017-1A/GA733, protein tyrosine kinase 7 (PTK7), receptor protein tyrosine kinase 3 (TYRO-3), nectins (e.g. nectin-4), major histocompatibility complex class I-related chain A and B polypeptides (MICA and MICB), proteins of the UL16-binding protein (ULBP) family, proteins of the retinoic acid early transcript-1 (RAET1) family, carcinoembryonic antigen (CEA) and its immunogenic epitopes CAP-1 and CAP-2, etv6, aml1, prostate specific antigen (PSA), T-cell receptor/CD3-zeta chain, MAGE-family of tumor antigens, GAGE-family of tumor antigens, anti-Müllerian hormone Type II receptor, delta-like ligand 4 (DLL4), DR5, ROR1 (also known as Receptor Tyrosine Kinase-Like Orphan Receptor 1 or NTRKR1 (EC 2.7.10.1), BAGE, RAGE, LAGE-1, NAG, GnT-V, MUM-1, CDK4, MUC family, VEGF, VEGF receptors, Angiopoietin-2, PDGF, TGF-alpha, EGF, EGF receptor, members of the human EGF-like receptor family, e.g., HER-2/neu, HER-3, HER-4 or a heterodimeric receptor comprised of at least one HER subunit, gastrin releasing peptide receptor antigen, Muc-1, CA125, integrin receptors, αvß3 integrins, α5ß1 integrins, αIIbß3-integrins, PDGF beta receptor, SVE-cadherin, hCG, CSF1R (tumor-associated monocytes and macrophages), α-fetoprotein, E-cadherin, α-catenin, β-catenin and γ-catenin, p120ctn, PRAME, NY-ESO-1, cdc27, adenomatous polyposis coli protein (APC), fodrin, Connexin 37, Ig-idiotype, p15, gp75, GM2 and GD2 gangliosides, viral products such as human papillomavirus proteins, imp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, and c-erbB-2, although this is not intended to be exhaustive.

Optionally, a multispecific protein can be specified as excluding or not requiring a stromal modifying moiety, e.g., a moiety capable of altering or degrading a component of, the stroma such as an ECM component, e.g., a glycosaminoglycan, e.g., hyaluronan (also known as hyaluronic acid or HA), chondroitin sulfate, chondroitin, dermatan sulfate, heparin sulfate, heparin, entactin, tenascin, aggrecan and keratin sulfate; or an extracellular protein, e.g., collagen, laminin, elastin, fibrinogen, fibronectin, and vitronectin. For example, the stromal modifying moiety can be a hyaluronan degrading enzyme, an agent that inhibits hyaluronan synthesis, or an antibody molecule against hyaluronic acid. Optionally, a multispecific protein can be specified as excluding a mesothelin targeting moiety or mesothelin-binding ABD.

Optionally, a multispecific protein can be specified as excluding a PD-L1 targeting moiety, a HER3 targeting moiety, an IGFIR targeting moiety or a hyaluronidase 1 targeting moiety, or a combination a stroma targeting moiety or ABD and a cancer-antigen targeting moiety.

Optionally a cancer antigen or antigen of interest can be specified as being other than a PD-L1, a HER3, an IGFIR or hyaluronidase 1.

By way of examples, when the ABD that binds an antigen of interest binds to a HER2 polypeptide, exemplary VH and VL pairs can be selected from antibodies trastuzumab, pertuzumab or margetuximab:

(SEQ ID NO: 132)
Trastuzumab heavy chain variable region
EVQLVESGGG LVQPGGSLRL SCAASGFNIK DTYIHWVRQA
PGKGLEWVAR IYPTNGYTRY ADSVKGRFTI SADTSKNTAY
LQMNSLRAED TAVYYCSRWG GDGFYAMDYW GQGTLVTVSS.
(SEQ ID NO: 133)
Trastuzumab light chain variable region
DIQMTQSPSS LSASVGDRVT ITCRASQDVN TAVAWYQQKP
GKAPKLLIYS ASFLYSGVPS RFSGSRSGTD FTLTISSLQP
EDFATYYCQQ HYTTPPTFGQ GTKVEIK.
Margetuximab VH:
(SEQ ID NO: 134)
QVQLQQSGPE LVKPGASLKL SCTASGFNIK DTYIHWVKQR
PEQGLEWIGRIYPTNGYTRY DPKFQDKATI TADTSSNTAYL
QVSRLTSED TAVYYCSRWG GDGFYAMDYW GQGASVTVSS.
Margetuximab VL:
(SEQ ID NO: 135)
DIVMTQSHKF MSTSVGDRVS ITCKASQDVN TAVAWYQQKP
GHSPKLLIYS ASFRYTGVPD RFTGSRSGTD FTFTISSVQA
EDLAVYYCQQ HYTTPPTFGG GTKVEIK.

In another example, when the ABD that binds an antigen of interest binds to a CD19 polypeptide, exemplary VH and VL pairs can be selected from the VH and VL pair from blinatumomab.

Blinatumomab VH:
(SEQ ID NO: 136)
QVQLQQSGAELVRPGSSVKISCKASGYAFSSYWMNWVKQRPGQGLEWIGQ
IWPGDGDTNYNGKFKGKATLTADESSSTAYMQLSSLASEDSAVYFCARRE
TTTVGRYYYAMDYWGQGTTVTVSS.
Blinatumomab VL:
(SEQ ID NO: 137)
DIQLTQSPASLAVSLGQRATISCKASQSVDYDGDSYLNWYQQIPGQPPKL
LIYDASNLVSGIPPRFSGSGSGTDFTLNIHPVEKVDAATYHCQQSTEDPW
TFGGGTKLEIK.

In another example, when the ABD that binds an antigen of interest binds to a CD20 polypeptide, exemplary VH and VL pairs can be selected from VH and VL pair from rituximab and obinutuzumab:

### Rituximab VH:

(SEQ ID NO: 138)
QVQLQQPGAELVKPGASVKMSCKASGYTFTSYNMHWVKQTPGRGL
EWIGAIYPGNGDTSYNQKFKGKATLTADKSSSTAYMQLSSLTSED
SAVYYCARSTYYGGDWYFNVWGAGTTVTVSA.
Rituximab VL:
(SEQ ID NO: 139)
QIVLSQSPAILSASPGEKVTMTCRASSSVSYIHWFQQKPGSSPKP
WIYATSNLASGVPVRFSGSGSGTSYSLTISRVEAEDAATYYCQQW
TSNPPTFGGGTKLEIK.
Obinutuzumab VH:
(SEQ ID NO: 140)
QVQLVQSGAEVKKPGSSVKVSCKASGYAFSYSWINWVRQAPGQGL
EWMGRIFPGDGDTDYNGKFKGRVTITADKSTSTAYMELSSLRSED
TAVYYCARNVFDGYWLVYWGQGTLVTVSS.
Obinutuzumab VL:
(SEQ ID NO: 141)
DIVMTQTPLSLPVTPGEPASISCRSSKSLLHSNGITYLYWYLQKP
GQSPQLLIYQMSNLVSGVPDRFSGSGSGTDFTLKISRVEAEDVGV
YYCAQNLELPYTFGGGTKVEIK.

In another example, when the ABD that binds an antigen of interest binds to a EGFR polypeptide, exemplary VH and VL pairs can be selected from the EGFR-binding VH and VL pair from cetuximab, panitumumab, nimotuzumab, depatuxizumab and necitumumab:

Cetuximab VH:
(SEQ ID NO: 142)
QVQLKQSGPGLVQPSQSLSITCTVSGFSLTNYGVHWVRQSPGKGLEWLGV
IWSGGNTDYNTPFTSRLSINKDNSKSQVFFKMNSLQSNDTAIYYCARALT
YYDYEFAYWGQGTLVTVSA.
Cetuximab VL:
(SEQ ID NO: 143)
DILLTQSPVILSVSPGERVSFSCRASQSIGTNIHWYQQRTNGSPRLLIKY
ASESISGIPSRFSGSGSGTDFTLSINSVESEDIADYYCQQNNNWPTTFGA
GTKLELK.
Panitumumab VH:
(SEQ ID NO: 144)
QVQLQESGPGLVKPSETLSLTCTVSGGSVSSGDYYWTWIRQSPGKGLEWI
GHIYYSGNTNYNPSLKSRLTISIDTSKTQFSLKLSSVTAADTAIYYCVRD
RVTGAFDIWGQGTMVTVSS.
Panitumumab VL:
(SEQ ID NO: 145)
DIQMTQSPSSLSASVGDRVTITCQASQDISNYLNWYQQKPGKAPKLLIYD
ASNLETGVPSRFSGSGSGTDFTFTISSLQPEDIATYFCQHFDHLPLAFGG
GTKVEIK.
Nimotuzumab VH:
(SEQ ID NO: 146)
QVQLQQSGAEVKKPGSSVKVSCKASGYTFTNYYIYWVRQAPGQGLEWIGG
INPTSGGSNFNEKFKTRVTITADESSTTAYMELSSLRSEDTAFYFCTRQG
LWFDSDGRGFDFWGQGTTVTVSS.
Nimotuzumab VL:
(SEQ ID NO: 147)
DIQMTQSPSSLSASVGDRVTITCRSSQNIVHSNGNTYLDWYQQTPGKAPK
LLIYKVSNRFSGVPSRFSGSGSGTDFTFTISSLQPEDIATYYCFQYSHVP
WTFGQGTKLQI.
Necitumumab VH:
(SEQ ID NO: 148)
QVQLQESGPGLVKPSQTLSLTCTVSGGSISSGDYYWSWIRQPPGKGLEWI
GYIYYSGSTDYNPSLKSRVTMSVDTSKNQFSLKVNSVTAADTAVYYCARV
SIFGVGTFDYWGQGTLVTVSS.
Necitumumab VL:
(SEQ ID NO: 149)
EIVMTQSPATLSLSPGERATLSCRASQSVSSYLAWYQQKPGQAPRLLIYD
ASNRATGIPARFSGSGSGTDFTLTISSLEPEDFAVYYCHQYGSTPLTFGG
GTKAEIK.
Depatuxizumab VH:
(SEQ ID NO: 150)
QVQLQESGPGLVKPSQTLSLTCTVSGYSISSDFAWNWIRQPPGKGLEWMG
YISYSGNTRYQPSLKSRITISRDTSKNQFFLKLNSVTAADTATYYCVTAG
RGFPYWGQGTLVTVSS.
Depatuxizumab VL:
(SEQ ID NO: 151)
DIQMTQSPSSMSVSVGDRVTITCHSSQDINSNIGWLQQKPGKSFKGLIYH
GTNLDDGVPSRFSGSGSGTDYTLTISSLQPEDFATYYCVQYAQFPWTFGG
GTKLEIK.

In another example, when the ABD that binds an antigen of interest binds to a BCMA polypeptide, exemplary VH and VL pairs can be selected from the BCMA-binding VH and VL pair from belantamab, teclistamab, elranatamab or pavurutamab:

Belantamab VH:
(SEQ ID NO: 152)
QVQLVQSGAEVKKPGSSVKVSCKASGGTFSNYWMHWVRQAPGQGLEWMGA
TYRGHSDTYYNQKFKGRVTITADKSTSTAYMELSSLRSEDTAVYYCARGA
IYDGYDVLDNWGQGTLVTVSS.
Belantamab VL:
(SEQ ID NO: 153)
DIQMTQSPSSLSASVGDRVTITCSASQDISNYLNWYQQKPGKAPKLLIYY
TSNLHSGVPSRFSGSGSGTDFTLTISSLQPEDFATYYCQQYRKLPWTFGQ
GTKLEIK.
Pavurutamab VH:
(SEQ ID NO: 154)
QVQLVQSGAEVKKPGASVKVSCKASGYTFTNHIIHWVRQAPGQCLEWMGY
INPYPGYHAYNEKFQGRATMTSDTSTSTVYMELSSLRSEDTAVYYCARDG
YYRDTDVLDYWGQGTLVTVSS.
Pavurutamab VL:
(SEQ ID NO: 155)
DIQMTQSPSSLSASVGDRVTITCQASQDISNYLNWYQQKPGKAPKLLIYY
TSRLHTGVPSRFSGSGSGTDFTFTISSLEPEDIATYYCQQGNTLPWTFGC
GTKVEIK.

In another example, when the ABD that binds an antigen of interest binds to a PD-L1 polypeptide, exemplary VH and VL pairs can be selected from the PD-L1-binding VH and VL pair from antibodies 3G10, 12A4, 10A5, 5F8, 10H10, 1B12, 7H1, 11E6, 12B7, and 13G4 shown in U.S. Pat. No. 7,943,743, the disclosure of which is incorporated herein by reference, or of any of the antibodies MPDL3280A (atezolizumab, Tecentriq™, see, e.g., U.S. Pat. No. 8,217,149, anti-PD-L1 from Roche/Genentech), MDX-1105 (anti-PD-L1 from Bristol-Myers Squibb), MSB0010718C (avelumab; anti-PD-L1 from Pfizer) and MED14736 (durvalumab; anti-PD-L₁ from AstraZeneca).

In another example, when the ABD that binds an antigen of interest binds to a B7-H3 polypeptide, exemplary VH and VL pairs can be selected from the B7-H3-binding VH and VL pairs of enoblituzumab, of TRL4542 shown in PCT publication no. WO2018/129090, of 8H9 shown in PCT publication no. WO2018/209346, or of any of the antibodies of PCT publication nos. WO2016/106004, WO2017/180813, WO2019/024911, WO2019/225787, WO2020/063673, WO2020/094120, WO2020/102779, WO2020/140094 and WO2020/151384. Examples of single domain B7H3 ABDs that can be used include Affibody™ formats described in PCT publication WO2020/041626 and single domain antibodies (sdAb) of PCT publication nos. WO2020/076970 and WO2021/247794. The disclosures of VH, VL and CDRs sequences of the above are incorporated herein by reference.

In another example, when the ABD that binds an antigen of interest binds to a B7-H6 polypeptide, exemplary VH and VL pairs can be selected from the B7-H6-binding VH and VL pairs shown in U.S. Pat. Nos. 11,034,766; 8,822,652; 9,676,855; 11,034,766; 11,034,767 or in PCT publication nos. WO2013/037727 or WO2021/064137.

In another example, when the ABD that binds an antigen of interest binds to a B7-H4 polypeptide, exemplary VH and VL pairs can be selected from the B7-H4-binding VH and VL of alsevalimab or the VH and VL pairs shown in U.S. Pat. Nos. 10,626,176; 9,676,854; 9,574,000; 10,150,813; 10,814,011 or in PCT publication nos. WO2009/073533, WO2019/165077, WO2019/169212, WO2019/147670, WO2021/155307, WO2022/039490, WO2019/154315 or WO2021/185934.

In one embodiment, the ABD that binds an antigen of interest binds to a cancer antigen, a viral antigen, a microbial antigen, or an antigen present on an infected cell (e.g. virally infected) or on a pro-inflammatory immune cell. In one embodiment, said antigen is a polypeptide selectively expressed or overexpressed on a tumor cell, and infected cell or a pro-inflammatory cell. In one embodiment, said antigen is a polypeptide that when inhibited, decreases the proliferation and/or survival of a tumor cell, an infected cell or a pro-inflammatory cell.

The ABDs which are incorporated into the polypeptides can be tested for any desired activity prior to inclusion in a multispecific NKp46-binding protein, for example the ABD can be tested in a suitable format (e.g. as conventional IgG antibody, fab, Fab′2 or scFv) for binding to (e.g. binding affinity) for its binding partner.

An ABD derived from an antibody will generally comprise at minimum a hypervariable region sufficient to confer binding activity. It will be appreciated that an ABD may comprise other amino acids or functional domains as may be desired, including but not limited to linker elements (e.g. linker peptides, CH1, Cκ or Cλ domains, hinges, or fragments thereof). In one example an ABD comprises a scFv, a V_{H} domain and a V_{L} domain, or a single domain antibody (nanobody or dAb) such as a V-NAR domain or a V_{H}H domain. ABDs can be made of a V_{H} and a V_{L} domain that associate with one another to form the ABD.

In one embodiment, one or both of the V_{H} and V_{L} pairs that form an ABD for NKp46 and antigen of interest are within a tandem variable region (a V_{H} and V_{L} domain separated by a flexible polypeptide linker), such as an scFv.

In one embodiment, one or both ABDs for NKp46 and antigen of interest can have a conventional or non-conventional Fab structure. A Fab structure can be characterized as a VH or VL variable domain linked to a CH1 domain and a complementary variable domain (VL or VH, respectively) linked to a complementary Cκ (or Cλ) constant domain, wherein the CH1 and Cκ (or Cλ) constant domains associate (dimerize). For example a Fab can be formed from a VH-CH1 unit (VH fused to a CH1) on a first polypeptide chain that dimerizes with a VL-Cκ unit (VL fused to a Cκ) on a second chain. Alternatively, a Fab can be formed from a VH-Cκ unit (VH fused to a Cκ) on a first polypeptide chain that dimerizes with a VL-CH1 unit (VL fused to a CH1) on a second chain.

In some embodiments, one of the ABDs for NKp46 and antigen of interest comprises a Fab structure, in which a variable domain is linked to a CH1 domain and a complementary variable domain is linked to a complementary Cκ (or Cλ) constant domain, wherein the CH1 and Cκ (or Cλ) constant domains associate to form a heterodimeric protein, and the other ABD comprise or consists of an scFv or a single binding domain (e.g. VhH domain, Affibody™, DARPin). The scFv or a single binding domain can optionally be fused to a Cκ or CA domain or hinge domain.

The CH1 and/or Cκ domains can then be linked to a CH2 domain, optionally in each case via a hinge region (or a suitable domain linker). The CH2 domain(s) is/are then linked to a CH3 domain. The CH2-CH3 domains can thus optionally be embodied as a full-length Fc domain (optionally a full-length Fc domain, except that the CH3 domain that lacks the C-terminal lysine).

The CD16 ABD, when present, can readily be embodied as a Fc domain dimer that is capable of binding to human CD16A and optionally other Fcγ receptors, e.g., CD16B, CD32A, CD32B and/or CD64). In one embodiment, an Fc moiety may be obtained by production of the polypeptide in a host cell or by a process that yields N297-linked glycosylation, e.g. a mammalian cell. In one embodiment, an Fc moiety comprises a human gamma isotype constant region comprising one or more amino acid modifications, e.g. in the CH2 domain, that increases binding to CD16 or CD16A.

Alternatively, the CD16A ABD, when present, can comprise the amino acid sequences of the CD16A-binding VH and VL pair of SEQ ID NOS: 504 and 505, or of the CD16A-binding VH and VL pair of SEQ ID NOS: 506 and 507, or can comprise the heavy and light chain Kabat CDRs thereof. Yet further, the alternatively, the CD16A ABD, when present, can be embodied as a CD16A-binding single VH domain (see, e.g., Genbank accession no. ABQ52435; Behar et al., (2008) Protein Eng Des Sel. (1):1-10) having the amino acid sequence shown in SEQ ID NO: 508.

The cytokine receptor antigen binding domain can readily be embodied as a cytokine, (e.g. a type 1 cytokine such as an IL-2, IL-15, IL-21, IL-7, IL-27 or IL-12 cytokine, an IL-18 cytokine or a type 1 interferon such as IFN-α or IFN-β). Exemplary cytokine receptor ABDs and modified cytokines are further described herein.

Once appropriate antigen binding domains having desired specificity and/or activity are identified, nucleic acids encoding each of the or ABD can be separately placed, in suitable arrangements, in an appropriate expression vector or set of vectors, together with DNA encoding any elements such as CH1, CK, CH2 and CH3 domains or portions thereof, mutant IL2 polypeptides and any other optional elements (e.g. DNA encoding a hinge-derived or linker elements) for transfection into an appropriate host. ABDs will be arranged in an expression vector, or in separate vectors as a function of which type of polypeptide is to be produced, so as to produce the Fc-polypeptides having the desired domains operably linked to one another. The host is then used for the recombinant production of the multispecific polypeptide.

For example, a polypeptide fusion product can be produced from a vector in which one ABD or a part thereof (e.g. a VH, VL or a VH/VL pair) is operably linked (e.g. directly, or via a CH1, Cκ or Cλ constant region and/or hinge region) to the N-terminus of a CH2 domain, and the CH2 domain is operably linked at its C-terminus to the N-terminus a CH3 domain. Another ABD or part thereof can be on a second polypeptide chain that forms a dimer, e.g. heterodimer, with the polypeptide comprising the first ABD.

The multispecific polypeptide can then be produced in an appropriate host cell or by any suitable synthetic process. A host cell chosen for expression of the multispecific polypeptide is an important contributor to the final composition, including, without limitation, the variation in composition of the oligosaccharide moieties decorating the protein in the immunoglobulin CH2 domain. Thus, one aspect of the invention involves the selection of appropriate host cells for use and/or development of a production cell expressing the desired therapeutic protein such that the multispecific polypeptide retains FcRn and CD16 binding. The host cell may be of mammalian origin or may be selected from COS-1, COS-7, HEK293, BHK21, CHO, BSC-1, Hep G2, 653, SP2/0, 293, HeLa, myeloma, lymphoma, yeast, insect or plant cells, or any derivative, immortalized or transformed cell thereof. The host cell may be any suitable species or organism capable of producing N-linked glycosylated polypeptides, e.g. a mammalian host cell capable of producing human or rodent IgG type N-linked glycosylation.

Protein Formats

Multimeric, multispecific proteins such as heterodimers, heterotrimers and hetero-tetramers can be produced according to a variety of formats. Different domains onto different polypeptide chain that associate to form a multimeric protein. Accordingly, a wide range of protein formats can be constructed around Fc domain dimers that are capable of binding to human FcRn polypeptide (neonatal Fc receptor), with or without additionally binding to CD16 or CD16A and optionally other Fcγ receptors, e.g., CD16B, CD32A, CD32B and/or CD64), depending on whether or not the CD16 binding ABD is desired to be present. As shown herein, greatest potentiation of NK cell cytotoxicity can be obtained through use of Fc moieties that have substantial binding to the activating human CD16 receptor (CD16A) binding; such CD16 binding can be obtained through the use of suitable CH2 and/or CH3 domains, as further described herein. In one embodiment, an Fc moiety is derived from a human IgG1 isotype constant region. Use of modified CH3 domains also contributes to the possibility of use a wide range of heteromultimeric protein structures. Accordingly, a protein comprises a first and a second polypeptide chain each comprising a variable domain fused to a human Fc domain monomer (i.e. a CH2-CH3 unit), optionally a Fc domain monomer comprising a CH3 domain capable of undergoing preferential CH3-CH3 hetero-dimerization, wherein the first and second chain associate via CH3-CH3 dimerization and the protein consequently comprises a Fc domain dimer. The variable domains of each chain can be part of the same or different antigen binding domains.

Multispecific proteins can thus be conveniently constructed using VH and VL pairs arranged as scFv or Fab structures, together with CH1 domains, CL domain, Fc domains and cytokines, and domain linkers. Preferably, the proteins will use minimal non-natural sequences, e.g. minimal use of non-Ig linkers, optionally no more than 5, 4, 3, 2 or 1 domain linker(s) that is not an antibody-derived sequence, optionally wherein domain linker(s) are no more than 15, 10 or 5 amino acid residues in length. In one embodiment, the CD16 ABD is a Fc domain dimer. Fc domain dimerFc domain dimer.

In some embodiment, the multispecific proteins (e.g. dimers, trimers, tetramers) may comprise a domain arrangement of any of the following in which domains can be placed on any of the 2, 3 or 4 polypeptide chains, wherein the NKp46 ABD is interposed between the Fc domain and the cytokine receptor ABD (e.g. the protein has a terminal or distal cytokine receptor ABD at the C-terminal end and a terminal or distal antigen of interest (Antigen) ABD at the topological N-terminal end), wherein the NKp46 ABD is connected to one of the polypeptide chains of the Fc domain dimer via a hinge polypeptide or a flexible linker, and wherein the ABD that binds the cytokine receptor is connected to NKp46 ABD (e.g. to one of the polypeptide chains thereof when the NKp46 ABD is contained on two chains) via a flexible linker (e.g. a linker comprising G and S residues), wherein “n” is 1 or 2:

(Anti-Antigen ABD)ₙ-(Fc domain dimer)-(NKp46ABD)-(cytokine receptor ABD).

The cytokine receptor ABD can be an IL2, IL15, IL18, IL21 or IFN-α polypeptide. The Fc domain can be specified to be a Fc domain dimer (e.g. that binds human FcRn and/or Fcγ receptors). In one embodiment, one or both of the antigen of interest (e.g. cancer antigen) ABD and NKp46 ABD is formed from two variable regions present within tandem variable regions, wherein the variable regions that associate to form a particular ABD can be on the same polypeptide chain or on different polypeptide chains. In another embodiment, one or both of the antigen of interest ABD and NKp46 ABD comprises a tandem variable region and the other comprises a Fab structure. In another embodiment, both of the antigen of interest and NKp46 ABD comprises a Fab structure. In another embodiment one of the antigen of interest and NKp46 ABD comprises a Fab structure and the other comprises an scFv structure.

Heterodimers and Heterotrimers

The present disclosure provides advantageous approaches of making multimeric multispecific proteins which bind to the antigen of interest (monovalently or bivalently) and monovalently to each of NKp46, CD16A and cytokine receptor. The approaches readily allow domain configurations where the NKp46 ABD is positioned between the Fc domain and a cytokine polypeptide. These configurations can be achieved through the assembly of different polypeptide chains described herein that each comprise at least one heavy or light chain variable domain fused to a human CH1 or Cκ constant domain (a V-(CH1/Cκ) unit), wherein the protein chains undergo CH1-Cκ dimerization and are bound to one another by non-covalent interactions and optionally further disulfide bonds formed between respective CH1 and Cκ domains.

Exemplary heterodimeric or heterotrimeric polypeptides have an NKp46 binding ABD, an antigen of interest binding ABD, a cytokine receptor-binding ABD (e.g. IL-2, IL-15, IL-21, IL-7, IL-27, IL-12, IL-18, IFN-α or IFN-β polypeptide) and a Fc domain dimer can optionally be produced as one or more chains that each associate with a central chain, e.g. by CH1-Cκ heterodimerization and/or by CH3-CH3 dimerization. Different variants can be produced, as illustrated in the Examples herein.

In one embodiment, an isolated or purified heterodimeric or heterotrimeric protein comprises at least two or three polypeptide chains, each comprising a V-(CH1/Cκ) unit, whereby the chains are bound to one another by non-covalent interactions and optionally further bound via disulfide bonds between CH1 and Cκ domains, and still further optionally, whereby the chains are bound by non-covalent interactions between the respective variable regions, CH1 and Cκ domains, and CH3 domains of the Fc portion.

In one example, the protein comprises a first and a second polypeptide chain each comprising a variable domain fused to a CH1 or Cκ domain (a V-(CH1/Cκ) unit), in turn fused at its C-terminus to a human Fc domain monomer comprising a CH2 domain and a CH3 domain capable of undergoing CH3-CH3 dimerization, wherein the first and second chain associate via CH1-Cκ and CH3-CH3 dimerization such that the protein comprises a Fc domain dimer. The variable domains of each chain can be part of the same or different antigen binding domains.

The variable and constant regions can be selected and configured such that each chain will preferentially associate with its desired complementary partner chain. The resulting multimeric protein can be produced reliably and with high productivity using conventional production methods using recombinant host cells. The choice of which V_{H} or V_{L} to associate with a CH1 and Cκ in a unit is based on affinity between the units to be paired so as to drive the formation of the desired multimer. The resulting multimer will be bound by non-covalent interactions between complementary V_{H} and V_{L} domains, by non-covalent interactions between complementary CH1 and Cκ domains, and optionally by further disulfide bonding between complementary CH1 and Cκ domains (and optionally further disulfide bonds between complementary hinge domains). V_{H}-V_{L} associations are stronger than V_{H}-V_{H} or V_{L}-V_{L}, consequently, as shown herein, one can place a V_{H} or a V_{L} next to either a CH1 or a Cκ, and the resulting V-C unit will partner preferably with its V-C counterpart. For example V_{H}-Cκ will pair with V_{L}-CH1 preferentially over V_{H}-CH1. Additionally, by including an Fc domain, preferred chain pairing is further improved, as the two Fc monomer-containing chains are bound by non-covalent interactions between CH3 domains of the Fc domain monomers. The different V-C combinations, optionally further combined with Fc pairing thereby provides tools to make heteromultimeric proteins comprising a cytokine (e.g. IL-2, IL-15, IL-21, IL-7, IL-27, IL-12, IL-18, IFN-α or IFN-β polypeptide), represented as “Cyt” in the domain arrangements.

In one example, the multispecific protein is a heterodimer comprising a first and a second polypeptide chain each comprising a variable domain fused to a CH1 or Cκ domain (a V-(CH1/Cκ) unit), in turn fused at its C-terminus to a human Fc domain monomer, wherein the V-(CH1/Cκ) unit of the first chain has undergone CH1-Cκ dimerization with the V-(CH1/Cκ) unit of the second chain thereby forming a first antigen binding domain (ABD₁) and a Fc domain dimer, wherein one of the polypeptide chains further comprises an antigen binding domain that forms a second antigen binding domain (ABD₂), and wherein the Fc domain dimer binds to a human CD16 polypeptide, wherein one of ABD₁ and ABD₂ binds NKp46 and the other binds the antigen of interest (e.g. tumor antigen).

In one example, the protein has a domain arrangement:

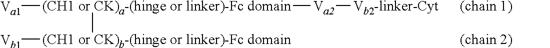

wherein Vₐ₋₁, V_{b-1}, Vₐ₋₂ and V_{b-2} are each a V_{H} domain or a V_{L} domain, and wherein one of Vₐ₋₁ and V_{b-1} is a V_{H} and the other is a V_{L} such that Vₐ₋₁ and V_{b-1} form a first antigen binding domain (ABD), wherein one of Vₐ₋₂ and V_{b-2} is a V_{H} and the other is a V_{L} such that Vₐ₋₂ and V_{b-2} form a second antigen binding domain, wherein one of the ABD binds NKp46 and the other binds an antigen of interest.

In other examples, the protein is a heterotrimer and comprises three polypeptide chains, each comprising a variable domain fused to a CH1 or Cκ domain (a V-(CH1/Cκ) unit), wherein a first (central) chain comprises two V-(CH1/Cκ) units and a human Fc domain interposed between the units, the second chain comprises one V-(CH1/Cκ) unit and a human Fc domain monomer, and the third chain comprises one V-(CH1/Cκ) unit and a cytokine polypeptide (Cyt), wherein one of the V-(CH1/Cκ) units of the central chain has undergone CH1-Cκ dimerization with the V-(CH1/Cκ) unit of the second chain thereby forming a first antigen binding domain (ABD₁) and a Fc domain dimer, and wherein the other of the V-(CH1/Cκ) units of the central chain has undergone CH1-Cκ dimerization with the V-(CH1/Cκ) unit of the third chain thereby forming a second antigen binding domain (ABD₂), and wherein the Fc domain binds to a human CD16 polypeptide. In one embodiment, the Fc domain comprises N-linked glycosylation at residue N297 (Kabat EU numbering).

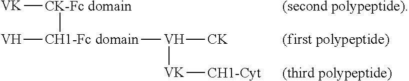

In one example, the protein has a domain arrangement:

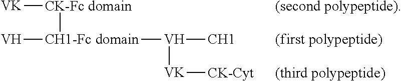

In another example, the protein has a domain arrangement:

In one specific example of the above heterotrimeric proteins, the Fab structure interposed between the Fc domain and the cytokine is the NKp46 binding ABD (i.e. the NKp46 binding ABD is interposed between the Fc domain and the C-terminal cytokine). The Fc domain in the first polypeptide is connected (e.g. fused) at its C terminus to the N-terminus of the VH domain via a linker. The constant domain (the CH1 or Cκ domain in the respective domain arrangements) in the third polypeptide is connected (e.g. fused) at its C terminus to the N-terminus of the cytokine polypeptide via a linker. Each constant domain (the CH1 or Cκ domain in the respective domain arrangements) that is N-terminal to the Fc domain is fused at the C terminus of the constant domain to the N-terminus of the Fc domain via a hinge region.

Optionally, any of the multispecific proteins of the invention may include CH1, CL or CH3 domains which comprise amino acid modifications (e.g. substitutions) to promote heterodimerization. For example, heterodimerization modifications often involve steric repulsion, charge steering interaction, or interchain disulfide bond formation, wherein the CH3 domain interface of the antibody Fc region is mutated to create altered charge polarity across the Fc dimer interface such that co-expression of electrostatically matched Fc chains supports favorable attractive interactions thereby promoting desired Fc heterodimer formation, whereas unfavorable repulsive charge interactions suppress unwanted Fc homodimer formation.

In one example, the first (central) polypeptide chain will provide one variable domain that will, together with a complementary variable domain on a second polypeptide chain, form a first antigen binding domain (e.g. the ABD that binds the antigen of interest), and an Fc domain. The first (central) polypeptide chain will also provide a second variable domain (e.g., placed on the opposite end of the interposed Fc domain from the first variable domain, at the C-terminus of the Fc domain) that will be paired with a complementary variable domain to form a second antigen binding domain (e.g. the ABD that binds NKp46); the variable domain that is complementary to the second variable domain can be placed on the central polypeptide (e.g. adjacent to the second variable domain in a tandem variable domain construct such as an scFv), or can be placed on a separate polypeptide chain, notably a third polypeptide chain. The second (and third, if present) polypeptide chains will associate with the central polypeptide chain by CH1-Cκ heterodimerization, forming non-covalent interactions and optionally further interchain disulfide bonds between complementary CH1 and Cκ domains (and optionally interchain disulfide bonds between hinge regions), with a primary multimeric polypeptide being formed so long as CH/CK and V_{H}/Vκ domains are chosen to give rise to a preferred dimerization configuration that results preferentially in the desired V_{H}-V_{L} pairings. Remaining unwanted pairings can remain minimal during production and/or are removed during purification steps. In a trimer, or when polypeptides are constructed for preparation of a trimer, there will generally be one polypeptide chain that comprises a non-naturally occurring VH-CK or VK-CH1 domain arrangement. A cytokine (e.g., IL-2, IL-15, IL-21, IL-7, IL-27, IL-12, IL-18, IFN-α or IFN-β) can then be placed at the C-terminus of one of polypeptide chains. The cytokine can be fused via a domain linker, and while not shown in certain domain arrangements herein, any domain arrangement can be specified as comprising a domain linker separating two domains. For example, in these structures, the cytokine can be placed at the C-terminus of the first (central) polypeptide chain or at the C-terminus of the third polypeptide chain (when such third chain is present).

Examples of the domain arrangements (N- to C-terminus, left to right) of central polypeptide chains for use in heterodimeric proteins in which the NKp46 ABD is interposed between the Fc domain and cytokine (Cyt) and wherein the cytokine moiety is placed on a different polypeptide chain (e.g. the second polypeptide chain) include any of the following, wherein each V is a variable domain:

Vₐ₋₁-(CH1 or Cκ)-Fc domain-Vₐ₋₂-V_{b-2} (first/central chain)

or

Vₐ₋₁-V_{b}-Fc domain-Vₐ₋₂-(CH1 or Cκ) (first/central chain)

Further examples of the domain arrangements of central polypeptide chains for use in heterodimeric proteins wherein the cytokine moiety is placed on the central chain include:

Vₐ₋₁-(CH1 or Cκ)-Fc domain-Vₐ₋₂-V_{b-2}-Cyt (first/central chain)

or

Vₐ₋₁-V_{b-1}-Fc domain-Vₐ₋₂-(CH1 or Cκ)-Cyt (first/central chain)

Examples of domain arrangements (N- to C-termini from left to right) of central polypeptide chains for use in heterotrimeric proteins in which the NKp46 ABD is interposed between the Fc domain and cytokine (Cyt), and wherein the cytokine moiety is placed on a different polypeptide chain (e.g. the second or third polypeptide chain) can include:

Vₐ₋₁-(CH1 or Cκ)ₐ-Fc domain-Vₐ₋₂-(CH1 or Cκ) (first/central chain)

Further examples of the domain arrangements of central polypeptide chains for use in heterotrimeric proteins wherein the cytokine moiety is placed on the central chain include:

Vₐ₋₁-(CH1 or Cκ)ₐ-Fc domain-Vₐ₋₂-(CH1 or Cκ)-Cyt (first/central chain)

In the above examples, when V domains are arranged immediately adjacent to one another in tandem on a chain, one of the V is a light chain and the other V is heavy chain variable domain, and the two V domains are separated by a flexible polypeptide linker and together form a scFv.

Further examples of central polypeptide chains include:

Vₐ₋₁-(CH1 or Cκ)ₐ-Fc domain-V₂-Cyt

or

V₂-Fc domain-Vₐ₋₁-(CH1 or Cκ)-Cyt
wherein V₂ is a single domain ABD (e.g. dAb, VHH, DARPin).

The Fc domain of the central chain may be a full Fc domain or a portion thereof sufficient to confer the desired functionality (e.g. binding to FcRn, binding to CD16, CH3-CH3 dimerization) when it forms a dimeric Fc with the Fc domain of a second polypeptide chain.

A second polypeptide chain can then be configured which will comprise an immunoglobulin variable domain and a CH1 or Cκ constant region, e.g., a (CH1 or Cκ)_{b} unit, selected so as to permit CH1-Cκ heterodimerization with the central polypeptide chain; the immunoglobulin variable domain will be selected so as to complement the variable domain of the central chain that is adjacent to the CH1 or Cκ domain, whereby the complementary variable domains form an antigen binding domain for a first antigen of interest.

For example, a second polypeptide chain for use in a protein in which the NKp46 ABD is interposed between Fc and cytokine can comprise a domain arrangement:

V_{b-1}-(CH1 or Cκ)_{b}-Fc domain
such that the (CH1 or Cκ)_{b} dimerizes with the (CH1 or Cκ)ₐ on the central chain, and the V_{b-1} forms an antigen binding domain together with Vₐ₋₁ of the central chain. If the Vₐ₋₁ of the central chain is a light chain variable domain, then V_{b-1} will be a heavy chain variable domain; and if Vₐ₋₁ of the central chain is a heavy chain variable domain, then V_{b-1} will be a light chain variable domain.

In heterodimers, the antigen binding domain for the second antigen of interest can then be formed from Vₐ₋₂ and V_{b-2} which are configured as tandem variable domains on the central chain forming an ABD (e.g. forming an scFv unit).

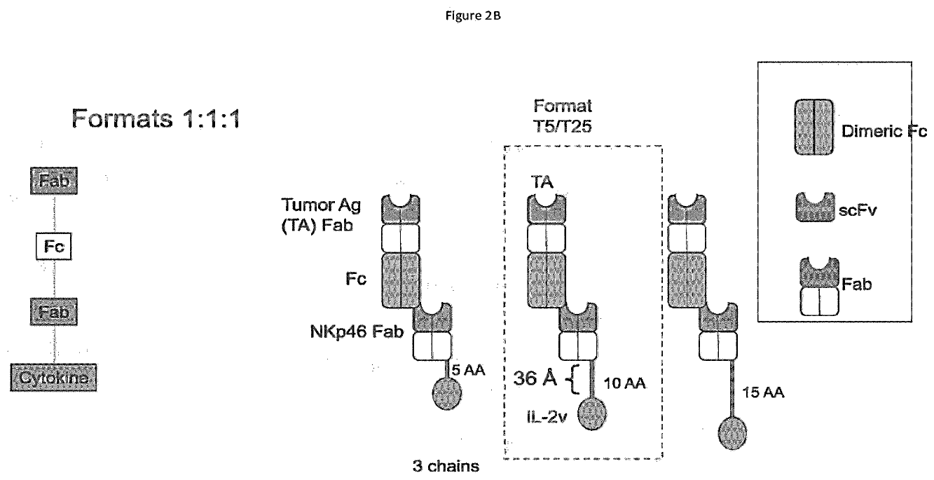

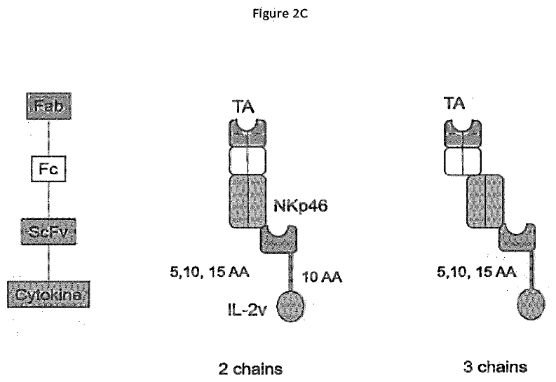

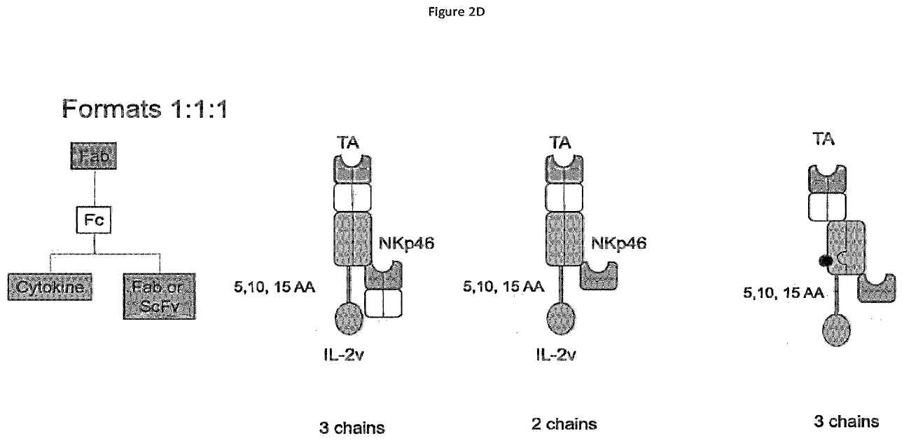

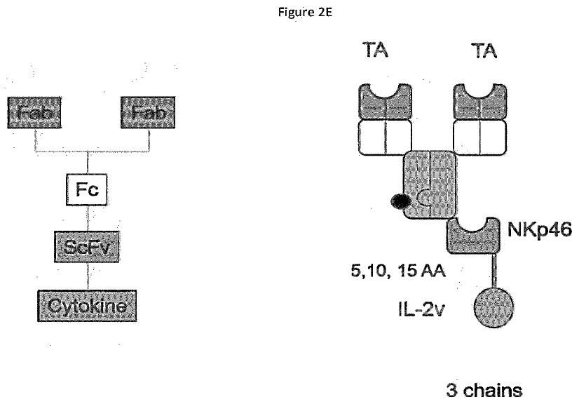

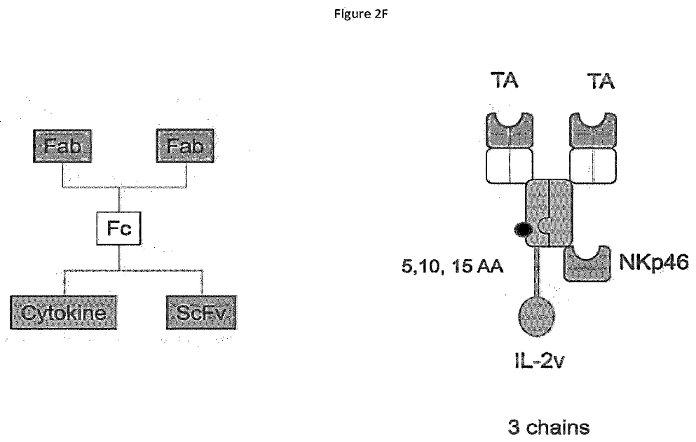

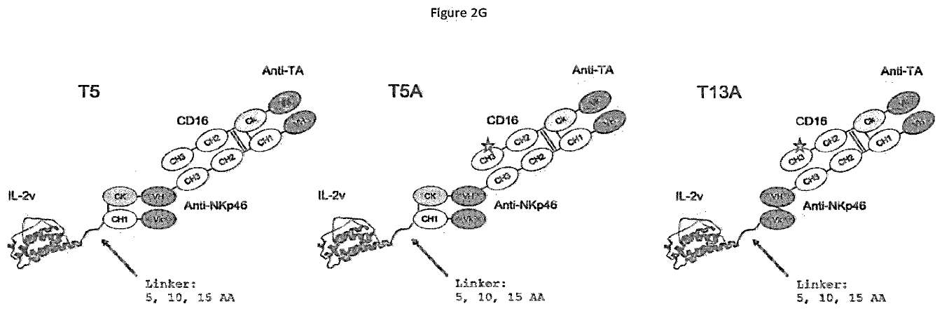

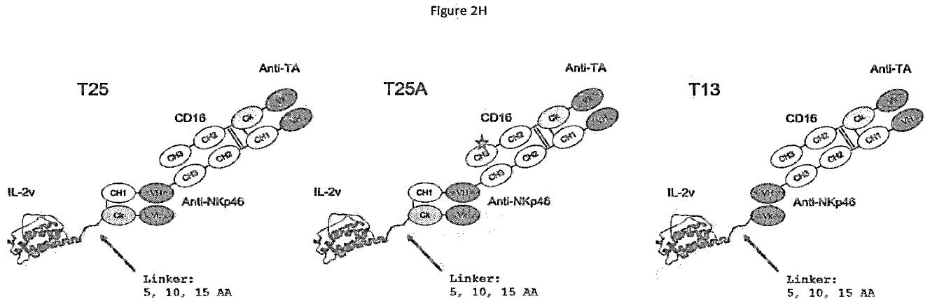

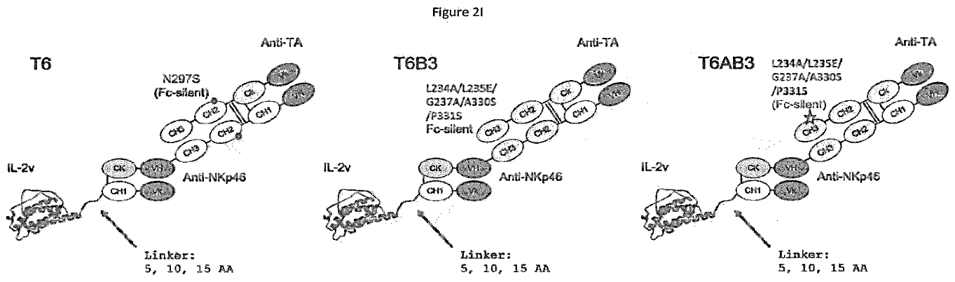

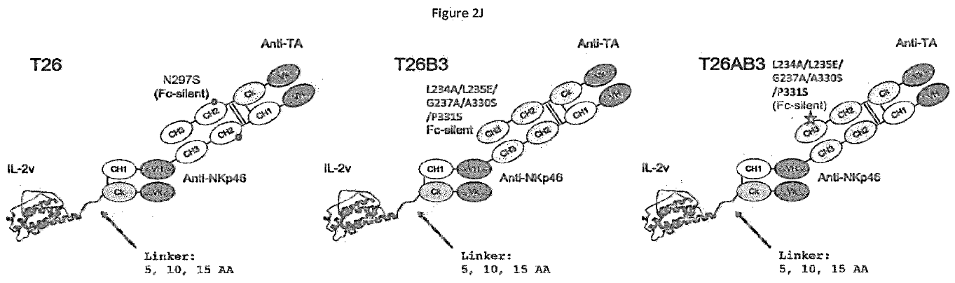

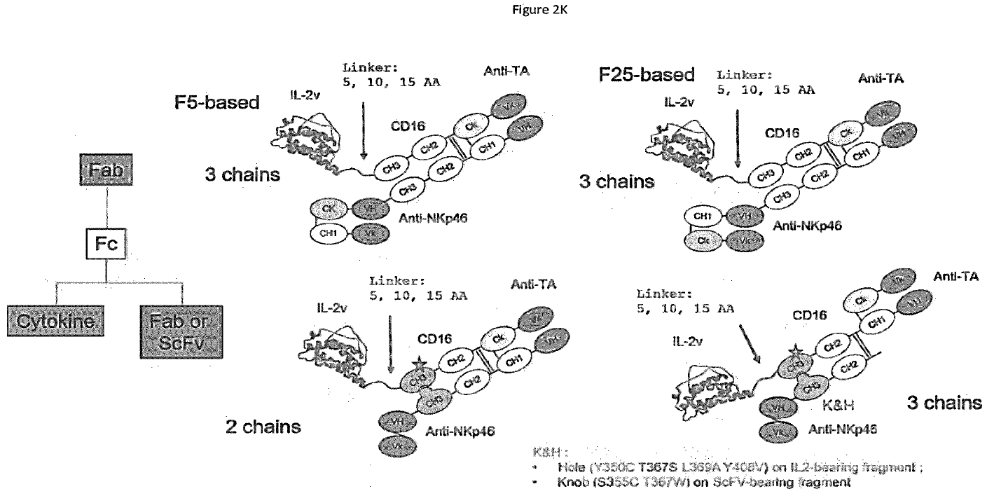

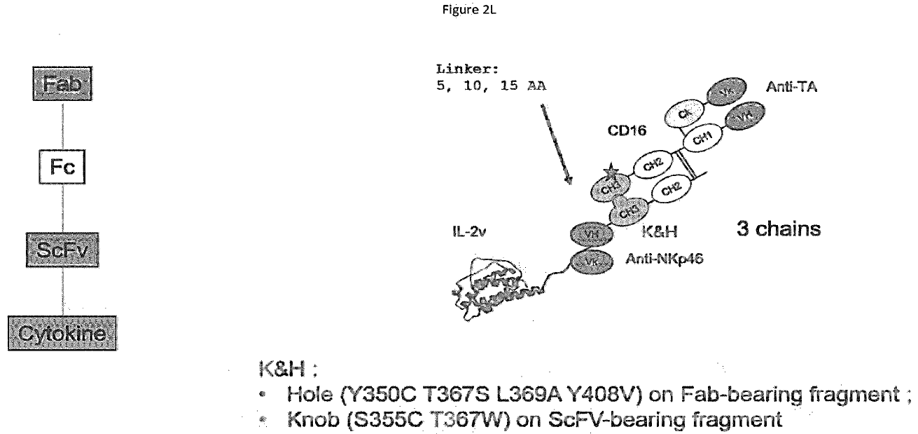

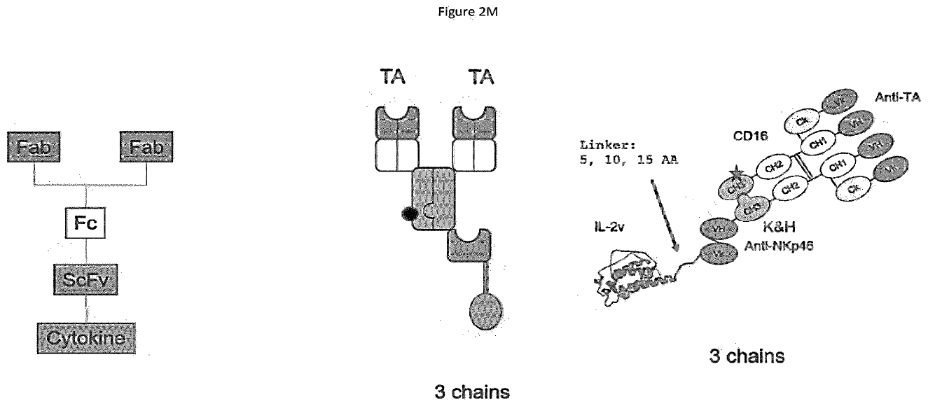

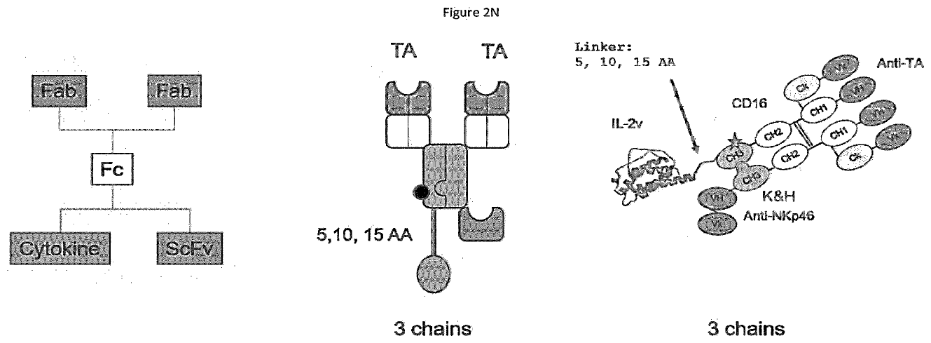

The resulting heterodimer can, for example, have the following configuration (see further examples of such proteins shown as formats T13 and T13A shown in FIGS. 2C, 2D and 2G and 2H:

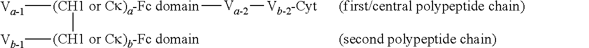

wherein one of Vₐ₋₁ of the first polypeptide chain and V_{b-1} of the second polypeptide chain is a light chain variable domain and the other is a heavy chain variable domain, and wherein one of Vₐ₋₂ and V_{b-2} is a light chain variable domain and the other is a heavy chain variable domain. Vₐ₋₂ and V_{b-2} can be specified as being separated by a polypeptide linker (Vₐ₋₂ and V_{b-2} form an scFv). Vₐ₋₂ and V_{b-2} forms the ABD that binds NKp46 and Vₐ₋₁ and V_{b-1} forms the ABD that binds the antigen of interest (e.g. cancer antigen).

Examples of domain arrangements of heteromultimeric proteins include the following, optionally wherein one or both of the hinge domains are replaced by a flexible linker polypeptide, wherein the NKp46 ABD is an scFv or a single domain ABD (e.g. dAb, VHH, DARPin) and the Fc domain is fused to the NKp46 ABD a linker polypeptide, and wherein the NKp46 ABD is fused to the cytokine polypeptide by a domain linker (e.g. a flexible polypeptide linker):

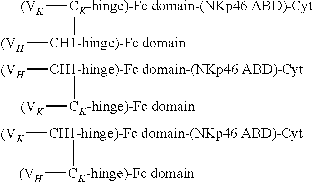

Heterotrimeric proteins in which the NKp46 ABD is interposed between the Fc domain and the cytokine polypeptide can for example be formed by using a central (first) polypeptide chain comprising a first variable domain (V) fused to a first CH1 or Cκ constant region, a second variable domain (V) fused to a second CH1 or Cκ constant region, and an Fc domain or portion thereof interposed between the first and second variable domains (i.e. the Fc domain is interposed between the first and second (V-(CH1/Cκ) units. For example, a central polypeptide chain for use in a heterotrimeric protein according to the invention can have the domain arrangements (N- to C-termini) as follows:

Vₐ₋₁-(CH1 or Cκ)ₐ-Fc domain-Vₐ₋₂-(CH1 or Cκ)_{b}.

The first polypeptide chain can optionally further have a Cyt is placed at its C-terminus.

A second polypeptide chain can then comprise a domain arrangement (N- to C-termini from left to right):

V_{b-1}-(CH1 or Cκ)_{c}-Fc domain
such that the (CH1 or Cκ)_{c} dimerizes with the (CH1 or Cκ)ₐ on the central chain, and the Vₐ₋₁ and V_{b-1} form an antigen binding domain that binds the antigen of interest.

A third polypeptide chain can then comprise the following domain arrangement (N- to C-termini from left to right):

V_{b-2}-(CH1 or Cκ)_{d}-Cyt.
such that the (CH1 or Cκ)_{d} dimerizes with the (CH1 or Cκ)_{b} unit on the central chain, and the Vₐ₋₂ and V_{b-2} form the NKp46 binding domain.

Optionally, where the Cyt is placed at the C-terminus of the first polypeptide chain, the third polypeptide chain can then comprise the following domain arrangement (N- to C-termini from left to right):

V_{b-2}-(CH1 or Cκ)_{d}

An example of a domain configuration of a resulting heterotrimer where the Cyt is placed on the third polypeptide chain (also shown as formats T5, T6 in FIG. 2) is shown below:

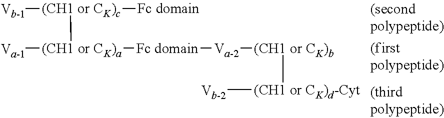

A domain configuration of a resulting heterotrimer where the Cyt is placed on the first polypeptide chain is shown below:

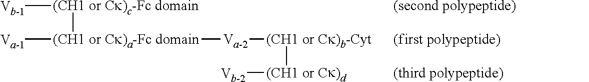

Thus, in a trimeric polypeptide in which the NKp46 ABD is interposed between the Fc domain and the cytokine polypeptide, the first polypeptide can have two variable domains that each form an antigen binding domain with a variable domain on a separate polypeptide chain (i.e. the variable domain of the second and third chains), the second polypeptide chain has one variable domain, and the third polypeptide has one variable domain, and one of the polypeptide chains comprises a cytokine polypeptide fused to its C-terminus.

A trimeric polypeptide can optionally comprise three polypeptide chains characterized as follows:
(a) a first polypeptide chain comprising from N-terminus to C-terminus: a first variable domain (V) fused to a first CH1 or Cκ constant region, a hinge domain or portion thereof, an Fc domain or portion thereof, and a second variable domain (V) fused to a second CH1 or Cκ constant region;
(b) a second polypeptide chain comprising from N-terminus to C-terminus: a variable domain fused to a CH1 or Cκ constant region selected to be complementary to the first CH1 or Cκ constant region of the first polypeptide chain such that the first and second polypeptides form a CH1-Cκ heterodimer, a hinge domain or portion thereof, and an Fc domain; and
(c) a third polypeptide chain comprising from N-terminus to C-terminus: a variable domain fused to a CH1 or Cκ constant region, and a cytokine polypeptide (e.g. fused to the constant region via a flexible polypeptide linker), wherein the variable domain and the constant region are selected to be complementary to the second variable domain and second CH1 or Cκ constant region of the first polypeptide chain such that the first and third polypeptides form a CH1-Cκ heterodimer bound by non-covalent interactions and optionally further disulfide bond(s) formed between the CH1 or Cκ constant region of the third polypeptide and the second CH1 or Cκ constant region of the first polypeptide, but not between the CH1 or Cκ constant region of the third polypeptide and the first CH1 or Cκ constant region of the first polypeptide
wherein the first, second and third polypeptides form a heterotrimer, and wherein the first variable domain of the first polypeptide chain and the variable domain of the second polypeptide chain form an antigen binding domain specific for an antigen of interest (e.g. on a target cell, a cancer antigen), and the second variable domain of the first polypeptide chain and the variable domain on the third polypeptide chain form an antigen binding domain that binds NKp46.

Examples of potential domain arrangements for such trimeric bispecific polypeptides include but are not limited to those shown in Table 2 below:

TABLE 2

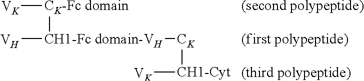

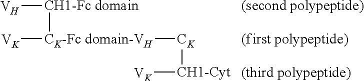

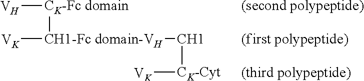

In another aspect, a trimeric polypeptide can optionally be characterized as comprising three polypeptide chains:
(a) a first polypeptide chain comprising from N-terminus to C-terminus: a first variable domain (V) fused to a first CH1 or Cκ constant region, a hinge domain or portion thereof, an Fc domain or portion thereof, and a second variable domain (V) fused to a second CH1 or Cκ constant region, and a cytokine polypeptide (e.g. fused to the second CH1 or Cκ constant region via a flexible polypeptide linker);
(b) a second polypeptide chain comprising from N-terminus to C-terminus: a variable domain fused to a CH1 or Cκ constant region selected to be complementary to the first CH1 or Cκ constant region of the first polypeptide chain such that the first and second polypeptides form a CH1-Cκ heterodimer, a hinge domain or portion thereof, and an Fc domain; and
(c) a third polypeptide chain comprising from N-terminus to C-terminus: a variable domain fused to a CH1 or Cκ constant region, wherein the variable domain and the constant region are selected to be complementary to the second variable domain and second CH1 or Cκ constant region of the first polypeptide chain such that the first and third polypeptides form a CH1-Cκ heterodimer bound by non-covalent interactions and optionally further disulfide bond(s) formed between the CH1 or Cκ constant region of the third polypeptide and the second CH1 or Cκ constant region of the first polypeptide, but not between the CH1 or Cκ constant region of the third polypeptide and the first CH1 or Cκ constant region of the first polypeptide
wherein the first, second and third polypeptides form a CH1-Cκ heterotrimer, and wherein the first variable domain of the first polypeptide chain and the variable domain of the second polypeptide chain form an antigen binding domain specific for an antigen of interest (e.g. on a target cell, a cancer antigen), and the second variable domain of the first polypeptide chain and the variable domain on the third polypeptide chain form an antigen binding domain that binds NKp46.

Examples of potential domain arrangements for such trimeric bispecific polypeptides include but are not limited to those shown in Table 3 below:

TABLE 3

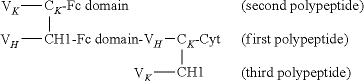

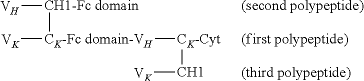

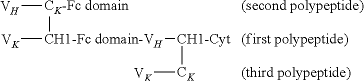

Using similar architecture, yet further multispecific proteins can be produced that binds the antigen of interest bivalently, binds NKp46 monovalently and binds the cytokine receptor monovalently, i.e., the multispecific protein has a 2:1:1 configuration. Examples are shown in FIG. 2.

In one example of a multispecific protein having two ABDs that each binds an antigen of interest, a heterodimer protein comprises the domain arrangement:

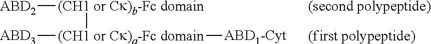

In these structures, the Fc domains of the first and second chains associate via CH3-CH3 dimerization, and (CH1 or Cκ)_{b} on the second chain and the (CH1 or Cκ)ₐ on the first chain undergo CH1-Cκ dimerization, wherein ABD₁ that binds NKp46 and ABD₂ and ABD₃ are each self-contained antigen binding domains that can bind an antigen of interest (e.g. a cancer antigen) without association with a complementary domain on a different polypeptide chain, wherein each (CH1 or Cκ)_{b} and (CH1 or Cκ)ₐ is fused to the Fc domain via an immunoglobulin hinge amino acid sequence, and wherein Cyt is a cytokine polypeptide (e.g. fused to the ABD₁ via a flexible polypeptide linker). Another representation of the heterodimeric protein is:

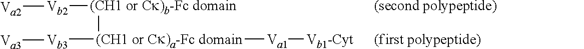

where each V domain pair Vₐ₁-V_{b1}, Vₐ₂-V_{b2} and Vₐ₃-V_{b3} comprises a VH and VL fused via a domain linker such the pair forms an scFv antigen binding domain.

In an example of a multispecific protein having two ABDs that each binds an antigen of interest, a heterotrimer protein comprises the domain arrangement:

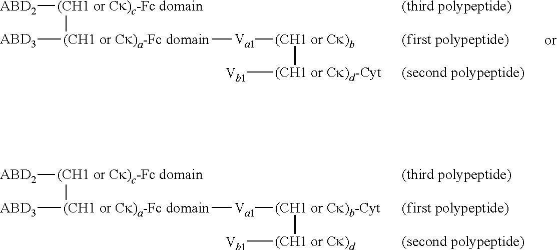

In these structures, the Fc domains of the first and third chains associate via CH3-CH3 dimerization, (CH1 or Cκ)_{c} on the third chain and the (CH1 or Cκ)ₐ on the central chain undergo CH1-Cκ dimerization, and the (CH1 or Cκ)_{b} on the first chain and the (CH1 or Cκ)_{d} on the second chain undergo CH1-Cκ dimerization. The Vₐ₁ and V_{b1} form a first antigen binding domain that bind NKp46. ABD₂ and ABD₃ are each self-contained antigen binding domains that can bind to an antigen of interest (e.g. a cancer antigen), e.g. without requiring association with a complementary variable domain on a different polypeptide chain. ABD₂ and ABD₃ can for example each comprise a single domain ABD or a VH and a Vκ pair (in any desired order), placed on a single chain and separated by a flexible peptide linker (e.g. as an scFv), such that a heterotrimer protein can comprise:

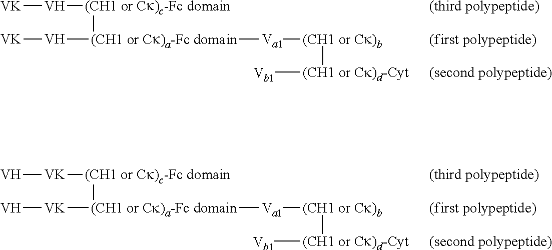

Examples of possible configurations of a resulting heterotrimer are structures having domain arrangement, from N- to C-terminus:

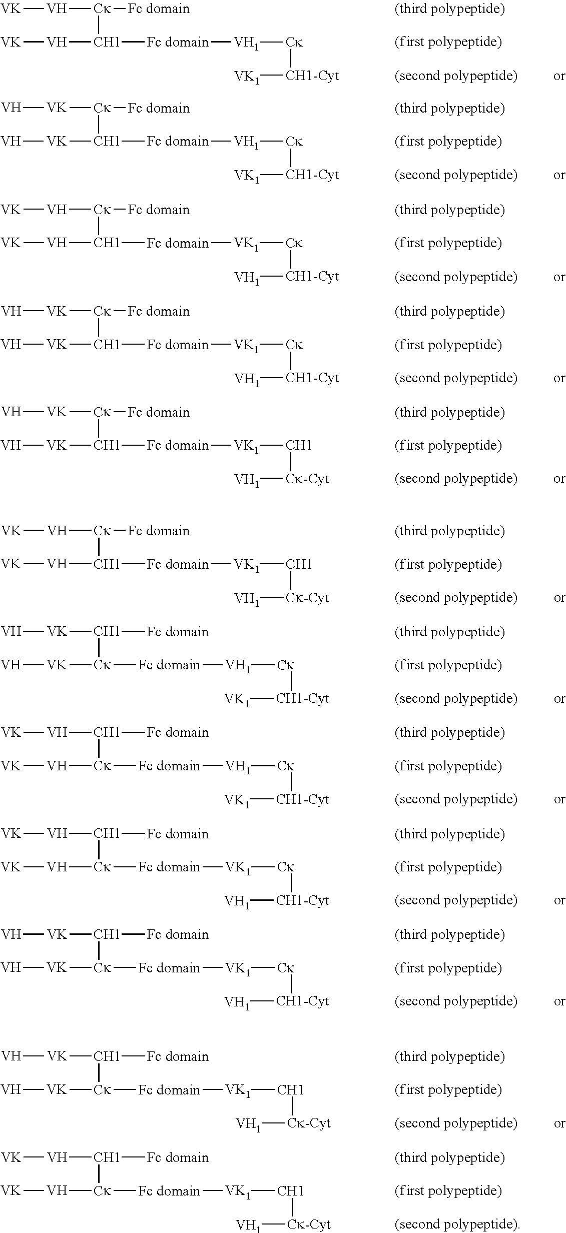

In another example of a multispecific protein having two ABDs that each binds an antigen of interest, a heterotetramer protein can be constructed in which the NKp46 ABD is interposed between the Fc domain and the cytokine, for example molecules having the following domain arrangement, wherein a domain:

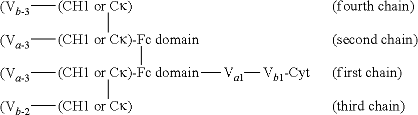

wherein the first chain and the second chain associate by CH3-CH3 dimerization and the first chain and the third chain associate by the CH1 or Cκ dimerization, wherein the domains of the first chain and the third chain are selected to be complementary to permit the first and third chains to associate by CH1-Cκ dimerization, wherein the domains of the second chain and the fourth chain are selected to be complementary to permit the second and fourth chains to associate by CH1-Cκ dimerization, and wherein each V domain pair Vₐ₁ and V_{b1}, Vₐ₂ and V_{b2} and Vₐ₃ and V_{b3} comprises a VH and a VL such the pair forms ABD, wherein Vₐ₋₁ and V_{b-1} are separated by a domain linker to form an scFv that binds NKp46, and wherein the Vₐ₁ and V_{b1} pair and the Vₐ₂ and V_{b2} pair each forms and ABD that binds an antigen of interest.

Examples of such configurations include shown in the Table 4 below:

TABLE 4

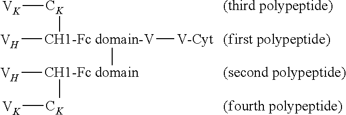

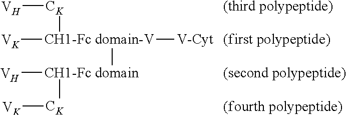

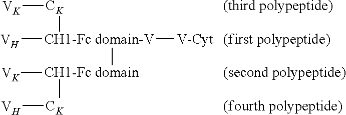

The domain arrangements can also be respectively represented as follows, in which each L is a domain linker:

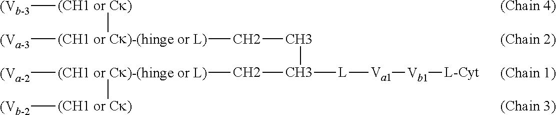

When the NKp46 ABD and the cytokine receptor ABD are positioned in “cis” with respect to the N- and C-termini of Fc domain (e.g., both are placed on the C-terminal side of the dimeric Fc) they will preferably be positioned so as to enhance the ability to bind NKp46, CD16A and cytokine receptor in a membrane planar binding conformation. In one embodiment, a cis configuration is obtained by positioning the NKp46 ABD (e.g. as a scFv) on a polypeptide chain comprising an Fc domain, where the NKp46 ABD is positioned at the C-terminus of the Fc domain (cis), and positioning the cytokine receptor ABD (e.g. cytokine) topologically C-terminal to the NKp46 ABD. In one embodiment, a cis configuration (with respect to the termini of the Fc domain) is obtained by positioning a portion of a NKp46 binding Fab on a first polypeptide chain comprising an Fc domain, and positioning the cytokine receptor ABD (e.g. cytokine) on the first chain or on a second polypeptide chain that associates with the first chain and comprises a complementary portion of the NKp46 binding Fab, optionally wherein the second polypeptide chain lacks an Fc domain. In an exemplary cis configuration, a first portion of a NKp46 ABD can be positioned on a first polypeptide chain comprising an Fc domain, and the cytokine receptor ABD (e.g. cytokine) is positioned on a different second polypeptide chain that lacks an Fc domain but that has a complementary second portion of the NKp46 ABD, where the second polypeptide chain and second portion associates with the first polypeptide chain and first portion to form a NKp46 ABD (e.g. an NKp46 binding Fab). A portion of an ABD or Fab can for example be a VH or VL domain thereof, a VH-CH1, Vκ-CH1, VL-CL or VL-CL domain. It can optionally be specified that the cytokine receptor ABD is positioned adjacent and C-terminal of the complementary VH-CH1, Vκ-CH1, VL-CL or VL-CL component of the NKp46 ABD on the first or second chain, or adjacent and C-terminal of the Fc domain on the first chain.

In any embodiment, it may be specified that the protein has a Fc domain dimer comprised of a first and second Fc domain monomer placed on separate chains that dimerize via CH3-CH3 association, wherein one of the Fc domain monomers is connected to the both the anti-NKp46 ABD and the cytokine, and the other (second) Fc domain monomer has a free C-terminus (no anti-NKp46 ABD or cytokine fused to its C-terminus).

Optionally in any embodiment herein, fusions or linkages on the same polypeptide chain between different domains (e.g., between two V domains placed in tandem, between V domains and CH1 or Cκ domains, between CH1 or Cκ domains and Fc domains, between Fc domains monomers and V domains, between Fc domain monomers and cytokine) may occur via intervening amino acid sequences, for example via a hinge region or linker peptide. Generally, domain arrangements or structures herein are depicted without showing domain linkers, and it will be appreciated that the domain arrangements can be specified as having domain linkers between a specified domain. For example, the cytokine can be specified as being fused to an adjacent domain via a domain linker, and a domain linker can be inserted in the relevant domain arrangement or structure. In another example, tandem variable domains (e.g. in an scFv) can be specified as being fused to one another via a domain linker, and a domain linker can be inserted between the two V regions in the relevant domain arrangement or structure. In another example, a CH1 or CL (or Cκ) constant region can be fused to an Fc domain or CH2 domain thereof via a domain linker or hinge domain or portion thereof, and accordingly a domain linker or hinge domain or portion thereof can be inserted between CH1 or CL domain and the Fc domain or CH2 domain in the relevant domain arrangement or structure. An example of the domain arrangement of a multispecific protein with linkers shown is shown in FIG. 2A for the representative heterotrimer in format “T5”, shows domain linkers such as hinge and glycine-serine linkers, and interchain disulfide bridges.

In any embodiment herein, a polypeptide chain (e.g., chain 1, 2, 3 or 4) can be specified as having a free N and/or C terminus (no other protein domains at the terminus of the polypeptide chain).

In any embodiment herein, the proteins domains described herein can optionally be specified as being indicated from N- to C-termini. Protein arrangements of the disclosure for purposes of illustration are shown from N-terminus (on the left) to C-terminus (on the right). Adjacent domains on a polypeptide chain can be referred to as being fused to one another (e.g. a domain can be said to be fused to the C-terminus of the domain on its left, and/or a domain can be said to be fused to the N-terminus of the domain on its right). The proteins domains described herein can be fused to one another directly (e.g. V domains fused directly to CH1 or CL domains) or via linkers or short intervening amino acid sequences that serve to connect the domains on a polypeptide chain (e.g. they may optionally be specified to lack other predetermined functionality, or to lack specific binding to a predetermined ligand). Two polypeptide chains will be bound to one another (indicated by “i”), by non-covalent interactions, and optionally can further be attached via interchain disulfide bonds, formed between cysteine residues within complementary CH1 and Cκ domains.

### Connections and Linkers

Generally, there are a number of suitable linkers that can be used in the multispecific proteins, including traditional peptide bonds, generated by recombinant techniques. In some embodiments, the linker is a “domain linker”, used to link any two domains as outlined herein together. Adjacent protein domains can be specified as being connected or fused to one another by a domain linker. An exemplary domain linker is a (poly)peptide linker, optionally a flexible (poly)peptide linker. Peptide linkers or polypeptide linkers, used interchangeably herein, may have a subsequence derived from a particular domain such as a hinge, CH1 or CL domain, or may predominantly include the following amino acid residues: Gly, Ser, Ala, or Thr. The linker peptide should have a length that is adequate to link two molecules in such a way that they assume the correct conformation relative to one another so that they retain the desired activity. In one embodiment, the linker is from about 1 to 50 amino acids in length, preferably about 2 to 30 amino acids in length. In one embodiment, linkers of 4 to 20 amino acids in length may be used, with from about 5 to about 15 amino acids finding use in some embodiments. While any suitable linker can be used, many embodiments, linkers (e.g. flexible linkers) can utilize a glycine-serine polypeptide or polymer, including for example comprising (GS)ₙ, (GSGGS)ₙ, (GGGGS)ₙ, (GSSS)ₙ, (GSSSS)ₙ and (GGGS)ₙ, where n is an integer of at least one (optionally n is 1, 2, 3 or 4), glycine-alanine polypeptide, alanine-serine polypeptide, and other flexible linkers. Linkers comprising glycine and serine residues generally provides protease resistance. One example of a (GS)₁ linker is a linker having the amino acid sequence STGS; such a linker can be useful to fuse a domain to the C-terminus of an Fc domain (or a CH3 domain thereof). In some embodiments peptide linkers comprising (G₂S)ₙ are used, wherein, for example, n=1-20, e.g., (G₂S), (G₂S)₂, (G₂S)₃, (G₂S)₄, (G₂S)₅, (G₂S)₆, (G₂S)₇ or (G₂S)₈, or (G₃S)ₙ, wherein, for example, n is an integer from 1-15. In one embodiment, a domain linker comprises a (G₄S)ₙ peptide, wherein, for example, n is an integer from 1-10, optionally 1-6, optionally 1-4. In some embodiments peptide linkers comprising (GS₂)ₙ (GS₃)ₙ or (GS₄)ₙ are used, wherein, for example, n=1-20, e.g., (GS₂), (GS₂)₂, (GS₂)₃, (GS₃)₁, (GS₃)₂, (GS₃)₃, (GS₄)₁, (GS₄)₂, (GS₄)₃, wherein, for example, n is an integer from 1-15. In one embodiment, a domain linker comprises a (GS₄)ₙ peptide, wherein, for example, n is an integer from 1-10, optionally 1-6, optionally 1-4. In one embodiment, a domain linker comprises a C-terminal GS dipeptide, e.g., the linker comprises (GS₄) and has the amino acid sequence a GSSSS, GSSSSGSSSS, GSSSSGSSSSGS or GSSSSGSSSSGSSSS.

Any of the peptide or domain linkers may be specified to comprise a length of at least 2 residues, 3 residues, 4 residues, at least 5 residues, at least 10 residues, at least 15 residues, at least 20 residues, or more. In other embodiments, the linkers comprise a length of between 2-4 residues, between 2-4 residues, between 2-6 residues, between 2-8 residues, between 2-10 residues, between 2-12 residues, between 2-14 residues, between 3-15 residues, between 4-15 residues, between 2-16 residues, between 2-18 residues, between 2-20 residues, between 2-22 residues, between 2-24 residues, between 2-26 residues, between 2-28 residues, between 2-30 residues, between 2 and 50 residues, or between 10 and 50 residues.

Examples of polypeptide linkers may include sequence fragments from CH1 or CL domains; for example the first 4-12 or 5-12 amino acid residues of the CL/CH1 domains are particularly useful for use in linkages of scFv moieties. Linkers can be derived from immunoglobulin light chains, for example CK or Cλ. Linkers can be derived from immunoglobulin heavy chains of any isotype, including for example Cy1, Cy2, Cy3, Cy4 and Cμ. Linker sequences may also be derived from other proteins such as Ig-like proteins (e.g. TCR, FcR, KIR), hinge region-derived sequences, and other natural sequences from other proteins. In certain domain arrangements, V_{H} and V_{L} domains are linked to another in tandem separated by a linker peptide (e.g. a scFv) and in turn be fused to the N- or C-terminus of an Fc domain (or CH2 domain thereof). Such tandem variable regions or scFv can be connected to the Fc domain via a hinge region or a portion thereof, an N-terminal fragment of a CH1 or CL domain, or a glycine- and serine-containing flexible polypeptide linker.

Fc domains can be connected to other domains via immunoglobulin-derived sequence or via non-immunoglobulin sequences, including any suitable linking amino acid sequence. Advantageously, immunoglobulin-derived sequences can be readily used between CH1 or CL domains and Fc domains, in particular, where a CH1 or CL domain is fused at its C-terminus to the N-terminus of an Fc domain (or CH2 domain). An immunoglobulin hinge region or portion of a hinge region can and generally will be present on a polypeptide chain between a CH1 domain and a CH2 domain. A hinge or portion thereof can also be placed on a polypeptide chain between a CL (e.g. Cκ) domain and the CH2 domain of an Fc domain when a CL is adjacent to an Fc domain on the polypeptide chain. However, it will be appreciated that a hinge region can optionally be replaced for example by a suitable linker peptide, e.g. a flexible polypeptide linker.

The NKp46 ABD and cytokine receptor ABD (e.g., a cytokine) are advantageously linked to the rest of the multispecific protein (e.g. or to a constant domain or Fc domain thereof) via a flexible linker (e.g. polypeptide linker) that leads to less structural rigidity or stiffness (e.g. between or amongst the ABD and Fc domain) compared to a conventional (e.g. wild-type full length human IgG) antibody. For example, the multispecific protein may have a structure or a flexible linker between the NKp46 ABD and constant domain or Fc domain that permits an increased range of domain motion compared to the two ABDs in a conventional (e.g. wild-type full length human IgG) antibody. In particular, the structure or a flexible linker can be configured to confer on the antigen binding sites greater intrachain domain movement compared to antigen binding sites in a conventional human IgG1 antibody. Rigidity or domain motion/interchain domain movement can be determined, e.g., by computer modeling, electron microscopy, spectroscopy such as Nuclear Magnetic Resonance (NMR), X-ray crystallography, or Sedimentation Velocity Analytical ultracentrifugation (AUC) to measure or compare the radius of gyration of proteins comprising the linker or hinge. A test protein or linker may have lower rigidity relative to a comparator protein if the test protein has a value obtained from one of the tests described in the previous sentence differs from the value of the comparator, e.g., an IgG1 antibody or a hinge, by at least 5%, 10%, 25%, 50%, 75%, or 100%. A cytokine can for example be fused to the C-terminus of a CH3 domain by a linker selected from GSSSS (SEQ ID NO: 171), GSSSSGSSSS (SEQ ID NO: 172), GSSSSGSSSSGS (SEQ ID NO: 173) or GSSSSGSSSSGSSSS (SEQ ID NO: 174).

In one embodiment, the multispecific protein may have a structure or a flexible linker between the NKp46 ABD and Fc domain that permits the NKp46 ABD and the ABD which binds an antigen of interest to have a spacing between said ABDs comprising less than about 80 angstroms, less than about 60 angstroms or ranges from about 40-60 angstroms.

At its C-terminus, an Fc domain (or a CH3 domain thereof) can be connected to the N-terminus of an NKp46 ABD or a cytokine polypeptide via a polypeptide linker, for example a glycine-serine-containing linker, optionally a linker having the amino acid sequence STGS.

In certain embodiments, a CH1 or CL domain of a Fab (e.g. of an NKp46 ABD) is fused at its C-terminus to the N-terminus of the cytokine via a flexible polypeptide linker, for example a glycine-serine-containing linker. Preferably, the linker will have a chain length of at least 4 amino acid residues, optionally the linker has a length of 5, 6, 7, 8, 9 or 10 amino acid residues.

In certain embodiments, the NKp46 ABD is placed C-terminal to the Fc domain, and the NKp46 is positioned between an Fc domain and the cytokine polypeptide in the multispecific protein. The NKp46 ABD will be connected or fused at its N-terminus (at the N-terminus of a VH or a VL domain) to the C-terminus of the Fc domain via a linker (e.g. a glycine and serine containing linker, a linker having the sequence STGS, a flexible polypeptide linker) of sufficient length to enable the NKp46 binding ABD to fold and/or adopt an orientation in such a way as to permit binding to Nkp46 at the surface of an NK cell, while at the same time possesses a sufficient distance and range of motion relative to the adjacent Fc domain (or more generally to rest of the multispecific protein) such that the Fc domain can also simultaneously be found by CD16 expressed at the surface of the same NK cell. Additionally, when the NKp46 ABD is placed between an Fc domain and an cytokine polypeptide in the multispecific protein, the C-terminus of a VH or VL of an scFv NKp46 ABD, or the CH1 or CL domain of a Fab NKp46 ABD will be connected or fused to the N-terminus of the cytokine polypeptide via a flexible linker (e.g. a flexible polypeptide linker) of sufficient length to enable the NKp46 binding ABD to fold and/or adopt an orientation in such a way as to permit binding to Nkp46 at the surface of an NK cell, while at the same time providing a sufficient distance and range of motion relative to the adjacent cytokine polypeptide such that the cytokine polypeptide can also simultaneously be bound by its cytokine receptor expressed at the surface of the NK cell. Preferably, the linker will have a chain length of at least 4 amino acid residues, optionally the linker has a length of 5, 6, 7, 8, 9 or 10 amino acid residues.

In tandem variable regions (e.g. scFv), two V domains (e.g. a V_{H} domain and V_{L} domains are generally linked together by a linker of sufficient length to enable the ABD to fold in such a way as to permit binding to the antigen for which the ABD is intended to bind. Examples of linkers include linkers comprising glycine and serine residues, e.g., the amino acid sequence GEGTSTGSGGSGGSGGAD (SEQ ID NO: 509). In another specific embodiment, the V_{H} domain and V_{L} domains of a scFv are linked together by the amino acid sequence (G₄S)₃.

In one embodiment, a (poly)peptide linker used to link a VH or VL domain of an scFv to a CH2 domain of an Fc domain comprises a fragment of a CH1 domain or CL domain and/or hinge region. For example, an N-terminal amino acid sequence of CH1 can be fused to a variable domain in order to mimic as closely as possible the natural structure of a wild-type antibody. In one embodiment, the linker comprises an amino acid sequence from a hinge domain or an N-terminal CH1 amino acid. In one embodiment, the linker peptide mimics the regular VK-CK elbow junction, e.g., the linker comprises or consists of the amino acid sequence RTVA.

In one embodiment, the hinge region used to connect the C-terminal end of a CH1 or CK domain (e.g. of a Fab) with the N-terminal end of a CH2 domain will be a fragment of a hinge region (e.g. a truncated hinge region without cysteine residues) or may comprise one or more amino acid modifications which remove (e.g. substitute by another amino acid, or delete) a cysteine residue, optionally both cysteine residues in a hinge region. Removing cysteines can be useful to prevent undesired disulfide bond formation, e.g., the formation of disulfide bridges in a monomeric polypeptide.

A “hinge” or “hinge region” or “antibody hinge region” herein refers to the flexible polypeptide or linker between the first and second constant domains of an antibody. Structurally, the IgG CH1 domain ends at EU position 220, and the IgG CH2 domain begins at residue EU position 237. Thus for an IgG the hinge generally includes positions 221 (D221 in IgG1) to 236 (G236 in IgG1), wherein the numbering is according to the EU index as in Kabat. References to specific amino acid residues within constant region domains found within the polypeptides shall be, unless otherwise indicated or as otherwise dictated by context, be defined according to Kabat, in the context of an IgG antibody.

In one embodiment, the hinge region (or fragment thereof) is derived form a hinge domain of a human IgG1 antibody. For example a hinge domain may comprise the amino acid sequence: THTCPPCPAPELL (SEQ ID NO: 166) or a fragment comprising the first 8 resides thereof, or an amino acid sequence at least 60%, 70%, 80% or 90% identical to any of the foregoing, optionally wherein one or both cysteines are deleted or substituted by a different amino acid residue, optionally a serine.

In one embodiment, the hinge region (or fragment thereof) is derived from a Cμ2-C Cμ3 hinge domain of a human IgM antibody. For example a hinge domain may comprise the amino acid sequence: NASSMCVPSPAPELL (SEQ ID NO: 167), or an amino acid sequence at least 60%, 70%, 80% or 90% identical thereto, optionally wherein one or both cysteines are deleted or substituted by a different amino acid residue.

Polypeptide chains that dimerize and associate with one another via non-covalent bonds or interactions may or may not additionally be bound by an interchain disulfide bond formed between respective CH1 and Cκ domains, and/or between respective hinge domains on the chains. CH1, Cκ and/or hinge domains (or other suitable linking amino acid sequences) can optionally be configured such that interchain disulfide bonds are formed between chains such that the desired pairing of chains is favored and undesired or incorrect disulfide bond formation is avoided. For example, when two polypeptide chains to be paired each possess a CH1 or Cκ adjacent to a hinge domain, the polypeptide chains can be configured such that the number of available cysteines for interchain disulfide bond formation between respective CH1/Cκ-hinge segments is reduced (or is entirely eliminated). For example, the amino acid sequences of respective CH1, Cκ and/or hinge domains can be modified to remove cysteine residues in both the CH1/Cκ and the hinge domain of a polypeptide; thereby the CH1 and Cκ domains of the two chains that dimerize will associate via non-covalent interaction(s).

In another example, the CH1 or Cκ domain adjacent to (e.g., N-terminal to) a hinge domain comprises a cysteine capable of interchain disulfide bond formation, and the hinge domain which is placed at the C-terminus of the CH1 or Cκ comprises a deletion or substitution of one or both cysteines of the hinge (e.g. Cys 239 and Cys 242, as numbered for human IgG1 hinge according to Kabat). In one embodiment, the hinge region (or fragment thereof) comprise the amino acid sequence: THTSPPSPAPELL (SEQ ID NO: 168), or an amino acid sequence at least 60%, 70%, 80% or 90% identical thereto.

In another example, the CH1 or Cκ domain adjacent (e.g., N-terminal to) a hinge domain comprises a deletion or substitution at a cysteine residue capable of interchain disulfide bond formation, and the hinge domain placed at the C-terminus of the CH1 or Cκ comprises one or both cysteines of the hinge (e.g. Cys 239 and Cys 242, as numbered for human IgG1 hinge according to Kabat). In one embodiment, the hinge region (or fragment thereof) comprises the amino acid sequence: THTCSSCPAPELL (SEQ ID NO: 169), or an amino acid sequence at least 60%, 70%, 80% or 90% identical thereto.

In another example, a hinge region is derived from an IgM antibody. In such embodiments, the CH1/CK pairing mimics the Cμ2 domain homodimerization in IgM antibodies. For example, the CH1 or Cκ domain adjacent (e.g., N-terminal to) a hinge domain comprises a deletion or substitution at a cysteine capable of interchain disulfide bond formation, and an IgM hinge domain which is placed at the C-terminus of the CH1 or Cκ comprises one or both cysteines of the hinge. In one embodiment, the hinge region (or fragment thereof) comprises the amino acid sequence: THTCSSCPAPELL (SEQ ID NO: 170), or an amino acid sequence at least 60%, 70%, 80% or 90% identical thereto.

Alternatively to the polypeptide linkers, a variety of nonproteinaceous polymer or chemical linkers may find use in the multispecific proteins. For example nonproteinaceous polymers including but not limited to polyethylene glycol (PEG), polypropylene glycol, polyoxyalkylenes, or copolymers of polyethylene glycol and polypropylene glycol, may find use as linkers. In some examples, an amino acid sequence in a polypeptide chain of a multispecific protein may be modified to introduce a reactive group, optionally a protected reactive group, and the so-modified protein or chain is then reacted with a linker or a polypeptide comprising a complementary reactive group. In some examples, an amino acid residue in a polypeptide chain of a multispecific protein can be bound to a linker comprising a reactive group (for further reaction with a second polypeptide functionalized with a linker with a complementary reactive group) or directly a second polypeptide via an enzyme catalyzed reaction. For example, a polypeptide comprising an acceptor glutamine or lysine can reacted with linker comprising a primary amine in the presence of a transglutamine enzyme (e.g. Bacterial Transglutaminase, BTG) such that the transglutaminase enzyme catalyzes the conjugation of the linker to an acceptor glutamine residue within the primary structure of the polypeptide, for example within an immunoglobulin constant domain or within a TGase recognition tag inserted or appended to (e.g., fused to) a constant region. A second polypeptide can also be functionalized with a linker in a similar manner, and when the conjugated linkers each bear complementary reactive groups (e.g. R on the linker of one polypeptide and R′ on the linker of the other polypeptide), the two functionalized polypeptides can be reacted such that they are bound via the linker comprising the residue of the reaction or R with R′. Examples of reactive group pairs R and R′ include a range of groups capable of biorthogonal reaction, for example 1,3-dipolar cycloaddition between azides and cyclooctynes (copper-free click chemistry), between nitrones and cyclooctynes, oxime/hydrazone formation from aldehydes and ketones and the tetrazine ligation (see also WO2013/092983). The resulting linker and functionalized antibody, or the Y element thereof, can thus comprise a RR′ group resulting from the reaction of R and R′, for example a triazole. Methods and linkers for use in BTG-mediated conjugation to antibodies is described in PCT publication no. WO2014/202773, the disclosure of which is incorporated by reference. “Transglutaminase”, used interchangeably with “TGase” or “TG”, refers to an enzyme capable of cross-linking proteins through an acyl-transfer reaction between the γ-carboxamide group of peptide-bound glutamine and the ε-amino group of a lysine or a structurally related primary amine such as amino pentyl group, e.g. a peptide-bound lysine, resulting in a ε-(γ-glutamyl)lysine isopeptide bond. TGases include, inter alia, bacterial transglutaminase (BTG) such as the enzyme having EC reference EC 2.3.2.13 (protein-glutamine-γ-glutamyltransferase). The term “acceptor glutamine” residue, when referring to a glutamine residue of an antibody, means a glutamine residue that is recognized by a TGase and can be cross-linked by a TGase through a reaction between the glutamine and a lysine or a structurally related primary amine such as amino pentyl group. Preferably the acceptor glutamine residue is a surface-exposed glutamine residue. The term “TGase recognition tag” refers to a sequence of amino acids comprising an acceptor glutamine residue and that when incorporated into (e.g. appended to) a polypeptide sequence, under suitable conditions, is recognized by a TGase and leads to cross-linking by the TGase through a reaction between an amino acid side chain within the sequence of amino acids and a reaction partner. The recognition tag may be a peptide sequence that is not naturally present in the polypeptide comprising the enzyme recognition tag. Examples of TGase recognition tags include the amino acid sequences disclosed in WO2012/059882 and WO2014/072482, the disclosure of which sequences are incorporated herein by reference.

Constant Regions

Constant region domains can be derived from any suitable human antibody, particularly human antibodies of gamma isotype, including, the constant heavy (CH1) and light (CL, Cκ or Cλ) domains, hinge domains, CH2 and CH3 domains.

With respect to heavy chain constant domains, “CH1” generally refers to positions 118-220 according to the EU index as in Kabat. Depending on the context, a CH1 domain (e.g. as shown in the domain arrangements), can optionally comprise residues that extend into the hinge region such that the CH1 comprises at least part of a hinge region. For example, when positioned C-terminal on a polypeptide chain and/or or C-terminal to the Fc domain, and/or within a Fab structure that is or C-terminal to the Fc domain, the CH1 domain can optionally comprise at least part of a hinge region, for example CH1 domains can comprise at least an upper hinge region, for example an upper hinge region of a human IgG1 hinge, optionally further in which the terminal threonine of the upper hinge can be replaced by a serine. Such a CH2 domain can therefore comprise at its C-terminus the amino acid sequence: EPKSCDKTHS (SEQ ID NO: 440).

Exemplary human CH1 domain amino acid sequences include:

(SEQ ID NO: 156)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRV
or
(SEQ ID NO: 157)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHS.
or
(SEQ ID NO: 158)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHT.

Exemplary human Cκ domain amino acid sequences include:

(SEQ ID NO: 159)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC.

In some exemplary configurations, the multispecific protein can be a heterodimer, a heterotrimer or a heterotetramer comprising one or two Fabs (e.g. one Fab binding NKp46 and the other binding the antigen of interest), in which variable regions, CH1 and/or CL domains are engineered by introducing amino acid substitutions in a knob-into-holes or electrostatic steering approach to promote the desired chain pairings of CH1 domains with CK domains. In some exemplary configurations, the multispecific protein can be a heterodimer, a heterotrimer or a heterotetramer comprising one or two Fabs (e.g. one Fab binding NKp46 and the other binding the antigen of interest), wherein a Fab has a VH/VL crossover (VH and VL replace one another) or a CH1/CL crossover (the CH1 and CL replace one another), and wherein the CH1 and/or CL domains comprise amino acid substitutions to promote correct chain association by knob-into-holes or electrostatic steering.

“CH2” generally refers to positions 237-340 according to the EU index as in Kabat, and “CH3” generally refers to positions 341-447 according to the EU index as in Kabat. CH2 and CH3 domains can be derived from any suitable antibody. Such CH2 and CH3 domains can be used as wild-type domains or may serve as the basis for a modified CH2 or CH3 domain. Optionally the CH2 and/or CH3 domain is of human origin or may comprise that of another species (e.g., rodent, rabbit, non-human primate) or may comprise a modified or chimeric CH2 and/or CH3 domain, e.g., one comprising portions or residues from different CH2 or CH3 domains, e.g., from different antibody isotypes or species antibodies.

In any of the domain arrangements, the Fc domain monomer may comprise a CH2-CH3 unit (a full length CH2 and CH3 domain or a fragment thereof). In heterodimers or heterotrimers comprising two chains with Fc domain monomers (i.e. the heterodimers or heterotrimers comprise a Fc domain dimer), the CH3 domain will be capable of CH3-CH3 dimerization (e.g. it will comprise a wild-type CH3 domain or a CH3 domain with modifications to promote a desired CH3-CH3 dimerization).

Exemplary human IgG1 CH2-CH3 (Fc) domain amino acid sequences include:

(SEQ ID NO: 160)
APELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHE
ALHNHYTQKSLSLSPG.

An Fc domain may optionally further comprise a C-terminal lysine (K). In some exemplary configurations, the multispecific protein can be a heterodimer, a heterotrimer or a heterotetramer, wherein the polypeptide chains are engineered for heterodimerization among each other so as to produce the desired protein. In embodiments where the desired chain pairings are not driven by CH1-Cκ dimerization or generally where enhancement of pairing is desired, the chains may comprise constant or Fc domains with amino acid modifications (e.g., substitutions) that favor the preferential chain pairing, e.g. favor a desired hetero-dimerization of the two different chains over the homo-dimerization of two identical chains.

In some embodiments, a “knob-into-holes” approach is used in which the domain interfaces (e.g. CH3 domain interface of the antibody Fc region) are mutated so that the antibodies preferentially heterodimerize. Mutations can be introduced to create altered charge polarity across the interface (e.g. Fc dimer interface) such that co-expression of electrostatically matched chains (e.g. Fc-containing chains) support favorable attractive interactions thereby promoting desired heterodimer (e.g. Fc heterodimer) formation, whereas unfavorable repulsive charge interactions suppress unwanted heterodimer (e.g., Fc homodimer) formation. See for example mutations and approaches reviewed in Brinkmann and Kontermann, 2017 MAbs, 9(2): 182-212, the disclosure of which is incorporated herein by reference. For example one heavy chain comprises a T366W substitution and the second heavy chain comprises a T366S, L368A and Y407V substitution, see, e.g. Ridgway et al (1996) Protein Eng., 9, pp. 617-621; Atwell (1997) J. Mol. Biol., 270, pp. 26-35; and WO2009/089004, the disclosures of which are incorporated herein by reference. For example the “Hole” mutations on a first Fc monomer can comprise Y349C/T366S/L368A/Y407V and the complementary “Knob” mutations on the second Fc monomer can comprise S354C/T366W (Kabat EU numbering). In another approach, one heavy chain comprises a F405L substitution and the second heavy chain comprises a K409R substitution, see, e.g., Labrijn et al. (2013) Proc. Natl. Acad. Sci. U.S.A., 110, pp. 5145-5150. In another approach, one heavy chain comprises T350V, L351Y, F405A, and Y407V substitutions and the second heavy chain comprises T350V, T366S, K392L, and T394W substitutions, see, e.g. Von Kreudenstein et al., (2013) mAbs 5:646-654. In another approach, one heavy chain comprises both K409D and K392D substitutions and the second heavy chain comprises both D399K and E356K substitutions, see, e.g. Gunasekaran et al., (2010) J. Biol. Chem. 285:19637-19646. In another approach, one heavy chain comprises D221E, P228E and L368E substitutions and the second heavy chain comprises D221R, P228R, and K409R substitutions, see, e.g. Strop et al., (2012) J. Mol. Biol. 420: 204-219. In another approach, one heavy chain comprises S364H and F405A substitutions and the second heavy chain comprises Y349T and, T394F substitutions, see, e.g. Moore et al., (2011) mAbs 3: 546-557. In another approach, one heavy chain comprises a H435R substitution and the second heavy chain optionally may or may not comprise a substitution, see, e.g. U.S. Pat. No. 8,586,713. When such hetero-multimeric antibodies have Fc regions derived from a human IgG2 or IgG4, the Fc regions of these antibodies can be engineered to contain amino acid modifications that permit CD16 binding. In some embodiments, the antibody may comprise mammalian antibody-type N-linked glycosylation at residue N297 (Kabat EU numbering).

In some embodiments, a multispecific protein comprises one or more amino acid modifications (e.g. substitutions) in a CH3 domain that affect binding to an affinity purification medium, e.g. Protein A. Introduction into one of the CH3 domains of mutations that diminish binding to Protein A can be used to distinguish unwanted chain pairings from the desired protein. For example, mutations can be introduced at amino acids H435 and Y436 (Kabat EU numbering), for example H435R and Y436F. In one embodiment, in a multispecific protein that has a dimeric Fc domain modified to retain binding to CD16A, a first Fc monomer (e.g. on one of the polypeptide chains) comprises an amino acid sequence at least 90%, 95% or 99% identical to SEQ ID NO: 161 and a second Fc monomer (e.g. on another of the polypeptide chains) comprises an amino acid sequence at least 90%, 95% or 99% identical to SEQ ID NO: 162. Optionally, each of the Fc monomers is fused at its N-terminus to the hinge amino acid sequence of SEQ ID NO: 166.

In some embodiments, one or more pairs of disulfide bonds such as A287C and L306C, V259C and L306C, R292C and V302C, and V323C and 1332C are introduced into the Fc region to increase stability, for example further to a loss of stability caused by other Fc modifications. Additional example includes introducing K3381, A339K, and K340S mutations to enhance Fc stability and aggregation resistance (Gao et al, 2019 Mol Pharm. 2019; 16:3647).

In some embodiments, where a multispecific protein is intended to have reduced binding to a human Fc gamma receptor. In some embodiments, where a multispecific protein is intended to have reduced binding to a human CD16A polypeptide (and optionally further reduced binding to CD32A, CD32B and/or CD64), the Fc domain is a human IgG4 Fc domain, optionally further wherein the Fc domain comprises a S228P mutation to stabilize the hinge disulfide. In one embodiment, the Fc domain has an amino acid sequence at least 90%, 95% or 99% identical to a human IgG4 Fc domain, optionally further comprising a Kabat S228P mutation.

In embodiments, where a multispecific protein is intended to have reduced binding to human CD16A polypeptide (and optionally further reduced binding to CD32A, CD32B and/or CD64), a CH2 and/or CH3 domain (or Fc domain comprising same) may comprise a modification to decrease or abolish binding to FcγRIIIA (CD16). For example, CH2 mutations in a Fc domain dimer proteins at reside N297 (Kabat numbering) can substantially eliminate CD16A binding. However the person of skill in the art will appreciate that other configurations can be implemented. For example, substitutions into human IgG1 or IgG2 residues at positions 233-236 and/or residues at positions 327, 330 and 331 were shown to greatly reduce binding to Fcγ receptors and thus ADCC and CDC. Furthermore, Idusogie et al. (2000) J. Immunol. 164(8):4178-84 demonstrated that alanine substitution at different positions, including K322, significantly reduced complement activation.

In one embodiment, the asparagine (N) at Kabat heavy chain residue 297 can be substituted by a residue other than an asparagine (e.g. a glutamine, a residue other than glutamine, for example a serine).

In one embodiment, an Fc domain modified to reduce binding to CD16A comprises a substitution in the Fc domain at Kabat residues 234, 235 and 322. In one embodiment, the protein comprises a substitution in the Fc domain at Kabat residues 234, 235 and 331. In one embodiment, the protein comprises a substitution in the Fc domain at Kabat residues 234, 235, 237 and 331. In one embodiment, the protein comprises a substitution in the Fc domain at Kabat residues 234, 235, 237, 330 and 331. In one embodiment, the Fc domain is of human IgG1 subtype. Amino acid residues are indicated according to EU numbering according to Kabat.

In one embodiment, an Fc domain modified to reduce binding to CD16A comprises an amino acid modification (e.g. substitution) at one or more of Kabat residue(s) 233-236, optionally one or more of residues 233-237, or at one, two or three of residues 234, 235 and/or 237, and an amino acid modification (e.g. substitution) at Kabat residue(s) 330 and/or 331. One example of such an Fc domain comprises substitutions at Kabat residues L234, L235 and P331 (e.g., L234A/L235E/P331S or (L234F/L235E/P331S). Another example of such an Fc domain comprises substitutions at Kabat residues L234, L235, G237 and P331 (e.g., L234A/L235E/G237A/P331S). Another example of such an Fc domain comprises substitutions at Kabat residues L234, L235, G237, A330 and P331 (e.g., L234A/L235E/G237A/A330S/P331S). In one embodiment, an antibody comprises an human IgG1 Fc domain comprising L234A/L235E/N297X/P331S substitutions, L234F/L235E/N297X/P331S substitutions, L234A/L235E/G237A/N297X/P331S substitutions, or L234A/L235E/G237A/N297X/A330S/P331S substitutions, wherein X can be any amino acid other than an asparagine. In one embodiment, X is a glutamine; in another embodiment, X is a residue other than a glutamine (e.g. a serine).

In one embodiment, an Fc domain that has low or reduced binding to CD16A comprises a human IgG4 Fc domain, wherein the Fc domain has the amino acid sequence below (human IgG4 with S228P substitution), or an amino acid sequence at least 90%, 95% or 99% identical thereto.

In one embodiment, an Fc domain modified to reduce binding to CD16A comprises the amino acid sequence below, or an amino acid sequence at least 90%, 95% or 99% identical thereto but retaining the amino acid residues at Kabat positions 234, 235 and 331 (underlined):

(SEQ ID NO: 163)
A S T K G P S V F P L A P S S K S T S G G
T A A L G C L V K D Y F P E P V T V S W N
S G A L T S G V H T F P A V L Q S S G L Y
S L S S V V T V P S S S L G T Q T Y I C N
V N H K P S N T K V D K R V E P K S C D K
T H T C P P C P A P E F E G G P S V F L F
P P K P K D T L M I S R T P E V T C V V V
D V S H E D P E V K F N W Y V D G V E V H
N A K T K P R E E Q Y N S T Y R V V S V L
T V L H Q D W L N G K E Y K C K V S N K A
L P A S I E K T I S K A K G Q P R E P Q V
Y T L P P S R E E M T K N Q V S L T C L V
K G F Y P S D I A V E W E S N G Q P E N N
Y K T T P P V L D S D G S F F L Y S K L T
V D K S R W Q Q G N V F S C S V M H E A L
H N H Y T Q K S L S L S P G

In one embodiment, an Fc domain modified to reduce binding to CD16A comprises the amino acid sequence below, or an amino acid sequence at least 90%, 95% or 99% identical thereto but retaining the amino acid residues at Kabat positions 234, 235, 237, 330 and 331 (underlined):

(SEQ ID NO: 164)
A S T K G P S V F P L A P S S K S T S G G
T A A L G C L V K D Y F P E P V T V S W N
S G A L T S G V H T F P A V L Q S S G L Y
S L S S V V T V P S S S L G T Q T Y I C N
V N H K P S N T K V D K R V E P K S C D K
T H T C P P C P A P E A E G A P S V E L F
P P K P K D T L M I S R T P E V T C V V V
D V S H E D P E V K F N W Y V D G V E V H
N A K T K P R E E Q Y N S T Y R V V S V L
T V L H Q D W L N G K E Y K C K V S N K A
L P S S I E K T I S K A K G Q P R E P Q V
Y T L P P S R E E M T K N Q V S L T C L V
K G F Y P S D I A V E W E S N G Q P E N N
Y K T T P P V L D S D G S F F L Y S K L T
V D K S R W Q Q G N V F S C S V M H E A L
H N H Y T Q K S L S L S P G

Any of the above Fc domain sequences can optionally further comprise a C-terminal lysine (K), i.e. as in the naturally occurring sequence.

In certain embodiments herein where binding to CD16 (CD16A) is desired, a CH2 and/or CH3 domain (or Fc domain comprising same) may be a wild-type domain or a domain having CD16 interface residues from the wild-type human IgG1 domain, or may comprise one or more amino acid modifications (e.g. amino acid substitutions) which increase binding to human CD16 and optionally another receptor such as FcRn. Optionally, the modifications will not substantially decrease or abolish the ability of the Fc-derived polypeptide to bind to neonatal Fc receptor (FcRn), e.g. human FcRn. Typical modifications include modified human IgG1-derived constant regions comprising at least one amino acid modification (e.g. substitution, deletions, insertions), and/or altered types of glycosylation, e.g., hypofucosylation. Such modifications can affect interaction with Fc receptors: FcγRI (CD64), FcγRII (CD32), and FcγRIII (CD16). FcγRI (CD64), FcγRIIA (CD32A) and FcγRIII (CD 16) are activating (i.e., immune system enhancing) receptors while FcγRIIB (CD32B) is an inhibiting (i.e., immune system dampening) receptor. A modification may, for example, increase binding of the Fc domain to FcγRIIIa on effector (e.g. NK) cells and/or decrease binding to FcγRIIB. Examples of modifications are provided in PCT publication no. WO2014/044686, the disclosure of which is incorporated herein by reference. Specific mutations (in IgG1 Fc domains) which affect (enhance) FcγRIIIa or FcRn binding are also set forth below.

| Isotype | Species | Modification | Effector Function | Effect of Modification |
|---|---|---|---|---|
| IgG1 | Human | T250Q/M428L | Increased binding to | Increased half-life |
| | | | FcRn | |
| IgG1 | Human | 1M252Y/S254T/T256E+ | Increased binding to | Increased half-life |
| | | H433K/N434F | FcRn | |
| IgG1 | Human | E333A | Increased binding to | Increased ADCC and |
| | | | FcγRIIIa | CDC |
| IgG1 | Human | S239D/1332E or | Increased binding to | Increased ADCC |
| | | S239D/A330L/I332E | FcγRIIIa | |
| IgG1 | Human | P2571/Q311 | Increased binding to | Unchanged half-life |
| | | | FcRn | |
| IgG1 | Human | S239D/I332E/G236A | Increased | Increased macrophage |
| | | | FcγRIIa/FcγRIIb ratio | phagocytosis |

In some embodiments, the multispecific protein comprises a variant Fc region comprise at least one amino acid modification (for example, possessing 1, 2, 3, 4, 5, 6, 7, 8, 9, or more amino acid modifications) in the CH2 and/or CH3 domain of the Fc region, wherein the modification enhances binding to a human CD16 polypeptide. In other embodiments, the multispecific protein comprises at least one amino acid modification (for example, 1, 2, 3, 4, 5, 6, 7, 8, 9, or more amino acid modifications) in the CH2 domain of the Fc region from amino acids 237-341, or within the lower hinge-CH2 region that comprises residues 231-341. In some embodiments, the multispecific protein comprises at least two amino acid modifications (for example, 2, 3, 4, 5, 6, 7, 8, 9, or more amino acid modifications), wherein at least one of such modifications is within the CH3 region and at least one such modifications is within the CH2 region. Encompassed also are amino acid modifications in the hinge region. In one embodiment, encompassed are amino acid modifications in the CH1 domain, optionally in the upper hinge region that comprises residues 216-230 (Kabat EU numbering). Any suitable functional combination of Fc modifications can be made, for example any combination of the different Fc modifications which are disclosed in any of U.S. Pat. Nos. 7,632,497; 7,521,542; 7,425,619; 7,416,727; 7,371,826; 7,355,008; 7,335,742; 7,332,581; 7,183,387; 7,122,637; 6,821,505 and 6,737,056; and/or in PCT Publications Nos. WO2011/109400; WO 2008/105886; WO 2008/002933; WO 2007/021841; WO 2007/106707; WO 06/088494; WO 05/115452; WO 05/110474; WO 04/1032269; WO 00/42072; WO 06/088494; WO 07/024249; WO 05/047327; WO 04/099249 and WO 04/063351; and/or in Lazar et al. (2006) Proc. Nat. Acad. Sci. USA 103(11): 405-410; Presta, L. G. et al. (2002) Biochem. Soc. Trans. 30(4):487-490; Shields, R. L. et al. (2002) J. Biol. Chem. 26; 277(30):26733-26740 and Shields, R. L. et al. (2001) J. Biol. Chem. 276(9):6591-6604).

In some embodiments, the multispecific protein comprises an Fc domain comprising at least one amino acid modification (for example, 1, 2, 3, 4, 5, 6, 7, 8, 9, or more amino acid modifications) relative to a wild-type Fc region, such that the molecule has an enhanced binding affinity for human CD16 relative to the same molecule comprising a wild-type Fc region, optionally wherein the variant Fc region comprises a substitution at any one or more of positions 221, 239, 243, 247, 255, 256, 258, 267, 268, 269, 270, 272, 276, 278, 280, 283, 285, 286, 289, 290, 292, 293, 294, 295, 296, 298, 300, 301, 303, 305, 307, 308, 309, 310, 311, 312, 316, 320, 322, 326, 329, 330, 332, 331, 332, 333, 334, 335, 337, 338, 339, 340, 359, 360, 370, 373, 376, 378, 392, 396, 399, 402, 404, 416, 419, 421, 430, 434, 435, 437, 438 and/or 439 (Kabat EU numbering).

In one embodiment, the multispecific protein comprises an Fc domain comprising at least one amino acid modification (for example, 1, 2, 3, 4, 5, 6, 7, 8, 9, or more amino acid modifications) relative to a wild-type Fc region, such that the molecule has enhanced binding affinity for human CD16 relative to a molecule comprising a wild-type Fc region, optionally wherein the variant Fc region comprises a substitution at any one or more of positions 239, 298, 330, 332, 333 and/or 334 (e.g. S239D, S298A, A330L, 1332E, E333A and/or K334A substitutions), optionally wherein the variant Fc region comprises a substitution at residues S239 and 1332, e.g. a S239D and 1332E substitution (Kabat EU numbering).

In some embodiments, the multispecific protein comprises an Fc domain comprising N-linked glycosylation at Kabat residue N297. In some embodiments, the multispecific protein comprises an Fc domain comprising altered glycosylation patterns that increase binding affinity for human CD16. Such carbohydrate modifications can be accomplished by, for example, by expressing a nucleic acid encoding the multispecific protein in a host cell with altered glycosylation machinery. Cells with altered glycosylation machinery are known in the art and can be used as host cells in which to express recombinant antibodies to thereby produce an antibody with altered glycosylation. See, for example, Shields, R. L. et al. (2002) J. Biol. Chem. 277:26733-26740; Umana et al. (1999) Nat. Biotech. 17:176-1, as well as, European Patent No: EP 1176195; PCT Publications WO 06/133148; WO 03/035835; WO 99/54342, each of which is incorporated herein by reference in its entirety. In one aspect, the multispecific protein contains one or more hypofucosylated constant regions. Such multispecific protein may comprise an amino acid alteration or may not comprise an amino acid alteration and/or may be expressed or synthesized or treated under conditions that result in hypofucosylation. In one aspect, a multispecific protein composition comprises a multispecific protein described herein, wherein at least 20, 30, 40, 50, 60, 75, 85, 90, 95% or substantially all of the antibody species in the composition have a constant region comprising a core carbohydrate structure (e.g. complex, hybrid and high mannose structures) which lacks fucose. In one embodiment, provided is a multispecific protein composition which is free of N-linked glycans comprising a core carbohydrate structure having fucose. The core carbohydrate will preferably be a sugar chain at Asn297.

Optionally, a multispecific protein comprising a Fc domain dimer can be characterized by having a binding affinity to a human CD16A polypeptide that is within 1-log of that of a conventional human IgG1 antibody, e.g., as assessed by surface plasmon resonance.

In one embodiment, the multispecific protein comprising a Fc domain dimer in which an Fc domain is engineered to enhance Fc receptor binding can be characterized by having a binding affinity to a human CD16A polypeptide that is at least 1-log greater than that of a conventional or wild-type human IgG1 antibody, e.g., as assessed by surface plasmon resonance.

In one embodiment, a multispecific protein comprising a Fc domain dimer can be characterized by having a binding affinity to a human FcRn (neonatal Fc receptor) polypeptide that is within 1-log of that of a conventional human IgG1 antibody, e.g., as assessed by surface plasmon resonance.

Optionally a multispecific protein comprising a Fc domain dimer can be characterized by a Kd for binding (monovalent) to a human Fc receptor polypeptide (e.g., CD16A) of less than 10⁻⁵ M (10 μmolar), optionally less than 10⁻⁶ M (1 μmolar), as assessed by surface plasmon resonance (e.g. as in the Examples herein, SPR measurements performed on a Biacore T100 apparatus (Biacore GE Healthcare), with bispecific antibodies immobilized on a Sensor Chip CM5 and serial dilutions of soluble CD16 polypeptide injected over the immobilized bispecific antibodies.

Cytokine Receptor ABD

The antigen binding domain that binds to a cytokine receptor on NK cells (cytokine receptor ABD) can advantageously comprise a suitable cytokine polypeptide or polypeptide fragment such that the cytokine receptor ABD binds the cytokine receptor on the surface of an NK cell. The cytokine can for example be a full-length wild-type IL-2, IL-15, IL-21, IL-7, IL-27, IL-12, IL-18, IFN-α or IFN-β polypeptide, a fragment thereof sufficient to bind to the NK cell receptor for such cytokine, or a variant of any of the foregoing. The cytokine molecule can be a fragment comprising at least 20, 30, 40, 50, 60, 70, 80 or 100 contiguous amino acids of a human cytokine, wherein the cytokine retains the ability to bind its cytokine receptor present on the surface of an NK cell. In certain embodiments, the cytokine is a variant of a human cytokine comprising one or more amino acid modifications (e.g. amino acid substitutions) compared to the wild-type human cytokine, for example to decrease binding affinity to a receptor present on non-NK cells, for example Treg cells, CD4 T cells, CD8 T cells. The cytokine can for example be a type I cytokine and a member of the common cytokine receptor gamma-chain (cg-chain) cytokine family, that signals via a heteromultimeric or heterdimeric receptor complex comprised of a receptor subunit (e.g., IL-2Rβ/IL-15Rβ or IL-21R) subunit that associates with the common gamma-chain (CD132).

In one embodiment, the multispecific proteins that binds to NKp46 and optionally further CD16A incorporates a cytokine (or fragment of variant thereof) which is modified to attenuate (reduce) binding affinity at the cytokine receptor expressed on NK cells, in comparison to the human wild-type cytokine counterpart. The modified cytokine (or fragment of variant thereof) retains partial activity and/or binding affinity at the cytokine receptor expressed on NK cells, in comparison to the human wild-type cytokine counterpart. In one embodiment, the cytokine retains at least 5%, 10%, 20% or 50% of the ability of a wild-type cytokine counterpart to induce signaling through its receptor on NK cells.

In one embodiment, the multispecific proteins that binds to NKp46 and optionally further CD16A permits the incorporation of a wild-type cytokine (or fragment of variant thereof) that retain substantially full activity and/or binding affinity at the cytokine receptor expressed on NK cells, in comparison to the human wild-type cytokine counterpart. In one embodiment, the cytokine is a wild-type cytokine or fragment thereof, or is a modified cytokine, wherein the cytokine is does not have a substantially reduced ability to induce signaling and/or does not have a substantially reduced binding affinity at its receptor on NK cells (e.g. CD122, IL-21R, IL-7Ra, IL-27Ra, IL-12R, IL-18R). In one embodiment, the cytokine does not comprise a modification (e.g., substitution, deletion, etc.) that substantially reduces its ability to induce signaling through its receptor on NK cells (e.g. CD122, IL-21R, IL-7Ra, IL-27Ra, IL-12R, IL-18R). In one embodiment, the cytokine retains at least 80%, 90% of the ability of a wild-type cytokine counterpart to induce signaling through its receptor on NK cells (e.g. CD122, IL-21R, IL-7Ra, IL-27Ra, IL-12R, IL-18R). Optionally, signaling is assessed by bringing the cytokine (e.g. as a recombinant protein domain or within a multispecific protein of the disclosure) into contact with an NK cell and measuring signaling, e.g. measuring STAT phosphorylation in the NK cells.

In some embodiments, when the exemplary anti-NKp46 VH/VL pairs disclosed herein having a KD for NKp46 in the range of about 15 nM, or functionally conservative variants thereof, are used in the multispecific proteins, the cytokine or cytokine receptor ABD (either as a free cytokine or as incorporated into a multispecific protein) can be specified as binding its receptor, as determined by SPR, with a binding affinity (KD) of 200 nM or less, 100 nM or less, 50 nM or less or 25 nM or less. In one embodiment, the cytokine or cytokine receptor ABD binds its receptor, as determined by SPR, with a binding affinity (KD) that is 1 nM or higher than 1 nM, optionally that is higher than 10 nM optionally that is higher than 15 nM. In one embodiment, the cytokine or cytokine receptor ABD binds its receptor, as determined by SPR, with a binding affinity (KD) between about 1 nm and about 200 nm, optionally) between about 1 nm and about 100 nm optionally between about 10 nM and about 200 nM, optionally between about 10 nM and about 100 nM optionally between about 15 nM and about 100 nM.

When the cytokine-binding ABD is a CD122-binding ABD, the ABD can be or comprise a suitable interleukin-2 (IL-2) polypeptide such that the CD122 ABD binds CD122. As exemplified herein, the ABD is advantageously a variant or modified IL-2 polypeptide that has reduced binding to CD25 (IL-2Rα) (e.g. reduced or abolished binding affinity, for example as determined by SPR) compared to a wild-type human interleukin-2. Such a variant or modified IL-2 polypeptide is also referred herein to as an “IL2v” or a “not-alpha IL-2”. The CD122-binding ABD can optionally be specified to have a binding affinity for human CD122 that is substantially equivalent to that of wild-type human IL-2, or that is reduced compared to wild-type human IL-2. The CD122-binding ABD can optionally be specified to have an ability to induce CD122 signaling and/or binding affinity for CD122 that is substantially equivalent to that of wild-type human IL-2. In one embodiment, the CD122-binding ABD has a reduction in binding affinity for CD25 that is greater than the reduction in binding affinity for CD122, for example a reduction of at least 1-log, 2-log or 3-log in binding affinity for CD25 and a reduction in binding affinity for CD122 that is less than 1-log.

For example, a heteromultimeric multispecific protein can be specified as being capable of binding to NKp46 and CD122, and optionally further CD16A, on an NK cell, and which is capable of potentiating NK cell cytotoxicity toward a target cell expressing an antigen of interest, and comprising:
(a) an ABD that binds to an antigen of interest,
(b) an ABD that binds to a human NKp46 polypeptide,
(c) an Fc domain or portion thereof capable of binding FcRn, and optionally further to CD16A, and
(d) an ABD that binds a human CD122 polypeptide, wherein the ABD is placed at the C-terminus of the polypeptide chain which comprises such ABD, optionally wherein such ABD comprises an IL-2 polypeptide or portion thereof that binds CD122 and displays reduced binding affinity for human CD25 respectively, compared to a human wild-type IL-2 polypeptide. The ABD that binds CD122 can thus be placed for example C-terminal and/or adjacent to the Fc domain and/or ABD that binds NKp46 on a polypeptide chain.

IL-2 is believed to bind IL-2Rβ (CD122) in its form as a monomeric IL-2 receptor (IL-2R), followed by recruitment of the IL-2Rγ (CD132; also termed common γ chain) subunit. In cells that do not express CD25 at their surface, binding (e.g. reduced binding) to CD122 can therefore optionally be specified as being binding in or to a CD122:CD132 complex. The CD122 (or CD122:CD132 complex) can optionally be specified as being present at the surface of an NK cell. In cells that express CD25 at their surface, IL-2 is believed to bind CD25 (IL-2Rα) in its form as a monomeric IL-2 receptor, followed by association of the subunits IL-2Rβ and IL-2Rγ. Binding (e.g. reduced binding, partially reduced binding) to CD25 can therefore optionally be specified as being binding in or to a CD25:CD122 complex or a CD25:CD122:CD132 complex.

In a multispecific protein herein, the multispecific protein can optionally be specified as being configured and/or in a conformation (or capable of adopting a conformation) in which the CD122 ABD (e.g. IL2v) is capable of binding to CD122 at the surface of a cell (e.g. an NK cell, a CD122+CD25− cell) when the multispecific protein is bound to NKp46 (and optionally further to CD16) at the surface of said cell. Optionally further, the multispecific protein:CD122 complex is capable of binding to CD132 at the surface of said cell.

The CD122 ABD or IL2v can be a modified IL-2 polypeptide, for example a monomeric IL-2 polypeptide modified by introducing one more amino acid substitutions, insertions or deletions that decrease binding to CD25, with or without a decrease in binding to CD122.

In some embodiments, where binding to CD25 is sought to be selectively decreased, a IL-2 polypeptide can be modified by binding or associating it with one or more other additional molecules such as polymers or (poly)peptides that result in a further decrease of or abolished binding to CD25. For example a wild-type or mutated IL-2 polypeptide can be modified or further modified by binding to it another moiety that shields, masks, binds or interacts with CD25-binding site of human IL-2, thereby decreasing binding to CD25. In some examples, molecules such as polymers (e.g. PEG polymers) are conjugated to an IL-2 polypeptide to shield or mask the epitope on IL-2 that is bound by CD25, for example by introduction (e.g. substitution) to install an amino acid containing a dedicated chemical hook at a specific site on the IL-2 polypeptide. In other examples, a wild-type or variant IL-2 polypeptide is bound to anti-IL-2 monoclonal antibody or antibody fragment that binds or interacts with CD25-binding site of human IL-2, thereby decreasing binding to CD25.

In any embodiment, an IL2 polypeptide can be a full-length IL-2 polypeptide or it can be an IL-2 polypeptide fragment, so long as the fragment or IL2v that comprises it retains the specified activity (e.g. retaining at least partial CD122 binding, compared to wild-type IL-2 polypeptide).

As shown herein, an IL2v polypeptide can advantageously comprise an IL-2 polypeptide comprising one or more amino acid mutations designed to reduce its ability to bind to human CD25 (IL-2Rα), while retaining at least at least some, or optionally substantially full, ability to bind human CD122.

Various IL2v or not-alpha IL-2 moieties have been described which reduce the activation bias of IL-2 on CD25+ cells. Such IL2v reduce binding to IL-2Rα and maintain at least partial binding to IL-2Rβ. Several IL2v polypeptides have been described, many having mutations in amino acid residue regions 35-72 and/or 79-92 of the IL-2 polypeptide. For example, decreased affinity to IL-2Rα may be obtained by substituting one or more of the following residues in the sequence of a wild-type IL-2 polypeptide: R38, F42, K43, Y45, E62, P65, E68, V69, and L72 (amino acid residue numbering is with reference to the mature IL-2 polypeptide shown in SEQ ID NO: 404).

The wild-type mature human IL-2 protein and a wild-type mature IL-2p protein fragment lacking the three first residues APT are shown below in SEQ ID NOS: 404 and 405, respectively:

Wild-type mature human IL-2
(SEQ ID NO: 404)
APTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGSE
TTFMCEYADETATIVEFLNRWITFCQSIISTLT
Wild-type mature IL-2p:
(SEQ ID NO: 405)
SSSTKKTQLQLEHLLLDLQMILNGINNYKNPKLTRMLTFKFYMPKKATEL
KHLQCLEEELKPLEEVLNLAQSKNFHLRPRDLISNINVIVLELKGSETTF
MCEYADETATIVEFLNRWITFCQSIISTLT

An exemplary IL2v (also referred to herein as IL2v in the Examples) can have the amino acid of wild-type IL-2 with the five amino acid substitutions T3A, F42A, Y45A, L72G and C125A, as shown below, optionally further with deletion of the three N-terminal residues APA:

(SEQ ID NO: 406)
APASSSTKKTQLQLEHLLLDLQMILNGINNYKNPKLTRMLTAKFAMPKKA
TELKHLQCLEEELKPLEEVLNGAQSKNFHLRPRDLISNINVIVLELKGSE
TTFMCEYADETATIVEFLNRWITFAQSIISTLT

As few as one or two mutations can reduce binding to IL-2Rα and L-2Rβ. For example, as exemplified in the multispecific proteins herein, the IL2v polypeptide having two amino acid substitutions R38A and F42K in the wild-type IL-2p amino acid sequence displayed suitable reduced binding to IL-2Rα, with retention of binding to IL-2Rβ resulting in highly active multispecific proteins, referred to herein as IL2v2.

IL2v2 (R38A/F42K substitutions):
(SEQ ID NO: 407)
SSSTKKTQLQLEHLLLDLQMILNGINNYKNPKLTAMLTKKFY
MPKKATELKHLQCLEEELKPLEEVLNLAQSKNFHLRPRDLIS
NINVIVLELKGSETTFMCEYADETATIVEFLNRWITFCQSII
STLT

In one embodiment, an IL2v polypeptide has the wild-type IL-2p amino acid sequence with the three amino acid substitutions R38A, F42K and T41A, as shown below, referred to herein as IL2v3:

IL2v3 (R38A/T41A/F42K substitutions):
(SEQ ID NO: 408)
SSSTKKTQLQLEHLLLDLQMILNGINNYKNPKLTAMLAKKFY
MPKKATELKHLQCLEEELKPLEEVLNLAQSKNFHLRPRDLIS
NINVIVLELKGSETTFMCEYADETATIVEFLNRWITFCQSII
STLT

Thus, in one embodiment, an IL2 variant comprises at least one or at least two amino acid modifications (e.g. substitution, insertion, deletion) compared to a human wild type IL-2 polypeptide. In one embodiment, an IL2v comprises a R38 substitution (e.g. R38A) and an F42 substitution (e.g., F42K), compared to a human wild type IL-2 polypeptide. In one embodiment, an IL2v comprises a R38 substitution (e.g. R38A), an F42 substitution (e.g., F42K) and a T41 substitution (e.g. T41A), compared to a human wild type IL-2 polypeptide. In one embodiment, an IL2v comprises a T3 substitution (e.g. T3A), an F42 substitution (e.g., F42A), a Y45 substitution (e.g. Y45A), a L72 substitution (e.g. L72G) and a C125 substitution (e.g. C125A), compared to a human wild type IL-2 polypeptide. Optionally the IL2v comprises an amino acid sequence identical to or at least 70%, 80%, 90%, 95%, 98% or 99% identical to the polypeptide of SEQ ID NOS: 404-409. Optionally the IL2v comprises a fragment of a human IL-2 polypeptide, wherein the fragment has an amino sequence identical to or at least 70%, 80%, 90%, 95%, 98% or 99% identical to a contiguous sequence of 40, 50, 60, 70 or 80 amino acids of the polypeptide of SEQ ID NOS: 404-409.

Any combination of the positions can be modified. In some embodiments, the IL-2 variant comprises two or more modification. In some embodiments, the IL-2 variant comprises three or more modification. In some embodiments, the IL-2 variant comprises four, five, or six or more modifications.

IL2 variant polypeptides can for example comprise two, three, four, five, six or seven amino acid modifications (e.g. substitutions). For example, U.S. Pat. No. 5,229,109, the disclosure of which is incorporated herein by reference, provides a human IL2 polypeptide having a R38A and F42K substitution. U.S. Pat. No. 9,447,159, the disclosure of which is incorporated herein by reference, describes human IL2 polypeptides having substitutions T3A, F42A, Y45A, and L72G substitutions. U.S. Pat. No. 9,266,938, the disclosure of which is incorporated herein by reference, describes human IL2 polypeptides having substitutions at residue L72 (e.g. L72G, L72A, L72S, L72T, L72Q, L72E, L72N, L72D, L72R, and L72K), residue F42 (e.g. F42A, F42G, F42S, F42T, F42Q, F42E, F42N, F42D, F42R, and F42K); and at residue Y45 (e.g., Y45A, Y45G, Y45S, Y45T, Y45Q, Y45E, Y45N, Y45D, Y45R and Y45K), including for example the triple mutation F42A/Y45A/L72G to reduce or abolish the affinity for IL-2Rα receptor. Yet further WO2020/057646, the disclosure of which is incorporated herein by reference, relates to amino acid sequence of IL-2v polypeptides comprising amino acid substitutions in various combinations among amino acid residues K35, T37, R38, F42, Y45, E61 and E68. Yet further, WO2020252418, the disclosure of which is incorporated herein by reference, relates to amino acid sequence of IL-2v polypeptides having at least one amino acid residues position R38, T41, F42, F44, E62, P65, E68, Y107, or S125 substituted with another amino acid, for example wherein the amino acid substitution is selected from the group consisting of: the substitution of L19D, L19H, L19N, L19P, L19Q, L19R, L19S, L19Y at position 19, the substitution of R38A, R38F, R38G at position 38, the substitution of T41A, T41G, and T41V at position 41, the substitution of F42A at position 42, the substitution of F44G and F44V at position 44, the substitution of E62A, E62F, E62H, and E62L at position 62, the substitution of P65A, P65E, P65G, P65H, P65K, P65N, P65Q, P65R at position 65, the substitution of E68E, E68F, E68H, E68L, and E68P at position 68, the substitution of Y107G, Y107H, Y107L and Y107V at position 107, and the substitution of S125I at position 125, the substitution of Q126E at position 126. Numbering of positions is with respect to Wild-type mature human IL-2.

A modified IL-2 can optionally be specified as exhibiting a KD for binding to CD25 or to a CD25:CD122:CD132 complex that is decreased by at least 1-log, optionally at least 2-log, optionally at least 3-log, compared to a wild-type human IL-2 polypeptide (e.g. comprising the amino acid sequence of SEQ ID NO: 404). A modified IL-2 can optionally be specified as exhibiting less than 20%, 30%, 40% or 50% of binding affinity to CD25 or to a CD25:CD122:CD132 complex compared to a wild-type human IL-2 polypeptide. An IL2 can optionally be specified as exhibiting at least 50%, 70%, 80% or 90% of binding affinity to CD122 or to a CD122:CD132 complex compared to a wild-type human IL-2 polypeptide. In some embodiments, an IL2 exhibits at least 50%, 60%, 70% or 80% but less than 100% of binding affinity to CD122 or to a CD122:CD132 complex compared to a wild-type human IL-2 polypeptide. In some embodiments, an IL2v exhibits less than 50% of binding affinity to CD25 and at least 50%, 60%, 70% or 80% of binding affinity to CD122, compared to wild-type IL-2 polypeptide.

Differences in binding affinity of wild-type and disclosed mutant polypeptide for CD25 and CD122 and complexes thereof can be measured, e.g., in standard surface plasmon resonance (SPR) assays that measure affinity of protein-protein interactions familiar to those skilled in the art.

Exemplary IL2 variant polypeptides have one or more, two or more, or three or more CD25-affinity-reducing amino acid substitutions relative to the wild-type mature IL-2 polypeptide having an amino acid sequence of SEQ ID NO: 404. In one embodiment, the exemplary IL2v polypeptides comprise one or more, two or more, or three or more substituted residues selected from the following group: Q11, H16, L18, L19, D20, D84, S87, Q22, R38, T41, F42, K43, Y45, E62, P65, E68, V69, L72, D84, S87, N88, V91, 192, T123, Q126, S127, 1129, and S130.

In one embodiment, the exemplary IL2 variant polypeptide has one, two, three, four, five or more of amino acid residues position R38, T41, F42, F44, E62, P65, E68, Y107, or S125 substituted with another amino acid.

In one embodiment, decreased affinity to CD25 or a protein complex comprising such (e.g., a CD25:CD122:CD132 complex) may be obtained by substituting one or more of the following residues in the sequence of the wild-type mature IL-2 polypeptide: R38, F42, K43, Y45, E62, P65, E68, V69, and L72.

In one embodiment, a CD122 ABD or IL-2 polypeptide is an IL-2 mimetic polypeptide. Synthetic IL-2/IL-15 polypeptide mimics can be computationally designed to bind to CD122, but not to CD25, for example as described in Silva et al, (2019) Nature 565(7738): 186-191 and WO2020/005819, the disclosures of which are incorporated herein by reference, also provides IL-2 and IL-15 mimetic polypeptides that bind CD122 but not CD25.

For example, an IL-2 mimetic polypeptide can be characterized as a non-naturally occurring polypeptide comprising domains X₁, X₂, X₃, and X₄, wherein:
(a) X, is a peptide comprising the amino acid sequence at least 85% identical to EHALYDAL (SEQ ID NO: 409);
(b) X₂ is a helical-peptide of at least 8 amino acids in length;
(c) X₃ is a peptide comprising the amino acid sequence at least 85% identical to YAFNFELI (SEQ ID NO: 410);
(d) X₄ is a peptide comprising the amino acid sequence at least 85% identical to ITILQSWIF (SEQ ID NO: 411);
wherein X₁, X₂, X₃, and X₄ may be in any order in the polypeptide,
wherein amino acid linkers may be present between any of the domains, and wherein the polypeptide binds to CD122 (or to the CD122:CD132 heterodimer). Optionally, the polypeptides bind the CD122:CD132 heterodimer with a binding affinity of 200 nM or less, 100 nM or less, 50 nM or less or 25 nM or less.

In one aspect, the invention provides non-naturally occurring polypeptides comprising domains X₁, X₂, X₃, and X₄, wherein:
(a) X₁ is a peptide comprising the amino acid sequence EHALYDAL (SEQ ID NO: 409);
(b) X₂ is a helical-peptide of at least 8 amino acids in length;
(c) X₃ is a peptide comprising the amino acid sequence YAFNFELI (SEQ ID NO: 410);
(d) X₄ is a peptide comprising the amino acid sequence ITILQSWIF (SEQ ID NO:411);
wherein X₁, X₂, X₃, and X₄ may be in any order in the polypeptide,
wherein amino acid linkers may be present between any of the domains, and wherein the polypeptide binds to CD122 (or to the CD122:CD132 heterodimer). Optionally, the polypeptides bind the CD122:CD132 heterodimer with a binding affinity of 200 nM or less, 100 nM or less, 50 nM or less or 25 nM or less, optionally between about 1 nm and about 100 nm, optionally between about 10 nM and about 200 nM, optionally between about 10 nM and about 100 nM optionally between about 15 nM and about 100 nM.

In one example, X₁, X₃, and X₄ may be any suitable length, meaning each domain may contain any suitable number of additional amino acids other than the peptides of SEQ ID NOS: 409, 410 and 411, respectively. In one embodiment, X₁ is a peptide comprising the amino acid sequence at least 25%, 27%, 30%, 35%, 40%, 45%, 50%, 55%, 60%, 65%, 70%, 75%, 80%, 85%, 90%, 95%, 98%, or 100% identical along its length to the peptide PKKKIQLHAEHALYDALMILNI (SEQ ID NO: 412); X₃ is a peptide comprising the amino acid sequence at least 25%, 27%, 30%, 35%, 40%, 45%, 50%, 55%, 60%, 65%, 70%, 75%, 80%, 85%, 90%, 95%, 98%, or 100% identical along its length the amino acid sequence LEDYAFNFELILEEIARLFESG (SEQ ID NO: 413); and X₄ is a peptide comprising the amino acid sequence at least 25%, 27%, 30%, 35%, 40%, 45%, 50%, 55%, 60%, 65%, 70%, 75%, 80%, 85%, 90%, 95%, 98%, or 100% identical along its length to the amino acid sequence EDEQEEMANAIITILQSWIFS (SEQ ID NO:414).

In one example, a computationally designed synthetic IL-2 polypeptide or mimetic (or CD122 ABD) comprises the amino acid sequence (neoleukin) shown below (with or without a (GS₄)₃ domain linker:

(SEQ ID NO: 415)
PKKKIQLHAEHALYDALMILNIVKTNSPPAEEKLEDYAFNFELILEEIAR
LFESGDQKDEAEKAKRMKEWMKRIKTTASEDEQEEMANAIITILQSWIFS
or
(SEQ ID NO: 416)
GSSSSGSSSSGSSSSPKKKIQLHAEHALYDALMILNIVKTNSPPAEEKLE
DYAFNFELILEEIARLFESGDQKDEAEKAKRMKEWMKRIKTTASEDEQEE
MANAIITILQSWIFS.

In yet other examples, an IL-2 polypeptide is modified by connecting, fusing, binding or associating it with one or more other additional compounds, chemical compounds, polymer (e.g. PEG), or polypeptides or polypeptide chains that result in a decrease of binding to CD25. For example a wild-type IL-2 polypeptide or fragment thereof can be modified by binding to it a CD25 binding peptide or polypeptide, including but not limited to an anti-IL-2 monoclonal antibody or antibody fragment thereof that binds or interacts with CD25-binding site of human IL-2, thereby decreasing binding to CD25.

In one example, an IL-2 further comprises a receptor domain, e.g., a cytokine receptor domain. In one embodiment, the cytokine molecule comprises an IL-2 receptor, or a fragment thereof (e.g., an IL-2 binding domain of an IL-2 receptor alpha). In one example, a CD25-derived polypeptide is fused to an IL-2 polypeptide, as described in Lopes et al, J Immunother Cancer. 2020; 8(1), the disclosure of which is incorporated herein by reference. In one example, the IL-2 is a variant fusion protein comprising a circularly permuted (cp) IL-2 fused to a CD25 polypeptide (see e.g., PCT publication no. WO2020/249693, the disclosure of which is incorporated herein by reference). Where the CD122 ABD comprises a circularly permuted (cp) IL-2 fused to a CD25 polypeptide, the ABD can comprise a cpIL-2:IL-2Ra polypeptide or protein of described in PCT publication no. WO2013/184942, the disclosure of which is incorporated herein by reference. For example, the permuted (cp) IL-2 variant fused to a CD25 polypeptide can have the amino acid sequence:

(SEQ ID NO: 417)
SKNFHLRPRDLISNINVIVLELKGSETTFMCEYADETATIVEFLNRWITF
SQSIISTLTGGSSSTKKTQLQLEHLLLDLQMILNGINNYKNPKLTRMLTF
KFYMPKKATELKHLQCLEEELKPLEEVLNLAQGSGGGSELCDDDPPEIPH
ATFKAMAYKEGTMLNCECKRGFRRIKSGSLYMLCTGNSSHSSWDNQCQCT
SSATRNTTKQVTPQPEEQKERKTTEMQSPMQPVDQASLPGHCREPPPWEN
EATERIYHFWGQMVYYQCVQGYRALHRGPAESVCKMTHGKTRWTQPQLIC
TG,

or an amino acid sequence having sequence identity that is about 75%, 80%, 85%, 90%, 91%, 92%, 93%, 94%, 95%, 96%, 97%, 98%, 99% or higher over a contiguous stretch of about 20 amino acids up to the full length of SEQ ID NO: 417.

In one example, IL-2 is associated with a specific anti-IL-2 monoclonal antibody (mAb), thus forming an IL-2/anti-IL-2 mAb complex (IL-2cx). Such complexes have been shown to overcome the CD25 binding (Boyman et al., Science 311, 1924-1927 (2006). An exemplary anti-IL2 antibody is antibody NARA1. PCT publication no. WO2017/122130, the disclosure of which is incorporated herein by reference, describes fusion proteins in which flexible linkers are used to connect IL-2 to the variable region of the light or heavy chain of NARA1. Sahin et al. (Nature Communications volume 11, Article number: 6440 (2020)) describe an improved construct in which IL-2 is brought into contact with and bound to the complementarity-determining region 1 of the light chain (L-CDR1) of NARA1, which resulted in a protein complex in which the IL-2 is bound to its antigen-binding groove on the antibody fragment (or the polypeptide chain(s) of the fragment).

In other examples, an IL-2 polypeptide or fragment thereof can be modified by binding to it a moiety of interest (e.g. a compound, chemical compounds, polymer, linear or branched PEG polymer), covalently attached to a natural amino acid or to an unnatural amino acid installed at a selected position. Such a modified interleukin 2 (IL-2) polypeptide can comprise at least one unnatural amino acid at a position on the polypeptide that reduces binding between the modified IL-2 polypeptide and CD25 but retains significant binding to the CD122:CD132 signaling complex, wherein the reduced binding to CD25 is compared to binding between a wild-type IL-2 polypeptide and CD25. An unnatural amino acid can be positioned at any one or more of residues K35, T37, R38, T41, F42, K43, F44, Y45, E60, E61, E62, K64, P65, E68, V69, N71, L72, M104, C105, and Y107 of IL-2. As disclosed in PCT publication nos. WO2019/028419 and WO2019/014267, the disclosures of which are incorporated herein by reference, the unnatural amino acid can be incorporated into the modified IL-2 polypeptide by an orthogonal tRNA synthetase/tRNA pair. The unnatural amino acid can for example comprise a lysine analogue, an aromatic side chain, an azido group, an alkyne group, or an aldehyde or ketone group. The modified IL-2 polypeptide can then be covalently attached to a water-soluble polymer, a lipid, a protein, or a peptide through the unnatural amino acid. Examples of suitable polymers include polyethylene glycol (PEG), poly(propylene glycol) (PPG), copolymers of ethylene glycol and propylene glycol, poly(oxyethylated polyol), poly(olefinic alcohol), poly(vinylpyrrolidone), poly(hydroxyalkylmethacrylamide), poly(hydroxyalkylmethacrylate), poly(saccharides), poly(a-hydroxy acid), poly(vinyl alcohol), polyphosphazene, polyoxazolines (POZ), poly(N-acryloylmorpholine), or a combination thereof, or a polysaccharide such as dextran, polysialic acid (PSA), hyaluronic acid (HA), amylose, heparin, heparan sulfate (HS), dextrin, or hydroxyethyl-starch (HES).

In some examples, an exemplary IL2v/not-alpha IL-2 conjugate can comprise a full-length or fragment of an IL-2 polypeptide in which an amino acid residue in the IL-2 polypeptide (e.g. a residue at position selected from K35, F42, F44, K43, E62, P65, R38, T41, E68, Y45, V69, and L72) is replaced by a natural or non-natural amino acid residue attached to a polymer via a chemical linker. The polymer can be a PEG polymer, e.g. a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa.

A modified IL2 polypeptide can comprising at least one unnatural amino acid at a position on the polypeptide that reduces binding between the modified IL-2 polypeptide and CD25 but retains significant binding with CD122:CD132 signaling complex to form a CD122:CD132 complex, wherein the reduced binding to CD25 is compared to binding between a wild-type IL-2 polypeptide and CD25 An exemplary 112v/not-alpha IL-2 conjugate is THOR-707 (Synthorx inc).

For example, as described in PCT publication no. WO2020/163532, the disclosure of which is incorporated herein by reference, an amino acid residue in the IL-2 conjugate is replaced by the structure of Formula (I):

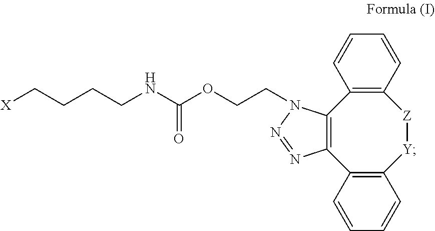

wherein:
Z is CH2 and Y is

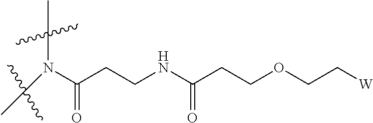

Y is CH2 and Z is

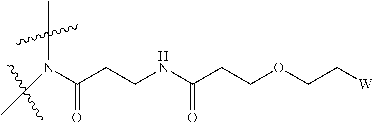

Z is CH2 and Y is

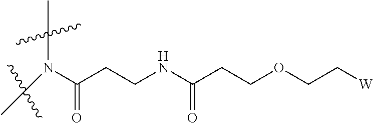

or,
Y is CH2 and Z is

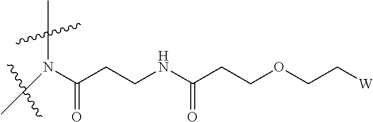

and wherein, W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa; and
X has the structure:

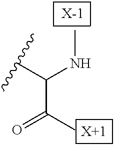

In one embodiment, an IL-2 comprises a releasable polymer (e.g. a releasable PEG polymer), e.g. the IL-2 is conjugated, linked or bound to a releasable polymer that results in a decrease in CD25 binding in vivo and/or in vitro. Example of such modified IL-2 include bempegaldesleukin or RSLAIL-2 (Nektar Therapeutics inc.), which exhibits about a 60-fold decrease in affinity to CD25 relative to IL-2, but only about a 5-fold decrease in affinity CD122 relative to IL-2. “Bempegaldesleukin” (CAS No. 1939126-74-5) is an IL-2 in which human interleukin-2 (des-1-alanine, 125-serine), is N-substituted with an average of six [(2,7-bis{[methylpoly(oxyethylene)iokD]carbamoyl}-9H-fluoren-9-yl)methoxy]carbonyl moieties at its amino residues. As disclosed in PCT publication no. WO2020/095183, the disclosure of which is incorporated herein by reference, the releasable PEG comprised can be based upon a 2,7,9-substituted fluorene, with poly(ethylene glycol) chains extending from the 2- and 7-positions on the fluorene ring via amide linkages (fluorene-C(O)—NH˜), and having releasable covalent attachment to IL-2 via attachment to a carbamate nitrogen atom attached via a methylene group (—CH2-) to the 9-position of the fluorene ring. The modified IL-2 can comprise compounds encompassed by the following formula:

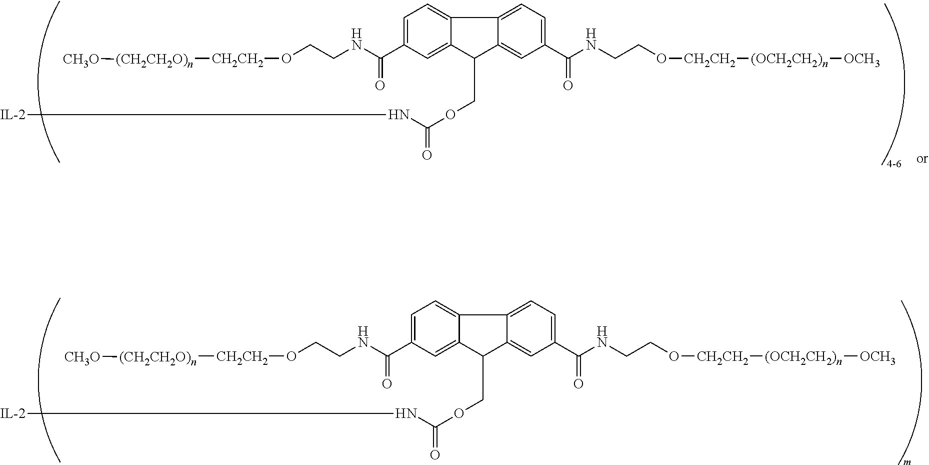

wherein each “n”, the number of CH₂CH₂O units) is an integer from about 3 to about 4000, or more preferably is an integer from about 200-300. “m”, referring to the number of polyethylene glycol moieties attached to IL-2 is an integer selected from the group consisting of 1, 2, 3, 7 and more than 7. In some embodiments, each “n” is approximately the same, i.e., the weight average molecular weight of each polyethylene glycol “arm” covalently attached to the fluorenyl core is about the same. Optionally, the weight average molecular weight of each PEG arm is about 10,000 daltons, such that the weight average molecular weight of the overall branched polymer moiety is about 20,000 daltons.

In another embodiment where the cytokine-binding ABD is a CD122-binding ABD, the ABD can be or comprise a suitable interleukin-15 (IL-15) polypeptide such that the CD122 ABD binds CD122. In some embodiments, the cytokine molecule is an IL-15 molecule, e.g., a full length, a fragment or a variant (IL-15v) of IL-15, e.g., human IL-15. In some embodiments, the IL-15 molecule comprises a wild-type human IL-15 amino acid sequence, e.g., having the amino acid sequence of SEQ ID NO: 418. In some embodiments, the IL-15 molecule comprises an amino sequence at least 70%, 80%, 90%, 95%, 98% or 99% identical to a mature wild-type human IL-15 amino acid sequence of SEQ ID NO: 418. In other embodiments, the IL-15 molecule is a variant of human IL-15, e.g., having one or more amino acid modifications. Optionally the IL-15 comprises a fragment of a human IL-15 polypeptide, wherein the fragment has an amino sequence is identical to or at least 70%, 80%, 90%, 95%, 98% or 99% identical to a contiguous sequence of 40, 50, 60, 70 or 80 amino acids of the wild-type mature human IL-15 polypeptide of SEQ ID NO: 418.

### Wild-Type Mature Human IL-15:

(SEQ ID NO: 418)
NW VNVISDLKKI EDLIQSMHID ATLYTESDVH PSCKVTAMKC
FLLELQVISL ESGDASIHDT VENLIILANN SLSSNGNVTE
SGCKECEELE EKNIKEFLQS FVHIVQMFIN TS

In some embodiments, an IL-15 variant comprises a modification (e.g. substitution) at position 45, 51, 52, or 72 (with reference to the sequence of human IL-15, SEQ ID NO: 418), e.g., as described in US 2016/0184399. In some embodiments, the IL-15 variant comprises four, five, or six or more modifications. In some embodiments, the IL-15 variant comprises one or more modification at amino acid position 8, 10, 61, 64, 65, 72, 101, or 108 (with reference to the sequence of human IL-15, SEQ ID NO: 418). In some embodiments the IL-15 variant possesses increased affinity for CD122 as compared with wild-type IL-15. In some embodiments the IL-15 variant possesses decreased affinity for CD122 as compared with wild-type IL-15. In some embodiments, the mutation is chosen from D8N, K10Q, D61N, D61H, E64H, N65H, N72A, N72H, Q101N, Q108N, or Q108H (with reference to the sequence of human IL-15, SEQ ID NO: 418). Any combination of the positions can be mutated. In some embodiments, the IL-15 variant comprises two or more mutations. In some embodiments, the IL-15 variant comprises three or more mutations. In some embodiments, the IL-15 variant comprises four, five, or six or more mutations. In some embodiments the IL-15 variant comprises mutations at positions 61 and 64. In some embodiments the mutations at positions 61 and 64 are D61N or D61H and E64Q or E64H. In some embodiments the IL-15 variant comprises mutations at positions 61 and 108. In some embodiments the mutations at positions 61 and 108 are D61N or D61H and Q108N or Q108H.

The extracellular domain of IL-15Rα comprises a domain referred to as the sushi domain, which binds IL-15. The general sushi domain, also referred to as complement control protein (CCP) modules or short consensus repeats (SCR), is a protein domain found in several proteins, including multiple members of the complement system. The sushi domain adopts a beta-sandwich fold, which is bounded by the first and fourth cysteine of four highly conserved cysteine residues, comprising a sequence stretch of approximately 60 amino acids (Norman, Barlow, et al. J Mol Biol. 1991; 219(4):717-25). The amino acid residues bounded by the first and fourth cysteines of the sushi domain IL-15Rα comprise a 62 amino acid polypeptide referred to as the minimal domain. Including additional amino acids of IL-15Rα at the N- and C-terminus of the minimal sushi domain, such as inclusion of N-terminal lie and Thr and C-terminal Ile and Arg residues result in a 65 amino acid extended sushi domain.

The CD122 ABD can further comprise a receptor domain, e.g., a cytokine receptor domain. In one embodiment, the cytokine molecule comprises an IL-15 receptor, or a fragment thereof (e.g., an IL-15 binding domain of an IL-15 receptor alpha).

In some embodiments, the CD122 ABD binds an IL-15 receptor alpha (IL-15Rα) sushi domain, a first domain linker, and an IL-15 polypeptide, e.g. from N- to C-terminus, a IL-15Rα sushi domain fused to a domain linker, in turn fused to an IL-15 polypeptide. Optionally the IL-15 polypeptide is a variant IL-15 polypeptide, e.g., comprising one or more amino acid substitutions. In other embodiments, the variant IL-15 domain comprises the amino acid sequence of SEQ ID NO: 418 and amino acid substitutions selected from the group consisting of N4D/N65D, D30N/N65D, and D30N/E64Q/N65D.

A sushi domain as described herein may comprise one or more mutations relative to a wild-type sushi domain. In some embodiments, the IL-15Rα sushi domain comprises the amino acid sequence:

(SEQ ID NO: 419)
ITCPPPMSVEHADIWVKSYSLYSRERYICNSGF
KRKAGTSSLTECVLNKATNVAHWTTPSLKCIR

An IL-15 polypeptide can be modified by connecting, fusing, binding or associating it with one or more other additional compounds using any of several known techniques, for example by conjugation or binding to chemical compounds, polymer (e.g. PEG), or polypeptides or polypeptide chains that result in a decrease of binding to IL-15Rα. In one example, a wild-type IL-15 polypeptide or fragment thereof can be modified by binding to it a IL-15Rα binding peptide or polypeptide, including but not limited to an anti-IL-15 monoclonal antibody or antibody fragment thereof that binds or interacts with IL-15Rα-binding site of human IL-15, thereby decreasing binding to IL-15Rα.

In another example, an IL-15 polypeptide or fragment thereof can be modified by binding to it a moiety of interest (e.g. a compound, chemical compounds, polymer, linear or branched PEG polymer), covalently attached to a novel amino acid installed at a selected position. Such a modified IL-15 polypeptide can comprise at least one unnatural amino acid at a position on the polypeptide that reduces binding between the modified IL-15 polypeptide and IL-15Rα but retains significant binding with CD122:CD132 signaling complex to form an CD122:CD132 complex, wherein the reduced binding to IL-15Rα is compared to binding between a wild-type IL-15 polypeptide and IL-15Rα. The unnatural amino acid can be positioned at any one or more of residues N1, W2, V3, N4, 16, S7, D8, K10, K11, E13, D14, L15, Q17, S18, M19, H20, 121, D22, A23, T24, L25, Y26, E28, S29, D30, V31, H32, P33, S34, C35, K36, V37, T38, K41, L44, E46, Q48, V49, S51, L52, E53, S54, G55, D56, A57, S58, H60, D61, T62, V63, E64, N65, 167, 168, L69, N71, N72, S73, L74, S75, S76, N77, G78, N79, V80, T81, E82, S83, G84, C85, K86, E87, C88, E89, E90, L91, E92, E93, K94, N95, 196, K97, E98, L100, Q101, S102, V104, H105, Q108, M109, F110, 1111, N112, T113, and S114 of IL-15. As disclosed in WO2019165453, WO2019/028419 and WO2019/014267, the disclosures of which are incorporated herein by reference, the unnatural amino acid can be incorporated into the modified IL-2 polypeptide by an orthogonal tRNA synthetase/tRNA pair. The unnatural amino acid can for example comprise a lysine analogue, an aromatic side chain, an azido group, an alkyne group, or an aldehyde or ketone group. The modified IL-15 polypeptide can then be covalently attached to a water-soluble polymer, a lipid, a protein, or a peptide through the unnatural amino acid. Examples of suitable polymers include polyethylene glycol (PEG), poly(propylene glycol) (PPG), copolymers of ethylene glycol and propylene glycol, poly(oxyethylated polyol), poly(olefinic alcohol), poly(vinylpyrrolidone), poly(hydroxyalkylmethacrylamide), poly(hydroxyalkylmethacrylate), poly(saccharides), poly(a-hydroxy acid), poly(vinyl alcohol), polyphosphazene, polyoxazolines (POZ), poly(N-acryloylmorpholine), or a combination thereof, or a polysaccharide such as dextran, polysialic acid (PSA), hyaluronic acid (HA), amylose, heparin, heparan sulfate (HS), dextrin, or hydroxyethyl-starch (HES).

For example, as described in WO2020/163532, the disclosure of which is incorporated herein by reference, an amino acid residue in the IL-15 conjugate is replaced by the structure of Formula (I):

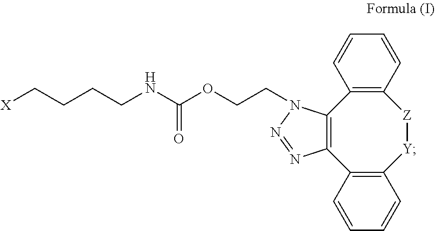

wherein:
Z is CH2 and Y is

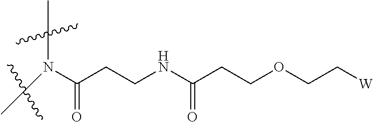

Y is CH2 and Z is

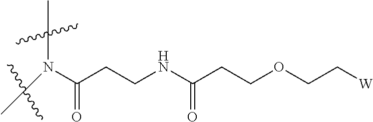

Z is CH2 and Y is

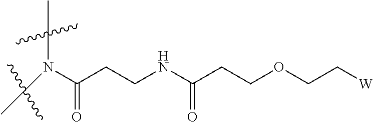

or,
Y is CH2 and Z is

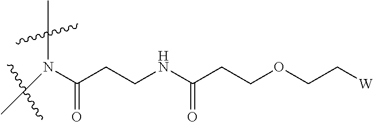

and wherein, W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa; and
X has the structure:

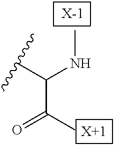

In one embodiment, an IL-15 comprises a releasable polymer (e.g. a releasable PEG polymer), e.g. the IL-15 is conjugated, linked or bound to a releasable polymer that results in a decrease in IL-15R binding in vivo and/or in vitro. Examples include compounds disclosed in PCT publication no. WO2020/097556, the disclosure of which is incorporated herein by reference. For example, the modified IL-15 can comprise comprising compound having the structure:

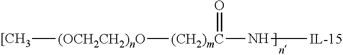

wherein (n) is an integer from about 150 to about 3,000, (m) is an integer selected from 2, 3, 4, and 5, (n′) is 1, and ˜NH˜ represents an amino group of the IL-15 polypeptide.

In another embodiment where the cytokine-binding ABD binds an IL-21 receptor (IL-21R)-binding ABD, the ABD can be or comprise a suitable interleukin-21 (IL-21) polypeptide such that the IL-21R ABD binds IL-21R on the surface of NK cells. IL-21R is similar in structure to the IL-2 receptor and the IL-15 receptor, in that each of these cytokine receptors comprises a common gamma chain (γc). In some embodiments, the cytokine molecule is an IL-21 molecule, e.g., a full length, a fragment or a variant of IL-21, e.g., human IL-21. In embodiments, the IL-21 molecule is a wild-type, human IL-21, e.g., having the amino acid sequence of SEQ ID NO: 420. In some embodiments, the IL-15 molecule comprises an amino sequence at least 70%, 80%, 90%, 95%, 98% or 99% identical to a mature wild-type human IL-21 amino acid sequence of SEQ ID NO: 420. In other embodiments, the IL-21 molecule is a variant of human IL-21, e.g., having one or more amino acid modifications. Optionally the IL-21 comprises a fragment of a human IL-21 polypeptide, wherein the fragment has an amino sequence is identical to or at least 70%, 80%, 90%, 95%, 98% or 99% identical to a contiguous sequence of 40, 50, 60, 70 or 80 amino acids of the polypeptide of SEQ ID NOS: 420.

### Wild-Type Mature Human IL-21:

(SEQ ID NO: 420)
HKSSSQ GQDRHMIRMR QLIDIVDQLK NYVNDLVPEF
LPAPEDVETN CEWSAFSCFQ KAQLKSANTG NNERIINVSI
KKLKRKPPST NAGRRQKHRL TCPSCDSYEK KPPKEFLERE
KSLLQKMIHQ HLSSRTHGSE DS

In some embodiments, the IL-21 variant can comprise an IL-21 polypeptide comprising one or more amino acid mutations designed to reduce its ability to bind to human IL-21R, while retaining substantial ability to bind human IL-21R. For example, the IL-21 can be characterized as binding to human IL-21R with a KD that is greater than or is about 0.04 nM, as determined by SPR.

Examples of such IL-21 variants are provided in PCT publication no. WO2019028316, the disclosure of which is incorporated herein by reference. In exemplary aspects, the amino acid substitutions are located at two amino acid positions selected from the group consisting of 10, 14, 20, 75, 76, 77, 78 and 81 according to numbering of SEQ ID NO: 420 or at two amino acid positions selected from the group consisting of 5, 9, 15, 70, 71, 72, 73, and 76, according to the amino acid position numbering of SEQ ID NO: 421. In exemplary aspects, the IL-21 variant comprises the amino acid sequence:

(SEQ ID NO: 421)
QGQDX HMXXM XXXXX XVDXL KNXVN
DLVPE FLPAP EDVET NCEWS AFSCF
QKAQL KSANT GNNEX XIXXX XXXLX
XXXXX TNAGR RQKHR LTCPS CDSYE
KKPPK EFLXX FXXLL XXMXX QHXSS
RTHGS EDS,

wherein X represents any amino acid, and wherein the IL-21 variant amino acid sequence differs from the amino acid sequence of human IL-21 (SEQ ID NO: 420) by at least 1 amino acid.

In exemplary aspects, the IL-21 variant comprises the sequence of SEQ ID NO: 421, wherein SEQ ID NO: 421 differs from SEQ ID NO: 420 by at least one amino acid at a position designated by X in SEQ ID NO: 421. In exemplary aspects, the IL-21 variant has at least about 30%, at least about 40%, at least about 50%, at least about 60%, at least about 70%, at least about 80%, at least about 85%, at least about 90%, or has greater than about 90% (e.g., about 91%, about 92%, about 93%, about 94%, about 95%, about 96%, about 97%, about 98%, or about 99%) sequence identity to SEQ ID NO: 420.

In exemplary embodiments, the IL-21 variant comprises an amino acid substitution relative to the wild-type IL-21 amino acid sequence within the N-terminal half of the amino acid sequence, e.g. at a position within positions 10-30 or 13-28 (both inclusive), according to the amino acid position numbering of SEQ ID NO: 420. In other exemplary embodiments, the IL-21 variant comprises an amino acid substitution relative to the wild-type IL-21 amino acid sequence within the C-terminal half of the amino acid sequence, e.g., at a position within positions 105-138 or 114-128 (both inclusive), according to the amino acid position numbering of SEQ ID NO: 420. In other exemplary embodiments, the IL-21 variant comprises an amino acid substitution relative to the wild-type IL-21 amino acid sequence in the middle third of the amino acid sequence, e.g., at a position within positions 60-90 or 70-85 (both inclusive), according to the amino acid position numbering of SEQ ID NO: 420.

Optionally, the IL-21 variant comprises only one amino acid substitution, relative to the wild-type IL-21 amino acid sequence. Optionally, the amino acid substitution is located at an amino acid position selected from the group consisting of: 10, 13, 14, 16, 17, 18, 19, 20, 21, 24, 28, 70, 71, 73, 74, 75, 76, 77, 78, 80, 81, 82, 83, 84, 85, 114, 115, 117, 118, 121, 122, 124, 125, or 128, according to the amino acid position numbering of SEQ ID NO: 420.

In another embodiment where the cytokine-binding ABD binds an IL-18 receptor (IL-18Rα)-binding ABD, the ABD can be or comprise a suitable interleukin-18 (IL-18) polypeptide such that the IL-18R ABD binds IL-18Rα on the surface of NK cells. In some embodiments, the cytokine molecule is an IL-18 molecule, e.g., a full length, a fragment or a variant of IL-18, e.g., human IL-18. In embodiments, the IL-18 molecule is a wild-type, human IL-18, e.g., having the amino acid sequence of SEQ ID NO: 422. In some embodiments, the IL-18 molecule comprises an amino sequence at least 70%, 80%, 90%, 95%, 98% or 99% identical to a mature wild-type human IL-18 amino acid sequence of SEQ ID NO: 422. In other embodiments, the IL-18 molecule is a variant of human IL-18, e.g., having one or more amino acid modifications. Optionally the IL-18 comprises a fragment of a human IL-18 polypeptide, wherein the fragment has an amino sequence is identical to or at least 70%, 80%, 90%, 95%, 98% or 99% identical to a contiguous sequence of 40, 50, 60, 70 or 80 amino acids of the polypeptide of SEQ ID NO: 422.

### Wild-Type Mature Human IL-18:

(SEQ ID NO: 422)
YFGKLESKLSVIRNLNDQVLFIDQGNRPLFEDMTDSDCRDNAPRTI
FIISMYKDSQPRGMAVTISVKCEKISTLSCENKIISFKEMNPPDN
IKDTKSDIIFFQRSVPGHDNKMQFESSSYEGYFLACEKERDLEKL
ILKKEDELGDRSIMFTVQNED

In one embodiment, an IL-18 is modified to decrease its binding affinity for IL-18BP while not substantially decreasing affinity for IL-18Rα. For example, an IL-18 may comprise of a modification such as amino acid substitutions at positions M51, S55, R104 and/or N110 that are not involved in IL-18Rα binding, optionally further in combination with a substitution at K53 and/or M60 (positions are with reference to the wild-type mature IL-18 amino acid sequence). In one embodiment, the IL-18 has a M51S, S55A, R104Q, R104K or R104S and/or N110A substitution. In one embodiment, the IL-18 comprises a K53S or K53A substitution. In one embodiment, the IL-18 comprises a M60S or M60K substitution.

In another embodiment, the cytokine-binding ABD binds a type I interferon receptor, for example interferon-α receptor (IFN-αR). The ABD can be or comprise a suitable type I interferon, for example an interferon-α (IFN-α) or interferon-β (IFN-β) polypeptide such that the IFN-α ABD binds IFN-αR on the surface of NK cells. The interferon-α receptor is also known as the interferon α/β receptor (IFNAR), a heterodimeric transmembrane receptor that is composed of the two subunits IFNAR1 and IFNAR2. For type I IFNs, the main STAT signaling complex is formed by IFN-stimulated gene factor 3 consisting of STAT1, STAT2, and IFN regulatory factor (IRF)-9. In some embodiments, the cytokine molecule is an IFN-α or IFN-β molecule, e.g., a full length, a fragment or a variant of IFN-α or IFN-β, e.g., human IFN-α or IFN-β, for example a human IFN-α1, IFN-α2, IFN-α4, IFN-α5, IFN-α6, IFN-α7, IFN-α8, IFN-α10, IFN-α12, IFN-α14, IFN-α16 or IFN-α17 polypeptide. In some embodiments, the IFN-α or IFN-β molecule is a wild-type, human IFN-α or IFN-β, e.g., having the amino acid sequence of any of SEQ ID NOS: 423-434. In other embodiments, the IFN-α or IFN-β molecule is a variant of human IFN-α or IFN-β, e.g., having one or more amino acid modifications. In some embodiments, the IFN-α or IFN-β molecule comprises an amino sequence at least 70%, 80%, 90%, 95%, 98% or 99% identical to a mature wild-type human IFN-α or IFN-β amino acid sequence of SEQ ID NOS: 423-434, respectively. In other embodiments, the IFN-α or IFN-β molecule is a variant of human IFN-α or IFN-β, e.g., having one or more amino acid modifications. Optionally the IFN-α or IFN-β comprises a fragment of a human IFN-α or IFN-β polypeptide, wherein the fragment has an amino sequence is identical to or at least 70%, 80%, 90%, 95%, 98% or 99% identical to a contiguous sequence of 40, 50, 60, 70 or 80 amino acids of the polypeptide of SEQ ID NOS: 423-434.

### Wild-Type Human IFN-α Mature Proteins:

IFNα2
(SEQ ID NO: 423)
CDLPQTHSLGSRRTLMLLAQMRRISLESCLKDRHDFGFPQEEFGN
QFQKAETIPVLHEMIQQIENLESTKDSSAAWDETLLDKFYTELY
QQLNDLEACVIQGVGVTETPLMKEDSILAVRKYFQRITLYLKEKK
YSPCAWEVVRAEIMRSFSLSTNLQESLRSKE
IFNα1
(SEQ ID NO: 424)
CDLPETHSLDNRRTLMLLAQMSRISPSSCLMDRHDFGFPQEEFDG
NQFQKAPAISVLHELIQQIENLETTKDSSAAWDEDLLDKFCTEL
YQQLNDLEACVMQEERVGETPLMNADSILAVKKYFRRITLYLTEK
KYSPCAWEVVRAEIMRSLSLSTNLQERLRRKE
IFNα4
(SEQ ID NO: 425)
CDLPQTHSLGNRRALILLAQMGRISHFSCLKDRHDFGFPEEEFDG
HQFQKTQAISVLHEMIQQTENLESTEDSSAAWEQSLLEKFSTELY
QQLNDLEACVIQEVGVEETPLMNEDSILAVRKY FQRITLYLTEK
KYSPCAWEVVRAEIMRSLSESTNLQKRLRRKD
IFNα5
(SEQ ID NO: 426)
CDLPQTHSLSNRRTLMIMAQMGRISPFSCLKDRHDFGFPQEEFDG
NQFQKAQAISVLHEMIQQTENLESTKDSSATWDETLLDKFYTELY
QQLNDLEACMMQEVGVEDTPLMNVDSILTVRKYFQRITLYLTEK
KYSPCAWEVVRAEIMRSFSLSANLQERLRRKE
IFNα6
(SEQ ID NO: 427)
CDLPQTHSLGHRRTMMLLAQMRRISLESCLKDRHDFRFPQEEFDG
NQFQKAEAISVLHEVIQQTENLESTKDSSVAWDERLLDKLYTELY
QQLNDLEACVMQEVWVGGTPLMNEDSILAVRKYFQRITLYLTEK
KYSPCAWEVVRAEIMRSFSSSRNLQERLRRKE
IFNα7
(SEQ ID NO: 428)
CDLPQTHSLRNRRALILLAQMGRISPFSCLKDRHEFRFPEEEFDG
HQFQKTQAISVLHEMIQQTENLESTEDSSAAWEQSLLEKESTEL
YQQLNDLEACVIQEVGVEETPLMNEDFILAVRKYFQRITLYLME
KKYSPCAWEVVRAEIMRSFSFSTNLKKGLRRKD
IFNα8
(SEQ ID NO: 429)
CDLPQTHSLGNRRALILLAQMRRISPFSCLKDRHDFEFPQEEFDD
KQFQKAQAISVLHEMIQQTENLESTKDSSAALDETLLDEFYIE
LDQQLNDLESCVMQEVGVIESPLMYEDSILAVRKYFQRITLYLT
EKKYSSCAWEVVRAEIMRSESLSINLQKRLKSKE
IFNα10
(SEQ ID NO: 430)
CDLPQTHSLGNRRALILLGQMGRISPFSCLKDRHDFRIPQEEFDG
NQFQKAQAISVLHEMIQQTENLESTEDSSAAWEQSLLEKFSTELY
QQLNDLEACVIQEVGVEETPLMNEDSILAVRKYFQRITLYLIERK
YSPCAWEVVRAEIMRSLSFSTNLQKRLRRKD
IFNα14
(SEQ ID NO: 431)
CNLSQTHSLNNRRTLMLMAQMRRISPFSCLKDRHDFE FPQEEFD
GNQFQKAQAISVLHEMMQQTENLESTKNSSAAWDETLLEKFYIEL
FQQMNDLEACVIQEVGVEETPLMNEDSILAVKKY FQRITLYLME
KKYSPCAWEVVRAEIMRSLSFSTNLQKRLRRKD
IFNα16
(SEQ ID NO: 432)
CDLPQTHSLGNRRALILLAQMGRISHFSCLKDRYDFGFPQEVEDG
NQFQKAQAISAFHEMIQQTENLFSTKDSSAAWDETLLDKFYIELF
QQLNDLEACVTQEVGVEEIALMNEDSILAVRKYFQRITLYLMG
KKYSPCAWEVVRAEIMRSESFSTNLQKGLRRKD
IFNα17
(SEQ ID NO: 433)
CDLPQTHSLGNRRALILLAQMGRISPFSCLKDRHDFGLPQEEFDG
NQFQKTQAISVLHEMIQQTENLESTEDSSAAWEQSLLEKFSTELY
QQLNNLEACVIQEVGMEETPLMNEDSILAVRKYFQRITLYLTEKK
YSPCAWEVVRAEIMRSLSFSTNLQKILRRKD

In certain aspects, the IFN-α or IFN-R variant polypeptide has an amino acid sequence that has at least about 30%, at least about 40%, at least about 50%, at least about 60%, at least about 70%, at least about 80%, at least about 85%, at least about 90%, or has greater than about 90% (e.g., about 91%, about 92%, about 93%, about 94%, about 95%, about 96%, about 97%, about 98%, or about 99%) sequence identity to SEQ ID NOS: 423-434, respectively.

In some embodiments, the wild type or modified signaling agent is a modified interferon-α having decreased binding affinity for its receptor, particularly IFNAR2. In such embodiments, the modified IFNα1, IFNα2, IFNα4, IFNα5, IFNα6, IFNα7, IFNα8, IFNα10, IFNα12, IFNα14, IFNα16 or IFNα17 agent has reduced affinity for and/or induction of signaling at IFNAR (IFNAR1 and/or IFNAR2 chains).

With the exception of wild-type IFNα1, wild-type IFNs bind to IFNAR2 at affinities (KD), e.g., as determined by microcal or SPR, between 0.1 nM and 5 nM and to IFNAR1 at an affinity of about 1 μM. IFNα1 binds to IFNAR2 with a KD of about 200 nM. In some embodiments, an IFN is modified so as to have an affinity for IFNAR1 and/or IFNAR2 that is equal or less than that of the NKp46 ABD for NKp46. In some embodiments, an IFN is modified so as to have an affinity for IFNAR1 and/or IFNAR2 that is at least 1-log less than that of the NKp46 ABD for NKp46.

For example, in the exemplary NKp46 ABDs shown herein, the NKp46 ABD has a KD for NKp46 binding of about 15 nM. An IFN can thus be modified by introduction of a modification that causes a reduction of binding affinity of between 10-fold (1-log) and 1000-fold (3-log) (an increase in KD of between 1-log and 3-log). An IFN can include any of the amino acid substitutions shown in the table below. The table below shows exemplary single amino acid substitutions that decrease binding affinity of IFN-α polypeptides to IFNAR2, with a cut-off of a decrease in affinity (higher KD) of at least 1 log compared to the wild-type counterpart and no more than 3-log compared to the wild-type counterpart. The table shows the relative affinity based on KID values for IFNAR2 of the mutated cytokine compared to the wild-type cytokine.

| | IFNAR2 | | | | | | | | | | |
|---|---|---|---|---|---|---|---|---|---|---|---|
| | relative | | | | | | | | | | |
| IFNα2 | affinity | IFNα6 | IFNα4 | IFNα17 | IFNα10 | IFNα7 | IFNα5 | IFNα14 | IFNα16 | IFNα1 | IFNα8 |
| L15A | 0.1 | M15A | L15A | L15A | L15A | L15A | L15A | L15A | L15A | L15A | L15A |
| L30A | 0.0013 | L30A | L30A | L30A | L30A | L30A | L30A | L30A | L30A | L30A | L30A |
| L30V | 0.023 | L30V | L30V | L30V | L30V | L30V | L30V | L30V | L30V | L30V | L30V |
| R144A | 0.03 | R145A | R145A | R145A | R145A | R145A | R145A | R145A | R145A | R145A | R145A |
| A145M | 0.16 | A146M | A146M | A146M | A146M | A146M | A146M | A146M | A146M | A146M | A146M |
| A145G | 0.03 | A146G | A146G | A146G | A146G | A146G | A146G | A146G | A146G | A146G | A146G |
| M148A | 0.03 | M149A | M149A | M149A | M149A | M149A | M149A | M149A | M149A | M149A | M149A |
| R149A | 0.01 | R150A | R150A | R150A | R150A | R150A | R150A | R150A | R150A | R150A | R150A |
| S152A | 0.1 | S153A | S153A | S153A | S153A | S153A | S153A | S153A | S153A | S153A | S153A |
| L153A | 0.1 | S154A | F154A | F154A | F154A | F154A | L154A | F154A | F154A | L154A | L154A |

The table below shows exemplary single amino acid substitutions decreasing binding affinity of IFN-α polypeptides to IFNAR1, with an at least 2-fold decrease in affinity. The table shows the relative affinity based on KID values for IFNAR1 of the mutated cytokine compared to the wild-type cytokine.

| | IFNAR1 | | | | | | | | | | |
|---|---|---|---|---|---|---|---|---|---|---|---|
| | relative | | | | | | | | | | |
| IFNα2 | affinity | IFNα | IFNα4 | IFNα17 | IFNα10 | IFNα7 | IFNα5 | IFNα14 | IFNα16 | IFNα1 | IFNα8 |
| F64A | 0.44 | F65A | F65A | F65A | F65A | F65A | F65A | F65A | F65A | F65A | F65A |
| N65A | 0.29 | N66A | N66A | N66A | N66A | N66A | N66A | N66A | N66A | N66A | N66A |
| T69A | 0.4 | T70A | T70A | T70A | T70A | T70A | T70A | T70A | T70A | T70A | T70A |
| S73A | 0.5 | S74A | S74A | S74A | S74A | S74A | S74A | S74A | S74A | S74A | S74A |
| L80A | 0.17 | L81A | L81A | L81A | L81A | L81A | L81A | L81A | L81A | L81A | L81A |
| Y85A | 0.35 | Y86A | Y86A | S86A | S86A | S86A | S86A | Y86A | Y86A | C86A | Y86A |
| Y89A | 0.25 | Y90A | Y90A | Y90A | Y90A | Y90A | Y90A | F90A | F90A | Y90A | D90A |
| N93A | 0.46 | N94A | N94A | N94A | N94A | N94A | N94A | N94A | N94A | N94A | N94A |
| E96A | 0.51 | E97A | E97A | E97A | E97A | E97A | E97A | E97A | E97A | E97A | E97A |
| L117A | 0.45 | L118A | L118A | L118A | L118A | L118A | L118A | L118A | L118A | L118A | L118A |
| R120A | <0.05 | R121A | R121A | R121A | R121A | R121A | R121A | K121A | R121A | K121A | R121A |

Mutant forms of IFNα2 are also described for example in POT publication nos. WO2008/124086, WO2010/030671, WO2013/059885, WO2013/107791, WO2015/007520 and WO2020/198661, the disclosures of which are incorporated hereby by reference.

In some embodiments, said IFNα2 mutant (IFNα2a or IFNα2b) is mutated at one or more amino acids at positions 144-154, such as amino acid positions 148, 149 and/or 153. In some embodiments, the IFNα2 mutant comprises one or more mutations selected from L153A, R149A, and M148A, described in WO2013/107791. In some embodiments, the IFNα2 mutants have reduced affinity and/or activity for IFNAR1. In some embodiments, the IFNα2 mutant comprises one or more mutations selected from F64A, N65A, T69A, L80A, Y85A, and Y89A, as described in WO2010/030671. In some embodiments, the IFNα2 mutant comprises one or more mutations selected from K133A, R144A, R149A, and L153A as described in WO2008/124086. In some embodiments, the IFNα2 mutant comprises one or more mutations selected from R120E and R120E/K121E, as described in WO2015/007520 and WO2010/030671. In one embodiment, the mutant human IFNα2 comprises an amino acid sequence having at least 95% identity with SEQ ID NO: 423, wherein the mutant IFNα2 has one or more mutations at positions L15, A19, R22, R23, L26, F27, L30, L30, K31, D32, R33, H34, D35, Q40, H57, E58, Q61, F64, N65, T69, L80, Y85, Y89, D 114, L117, R120, R125, K 133, K 134, R144, A145, M 148, R149, S 152, L153, and N156 with respect to SEQ ID NO: 423. In some embodiments, the human IFNα2 mutant comprises one or more mutations selected from, L15A, A19W, R22A, R23A, L26A, F27A, L30A, L30V, K31A, D32A, R33K, R33A, R33Q, H34A, D35A, Q40A, T106A, T106E, D114R, L117A, R120A, R125A, K134A, R144A, A145G, A145M, M148A, R149A, S152A, L153A, and N156A as disclosed in WO 2013/059885, for example in some embodiments, the human IFNα2 mutant comprises the mutations H57Y, E58N, Q61S, and/or L30A; the mutations H57Y, E58N, Q61S, and/or R33A; the mutations H57Y, E58N, Q61S, and/or M148A; the mutations H57Y, E58N, Q61S, and/or L153A; the mutations N65A, L80A, Y85A, and/or Y89A; or the mutations N65A, L80A, Y85A, Y89A, and/or D114A.

In embodiments, the wild type or modified signaling agent is a modified interferon-α having decreased binding affinity for its receptor, particularly IFNAR2. In such embodiments, the modified IFNα2 agent has reduced affinity for and/or induction of signaling at IFNAR (IFNAR1 and/or IFNAR2 chains).

In some embodiments, the IFNα1 interferon is modified to have a mutation at one or more amino acids at positions L15, A19, R23, S25, L30, D32, R33, H34, Q40, C86, D115, L118, K121, R126, E133, K134, K135, R145, A146, M149, R150, S153, L154, and N157 with reference to SEQ ID NO: 424. The mutations can optionally be a hydrophobic mutation and can be, e.g., selected from alanine, valine, leucine, and isoleucine. In some embodiments, the FNα1 interferon is modified to have a one or more mutations selected from L15A, A19W, R23A, S25A, L30A, L30V, D32A, R33K, R33A, R33Q, H34A, Q40A, C86S, C86A, D115R, 118A, K121A, K121 E, R126A, R126E, E133A, K134A, K135A, R145A, R145D, R145E, R145G, R145H, R1451, R145K, R145L, R145N, R145Q, R145S, R145T, R145V, R145Y, A146D, A146E, A146G, A146H, A1461, A146K, A146L, A146M, A146N, A146Q, A146R, A146S, A146T, A146V, A146Y, M149A, M149V, R150A, S153A, L154A, and N157A with reference to SEQ ID NO: 424. In some embodiments, the FNα1 mutant comprises one or more multiple mutations selected from L30A/H58Y/E59N/Q62S, R33A/H58Y/E59N/Q62S, M149A/H58Y/E59N/Q62S, L154A/H58Y/E59N/Q62S, R145A/H58Y/E59N/Q62S, D115A/R121A, L118A/R121A, L118A/R121A/K122A, R121A/K122A, and R121 E/K122E with reference to SEQ ID NO: 424. In some embodiments, the IFN-α1 is a variant that comprises one or more mutations which reduce undesired disulphide pairings wherein the one or more mutations are, e.g., at amino acid positions C1, C29, C86, C99, or C139 with reference to SEQ ID NO: 424. In some embodiments, the mutation at position C86 can be, e.g., C86S or C86A or C86Y.

In embodiments, the wild type or modified signaling agent is IFN-β. In some embodiments, the IFN-β is human having a sequence as shown below:

(SEQ ID NO: 434)
MSYNLLGFLQRSSNFQCQKLLWQLNGRLEYCLKDRMNFDIPEEIK
QLQQFQKEDAALTIYEMLQNIFAIFRQDSSSTGWNETIVENLLAN
VYHQINHLKTVLEEKLEKEDFTRGKLMSSLHLKRYYGRILHYLKA
KEYSHCAWTIVRVEILRNFYFINRLTGYLRN

In some embodiments, the human IFN-β is a non-glycosylated form of human IFN-β that has a Met-1 deletion and a Cys-17 to Ser mutation. In various embodiments, the modified IFN-β has one or more mutations that reduce its binding to or its affinity for the IFNAR1 subunit of IFNAR. In one embodiment, the modified IFN-β has reduced affinity and/or activity at IFNAR1. In various embodiments, the modified IFN-β is human IFN-β and has one or more mutations at positions F67, R71, L88, Y92, 195, N96, K123, and R124. In some embodiments, the one or more mutations are substitutions selected from F67G, F67S, R71A, L88G, L88S, Y92G, Y92S, 195A, N96G, K123G, and R124G.

In some embodiments, the modified IFN-β has one or more mutations that reduce its binding to or its affinity for the IFNAR2 subunit of IFNAR. In one embodiment, the modified IFN-β has reduced affinity and/or activity at IFNAR2. In various embodiments, the modified IFN-β is human IFN-β and has one or more mutations at positions W22, R27, L32, R35, V148, L151, R152, and Y155. In some embodiments, the one or more mutations are substitutions selected from W22G, R27G, L32A, L32G, R35A, R35G, V148G, L151G, R152A, R152G, and Y155G.

Exemplary IFN-β mutations are described in PCT publication nos. WO2020/198661, WO2000/023114 and US20150011732 the disclosures of which are incorporated hereby by reference.

In another embodiment where the cytokine-binding ABD binds an IL-7 receptor (IL-7R)-binding ABD, the ABD can be or comprise a suitable interleukin-7 (IL-7) polypeptide such that the IL-7R ABD binds IL-7Rα on the surface of NK cells. In some embodiments, the cytokine molecule is an IL-7 molecule, e.g., a full length, a fragment or a variant of IL-7, e.g., human IL-7. In embodiments, the IL-7 molecule is a wild-type, human IL-7, e.g., having the amino acid sequence of SEQ ID NO: 435. In some embodiments, the IL-7 molecule comprises an amino sequence at least 70%, 80%, 90%, 95%, 98% or 99% identical to a mature wild-type human IL-7 amino acid sequence of SEQ ID NO: 435. In other embodiments, the IL-7 molecule is a variant of human IL-7, e.g., having one or more amino acid modifications. Optionally the IL-7 comprises a fragment of a human IL-7 polypeptide, wherein the fragment has an amino sequence is identical to or at least 70%, 80%, 90%, 95%, 98% or 99% identical to a contiguous sequence of 40, 50, 60, 70 or 80 amino acids of the polypeptide of SEQ ID NO: 435.

### Wild-Type Mature Human IL-7:

(SEQ ID NO: 435)
DCDIEGKDGKQYESVLMVSIDQLLDSMKEIGSNCLNNEFNFFKRH
ICDANKEGMFLFRAARKLRQFLKMNSTGDFDLHLLKVSEGTTILL
NCTGQVKGRKPAALGEAQPTKSLEENKSLKEQKKLNDLCFLKRLL
QEIKTCWNKILMGTKEH.

Wild-type IL-7 bind to IL-7Rα with an affinity (KD), e.g., as determined by microcal or SPR, of between about 50-100 nM. In some embodiments, an IL-7 is modified so as to have an affinity for IL-7Rα that is equal or less than that of the NKp46 ABD for NKp46. In some embodiments, an IL-7 is modified so as to have an affinity for IL-7Rα that is at least 1-log less than that of the NKp46 ABD for NKp46. In some embodiments, an IL-7 is modified so as to have an affinity for IL-7Rα that is at least 1-log less than that of the NKp46 ABD for NKp46, but no more than 3-log, or optionally 2-log, less than that of the NKp46 ABD for NKp46. For example, in the exemplary NKp46 ABDs shown herein, the NKp46 ABD has a KD for NKp46 binding of about 15 nM. An IL-7 can thus be modified by introduction of a modification such as amino acid substitutions Q22A, D74A and/or K81A (with reference to the wild-type mature IL-7 amino acid sequence) that causes a reduction of affinity between IL-7 and IL-7Rα.

In another embodiment where the cytokine-binding ABD binds an IL-27 receptor (IL-27R)-binding ABD, the ABD can be or comprise a suitable interleukin-27 (IL-27) polypeptide such that the IL-27R ABD binds IL-27R (WSX-1 and/or gp130) on the surface of NK cells. In some embodiments, the cytokine molecule is an IL-27 molecule, e.g., a full length, a fragment or a variant comprising the P28 and EBI3 subunits, e.g., human single chain or heterodimeric IL-27 comprising the P28 and EBI3 subunits, optionally wherein the EBI3 and p28 subunits of IL-27 are linked by a domain linker (e.g., a flexible polypeptide linker, a linker containing glycine a serine residues, a (G₄S)₂ or (G₄S)₃ linker) into a single-chain format. Single-chain forms of IL-27 can be generated consisting of the p28 subunit linked to the EBI3 subunit by a flexible linker, either through the C-terminus of p28 linked to the N-terminus of EBI3 or vice versa. In embodiments, the IL-27 molecule is a wild-type, human IL-27, e.g., a single chain fusion product or a heterodimer comprising the amino acid sequences of SEQ ID NOS: 436 and 437 or a IL27R-binding fragment of any of the SEQ ID NOS: 436 and 437. In some embodiments, the IL-27 molecule comprises an amino sequence at least 70%, 80%, 90%, 95%, 98% or 99% identical to a mature wild-type human IL-27 p28 subunit amino acid sequence of SEQ ID NO: 436 and/or an amino sequence at least 70%, 80%, 90%, 95%, 98% or 99% identical to a mature wild-type human IL-27 EBI3 subunit amino acid sequence of SEQ ID NO: 437. In other embodiments, the IL-27 molecule is a variant of human IL-27, e.g., having one or more amino acid modifications. Optionally the IL-27 comprises a fragment of a human IL-27 p28 subunit polypeptide, wherein the fragment has an amino sequence that is identical to or at least 70%, 80%, 90%, 95%, 98% or 99% identical to a contiguous sequence of 40, 50, 60, 70 or 80 amino acids of the polypeptide of SEQ ID NO: 436, and/or a fragment of a human IL-27 EBI3 subunit polypeptide, wherein the fragment has an amino sequence that is identical to or at least 70%, 80%, 90%, 95%, 98% or 99% identical to a contiguous sequence of 40, 50, 60, 70 or 80 amino acids of the polypeptide of SEQ ID NO: 437. The p28 subunit can be specified as being linked at its N-terminus to the multispecific protein (or to the NKp46 ABD thereof). The EBI3 subunit can be specified as being linked, at its N-terminus, to the C-terminus of the p28 subunit, optionally via a domain linker, or can be specified as being placed on a separate polypeptide that associates with the p28 subunit.

### Wild-Type Mature Human IL-27 p28 Subunit:

(SEQ ID NO: 436)
FPRPPGRPQLSLQELRREFTVSLHLARKLLSEVRGQAHRFAESHL
PGVNLYLLPLGEQLPDVSLTFQAWRRLSDPERLCFISTTLQPFHA
LLGGLGTQGRWTNMERMQLWAMRLDLRDLQRHLRFQVLAAGENLP
EEEEEEEEEEEEERKGLLPGALGSALQGPAQVSWPQLLSTYRLLH
SLELVLSRAVRELLLLSKAGHSVWPLGFPTLSPQP

### Wild-Type Mature Human IL-27 EBI3 Subunit:

(SEQ ID NO: 437)
RKGPPAALTLPRVQCRASRYPIAVDCSWTLPPAPNSTSPVSFIAT
YRLGMAARGHSWPCLQQTPTSTSCTITDVQLESMAPYVLNVTAVH
PWGSSSSFVPFITEHIIKPDPPEGVRLSPLAERQLQVQWEPPGSW
PFPEIFSLKYWIRYKRQGAARFHRVGPIEATSFILRAVRPRARYY
VQVAAQDLTDYGELSDWSLPATATMSLGK

In some embodiments, an IL-27 is modified so as to have an affinity for WSX-1 and/or gp130 that is equal or less than that of the NKp46 ABD for NKp46. In some embodiments, an IL-27 is modified so as to have an affinity for WSX-1 and/or gp130 that is at least 1-log less than that of the NKp46 ABD for NKp46. In some embodiments, an IL-27 is modified so as to have an affinity for WSX-1 and/or gp130 that is at least 1-log less than that of the NKp46 ABD for NKp46, but no more than 3-log, or optionally 2-log, less than that of the NKp46 ABD for NKp46.

In another embodiment where the cytokine-binding ABD binds an IL-12 receptor (IL-12R)-binding ABD, the ABD can be or comprise a suitable interleukin-12 (IL-12) polypeptide such that the IL-12R ABD binds IL-12R (IL-12Rβ1 and/or IL-12Rβ2) on the surface of NK cells. In some embodiments, the cytokine molecule is an IL-12 molecule, e.g., a full length, a fragment or a variant comprising the P35 and P40 subunits, e.g., human single chain or heterodimeric IL-12 comprising the P35 and P40 subunits, optionally wherein the p40 and p35 subunits of IL-12 are linked by a domain linker (e.g., a flexible polypeptide linker, a linker containing glycine a serine residues, a (G₄S)₂ or (G₄S)₃ linker) into a single-chain format. Single-chain forms of IL-12 can be generated consisting of the p35 subunit linked to the p40 subunit by a flexible linker, either through the C-terminus of p35 linked to the N-terminus of p40 or vice versa. In embodiments, the IL-12 molecule is a wild-type, human IL-12, e.g., a single chain fusion product or a heterodimer comprising the amino acid sequences of SEQ ID NOS: 438 and 439 or a IL12R-binding fragment of any of the SEQ ID NOS: 438 or 439. In some embodiments, the IL-12 molecule comprises an amino sequence at least 70%, 80%, 90%, 95%, 98% or 99% identical to a mature wild-type human IL-12 p35 subunit amino acid sequence of SEQ ID NO: 438 and/or an amino sequence at least 70%, 80%, 90%, 95%, 98% or 99% identical to a mature wild-type human IL-12 p40 subunit amino acid sequence of SEQ ID NO: 439. In other embodiments, the IL-12 molecule is a variant of human IL-12, e.g., having one or more amino acid modifications. Optionally the IL-12 comprises a fragment of a human IL-12 p35 subunit polypeptide, wherein the fragment has an amino sequence that is identical to or at least 70%, 80%, 90%, 95%, 98% or 99% identical to a contiguous sequence of 40, 50, 60, 70 or 80 amino acids of the polypeptide of SEQ ID NO: 438, and/or a fragment of a human IL-12 p40 subunit polypeptide, wherein the fragment has an amino sequence that is identical to or at least 70%, 80%, 90%, 95%, 98% or 99% identical to a contiguous sequence of 40, 50, 60, 70 or 80 amino acids of the polypeptide of SEQ ID NO: 439. The p35 (alpha) and P40 (beta) can be specified as being linked by a disulphide bridge between Cys74 of the P35 subunit and the Cys177 of the P40 subunit. The p35 subunit can be specified as being linked at its N-terminus to the multispecific protein (or to the NKp46 ABD thereof). The p40 subunit can be specified as being linked, at its N-terminus, to the C-terminus of the p35 subunit, optionally via a domain linker, or can be specified as being placed on a separate polypeptide that associates with the p35 subunit.

Wild-type mature human IL-12 p35 subunit:

(SEQ ID NO: 438)
RNLPVATPDPGMFPCLHHSQNLLRAVSNMLQKARQTLEFYPCTSE
EIDHEDITKDKTSTVEACLPLELTKNESCLNSRETSFITNGSCLA
SRKTSEMMALCLSSIYEDLKMYQVEFKTMNAKLLMDPKRQIFLDQ
NMLAVIDELMQALNENSETVPQKSSLEEPDFYKTKIKLCILLHAF
RIRAVTIDRVMSYLNAS

Wild-type mature human IL-12 p40 subunit:

(SEQ ID NO: 439)
IWELKKDVYVVELDWYPDAPGEMVVLTCDTPEEDGITWTLDQSS
EVLGSGKTLTIQVKEFGDAGQYTCHKGGEVLSHSLLLLHKKEDG
IWSTDILKDQKEPKNKTFLRCEAKNYSGRFTCWWLTTISTDLTF
SVKSSRGSSDPQGVTCGAATLSAERVRGDNKEYEYSVECQEDSAC
PAAEESLPIEVMVDAVHKLKYENYTSSFFIRDIIKPDPPKNLQLK
PLKNSRQVEVSWEYPDTWSTPHSYFSLTFCVQVQGKSKREKKDRV
FTDKTSATVICRKNASISVRAQDRYYSSSWSEWASVPCS

Wild-type IL-12 dimer binds to IL-12Rβ1 and IL-12Rβ2 with an affinity (KD), e.g., as determined by microcal or SPR, of about 5-7 nM and 5 nM, respectively, and IL-12 dimer binds to IL12Rβ1:IL-12Rβ2 dimers with a KD of about 50 μM. In some embodiments, an IL-12 is modified so as to have an affinity for IL-12Rβ1 and/or IL-12Rβ2 that is equal or less than that of the NKp46 ABD for NKp46. In some embodiments, an IL-12 is modified so as to have an affinity for IL-12Rβ1 and/or IL-12Rβ2 that is at least 1-log less than that of the NKp46 ABD for NKp46. In some embodiments, an IL-12 is modified so as to have an affinity for IL-12Rβ1 and/or IL-12Rβ2 that is at least 1-log lower (1-log higher KD) than that of the NKp46 ABD for NKp46, but no more than 3-log, or optionally 2-log, lower than that of the NKp46 ABD for NKp46.

NKp46 Variable Region and CDR Sequences

In some embodiments, the multispecific protein or NKp46 ABD thereof (or the anti-NKp46 antibody from which the ABD is derived) binds the D1 domain of NKp46, the D2 domain of NKp46, or bind a region spanning the D1 and D2 domains (at the border of the D1 and D2 domains, the D1/D2 junction), of the NKp46 polypeptide of SEQ ID NO: 1. In some embodiments, the multispecific protein comprises VH/VL pair from an anti-NKp46 antibody having an affinity for human NKp46, as a full-length IgG antibody, characterized by a K_{D} of less than 10⁻⁸ M, less than 10⁻⁹ M, or less than 10⁻¹⁰ M. In some embodiments, the multispecific protein (or the NKp46-binding ABD thereof) has an affinity (KD) for human NKp46 of between 1 and 100 nM, optionally between 1 and 50 nM, optionally between 1 and 20 nM, optionally about 10 or 15 nM, as determined by SPR.

In one embodiment, the multispecific protein (or a NKp46-binding ABD or VH/VL pair thereof, for example as when configured in the multispecific protein or as a conventional full-length antibody) binds NKp46 at substantially the same region, site or epitope on NKp46 as antibody NKp46-1, NKp46-2, NKp46-3, NKp46-4, NKp46-6 or NKp46-9. In another embodiment, the antibodies at least partially overlaps, or includes at least one residue in the segment or epitope bound by NKp46-1, NKp46-2, NKp46-3, NKp46-4, NKp46-6 or NKp46-9.

In one embodiment, all key residues of the epitope are in a segment corresponding to domain D1 or D2. In one embodiment, the antibody or multispecific protein binds a residue present in the D1 domain as well as a residue present in in the D2 domain. In one embodiment, the antibodies bind an epitope comprising 1, 2, 3, 4, 5, 6, 7 or more residues in the segment corresponding to domain D1 or D2 of the NKp46 polypeptide of SEQ ID NO: 1. In one embodiment, the antibodies bind domain D1 and further bind an epitope comprising 1, 2, 3, or 4 of the residues R101, V102, E104 and/or L105.

In another embodiment, the antibodies or multispecific proteins bind NKp46 at the D1/D2 domain junction and bind an epitope comprising or consisting of 1, 2, 3, 4 or 5 of the residues K41, E42, E119, Y121 and/or Y194.

In another embodiment, the antibodies or multispecific proteins bind domain D2 and bind an epitope comprising 1, 2, 3, or 4 of the residues P132, E133, 1135, and/or S136.

The Examples section provided describes a series of mutant human NKp46 polypeptides. In the examples, the binding of multispecific protein to cells transfected with the NKp46 mutants was measured and compared to the ability of anti-NKp46 antibody to bind wild-type NKp46 polypeptide (SEQ ID NO:1). A reduction in binding between an anti-NKp46 antibody or NKp46 binding multispecific protein and a mutant NKp46 polypeptide as described herein means that there is a reduction in binding affinity (e.g., as measured by known methods such FACS testing of cells expressing a particular mutant, or by Biacore testing of binding to mutant polypeptides) and/or a reduction in the total binding capacity of the anti-NKp46 antibody (e.g., as evidenced by a decrease in Bmax in a plot of anti-NKp46 antibody concentration versus polypeptide concentration). A significant reduction in binding indicates that the mutated residue is directly involved in the binding to the anti-NKp46 antibody to NKp46 or is in close proximity to the binding protein when the anti-NKp46 antibody or NKp46 binding multispecific protein is bound to NKp46. An antibody epitope will thus preferably include such residue and may include additional residues adjacent to such residue.

In some embodiments, a significant reduction in binding means that the binding affinity and/or capacity between an NKp46 ABD or NKp46 binding multispecific protein and a mutant NKp46 polypeptide is reduced by greater than 40%, greater than 50%, greater than 55%, greater than 60%, greater than 65%, greater than 70%, greater than 75%, greater than 80%, greater than 85%, greater than 90% or greater than 95% relative to binding between the antibody and a wild type NKp46 polypeptide (e.g., the polypeptide shown in SEQ ID NO:1). In certain embodiments, binding is reduced below detectable limits. In some embodiments, a significant reduction in binding is evidenced when binding of an anti-NKp46 antibody to a mutant NKp46 polypeptide is less than 50% (e.g., less than 45%, 40%, 35%, 30%, 25%, 20%, 15% or 10%) of the binding observed between the anti-NKp46 antibody and a wild-type NKp46 polypeptide (e.g., the polypeptide shown in SEQ ID NO: 1 (or the extracellular domain thereof)). Such binding measurements can be made using a variety of binding assays known in the art. A specific example of one such assay is described in the Example section.

In some embodiments, NKp46 binding multispecific proteins exhibit significantly lower binding for a mutant NKp46 polypeptide in which a residue in a wild-type NKp46 polypeptide (e.g., SEQ ID NO:1) is substituted. In the shorthand notation used here, the format is: Wild type residue: Position in polypeptide: Mutant residue, with the numbering of the residues as indicated in SEQ ID NO: 1.

In some embodiments, an NKp46 binding multispecific binds a wild-type NKp46 polypeptide but has decreased binding to a mutant NKp46 polypeptide having a mutation (e.g., an alanine substitution) any one or more of the residues R101, V102, E104 and/or L105 (with reference to SEQ ID NO:1) compared to binding to the wild-type NKp46).

In some embodiments, a NKp46-binding multispecific protein binds a wild-type NKp46 polypeptide but has decreased binding to a mutant NKp46 polypeptide having a mutation (e.g., an alanine substitution) at one or more of residues K41, E42, E119, Y121 and/or Y194 (with reference to SEQ ID NO:1) compared to binding to the wild-type NKp46).

In some embodiments, a NKp46-binding multispecific protein binds a wild-type NKp46 polypeptide but has decreased binding to a mutant NKp46 polypeptide having a mutation (e.g., an alanine substitution) at one or more of residues P132, E133, 1135, and/or S136 (with reference to SEQ ID NO:1) compared to binding to the wild-type NKp46).

The amino acid sequence of the heavy chain variable region of antibodies NKp46-1, NKp46-2, NKp46-3, NKp46-4, NKp46-6 and NKp46-9 are listed herein in Table B (SEQ ID NOS: 3, 5, 7, 9, 11 and 13 respectively), the amino acid sequence of the light chain variable region of antibodies NKp46-1, NKp46-2, NKp46-3, NKp46-4, NKp46-6 and NKp46-9 are also listed herein in Table B (SEQ ID NOS: 4, 6, 8, 10, 12 and 14 respectively).

A NKp46-binding multispecific protein that binds essentially the same epitope or determinant as monoclonal antibody NKp46-1, NKp46-2, NKp46-3, NKp46-4, NKp46-6 or NKp46-9; optionally the antibody comprises a hypervariable region of antibody NKp46-1, NKp46-2, NKp46-3, NKp46-4, NKp46-6 or NKp46-9. In any of the embodiments herein, antibody NKp46-1, NKp46-2, NKp46-3, NKp46-4, NKp46-6 or NKp46-9 can be characterized by its amino acid sequence and/or nucleic acid sequence encoding it. In one embodiment, the antibody comprises the Fab or F(ab′)₂ portion of NKp46-1, NKp46-2, NKp46-3, NKp46-4, NKp46-6 or NKp46-9. Also provided is an antibody that comprises the heavy chain variable region of NKp46-1, NKp46-2, NKp46-3, NKp46-4, NKp46-6 or NKp46-9. According to one embodiment, an antibody comprises the three CDRs of the heavy chain variable region of NKp46-1, NKp46-2, NKp46-3, NKp46-4, NKp46-6 or NKp46-9. Also provided is a polypeptide that further comprises one, two or three of the CDRs of the light chain variable region of NKp46-1, NKp46-2, NKp46-3, NKp46-4, NKp46-6 or NKp46-9. Optionally any one or more of said light or heavy chain CDRs may contain one, two, three, four or five or more amino acid modifications (e.g. substitutions, insertions or deletions).

A multispecific protein or NKp46-binding ABD can for example comprise:
(a) the heavy chain variable region of NKp46-1, NKp46-2, NKp46-3, NKp46-4, NKp46-6 or NKp46-9 as set forth in Table B, optionally wherein one, two, three or more amino acids may be substituted by a different amino acid;
(b) the light chain variable region NKp46-1, NKp46-2, NKp46-3, NKp46-4, NKp46-6 or NKp46-9 as set forth in Table B, optionally wherein one, two, three or more amino acids may be substituted by a different amino acid;
(c) the heavy chain variable region of NKp46-1, NKp46-2, NKp46-3, NKp46-4, NKp46-6 or NKp46-9 as set forth in Table B, optionally wherein one or more of these amino acids may be substituted by a different amino acid; and the respective light chain variable region of NKp46-1, NKp46-2, NKp46-3, NKp46-4, NKp46-6 or NKp46-9 as set forth in Table B, optionally wherein one, two, three or more amino acids may be substituted by a different amino acid;
(d) the heavy chain CDR 1, 2 and 3 (HCDR1, HCDR2) amino acid sequence of NKp46-1, NKp46-2, NKp46-3, NKp46-4, NKp46-6 or NKp46-9 as shown in Table A, optionally wherein one, two, three or more amino acids in a CDR may be substituted by a different amino acid;
(e) the light chain CDR 1, 2 and 3 (LCDR1, LCDR2, LCDR3) amino acid sequence of NKp46-1, NKp46-2, NKp46-3, NKp46-4, NKp46-6 or NKp46-9 as shown in Table A, optionally wherein one, two, three or more amino acids in a CDR may be substituted by a different amino acid; or
(f) the heavy chain CDR 1, 2 and 3 (HCDR1, HCDR2, HCDR3) amino acid sequence of NKp46-1, NKp46-2, NKp46-3, NKp46-4, NKp46-6 or NKp46-9 as shown in Table A, optionally wherein one, two, three or more amino acids in a CDR may be substituted by a different amino acid; and the light chain CDRs 1, 2 and 3 (LCDR1, LCDR2, LCDR3) amino acid sequence of the respective NKp46-1, NKp46-2, NKp46-3, NKp46-4, NKp46-6 or NKp46-9 antibody as shown in Table A, optionally wherein one, two, three or more amino acids in a CDR may be substituted by a different amino acid.

In one embodiment, the aforementioned CDRs are according to Kabat, e.g. as shown in Table A. In one embodiment, the aforementioned CDRs are according to Chothia numbering, e.g. as shown in Table A. In one embodiment, the aforementioned CDRs are according to IMGT numbering, e.g. as shown in Table A.

In another aspect of any of the embodiments herein, any of the CHR2, CDR2 and CDR3 of the heavy and light chains may be characterized by a sequence of at least 4, 5, 6, 7, 8, 9 or 10 contiguous amino acids thereof, and/or as having an amino acid sequence that shares at least 50%, 60%, 70%, 80%, 85%, 90% or 95% sequence identity with the particular CDR or set of CDRs listed in the corresponding SEQ ID NO or Table A.

In another aspect, a multispecific protein competes for binding to an epitope on NKp46 with a monoclonal antibody according to (a) to (f), above.

The sequences of the CDRs, according to IMGT, Kabat and Chothia definitions systems, are summarized in Table A below. The sequences of the variable chains of the antibodies according to the invention are listed in Table B below. In any embodiment herein, a V_{L} or V_{H} sequence can be specified or numbered so as to contain or lack a signal peptide or any part thereof.

TABLE A

| | CDR | HCDR1 | | HCDR2 | | HCDR3 | |
|---|---|---|---|---|---|---|---|
| | defini- | SEQ | | SEQ | | SEQ | |
| mAb | tion | ID | Sequence | ID | Sequence | ID | Sequence |
| NKp46-1 | Kabat | 15 | DYVIN | 18 | EIYPGSGTNYYNEKFKA | 21 | RGRYGLYAMDY |
| | Chothia | 16 | GYTFTDY | 19 | PGSG | 22 | GRYGLYAMD |
| | IMGT | 17 | GYTFTDYV | 20 | GYTFTDYVIYPGSGTN | 23 | ARRGRYGLYAMDY |
| NKp46-2 | Kabat | 31 | SDYAWN | 34 | YITYSGSTSYNPSLES | 36 | GGYYGSSWGVFAY |
| | Chothia | 32 | GYSITSDY | | YSG | 37 | GYYGSSWGVFA |
| | IMGT | 33 | GYSITSDYA | 35 | ITYSGST | 38 | ARGGYYGSSWGVFAY |
| NKp46-3 | Kabat | 46 | EYTMH | 49 | GISPNIGGTSYNQKFKG | 51 | RGGSFDY |
| | Chothia | 47 | GYTFTEY | | PNIG | 52 | GGSFD |
| | IMGT | 48 | GYTFTEYT | 50 | ISPNIGGT | 53 | ARRGGSFDY |
| NKp46-4 | Kabat | 60 | SFTMH | 63 | YINPSSGYTEYNQKFKD | 65 | GSSRGFDY |
| | Chothia | 61 | GYTFTSF | | PSSG | 66 | SSRGED |
| | IMGT | 62 | GYTFTSFT | 64 | INPSSGYT | 67 | VRGSSRGFDY |
| NKp46-6 | Kabat | 73 | SSWMH | 76 | HIHPNSGISNYNEKFKG | 78 | GGRFDD |
| | Chothia | 74 | GYTFTSS | | PNSG | | GRED |
| | IMGT | 75 | GYTFTSSW | 77 | IHPNSGIS | 79 | ARGGREDD |
| NKp46-9 | Kabat | 85 | SDYAWN | 88 | YITYSGSTNYNPSLKS | 89 | CWDYALYAMDC |
| | Chothia | 86 | GYSITSDY | | YSG | 90 | WDYALYAMD |
| | IMGT | 87 | GYSITSDYA | 35 | ITYSGST | 91 | ARCWDYALYAMDC |
| Bab281 | Kabat | 97 | NYGMN | 100 | WINTNTGEPTYAEEFKG | 102 | DYLYYFDY |
| | Chothia | 98 | GYTFTNY | | TNTG | 103 | YLYYFD |
| | IMGT | 99 | GYTFTNYG | 101 | INTNTGEP | 104 | ARDYLYYFDY |
| | CDR | LCDR1 | | LCDR2 | | LCDR3 | |
| | defini- | SEQ | | SEQ | | SEQ | |
| mAb | tion | ID | Sequence | ID | Sequence | ID | Sequence |
| NKp46-1 | Kabat | 24 | RASQDISNYLN | 27 | YTSRLHS | 28 | QQGNTRPWT |
| | Chothia | 25 | SQDISNY | | YTS | 29 | YTSGNTRPW |
| | IMGT | 26 | QDISNY | | YTS | 30 | YTSQQGNTRPWT |
| NKp46-2 | Kabat | 39 | RVSENIYSYLA | 42 | NAKTLAE | 43 | QHHYGTPWT |
| | Chothia | 40 | SENIYSY | | NAK | 44 | HYGTPW |
| | IMGT | 41 | ENIYSY | | NAK | 45 | QHHYGTPWT |
| NKp46-3 | Kabat | 54 | RASQSISDYLH | 57 | YASQSIS | 58 | QNGHSFPLT |
| | Chothia | 55 | SQSISDY | | YAS | 59 | GHSFPL |
| | IMGT | 56 | QSISDY | | YAS | | QNGHSFPLT |
| NKp46-4 | Kabat | 68 | RASENIYSNLA | 70 | AATNLAD | 71 | QHFWGTPRT |
| | Chothia | | SENIYSN | | AAT | 72 | FWGTPR |
| | IMGT | 69 | ENIYSN | | AAT | | QHFWGTPRT |
| NKp46-6 | Kabat | 80 | RASQDIGSSLN | 81 | ATSSLDS | 82 | LQYASSPWT |
| | Chothia | | SQDIGSS | | ATS | 83 | YASSPWT |
| | IMGT | | QDIGSS | | ATS | 84 | LQYASSPWT |
| NKp46-9 | Kabat | 92 | RTSENIYSYLA | 93 | NAKTLAE | 94 | QHHYDTPLT |
| | Chothia | | SENIYSY | | NAK | 95 | NAKHYDTPL |
| | IMGT | | ENIYSY | | NAK | 96 | QHHYDTPLT |
| Bab281 | Kabat | 105 | KASENVVTYVS | 108 | GASNRYT | 109 | GQGYSYPYT |
| | Chothia | 106 | SENVVTY | | GAS | 110 | GYSYPY |
| | IMGT | 107 | ENVVTY | | GAS | 111 | GQGYSYPYT |

TABLE B

| | SEQ ID | |
|---|---|---|
| Antibody | NO | Amino acid sequence |
| NKp46-1 VH | 3 | QVQLQQSGPELVKPGASVKMSCKASGYTFTDYVINWGKQRSGQGLEWIGEI |
| | | YPGSGTNYYNEKFKAKATLTADKSSNIAYMQLSSLTSEDSAVYFCARRGRY |
| | | GLYAMDYWGQGTSVTVSS |
| NKp46-1 VL | 4 | DIQMTQTTSSLSASLGDRVTISCRASQDISNYLNWYQQKPDGTVKLLIYYT |
| | | SRLHSGVPSRFSGSGSGTDYSLTINNLEQEDIATYFCQQGNTRPWTFGGGT |
| | | KLEIK |
| NKp46-2 VH | 5 | EVQLQESGPGLVKPSQSLSLTCTVTGYSITSDYAWNWIRQFPGNKLEWMGY |
| | | ITYSGSTSYNPSLESRISITRDTSTNQFFLQLNSVTTEDTATYYCARGGYY |
| | | GSSWGVFAYWGQGTLVTVSA |
| NKp46-2 VL | 6 | DIQMTQSPASLSASVGETVTITCRVSENIYSYLAWYQQKQGKSPQLLVYNA |
| | | KTLAEGVPSRESGSGSGTQFSLKINSLQPEDFGSYYCQHHYGTPWTFGGGT |
| | | KLEIK |
| NKp46-3 VH | 7 | EVQLQQSGPELVKPGASVKISCKTSGYTFTEYTMHWVKQSHGKSLEWIGGI |
| | | SPNIGGTSYNQKFKGKATLTVDKSSSTAYMELRSLTSEDSAVYYCARRGGS |
| | | FDYWGQGTTLTVSS |
| NKp46-3 VL | 8 | DIVMTQSPATLSVTPGDRVSLSCRASQSISDYLHWYQQKSHESPRLLIKYA |
| | | SQSISGIPSRESGSGSGSDFTLSINSVEPEDVGVYYCQNGHSFPLTFGAGT |
| | | KLELK |
| NKp46-4 VH | 9 | QVQLQQSAVELARPGASVKMSCKASGYTFTSFTMHWVKQRPGQGLEWIGYI |
| | | NPSSGYTEYNQKFKDKTTLTADKSSSTAYMQLDSLTSDDSAVYYCVRGSSR |
| | | GFDYWGQGTLVTVSA |
| NKp46-4 VL | 10 | DIQMIQSPASLSVSVGETVTITCRASENIYSNLAWFQQKQGKSPQLLVYAA |
| | | TNLADGVPSRESGSGSGTQYSLKINSLQSEDFGIYYCQHFWGTPRTFGGGT |
| | | KLEIK |
| NKp46-6 VH | 11 | QVQLQQPGSVLVRPGASVKLSCKASGYTFTSSWMHWAKQRPGQGLEWIGHI |
| | | HPNSGISNYNEKFKGKATLTVDTSSSTAYVDLSSLTSEDSAVYYCARGGRE |
| | | DDWGAGTTVTVSS |
| NKp46-6 VL | 12 | DIQMTQSPSSLSASLGERVSLTCRASQDIGSSLNWLQQEPDGTIKRLIYAT |
| | | SSLDSGVPKRFSGSRSGSDYSLTISSLESEDFVDYYCLQYASSPWTFGGGT |
| | | KLEIK |
| NKp46-9 VH | 13 | DVQLQESGPGLVKPSQSLSLTCTVTGYSITSDYAWNWIRQFPGNKLEWMGY |
| | | ITYSGSTNYNPSLKSRISITRDTSKNQFFLQLNSVTTEDTATYYCARCWDY |
| | | ALYAMDCWGQGTSVTVSS |
| NKp46-9 VL | 14 | DIQMTQSPASLSASVGETVTITCRTSENIYSYLAWCQQKQGKSPQLLVYNA |
| | | KTLAEGVPSRESGSGSGTHESLKINSLQPEDFGIYYCQHHYDTPLTFGAGT |
| | | KLELK |

VH and VL pairs of an NKp46 ABD can optionally be function-conservative variants of the VH and VL of any of antibodies NKp46-1, NKp46-2, NKp46-3, NKp46-4, NKp46-6 or NKp46-9. “Function-conservative variants” are those in which a given amino acid residue in a protein (e.g. an antibody or antibody fragment) has been changed without altering the overall conformation and function of the protein, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide which has at least 60% amino acid identity with the antibody capable of specifically binding to a NKp46 polypeptide as defined hereinabove as determined by BLAST or FASTA algorithms, preferably at least 75%, more preferably at least 85%, still preferably at least 90%, and even more preferably at least 95%, and which has the same or substantially similar properties or functions as the antibodies capable of specifically binding to a NKp46 polypeptide as defined hereinabove.

Exemplary humanized VH and VL domains can comprise all of an antigen binding region of antibody NKp46-1, NKp46-2, NKp46-3, NKp46-4, NKp46-6 or NKp46-9, for example having the amino acids of the SEQ ID NOS shown in Table 5.

A light chain variable region of a NKp46-1, NKp46-2, NKp46-3, NKp-46-4, NKp46-6 or NKp46-9 antibody may comprise, for the respective antibody: a human light chain FR1 framework region; a LCDR1 region comprising an amino acid sequence as set forth in Table A, or a sequence of at least 4, 5, 6, 7, 8, 9 or 10 contiguous amino acids thereof, wherein one or more of these amino acids may be substituted by a different amino acid; a human light chain FR2 framework region; a LCDR2 region comprising an amino acid sequence as set forth in Table A, or a sequence of at least 4, 5, 6, 7, 8, 9 or 10 contiguous amino acids thereof, wherein one or more of these amino acids may be substituted by a different amino acid; a human light chain FR3 framework region; and a LCDR3 region comprising an amino acid sequence as set forth in Table A, or a sequence of at least 4, 5, 6, 7, 8, 9 or 10 contiguous amino acids thereof, wherein one or more of these amino acids may be deleted or substituted by a different amino acid. Optionally, the variable region further comprises a human light chain FR4 framework region. Humanization of NKp46-1, NKp46-2, NKp46-3, NKp-46-4, and NKp46-9 VH/VL domains is described in PCT publication no. WO2017114694, the disclosure of which is incorporated herein by reference, amino acid sequence shown below.

NKp46-1: “H1” heavy chain variable region
(SEQ ID NO: 112)
QVQLVQSGAEVKKPGSSVKVSCKASGYTFSDYVINWVRQAPGQGL
EWMGEIYPGSGTNYYNEKFKAKATITADKSTSTAYMELSSLRSED
TAVYYCARRGRYGLYAMDYWGQGTTVTVSS
NKp46-1: “H3” heavy chain variable region
(SEQ ID NO: 113)
QVQLVQSGAEVKKPGSSVKVSCKASGYTFTDYVINWGRQAPGQGL
EWIGEIYPGSGTNYYNEKFKAKATITADKSTSTAYMELSSLRSED
TAVYFCARRGRYGLYAMDYWGQGTTVTVSS
NKp46-1: “L1” light chain variable region
(SEQ ID NO: 114)
DIQMTQSPSSLSASVGDRVTITCRASQDISNYLNWYQQKPGKAPK
LLIYYTSRLHSGVPSRFSGSGSGTDFTFTISSLQPEDIATYFCQQ
GNTRPWTFGGGTKVEIK
NKp46-2: “H1” heavy chain variable region
(SEQ ID NO: 115)
QVQLQESGPGLVKPSQTLSLTCTVSGYSISSDYAWNWIRQPPGKG
LEWIGYITYSGSTSYNPSLESRVTISRDTSKNQFSLKLSSVTAAD
TAVYYCARGGYYGSSWGVFAYWGQGTLVTVSS
NKp46-2: “H2” heavy chain variable region
(SEQ ID NO: 116)
QVQLQESGPGLVKPSQTLSLTCTVSGYSISSDYAWNWIRQPPGKG
LEWMGYITYSGSTSYNPSLESRITISRDTSKNQFSLKLSSVTAAD
TAVYYCARGGYYGSSWGVFAYWGQGTLVTVSS
NKp46-2: “H3” heavy chain variable region
(SEQ ID NO: 117)
QVQLQESGPGLVKPSQTLSLTCTVSGYSITSDYAWNWIRQPPGKG
LEWMGYITYSGSTSYNPSLESRITISRDTSKNQFSLKLSSVTAAD
TAVYYCARGGYYGSSWGVFAYWGQGTLVTVSS
NKp46-2: “L1” light chain variable region
(SEQ ID NO: 118)
DIQMTQSPSSLSASVGDRVTITCRVSENIYSYLAWYQQKPGKAPK
LLVYNAKTLAEGVPSRESGSGSGTDFTLTISSLQPEDFATYYCQH
HYGTPWTFGGGTKVEIK
NKp46-3: “H1” heavy chain variable region
(SEQ ID NO: 119)
QVQLVQSGAEVKKPGSSVKVSCKASGYTFSEYTMHWVRQAPGQGL
EWMGGISPNIGGTSYNQKFKGRVTITADKSTSTAYMELSSLRSED
TAVYYCARRGGSFDYWGQGTTVTVSS
NKp46-3: “H3” heavy chain variable region
(SEQ ID NO: 120)
QVQLVQSGAEVKKPGSSVKVSCKASGYTFSEYTMHWVRQAPGQGL
EWIGGISPNIGGTSYNQKFKGRATITADKSTSTAYMELSSLRSED
TAVYYCARRGGSFDYWGQGTTVTVSS
NKp46-3: “H4” heavy chain variable region
(SEQ ID NO: 121)
QVQLVQSGAEVKKPGSSVKVSCKASGYTFSEYTMHWVRQAPGQGL
EWIGGISPNIGGTSYNQKFKGRATLTADKSTSTAYMELSSLRSED
TAVYYCARRGGSFDYWGQGTTVTVSS
NKp46-3: “L1” light chain variable region
(SEQ ID NO: 122)
EIVMTQSPATLSVSPGERATLSCRASQSISDYLHWYQQKPGQAPR
LLIKYASQSISGIPARFSGSGSGTDFTLTISSLEPEDFAVYYCQN
GHSFPLTFGQGTKLEIK
NKp46-4: “H1” heavy chain variable region
(SEQ ID NO: 123)
QVQLVQSGAEVKKPGASVKVSCKASGYTFTSFTMHWVRQAPGQGL
EWIGYINPSSGYTEYNQKFKDRVTITADKSTSTAYMELSSLRSED
TAVYYCVRGSSRGFDYWGQGTLVTVSS
NKp46-4: “H2” heavy chain variable region
(SEQ ID NO: 124)
QVQLVQSGAEVKKPGASVKVSCKASGYTFTSFTMHWVRQAPGQGL
EWIGYINPSSGYTEYNQKFKDRTTITADKSTSTAYMELSSLRSED
TAVYYCVRGSSRGFDYWGQGTLVTVSS
NKp46-4: “H3” heavy chain variable region
(SEQ ID NO: 125)
QVQLVQSGAEVKKPGASVKVSCKASGYTFTSFTMHWVRQAPGQGL
EWIGYINPSSGYTEYNQKFKDRTTLTADKSTSTAYMELSSLRSED
TAVYYCVRGSSRGFDYWGQGTLVTVSS
NKp46-4: “L2” light chain variable region
(SEQ ID NO: 126)
DIQMTQSPSSLSASVGDRVTITCRASENIYSNLAWFQQKPGKAPK
LLVYAATNLADGVPSRESGSGSGTDYTLTISSLQPEDFATYYCQH
FWGTPRTFGGGTKVEIK
NKp46-9: “H1” heavy chain variable region
(SEQ ID NO: 127)
QVQLQESGPGLVKPSQTLSLTCTVSGGSISSDYAWNWIRQPPGKG
LEWIGYITYSGSTNYNPSLKSRVTISRDTSKNQFSLKLSSVTAAD
TAVYYCARCWDYALYAMDCWGQGTTVTVSS
NKp46-9: “H2” heavy chain variable region
(SEQ ID NO: 128)
QVQLQESGPGLVKPSQTLSLTCTVSGYSISSDYAWNWIRQPPGKG
LEWIGYITYSGSTNYNPSLKSRVTISRDTSKNQFSLKLSSVTAAD
TAVYYCARCWDYALYAMDCWGQGTTVTVSS
NKp46-9: “H3” heavy chain variable region
(SEQ ID NO: 129)
QVQLQESGPGLVKPSQTLSLTCTVSGYSISSDYAWNWIRQPPGKG
LEWMGYITYSGSTNYNPSLKSRITISRDTSKNQFSLKLSSVTAAD
TAVYYCARCWDYALYAMDCWGQGTTVTVSS
NKp46-9: “L1” light chain variable region
(SEQ ID NO: 130)
DIQMTQSPSSLSASVGDRVTITCRTSENIYSYLAWCQQKPGKAPK
LLIYNAKTLAEGVPSRESGSGSGTDFTLTISSLQPEDFATYYCQH
HYDTPLTFGQGTKLEIK
NKp46-9: “L2” light chain variable region
(SEQ ID NO: 131)
DIQMTQSPSSLSASVGDRVTITCRTSENIYSYLAWCQQKPGKAPK
LLVYNAKTLAEGVPSRFSGSGSGTDFTLTISSLQPEDFATYYCQH
HYDTPLTFGQGTKLEIK

Examples of VH and VL combinations include:
(a) a VH comprising a CDR1, 2 and 3 of SEQ ID NO: 3 and a FR1, 2 and 3 derived from a human IGHV1-69 gene segment, and a VL comprising a CDR1, 2 and 3 of SEQ ID NO: 4 and a FR1, 2 and 3 derived from a human IGKV1-33 gene segment;
(b) a VH comprising a CDR1, 2 and 3 of SEQ ID NO: 5 and a FR1, 2 and 3 of a human IGHV4-30-4 gene segment, and a VL comprising a CDR1, 2 and 3 of SEQ ID NO: 6 and a FR1, 2 and 3 derived from a human IGKV1-39 gene segment;
(c) a VH comprising a CDR1, 2 and 3 of SEQ ID NO: 7 and a FR1, 2 and 3 derived from a human IGHV1-69 gene segment, and a VL comprising a CDR1, 2 and 3 of SEQ ID NO: 8 and a FR1, 2 and 3 derived from a human IGKV3-11 and/or IGKV3-15 gene segment;
(d) a VH comprising a CDR1, 2 and 3 of SEQ ID NO: 9 and a FR1, 2 and 3 derived from a human IGHV1-46 and/or a IGHV1-69 gene segment, and a VL comprising a CDR1, 2 and 3 of SEQ ID NO: 10 and a FR1, 2 and 3 derived from a human IGKV1-NL1 gene segment; or
(e) a VH comprising a CDR1, 2 and 3 of SEQ ID NO: 13 and a FR1, 2 and 3 derived from a human IGHV4-30-4 gene segment, and a VL comprising a CDR1, 2 and 3 of SEQ ID NO: 14 and a FR1, 2 and 3 derived from a human IGKV1-39 gene segment.

In another aspect, examples of humanized anti-NKp46 VH and VL combinations include:
(a) a VH comprising an amino acid sequence at least 70%, 80%, 90%, 95%, 98% or 100% identical to the amino acid sequence of the NKp46-1 H1 or H3 variable domain, and a VL comprising an amino acid sequence at least 70%, 80%, 90%, 95%, 98% or 100% identical to the amino acid sequence of the NKp46-1 L1 variable domain;
(b) a VH comprising an amino acid sequence at least 70%, 80%, 90%, 95%, 98% or 100% identical to the amino acid sequence of the NKp46-2 H1, H2 or H3 variable domain, and a VL comprising an amino acid sequence at least 70%, 80%, 90%, 95%, 98% or 100% identical to the amino acid sequence of the NKp46-2 L1 variable domain;
(c) a VH comprising an amino acid sequence at least 70%, 80%, 90%, 95%, 98% or 100% identical to the amino acid sequence of the NKp46-3 H1, H3 or H4 variable domain, and a VL comprising an amino acid sequence at least 70%, 80%, 90%, 95%, 98% or 100% identical to the amino acid sequence of the NKp46-3 L1 variable domain;
(d) a VH comprising an amino acid sequence at least 70%, 80%, 90%, 95%, 98% or 100% identical to the amino acid sequence of the NKp46-4 H1 variable domain, and a VL comprising an amino acid sequence at least 70%, 80%, 90%, 95%, 98% or 100% identical to the amino acid sequence of the NKp46-4 L2 variable domain;
(e) a VH comprising an amino acid sequence at least 70%, 80%, 90%, 95%, 98% or 100% identical to the amino acid sequence of the NKp46-9 H2 variable domain, and a VL comprising an amino acid sequence at least 70%, 80%, 90%, 95%, 98% or 100% identical to the amino acid sequence of the NKp46-9 L1 or L2 variable domain; or
(f) a VH comprising an amino acid sequence at least 70%, 80%, 90%, 95%, 98% or 100% identical to the amino acid sequence of the NKp46-9 H3 variable domain, and a VL comprising an amino acid sequence at least 70%, 80%, 90%, 95%, 98% or 100% identical to the amino acid sequence of the NKp46-9 L1 or L2 variable domain.

TABLE 5

| Antibody | VH (SEQ ID NO) | VL (SEQ ID NO) |
|---|---|---|
| NKp46-1 H1L1 | 112 | 114 |
| NKp46-1 H3L1 | 113 | 114 |
| NKp46-2 H1L1 | 115 | 118 |
| NKp46-2 H2L1 | 116 | 118 |
| NKp46-2 H3L1 | 117 | 118 |
| NKp46-3 H1L1 | 119 | 122 |
| NKp46-3 H3L1 | 120 | 122 |
| NKp46-3 H4L1 | 121 | 122 |
| NKp46-4 H1L2 | 123 | 126 |
| NKp46-4 H2L2 | 124 | 126 |
| NKp46-4 H3L2 | 125 | 126 |
| NKp46-9 H2L1 | 128 | 130 |
| NKp46-9 H2L2 | 128 | 131 |
| NKp46-9 H3L1 | 129 | 130 |
| NKp46-9 H3L2 | 129 | 131 |

Activity Testing

A multispecific protein can be assessed for biological activity, e.g., antigen binding, ability to elicit proliferation of NK cells, ability to elicit target cell lysis by NK and/or elicit activation of NK cells, including any specific signaling activities elicited thereby, for example cytokine production or cell surface expression of markers of activation. In one embodiment, provided are methods of assessing the biological activity, e.g., antigen binding, ability to elicit target cell lysis and/or specific signaling activities elicited thereby, of a multispecific protein of the disclosure. It will be appreciated that when the specific contribution or activity of one of the components of the multispecific protein is to be assessed (e.g. an NKp46 binding ABD, antigen-of-interest binding ABD, an Fc domain, cytokine receptor ABD, etc.), the multispecific format can be produced in a suitable format which allows for assessment of the component (e.g. domain) of interest. The present disclosure also provides such methods, for use in testing, assessing, making and/or producing a multispecific protein. For example, where the contribution or activity of a cytokine is assessed, the multispecific protein can be produced as a protein having the cytokine and another protein in which the cytokine is modified to delete it or otherwise modulate its activity (e.g., wherein the two multispecific proteins otherwise have the same or comparable structure), and tested in an assay of interest. For example, where the contribution or activity of an anti-NKp46 ABD is assessed, the multispecific protein can be produced as a protein having the ABD and another protein in which the ABD is absent or is replaced by an ABD that does not bind NKp46 (e.g., an ABD that binds an antigen not present in the assay system), wherein the two multispecific proteins otherwise have the same or comparable structure, and the two multispecific proteins are tested in an assay of interest. In another example, where the contribution or activity of an anti-antigen of interest (e.g. tumor antigen) ABD is assessed, the multispecific protein can be produced as a protein having the ABD and another protein in which the ABD is absent or is replaced by an ABD that does not bind the tumor antigen or anti-antigen of interest (e.g., an ABD that binds an antigen not present in the assay system, an ABD that bind to a different tumor antigen), wherein the two multispecific proteins otherwise have the same or comparable structure, and the two multispecific proteins are tested in an assay of interest.

In one aspect of any embodiment described herein, the multispecific protein is capable of inducing activation of an NKp46-expressing cell (e.g. an NK cell, a reporter cell) when the protein is incubated in the presence of the NKp46-expressing cell (e.g. purified NK cells) and a target cell (e.g. tumor cell) that expresses the antigen of interest (e.g. tumor antigen).

In one aspect of any embodiment described herein, the multispecific protein is capable of inducing NKp46 signaling in an NKp46-expressing cell (e.g. an NK cell, a reporter cell) when the protein is incubated in the presence of an NKp46-expressing cell (e.g. purified NK cells) and a target cell that expresses the antigen of interest). In one aspect of any embodiment described herein, the multispecific protein is capable of inducing CD16A signaling in an CD16A and NKp46-expressing cell (e.g. an NK cell, a reporter cell) when the protein is incubated in the presence of a CD16A and NKp46-expressing cell (e.g. purified NK cells) and a target cell that expresses the antigen of interest).

Optionally, NK cell activation or signaling in characterized by the increased expression of a cell surface marker of activation, e.g. CD107, CD69, Sca-1 or Ly-6A/E, KLRG1, etc.

In one aspect of any embodiment described herein, the multispecific protein is capable of inducing an increase of CD137 present on the cell surface of an NKp46- and/or a CD16-expressing cell (e.g. an NK cell, a reporter cell) when the protein is incubated in the presence of the NKp46- and/or a CD16-expressing cell (e.g. purified NK cells), optionally in the absence of target cells.

In one aspect of any embodiment described herein, the multispecific protein is capable of activating or enhancing the proliferation of NK cells by at least 10-fold, at least 50-fold, or at least 100-fold compared to the same multispecific protein lacking the cytokine receptor ABD (e.g. the cytokine, the CD122 ABD). Optionally the multispecific protein displays an EC50 for activation or enhancing the proliferation of NK cells that is at least 10-fold, 50-fold or 100-fold lower than its EC50 for activation or enhancing the proliferation of CD25-expressing T cells.

In one aspect of any embodiment described herein, the multispecific protein is capable of activating or enhancing the proliferation of NK cells over CD25-expressing T cells, by at least 10-fold, at least 50-fold, or at least 100-fold. Optionally, the CD25 expressing T cells are CD4 T cells, optionally Treg cells, or CD8 T cells.

Activation or enhancement of proliferation via cytokine receptor in cells (e.g. NK cells, CD4 T cells, CD8 Tcells or Treg cells) by the cytokine receptor ABD-containing protein can be determined by measuring the expression of pSTAT or the cell proliferation markers (e.g. Ki67) in said cells following the treatment with the multispecific protein. Activation or enhancement of proliferation via the IL-2R pathway in cells (e.g. NK cells, CD4 T cells, CD8 Tcells or Treg cells) by the CD122 ABD-containing protein can be determined by measuring the expression of pSTAT5 or the cell proliferation marker Ki67 in said cells following the treatment with the multispecific protein. IL-2 and IL-15 lead to the phosphorylation of the STAT5 protein, which is involved in cell proliferation, survival, differentiation and apoptosis. Phosphorylated STAT5 (pSTAT5) translocates into the nucleus to regulate transcription of the target genes including the CD25. STAT5 is also required for NK cell survival and NK cells are tightly regulated by the JAK-STAT signaling pathway. In one aspect of any embodiment described herein, the multispecific protein is capable of inducing STAT5 signaling in an NKp46-expressing cell (e.g. an NK cell) when the protein is incubated in the presence of an NKp46-expressing cell (e.g. purified NK cells). In one aspect of any embodiment described herein, the multispecific protein is capable of causing an increase of expression of pSTAT5 in NK cells over CD25-expressing T cells, by at least 10-fold, at least 50-fold, or at least 100-fold. Optionally the multispecific protein displays an EC₅₀ for induction of expression of pSTAT5 in NK cells that is at least 10-fold, 50-fold or 100-fold lower than its EC50 for induction of expression of pSTAT5 in CD25-expressing T cells. Similarly, cytokine receptor signal transduction can also be assessed for other cytokine/cytokine receptor pairs, such as IL-15 (STAT5), IL-21 (STAT3), IL-27 (STAT1), IL-12 (STAT4), etc.

Activity can be measured for example by bringing NKp46-expressing cells (or CD25-expressing cells, depending on the assay) into contact with the multispecific polypeptide, optionally further in presence of target cells (e.g. tumor cells). In some embodiments, activity is measured for example by bringing target cells and NK cells (i.e. NKp46-expressing cells) into contact with one another, in presence of the multispecific polypeptide. The NKp46-expressing cells may be employed either as purified NK cells or NKp46-expressing cells, or as NKp46-expressing cells within a population of peripheral blood mononuclear cell (PBMC). The target cells can be cells expressing the antigen of interest, optionally tumor cells or.

In one example, the multispecific protein can be assessed for the ability to cause a measurable increase in any property or activity known in the art as associated with NK cell activity, respectively, such as marker of cytotoxicity (CD107) or cytokine production (for example IFN-γ or TNF-α), increases in intracellular free calcium levels, the ability to lyse target cells, for example in a redirected killing assay, etc.

In the presence of target cells (target cells expressing the antigen of interest) and NK cells that express NKp46, the multispecific protein will be capable of causing an increase in a property or activity associated with NK cell activity (e.g. activation of NK cell cytotoxicity, CD107 expression, IFNγ production, killing of target cells) in vitro. For example, a multispecific protein according to the invention can be selected based on its ability to increase an NK cell activity by more than about 20%, preferably by least about 30%, at least about 40%, at least about 50%, or more compared to that achieved with the same effector: target cell ratio with the same NK cells and target cells that are not brought into contact with the multispecific protein, as measured by an assay that detects NK cell activity, e.g., an assay which detects the expression of an NK activation marker or which detects NK cell cytotoxicity, e.g., an assay that detects CD107 or CD69 expression, IFNγ production, or a classical in vitro chromium release test of cytotoxicity. Examples of protocols for detecting NK cell activation and cytotoxicity assays are described in the Examples herein, as well as for example, in Pessino et al, J. Exp. Med, 1998, 188 (5): 953-960; Sivori et al, Eur J Immunol, 1999. 29:1656-1666; Brando et al, (2005) J. Leukoc. Biol. 78:359-371; El-Sherbiny et al, (2007) Cancer Research 67(18):8444-9; and Nolte-'t Hoen et al, (2007) Blood 109:670-673). In a classical in vitro chromium release test of cytotoxicity, the target cells are labeled with ⁵¹Cr prior to addition of NK cells, and then the killing is estimated as proportional to the release of ⁵¹Cr from the cells to the medium, as a result of killing. Optionally, a multispecific protein according to the invention can be selected for or characterized by its ability to have greater ability to induce NK cell activity towards target cells, i.e., lysis of target cells compared to a conventional human IgG1 antibody that binds to the same antigen of interest, as measured by an assay of NK cell activity (e.g. an assay that detects NK cell-mediated lysis of target cells that express the antigen of interest).

As shown herein, a multispecific protein, the different ABDs contribute to the overall activity of the multispecific protein that ultimately manifests itself in potent anti-tumor activity in vivo. Testing methods exemplified herein allow the in vitro assessment of the activities of the different individual ABDs of the multispecific protein by making variants of the multispecific protein that lack a particular ABD and/or using cells that lack receptors for the particular ABD.

For example, a multispecific protein can be characterized by any of:
ability to cause activation of or enhancement of the proliferation of NK cells that is increased by at least 10-fold, at least 50-fold, or at least 100-fold (e.g. having an EC50 that is at least 10-fold, at least 50-fold, or at least 100-fold lower) compared to the same multispecific protein lacking the cytokine receptor ABD (e.g., determined by measuring the expression of pSTAT or the cell proliferation markers (e.g. Ki67) in said cells following the treatment with the multispecific protein);
ability to cause an increase in NK cell cytotoxicity toward tumor cells that is increased by at least 10-fold, at least 50-fold, or at least 100-fold (e.g. having an EC50 that is at least 10-fold, at least 50-fold, or at least 100-fold lower) compared to the same multispecific protein lacking a functional NKp46 ABD and/or lacking a CD16A domain (e.g. having an Fc region modified to reduce or abolish CD16A binding), as determined for example in an assay that detects CD107 or CD69 expression, IFNγ production, or a classical in vitro chromium release test of cytotoxicity;
ability to cause an increase in NK cell cytotoxicity toward tumor cells that is increased by at least 10-fold, at least 50-fold, or at least 100-fold (e.g. having an EC50 that is at least 10-fold, at least 50-fold, or at least 100-fold lower) compared to the same multispecific protein lacking the domain that binds an antigen of interest (e.g. tumor antigen), as determined for example in an assay that detects CD107 or CD69 expression, IFNγ production, or a classical in vitro chromium release test of cytotoxicity; and/or
ability to cause activation of or enhancement of the proliferation of NK cells that is increased by at least 10-fold, at least 50-fold, or at least 100-fold (e.g. having an EC50 that is at least 10-fold, at least 50-fold, or at least 100-fold lower) compared to the same multispecific protein lacking the NKp46 ABD and/or lacking a CD16A domain (e.g. having an Fc region modified to reduce or abolish CD16A binding), for example as determined by measuring the expression of pSTAT or the cell proliferation markers (e.g. Ki67) in said NK cells following the treatment of PBMC with the multispecific protein);
ability to cause activation of or enhancement of the proliferation of NK cells that is increased by at least 10-fold, at least 50-fold, or at least 100-fold (e.g. having an EC50 that is at least 10-fold, at least 50-fold, or at least 100-fold lower) compared to activation of or enhancement of the proliferation Treg cells, CD4 T cells and/or CD8 T cells (e.g., determined by measuring the expression of pSTAT or the cell proliferation markers (e.g. Ki67) in said NK, TReg, CD4 T cells and/or CD8 T cells following the treatment of PBMC with the multispecific protein).

As disclosed in the Examples, the lacking domains in the comparator proteins can be specified as being substitute domains (e.g. VH and VL pairs) that maintain the structure of the multispecific protein but do not bind to any antigen in the test system. As shown herein, a multispecific protein according to the disclosure, when it does not comprise the cytokine receptor ABD (e.g. the CD122 ABD) and when it possesses an Fc domain that does not bind CD16, does not, substantially induce NKp46 signaling (and/or NK activation that results therefrom) of NK cells when the protein is not bound to the antigen of interest on target cells (e.g. in the absence of the antigen of interest and/or target cells). Thus, the monovalent NKp46 binding component of the multispecific protein does not itself cause NKp46 signaling. Accordingly, in the case of multispecific proteins possessing an Fc domain that binds CD16, such multispecific protein can be produced in a configuration where the cytokine receptor ABD (e.g. CD122 ABD) is inactivated (e.g. modified, masked or deleted, thereby eliminating its ability to bind IL-2Rs) and the protein can be assessed for its ability to elicit NKp46 signaling or NKp46-mediated NK cell activation by testing the effect of this multispecific protein on NKp46 expression, by CD16-negative NK cells. The multispecific protein can optionally be characterized as not substantially causing (or increasing) NKp46 signaling by an NKp46-expressing, CD16-negative cell (e.g. a NKp46*CD16⁻ NK cell, a reporter cell) when the multispecific protein is incubated with such NKp46-expressing, CD16-negative cells (e.g., purified NK cells or purified reporter cells) in the absence of target cells.

In one aspect of any embodiment herein, a multispecific protein can for example be characterized by:
(a) capable of inducing cytokine receptor (e.g. CD122) signaling (e.g., as determined by assessing STAT signaling, for example assessing STAT phosphoylation) in an NKp46-expressing cell (e.g. an NK cell) when the multispecific protein is incubated in the presence of an NKp46-expressing cell (e.g. purified NK cells);
(b) being capable of inducing NK cells that express NKp46 (and optionally further CD16) to lyse target cells, when incubated in the presence of the NK cells and target cells; and
(c) lack of NK cell activation or cytotoxicity and/or lack of agonist activity at NKp46 when incubated with NK cells (optionally CD16-negative NK cells, NKp46-expressing NK cells that do not express CD16), in the absence of target cells, optionally wherein the NK cells are purified NK cells, when the multispecific protein is modified to lack the cytokine receptor ABD (e.g. CD122 ABD) or comprises an inactivated cytokine receptor ABD.

Assay conditions and methods, including but not limited to cell types and effector: target ratios can optionally be specified to be according to the Examples herein.

### Uses of Compounds

In one aspect, provided is the use of any of the multispecific proteins, cells which express the proteins (or a polypeptide chain thereof) and/or cells (e.g. NK cells) which have been loaded or incubated with the proteins, for the manufacture of a pharmaceutical preparation for the treatment, prevention or diagnosis of a disease in a mammal (e.g. human) in need thereof. Provided also are the use any of the compounds defined above as a medicament or an active component or active substance in a medicament. In a further aspect the invention provides methods for preparing a pharmaceutical composition containing a compound or composition as defined herein, to provide a solid or a liquid formulation for administration (e.g., by subcutaneous or intravenous injection). Such a method or process can be specified as comprising a step of mixing the compound with a pharmaceutically acceptable carrier.

In one aspect, provided is a method to treat, prevent or more generally affect a predefined condition in an individual or to detect a certain condition by using or administering a multispecific protein or antibody described herein, or a (pharmaceutical) composition comprising same.

For example, in one aspect, the invention provides a method of restoring or potentiating the activity and/or proliferation of NKp46-expressing cells, particularly NKp46⁺ NK cells (e.g. NKp46⁺CD16⁺ NK cells, NKp46⁺CD16− NK cells) in a patient in need thereof (e.g. a patient having a cancer, or a viral or bacterial infection), comprising the step of administering a multispecific protein described herein to said patient. In one aspect, the invention provides a method of selectively restoring or potentiating the activity and/or proliferation of NK cells over CD25-expressing lymphocytes, e.g. CD4 T cells, CD8 T cells, Treg cells. In one embodiment, the method is directed at increasing the activity of NKp46⁺ lymphocytes (e.g. NKp46⁺CD16⁺ NK cells, NKp46⁺CD16− NK cells) in patients having a disease in which increased lymphocyte (e.g. NK cell) activity is beneficial or which is caused or characterized by insufficient NK cell activity, such as a cancer, or a viral or microbial/bacterial infection.

In one aspect, the invention provides a method of restoring or potentiating the activity and/or proliferation of tumor-infiltrating NK cells or intra-tumoral NKp46-expressing cells, particularly NKp46⁺ NK cells (e.g. NKp46⁺CD16⁺ NK cells, NKp46⁺CD16⁻ NK cells) in a patient in need thereof (e.g. a patient having a solid tumor), comprising the step of administering a multispecific protein described herein to said patient.

In one aspect, the invention provides a method of increasing the number of tumor-infiltrating NK cells or intra-tumoral NKp46-expressing cells, particularly activated NKp46-expressing cells, particularly NKp46⁺ NK cells (e.g. NKp46⁺CD16⁺ NK cells, NKp46⁺CD16⁻ NK cells) in a patient in need thereof (e.g. a patient having a solid tumor), comprising the step of administering a multispecific protein described herein to said patient.

In another aspect, the invention provides a method of restoring or potentiating the activity and/or proliferation of NKp46⁺ NK cells (e.g. NKp46⁺CD16⁺ NK cells, NKp46⁺CD16⁻ NK cells) in a patient in need thereof (e.g. a patient having a cancer, or a viral, parasite or bacterial infection), comprising the step of contacting cells derived from the patient, e.g., immune cells and optionally target cells expressing an antigen of interest with a multispecific protein according to the invention and reinfusing the multispecific protein treated cells into the patient. In one embodiment, this method is directed at increasing the activity of NKp46⁺ lymphocytes (e.g. NKp46⁺CD16⁺ NK cells) in patients having a disease in which increased lymphocyte (e.g. NK cell) activity is beneficial or which is caused or characterized by insufficient NK cell activity, such as a cancer, or a viral or microbial, e.g., bacterial or parasite infection.

In another aspect, the invention provides a method of restoring or potentiating the activity and/or proliferation of NKp46⁺ NK cells (e.g. NKp46⁺CD16⁺ NK cells, NKp46⁺CD16⁻ NK cells) in a patient in need thereof (e.g. a patient having a cancer, or a viral, parasite or bacterial infection), comprising the step of contacting cells derived from the patient, e.g., immune cells with a multispecific protein according to the invention and reinfusing the multispecific protein-treated cells into the patient. In one embodiment, this method is directed at increasing the activity of NKp46⁺ lymphocytes (e.g. NKp46⁺CD16⁺ NK cells) in patients having a disease in which increased lymphocyte (e.g. NK cell) activity is beneficial or which is caused or characterized by insufficient NK cell activity, such as a cancer, or a viral or microbial, e.g., bacterial or parasite infection.

In another embodiment the subject multispecific proteins may be used or administered in combination with immune cells, particularly NK cells, derived from a patient who is to be treated or from a different donor, and these NK cells administered to a patient in need thereof such as a patient having a disease in which increased lymphocyte (e.g. NK cell) activity is beneficial or which is caused or characterized by insufficient NK cell activity, such as a cancer, or a viral or microbial, e.g., bacterial or parasite infection. As NK cells (unlike CAR-T cells) do not express TCRs, these NK cells, even those derived from different donors will not induce a GVHD reaction (see e.g., Glienke et al., “Advantages and applications of CAR-expressing natural killer cells”, Front. Pharmacol. 6, Art. 21:1-6 (2015); Hermanson and Kaufman, Front. Immunol. 6, Art. 195:1-6 (2015)).

In one embodiment, the multispecific protein disclosed herein that mediates NK cell activation, proliferation, tumor infiltration and/or target cell lysis via multiple activating receptors of effector cells, including NKp46, CD16 and CD122, can be used advantageously for treatment of individuals whose effector cells or tumor-infiltrating effector cells (e.g. NKp46+NK cells) cells are hypoactive, exhausted or suppressed, for example a patient who has a significant population of effector cells characterized by the expression and/or upregulation of one or multiple inhibitory receptors (e.g. TIM-3, PD1, CD96, TIGIT, etc.), or the downregulation or low level of expression of CD16 (e.g., presence of elevated proportion of NKp46⁺CD16⁻ NK cells).

The multispecific polypeptides or cells described herein can be used to prevent or treat disorders that can be treated with antibodies, such as cancers, solid and non-solid tumors, hematological malignancies, infections such as viral infections, and inflammatory or autoimmune disorders.

In one embodiment, the antigen of interest (the non-NKp46 antigen) is an antigen expressed on the surface of a malignant cell of a type of cancer selected from the group consisting of: carcinoma, including that of the bladder, head and neck, breast, colon, kidney, liver, lung, ovary, prostate, pancreas, stomach, cervix, thyroid and skin, including squamous cell carcinoma; hematopoietic tumors of lymphoid lineage, including leukemia, acute lymphocytic leukemia, acute lymphoblastic leukemia, B-cell lymphoma, T-cell lymphoma, Hodgkin's lymphoma, non-Hodgkin's lymphoma, hairy cell lymphoma and Burkett's lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xeroderma pigmentosum, keratoacanthoma, seminoma, thyroid follicular cancer and teratocarcinoma, hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) preferably of the T-cell type; Sezary syndrome (SS); Adult T-cell leukemia lymphoma (ATLL); a/d T-NHL hepatosplenic lymphoma; peripheral/post-thymic T cell lymphoma (pleomorphic and immunoblastic subtypes); angio immunoblastic T-cell lymphoma; angiocentric (nasal) T-cell lymphoma; anaplastic (Ki 1+) large cell lymphoma; intestinal T-cell lymphoma; T-lymphoblastic; and lymphoma/leukemia (T-Lbly/T-ALL).

In one embodiment, a multispecific protein is used to prevent or treat a cancer selected from the group consisting of: carcinoma, including that of the bladder, head and neck, breast, colon, kidney, liver, lung, ovary, prostate, pancreas, stomach, cervix, thyroid and skin, including squamous cell carcinoma; hematopoietic tumors of lymphoid lineage, including leukemia, acute lymphocytic leukemia, acute lymphoblastic leukemia, B-cell lymphoma, T-cell lymphoma, Hodgkin's lymphoma, non-Hodgkin's lymphoma, hairy cell lymphoma and Burkett's lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xeroderma pigmentosum, keratoacanthoma, seminoma, thyroid follicular cancer and teratocarcinoma. Other exemplary disorders that can be treated according to the invention include hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) preferably of the T-cell type; Sézary syndrome (SS); Adult T-cell leukemia lymphoma (ATLL); a/d T-NHL hepatosplenic lymphoma; peripheral/post-thymic T cell lymphoma (pleomorphic and immunoblastic subtypes); angio immunoblastic T-cell lymphoma; angiocentric (nasal) T-cell lymphoma; anaplastic (Ki 1+) large cell lymphoma; intestinal T-cell lymphoma; T-lymphoblastic; and lymphoma/leukaemia (T-Lbly/T-ALL).

In one example, the tumor antigen is an antigen expressed on the surface of a lymphoma cell or a leukemia cell, and the multispecific protein is administered to, and/or used for the treatment of, an individual having a lymphoma or a leukemia. Optionally, the tumor antigen is selected from HER2, CD19, CD20, CD22, CD30 or CD33.

In one embodiment, the inventive multispecific polypeptides or cell compositions described herein can be used to prevent or treat a cancer characterized by tumor cells that express the antigen of interest (e.g. tumor antigen) to which the multispecific protein of the disclosure specifically binds.

In one aspect, the methods of treatment comprise administering to an individual a multispecific protein described herein in a therapeutically effective amount, e.g., for the treatment of a disease as disclosed herein, for example any of the cancers identified above. A therapeutically effective amount may be any amount that has a therapeutic effect in a patient having a disease or disorder (or promotes, enhances, and/or induces such an effect in at least a substantial proportion of patients with the disease or disorder and substantially similar characteristics as the patient).

The multispecific protein may be used with our without a prior step of detecting the expression of the antigen of interest (e.g. tumor antigen) on target cells in a biological sample obtained from an individual (e.g. a biological sample comprising cancer cells, cancer tissue or cancer-adjacent tissue). In another embodiment, the disclosure provides a method for the treatment or prevention of a cancer in an individual in need thereof, the method comprising:
a) detecting cells (e.g. tumor cells) in a sample from the individual that express an antigen of interest (e.g. the antigen of interest to which the multispecific protein specifically binds via its antigen of interest ABD), and
b) upon a determination that cells which express an antigen of interest are comprised in the sample, optionally at a level corresponding at least to a reference level (e.g. corresponding to an individual deriving substantial benefit from a multispecific protein, or optionally at a level that is increased compared to a reference level (e.g. corresponding to a healthy individual or an individual not deriving substantial benefit from a protein described herein), administering to the individual a multispecific protein of the disclosure that binds to an antigen of interest, to NKp46, to cytokine receptor (e.g. CD122), and optionally to CD16A (e.g., via its Fc domain).

In some embodiments, the multispecific proteins are used to treat a tumor characterized by low levels of surface expression of the antigen of interest. Accordingly, in, the tumor or cancer can be characterized by cells expressing a low level of tumor antigen. Optionally, the level of the tumor antigen is less than 100,000 tumor antigen copies per cancer cell. In some aspects, the level of the tumor antigen is less than 90,000, less than 75,000, less than 50,000, or less than 40,000 tumor antigen copies per cancer cell. The uses optionally further comprise detecting the level of tumor antigen of one or more cancer cells of the subject.

The multispecific protein may be used with our without a prior step of detecting or characterizing NK cells from an individual to be treated. Optionally, in one embodiment, the invention provides a method for the treatment or prevention of a cancer in an individual in need thereof, the method comprising:
a) detecting NK cells (e.g. tumor-infiltrating NK cells) in a tumor sample from an individual (or within the tumor and/or within adjacent tissue), and
b) upon a determination that the tumor or tumor sample is characterized by a low number or activity of NK cells, optionally at a level or number that is decreased compared to a reference level (e.g. at a level corresponding to an individual deriving no, low or insufficient benefit from a conventional IgG antibody therapy such a conventional IgG1 antibody that binds to the same cancer antigen), administering to the individual a multispecific protein that binds to a cancer antigen, to NKp46 (e.g., monovalently), to cytokine receptor (e.g., CD122) and optionally to CD16A.

In some embodiments, an individual has a tumor characterized by a CD16 (e.g. CD16A) deficient tumor microenvironment. Optionally, the methods of treatment using a multispecific protein comprise a step of detecting the expression level of CD16 in a sample (e.g. a tumor sample) from the individual. Detecting the CD16 optionally comprises detecting the level of CD16A or CD16B. In some aspects, the CD16 deficient microenvironment is assessed in a patient having undergone a hematopoietic stem cell transplantation. Optionally, the CD16 deficient microenvironment comprises a population of infiltrating NK cells, and the infiltrating NK cells have less than 50% expression of CD16 as compared to a control NK cell. In some aspects, the infiltrating NK cells have less than 30%, less than 20%, or less than 10% expression of CD16 as compared to a control NK cell. Optionally, the CD16 deficient microenvironment comprises a population of infiltrating NK cells, and at least 10% of the infiltrating NK cells have reduced expression of CD16 as compared to a control NK cell. In some aspects, at least 20%, at least 30%, or at least 40% of the infiltrating NK cells have reduced expression of CD16 as compared to a control NK cell.

Optionally, in one embodiment, provided is a method for the treatment or prevention of a cancer in an individual in need thereof, the method comprising:
a) detecting CD16 expression in cells (e.g. in tumor-infiltrating NK cells) from a tumor or tumor sample (e.g., tumor and/or within adjacent tissue) from an individual, and
b) upon a determination that the tumor or tumor sample is characterized by a CD16 deficient microenvironment, administering to the individual a multispecific protein that binds to a cancer antigen, to NKp46, and to the cytokine receptor (e.g. CD122) (and optionally further to CD16A).

Optionally, in one embodiment, provided is a method for the treatment or prevention of a cancer in an individual in need thereof, the method comprising:
a) detecting CD16 expression at the surface of NK cells (e.g. tumor-infiltrating NK cells) in a tumor sample from an individual (or within the tumor and/or within adjacent tissue), and
b) upon a determination that the tumor or tumor sample is characterized by an elevated proportion of CD16⁻ NK cells, optionally at a level or number that is increased compared to a reference level, administering to the individual a multispecific protein that binds to a cancer antigen, to NKp46, and to the cytokine receptor (e.g. CD122) (and optionally further to CD16A).

In one embodiment, the disclosure provides a method for the treatment or prevention of a disease (e.g. a cancer) in an individual in need thereof, the method comprising:
a) detecting cell surface expression of one or a plurality inhibitory receptors on immune effector cells (e.g. NK cells, T cells) in a sample from the individual (e.g. in circulation or in the tumor environment), and
b) upon a determination of cell surface expression of one or a plurality inhibitory receptors on immune effector cells, optionally at a level that is increased compared to a reference level (e.g. at a level that is increased compared to a healthy individual, an individual not suffering from immune exhaustion or suppression, or an individual not deriving substantial benefit from a protein described herein), administering to the individual a multispecific protein (e.g. a multispecific protein) that binds to an antigen of interest (e.g. a cancer antigen), to NKp46 (e.g., monovalently), and to the cytokine receptor (e.g. CD122) (and optionally further to CD16A).

In some embodiments, the multispecific proteins are used to treat an individual having a gastric cancer or a prostate cancer. Decreased cell surface expression of NKG2D on immune effector cells has been observed in gastric cancer and prostate cancer.

In some embodiments, an individual has NK cells and/or T cells characterized by decreased expression of NKG2D, e.g. decreased cell surface expression. The level of expression can be for example compared to a reference value, for example a reference value corresponding to NKG2D levels observed on NK and/or T cells in healthy individuals. In some embodiments, an individual has NK and/or T cell characterized by decreased expression of NKG2D on NK and/or T cells in the tumor microenvironment. In some embodiments, an individual has presence (e.g. at increased levels) of soluble ligands of NKG2D in the tumor microenvironment, for example soluble MICA, MICB or ULBP proteins.

In one embodiment, the disclosure provides a method for the treatment or prevention of a disease (e.g. a cancer) in an individual in need thereof, the method comprising:
a) detecting cell surface expression of NKG2D polypeptides on immune effector cells (e.g. NK cells, T cells) in a sample from the individual (e.g. in circulation or in the tumor environment), and
b) upon a determination of decreased cell surface expression of one or a plurality inhibitory receptors on immune effector cells, optionally at a level that is decreased compared to a reference level (e.g. at a level that is increased compared to a healthy individual, an individual not suffering from immune exhaustion or suppression, or an individual not deriving substantial benefit from a protein described herein), administering to the individual a multispecific protein (e.g. a multispecific protein) that binds to an antigen of interest (e.g. a cancer antigen), to NKp46 (e.g., monovalently), and to the cytokine receptor (e.g. CD122) (and optionally further to CD16A).

In one embodiment, a multispecific protein may be used as a monotherapy (without other therapeutic agents), or in combined treatments with one or more other therapeutic agents that are administered separately. In one embodiment, a multispecific protein is administered in the absence of combined treatment with an IL-2, IL-15, IL-21, IL-7, IL-27, IL-12, IL-18, IFN-α or IFN-β polypeptide.

The multispecific proteins can also be included in kits. The kits may optionally further contain any number of polypeptides and/or other compounds, e.g., 1, 2, 3, 4, or any other number of multispecific proteins and/or other compounds. It will be appreciated that this description of the contents of the kits is not limiting in any way. For example, the kit may contain other types of therapeutic compounds. Optionally, the kits also include instructions for using the polypeptides, e.g., detailing the herein-described methods such as in the detection or treatment of specific disease conditions.

Also provided are pharmaceutical compositions comprising the subject multispecific proteins and optionally other compounds as defined above. A multispecific protein and optionally another compound may be administered in purified form together with a pharmaceutical carrier as a pharmaceutical composition. The form depends on the intended mode of administration and therapeutic or diagnostic application. The pharmaceutical carrier can be any compatible, nontoxic substance suitable to deliver the compounds to the patient. Pharmaceutically acceptable carriers are well known in the art and include, for example, aqueous solutions such as (sterile) water or physiologically buffered saline or other solvents or vehicles such as glycols, glycerol, oils such as olive oil or injectable organic esters, alcohol, fats, waxes, and inert solids. A pharmaceutically acceptable carrier may further contain physiologically acceptable compounds that act for example to stabilize or to increase the absorption of the compounds Such physiologically acceptable compounds include, for example, carbohydrates, such as glucose, sucrose or dextrans, antioxidants, such as ascorbic acid or glutathione, chelating agents, low molecular weight proteins or other stabilizers or excipients One skilled in the art would know that the choice of a pharmaceutically acceptable carrier, including a physiologically acceptable compound, depends, for example, on the route of administration of the composition Pharmaceutically acceptable adjuvants, buffering agents, dispersing agents, and the like, may also be incorporated into the pharmaceutical compositions. Non-limiting examples of such adjuvants include by way of example inorganic and organic adjuvants such as alum, aluminum phosphate and aluminum hydroxide, squalene, liposomes, lipopolysaccharides, double stranded (ds) RNAs, single stranded (s-s) DNAs, and TLR agonists such as unmethylated CpG's.

Multispecific proteins according to the invention can be administered parenterally. Preparations of the compounds for parenteral administration must be sterile. Sterilization is readily accomplished by filtration through sterile filtration membranes, optionally prior to or following lyophilization and reconstitution. The parenteral route for administration of compounds is in accord with known methods, e.g. injection or infusion by intravenous, intraperitoneal, intramuscular, intraarterial, or intralesional routes. The compounds may be administered continuously by infusion or by bolus injection. A typical composition for intravenous infusion could be made up to contain 100 to 500 ml of sterile 0.9% NaCl or 5% glucose optionally supplemented with a 20% albumin solution and 1 mg to 10 g of the compound, depending on the particular type of compound and its required dosing regimen.

Methods for preparing parenterally administrable compositions are well known in the art.

### EXAMPLES

### Preparation of Multispecific Proteins

The domain structure of an exemplary “T5” format multispecific protein comprising IL-2 variant as used in the Examples is shown in FIGS. 2A and 2G, incorporating a humanized NK46-1 VH and VL pair as the NKp46 ABD. FIG. 2A shows domain linkers such as hinge and glycine-serine linkers, and interchain disulfide bridges. The domain structure of the exemplary “T6” format, having a N297S mutation to substantially abolish CD16A binding but otherwise equivalent to format T5, is shown in FIG. 21. To build the T5 chain L (also referred to as chain 3) the CK domain normally associated with the NKp46-1 VK domain in the NKp46-binding ABD was replaced by a CH1 domain. The T25 format differs from the T5 format by replacement of the CH1 and CK of the NKp46-binding ABD such that the CK domain normally associated with the NKp46-1 VK domain and the CH1 normally associated with the VH remain associated with therewith. In order to ensure a correct pairing between Chain L (chain 3) and Chain H (chain1) and formation of a proper disulfide bond between H and L chains, the upper-hinge residues of human IgG1 were added at the C-terminus of CH1 domain of chain L upstream of the linker connecting chain L to IL-2 variant. Other protein formats are shown in FIGS. 2B-2N. The domain structures of exemplary “T5” format multispecific protein comprising IL-15, IL-18 (IL-18v) or IFN-α (IFNαv) variants as used in the Examples are shown in FIGS. 2O, 2P, 2Q and 2R, incorporating either humanized NKp46-1 or NKp46-4 VH and VK domains.

The sequences encoding each polypeptide chain for each multispecific antigen-binding protein were inserted into the pTT-5 vector between the HindIII and BamHI restriction sites. The three vectors (prepared as endotoxin-free midipreps or maxipreps) were used to cotransfect EXPI-293F cells (Life Technologies) in the presence of PEI (37° C., 5% CO₂, 150 rpm). The cells were used to seed culture flasks at a density of 1×106 cells per ml (EXP1293 medium, Gibco). As a reference, for the “T5” constructs, we used a DNA ratio of 0.1 μg/ml (polypeptide chain I), 0.4 μg/ml (polypeptide chain II), or 0.8 μg/ml (polypeptide chain III). Valproic Acid (final concentration 0.5 mM), glucose (4 g/L) and tryptone N1 (0.5%) were added. The supernatant was harvested after six days after and passed through a Stericup filter with 0.22 μm pores.

The multispecific antigen-binding proteins were purified from the supernatant following harvesting using rProtein A Sepharose Fast Flow (GE Healthcare, reference 17-1279-03). Size Exclusion Chromatography (SEC) purifications were then performed and the proteins eluted at the expected size were finally filtered on a 0.22 μm device.

The amino acid sequence of the polypeptide chains of the multispecific proteins produced are shown below in Table 6.

TABLE 6

| Sample | Frag M (chain 2) | Frag H (chain 1) | Frag L (chain 3) |
|---|---|---|---|
| GA101- | DIVMTQTPLSLPVTPGEPA | QVQLVQSGAEVKKPGSSVKVSCKASGYA | DIQMTQSPSSLSASV |
| T5- | SISCRSSKSLLHSNGITYL | FSYSWINWVRQAPGQGLEWMGRIFPGDG | GDRVTITCRASQDIS |
| NKp46- | YWYLQKPGQSPQLLIYQMS | DTDYNGKFKGRVTITADKSTSTAYMELS | NYLNWYQQKPGKAPK |
| 1-IL2v | NLVSGVPDRFSGSGSGTDF | SLRSEDTAVYYCARNVFDGYWLVYWGQG | LLIYYTSRLHSGVPS |
| 10aa | TLKISRVEAEDVGVYYCAQ | TLVTVSSASTKGPSVFPLAPSSKSTSGG | RFSGSGSGTDFTFTI |
| linker | NLELPYTFGGGTKVEIKRT | TAALGCLVKDYFPEPVTVSWNSGALTSG | SSLQPEDIATYFCQQ |
| | VAAPSVFIFPPSDEQLKSG | VHTFPAVLQSSGLYSLSSVVTVPSSSLG | GNTRPWTFGGGTKVE |
| | TASVVCLLNNFYPREAKVQ | TQTYICNVNHKPSNTKVDKRVEPKSCDK | IKASTKGPSVFPLAP |
| | WKVDNALQSGNSQESVTEQ | THTCPPCPAPELLGGPSVFLFPPKPKDT | SSKSTSGGTAALGCL |
| | DSKDSTYSLSSTLTLSKAD | LMISRTPEVTCVVVDVSHEDPEVKENWY | VKDYFPEPVTVSWNS |
| | YEKHKVYACEVTHQGLSSP | VDGVEVHNAKTKPREEQYNSTYRVVSVL | GALTSGVHTFPAVLQ |
| | VTKSFNRGECDKTHTCPPC | TVLHQDWLNGKEYKCKVSNKALPAPIEK | SSGLYSLSSVVTVPS |
| | PAPELLGGPSVFLFPPKPK | TISKAKGQPREPQVYTLPPSREEMTKNQ | SSLGTQTYICNVNHK |
| | DTLMISRTPEVTCVVVDVS | VSLTCLVKGFYPSDIAVEWESNGQPENN | PSNTKVDKRVEPKSC |
| | HEDPEVKFNWYVDGVEVHN | YKTTPPVLDSDGSFFLYSKLTVDKSRWQ | DKTHSGSSSSGSSSS |
| | AKTKPREEQYNSTYRVVSV | QGNVFSCSVMHEALHNHYTQKSLSLSPG | APASSSTKKTQLQLE |
| | LTVLHQDWLNGKEYKCKVS | STGSQVQLVQSGAEVKKPGSSVKVSCKA | HLLLDLQMILNGINN |
| | NKALPAPIEKTISKAKGQP | SGYTFSDYVINWVRQAPGQGLEWMGEIY | YKNPKLTRMLTAKFA |
| | REPQVYTLPPSREEMTKNQ | PGSGTNYYNEKFKAKATITADKSTSTAY | MPKKATELKHLQCLE |
| | VSLTCLVKGFYPSDIAVEW | MELSSLRSEDTAVYYCARRGRYGLYAMD | EELKPLEEVLNGAQS |
| | ESNGQPENNYKTTPPVLDS | YWGQGTTVTVSSRTVAAPSVFIFPPSDE | KNFHLRPRDLISNIN |
| | DGSFFLYSKLTVDKSRWQQ | QLKSGTASVVCLLNNFYPREAKVQWKVD | VIVLELKGSETTFMC |
| | GNVFSCSVMHEALHNHYTQ | NALQSGNSQESVTEQDSKDSTYSLSSTL | EYADETATIVEFLNR |
| | KSLSLSPGK (SEQ ID | TLSKADYEKHKVYACEVTHQGLSSPVTK | WITFAQSIISTLT |
| | NO: 175) | SFNRGEC (SEQ ID NO: 176) | (SEQ ID NO: 177) |
| GA101- | DIVMTQTPLSLPVTPGEPA | QVQLVQSGAEVKKPGSSVKVSCKASGYA | DIQMTQSPSSLSASV |
| T5- | SISCRSSKSLLHSNGITYL | FSYSWINWVRQAPGQGLEWMGRIFPGDG | GDRVTITCRASQDIS |
| NKp46- | YWYLQKPGQSPQLLIYQMS | DTDYNGKFKGRVTITADKSTSTAYMELS | NYLNWYQQKPGKAPK |
| 1-IL2v | NLVSGVPDRFSGSGSGTDF | SLRSEDTAVYYCARNVFDGYWLVYWGQG | LLIYYTSRLHSGVPS |
| short | TLKISRVEAEDVGVYYCAQ | TLVTVSSASTKGPSVFPLAPSSKSTSGG | RFSGSGSGTDFTFTI |
| linker | NLELPYTFGGGTKVEIKRT | TAALGCLVKDYFPEPVTVSWNSGALTSG | SSLQPEDIATYFCQQ |
| | VAAPSVFIFPPSDEQLKSG | VHTFPAVLQSSGLYSLSSVVTVPSSSLG | GNTRPWTFGGGTKVE |
| | TASVVCLLNNFYPREAKVQ | TQTYICNVNHKPSNTKVDKRVEPKSCDK | IKASTKGPSVFPLAP |
| | WKVDNALQSGNSQESVTEQ | THTCPPCPAPELLGGPSVFLFPPKPKDT | SSKSTSGGTAALGCL |
| | DSKDSTYSLSSTLTLSKAD | LMISRTPEVTCVVVDVSHEDPEVKENWY | VKDYFPEPVTVSWNS |
| | YEKHKVYACEVTHQGLSSP | VDGVEVHNAKTKPREEQYNSTYRVVSVL | GALTSGVHTFPAVLQ |
| | VTKSFNRGECDKTHTCPPC | TVLHQDWLNGKEYKCKVSNKALPAPIEK | SSGLYSLSSVVTVPS |
| | PAPELLGGPSVFLFPPKPK | TISKAKGQPREPQVYTLPPSREEMTKNQ | SSLGTQTYICNVNHK |
| | DTLMISRTPEVTCVVVDVS | VSLTCLVKGFYPSDIAVEWESNGQPENN | PSNTKVDKRVEPKSC |
| | HEDPEVKFNWYVDGVEVHN | YKTTPPVLDSDGSFFLYSKLTVDKSRWQ | DKTHSGSSSSAPASS |
| | AKTKPREEQYNSTYRVVSV | QGNVFSCSVMHEALHNHYTQKSLSLSPG | STKKTQLQLEHLLLD |
| | LTVLHQDWLNGKEYKCKVS | STGSQVQLVQSGAEVKKPGSSVKVSCKA | LQMILNGINNYKNPK |
| | NKALPAPIEKTISKAKGQP | SGYTFSDYVINWVRQAPGQGLEWMGEIY | LTRMLTAKFAMPKKA |
| | REPQVYTLPPSREEMTKNQ | PGSGTNYYNEKFKAKATITADKSTSTAY | TELKHLQCLEEELKP |
| | VSLTCLVKGFYPSDIAVEW | MELSSLRSEDTAVYYCARRGRYGLYAMD | LEEVLNGAQSKNFHL |
| | ESNGQPENNYKTTPPVLDS | YWGQGTTVTVSSRTVAAPSVFIFPPSDE | RPRDLISNINVIVLE |
| | DGSFFLYSKLTVDKSRWQQ | QLKSGTASVVCLLNNFYPREAKVQWKVD | LKGSETTFMCEYADE |
| | GNVFSCSVMHEALHNHYTQ | NALQSGNSQESVTEQDSKDSTYSLSSTL | TATIVEFLNRWITFA |
| | KSLSLSPGK (SEQ ID | TLSKADYEKHKVYACEVTHQGLSSPVTK | QSIISTLT (SEQ |
| | NO: 178) | SFNRGEC (SEQ ID NO: 179) | ID NO: 180) |
| GA101- | DIVMTQTPLSLPVTPGEPA | QVQLVQSGAEVKKPGSSVKVSCKASGYA | DIQMTQSPSSLSASV |
| T5- | SISCRSSKSLLHSNGITYL | FSYSWINWVRQAPGQGLEWMGRIFPGDG | GDRVTITCRASQDIS |
| NKp46- | YWYLQKPGQSPQLLIYQMS | DTDYNGKFKGRVTITADKSTSTAYMELS | NYLNWYQQKPGKAPK |
| 1-IL2v | NLVSGVPDRFSGSGSGTDF | SLRSEDTAVYYCARNVFDGYWLVYWGQG | LLIYYTSRLHSGVPS |
| long | TLKISRVEAEDVGVYYCAQ | TLVTVSSASTKGPSVFPLAPSSKSTSGG | RFSGSGSGTDFTFTI |
| linker | NLELPYTFGGGTKVEIKRT | TAALGCLVKDYFPEPVTVSWNSGALTSG | SSLQPEDIATYFCQQ |
| | VAAPSVFIFPPSDEQLKSG | VHTFPAVLQSSGLYSLSSVVTVPSSSLG | GNTRPWTFGGGTKVE |
| | TASVVCLLNNFYPREAKVQ | TQTYICNVNHKPSNTKVDKRVEPKSCDK | IKASTKGPSVFPLAP |
| | WKVDNALQSGNSQESVTEQ | THTCPPCPAPELLGGPSVFLFPPKPKDT | SSKSTSGGTAALGCL |
| | DSKDSTYSLSSTLTLSKAD | LMISRTPEVTCVVVDVSHEDPEVKENWY | VKDYFPEPVTVSWNS |
| | YEKHKVYACEVTHQGLSSP | VDGVEVHNAKTKPREEQYNSTYRVVSVL | GALTSGVHTFPAVLQ |
| | VTKSFNRGECDKTHTCPPC | TVLHQDWLNGKEYKCKVSNKALPAPIEK | SSGLYSLSSVVTVPS |
| | PAPELLGGPSVFLFPPKPK | TISKAKGQPREPQVYTLPPSREEMTKNQ | SSLGTQTYICNVNHK |
| | DTLMISRTPEVTCVVVDVS | VSLTCLVKGFYPSDIAVEWESNGQPENN | PSNTKVDKRVEPKSC |
| | HEDPEVKFNWYVDGVEVHN | YKTTPPVLDSDGSFFLYSKLTVDKSRWQ | DKTHSGSSSSGSSSS |
| | AKTKPREEQYNSTYRVVSV | QGNVFSCSVMHEALHNHYTQKSLSLSPG | GSSSSAPASSSTKKT |
| | LTVLHQDWLNGKEYKCKVS | STGSQVQLVQSGAEVKKPGSSVKVSCKA | QLQLEHLLLDLQMIL |
| | NKALPAPIEKTISKAKGQP | SGYTFSDYVINWVRQAPGQGLEWMGEIY | NGINNYKNPKLTRML |
| | REPQVYTLPPSREEMTKNQ | PGSGTNYYNEKFKAKATITADKSTSTAY | TAKFAMPKKATELKH |
| | VSLTCLVKGFYPSDIAVEW | MELSSLRSEDTAVYYCARRGRYGLYAMD | LQCLEEELKPLEEVL |
| | ESNGQPENNYKTTPPVLDS | YWGQGTTVTVSSRTVAAPSVFIFPPSDE | NGAQSKNFHLRPRDL |
| | DGSFFLYSKLTVDKSRWQQ | QLKSGTASVVCLLNNFYPREAKVQWKVD | ISNINVIVLELKGSE |
| | GNVFSCSVMHEALHNHYTQ | NALQSGNSQESVTEQDSKDSTYSLSSTL | TTFMCEYADETATIV |
| | KSLSLSPGK (SEQ ID | TLSKADYEKHKVYACEVTHQGLSSPVTK | EFLNRWITFAQSIIS |
| | NO: 181) | SFNRGEC (SEQ ID NO: 182) | TLT (SEQ ID NO: |
| | | | 183) |
| GA101- | DIVMTQTPLSLPVTPGEPA | QVQLVQSGAEVKKPGSSVKVSCKASGYA | DIQMTQSPSSLSASV |
| T6- | SISCRSSKSLLHSNGITYL | FSYSWINWVRQAPGQGLEWMGRIFPGDG | GDRVTITCRASQDIS |
| NKp46- | YWYLQKPGQSPQLLIYQMS | DTDYNGKFKGRVTITADKSTSTAYMELS | NYLNWYQQKPGKAPK |
| 1-IL2v | NLVSGVPDRFSGSGSGTDF | SLRSEDTAVYYCARNVEDGYWLVYWGQG | LLIYYTSRLHSGVPS |
| | TLKISRVEAEDVGVYYCAQ | TLVTVSSASTKGPSVFPLAPSSKSTSGG | RFSGSGSGTDFTFTI |
| | NLELPYTFGGGTKVEIKRT | TAALGCLVKDYFPEPVTVSWNSGALTSG | SSLQPEDIATYFCQQ |
| | VAAPSVFIFPPSDEQLKSG | VHTFPAVLQSSGLYSLSSVVTVPSSSLG | GNTRPWTFGGGTKVE |
| | TASVVCLLNNFYPREAKVQ | TQTYICNVNHKPSNTKVDKRVEPKSCDK | IKASTKGPSVFPLAP |
| | WKVDNALQSGNSQESVTEQ | THTCPPCPAPELLGGPSVFLFPPKPKDT | SSKSTSGGTAALGCL |
| | DSKDSTYSLSSTLTLSKAD | LMISRTPEVTCVVVDVSHEDPEVKENWY | VKDYFPEPVTVSWNS |
| | YEKHKVYACEVTHQGLSSP | VDGVEVHNAKTKPREEQYSSTYRVVSVL | GALTSGVHTFPAVLQ |
| | VTKSENRGECDKTHTCPPC | TVLHQDWLNGKEYKCKVSNKALPAPIEK | SSGLYSLSSVVTVPS |
| | PAPELLGGPSVFLFPPKPK | TISKAKGQPREPQVYTLPPSREEMTKNQ | SSLGTQTYICNVNHK |
| | DTLMISRTPEVTCVVVDVS | VSLTCLVKGFYPSDIAVEWESNGQPENN | PSNTKVDKRVEPKSC |
| | HEDPEVKFNWYVDGVEVHN | YKTTPPVLDSDGSFFLYSKLTVDKSRWQ | DKTHSGSSSSGSSSS |
| | AKTKPREEQYSSTYRVVSV | QGNVFSCSVMHEALHNHYTQKSLSLSPG | APASSSTKKTQLQLE |
| | LTVLHQDWLNGKEYKCKVS | STGSQVQLVQSGAEVKKPGSSVKVSCKA | HLLLDLQMILNGINN |
| | NKALPAPIEKTISKAKGQP | SGYTFSDYVINWVRQAPGQGLEWMGEIY | YKNPKLTRMLTAKFA |
| | REPQVYTLPPSREEMTKNQ | PGSGTNYYNEKFKAKATITADKSTSTAY | MPKKATELKHLQCLE |
| | VSLTCLVKGFYPSDIAVEW | MELSSLRSEDTAVYYCARRGRYGLYAMD | EELKPLEEVLNGAQS |
| | ESNGQPENNYKTTPPVLDS | YWGQGTTVTVSSRTVAAPSVFIFPPSDE | KNFHLRPRDLISNIN |
| | DGSFFLYSKLTVDKSRWQQ | QLKSGTASVVCLLNNFYPREAKVQWKVD | VIVLELKGSETTFMC |
| | GNVFSCSVMHEALHNHYTQ | NALQSGNSQESVTEQDSKDSTYSLSSTL | EYADETATIVEFLNR |
| | KSLSLSPGK (SEQ ID | TLSKADYEKHKVYACEVTHQGLSSPVTK | WITFAQSIISTLT |
| | NO: 184) | SFNRGEC (SEQ ID NO: 185) | (SEQ ID NO: 186) |
| GA101- | DIVMTQTPLSLPVTPGEPA | QVQLVQSGAEVKKPGSSVKVSCKASGYA | DIQMTQSPSSLSASV |
| T6- | SISCRSSKSLLHSNGITYL | FSYSWINWVRQAPGQGLEWMGRIFPGDG | GDRVTITCRASQDIS |
| NKp46- | YWYLQKPGQSPQLLIYQMS | DTDYNGKFKGRVTITADKSTSTAYMELS | NYLNWYQQKPGKAPK |
| 1-IL2v2 | NLVSGVPDRFSGSGSGTDF | SLRSEDTAVYYCARNVEDGYWLVYWGQG | LLIYYTSRLHSGVPS |
| | TLKISRVEAEDVGVYYCAQ | TLVTVSSASTKGPSVFPLAPSSKSTSGG | RFSGSGSGTDFTFTI |
| | NLELPYTFGGGTKVEIKRT | TAALGCLVKDYFPEPVTVSWNSGALTSG | SSLQPEDIATYFCQQ |
| | VAAPSVFIFPPSDEQLKSG | VHTFPAVLQSSGLYSLSSVVTVPSSSLG | GNTRPWTFGGGTKVE |
| | TASVVCLLNNFYPREAKVQ | TQTYICNVNHKPSNTKVDKRVEPKSCDK | IKASTKGPSVFPLAP |
| | WKVDNALQSGNSQESVTEQ | THTCPPCPAPELLGGPSVFLFPPKPKDT | SSKSTSGGTAALGCL |
| | DSKDSTYSLSSTLTLSKAD | LMISRTPEVTCVVVDVSHEDPEVKENWY | VKDYFPEPVTVSWNS |
| | YEKHKVYACEVTHQGLSSP | VDGVEVHNAKTKPREEQYSSTYRVVSVL | GALTSGVHTFPAVLQ |
| | VTKSFNRGECDKTHTCPPC | TVLHQDWLNGKEYKCKVSNKALPAPIEK | SSGLYSLSSVVTVPS |
| | PAPELLGGPSVFLFPPKPK | TISKAKGQPREPQVYTLPPSREEMTKNQ | SSLGTQTYICNVNHK |
| | DTLMISRTPEVTCVVVDVS | VSLTCLVKGFYPSDIAVEWESNGQPENN | PSNTKVDKRVEPKSC |
| | HEDPEVKFNWYVDGVEVHN | YKTTPPVLDSDGSFFLYSKLTVDKSRWQ | DKTHSGSSSSGSSSS |
| | AKTKPREEQYSSTYRVVSV | QGNVFSCSVMHEALHNHYTQKSLSLSPG | GSSSSTKKTQLQLEH |
| | LTVLHQDWLNGKEYKCKVS | STGSQVQLVQSGAEVKKPGSSVKVSCKA | LLLDLQMILNGINNY |
| | NKALPAPIEKTISKAKGQP | SGYTFSDYVINWVRQAPGQGLEWMGEIY | KNPKLTAMLTKKFYM |
| | REPQVYTLPPSREEMTKNQ | PGSGTNYYNEKFKAKATITADKSTSTAY | PKKATELKHLQCLEE |
| | VSLTCLVKGFYPSDIAVEW | MELSSLRSEDTAVYYCARRGRYGLYAMD | ELKPLEEVLNLAQSK |
| | ESNGQPENNYKTTPPVLDS | YWGQGTTVTVSSRTVAAPSVFIFPPSDE | NFHLRPRDLISNINV |
| | DGSFFLYSKLTVDKSRWQQ | QLKSGTASVVCLLNNFYPREAKVQWKVD | IVLELKGSETTFMCE |
| | GNVFSCSVMHEALHNHYTQ | NALQSGNSQESVTEQDSKDSTYSLSSTL | YADETATIVEFLNRW |
| | KSLSLSPGK (SEQ ID | TLSKADYEKHKVYACEVTHQGLSSPVTK | ITFCQSIISTLT |
| | NO: 187) | SFNQRGEC (SEQ ID NO: 188) | (SEQ ID NO: 189) |
| GA101- | DIVMTQTPLSLPVTPGEPA | QVQLVQSGAEVKKPGSSVKVSCKASGYA | DIQMTQSPSSLSASV |
| T6- | SISCRSSKSLLHSNGITYL | FSYSWINWVRQAPGQGLEWMGRIFPGDG | GDRVTITCRASQDIS |
| NKp46- | YWYLQKPGQSPQLLIYQMS | DTDYNGKFKGRVTITADKSTSTAYMELS | NYLNWYQQKPGKAPK |
| 1-IL2v3 | NLVSGVPDRFSGSGSGTDF | SLRSEDTAVYYCARNVFDGYWLVYWGQG | LLIYYTSRLHSGVPS |
| | TLKISRVEAEDVGVYYCAQ | TLVTVSSASTKGPSVFPLAPSSKSTSGG | RFSGSGSGTDFTFTI |
| | NLELPYTFGGGTKVEIKRT | TAALGCLVKDYFPEPVTVSWNSGALTSG | SSLQPEDIATYFCQQ |
| | VAAPSVFIFPPSDEQLKSG | VHTFPAVLQSSGLYSLSSVVTVPSSSLG | GNTRPWTFGGGTKVE |
| | TASVVCLLNNFYPREAKVQ | TQTYICNVNHKPSNTKVDKRVEPKSCDK | IKASTKGPSVFPLAP |
| | WKVDNALQSGNSQESVTEQ | THTCPPCPAPELLGGPSVFLFPPKPKDT | SSKSTSGGTAALGCL |
| | DSKDSTYSLSSTLTLSKAD | LMISRTPEVTCVVVDVSHEDPEVKENWY | VKDYFPEPVTVSWNS |
| | YEKHKVYACEVTHQGLSSP | VDGVEVHNAKTKPREEQYSSTYRVVSVL | GALTSGVHTFPAVLQ |
| | VTKSFNRGECDKTHTCPPC | TVLHQDWLNGKEYKCKVSNKALPAPIEK | SSGLYSLSSVVTVPS |
| | PAPELLGGPSVFLFPPKPK | TISKAKGQPREPQVYTLPPSREEMTKNQ | SSLGTQTYICNVNHK |
| | DTLMISRTPEVTCVVVDVS | VSLTCLVKGFYPSDIAVEWESNGQPENN | PSNTKVDKRVEPKSC |
| | HEDPEVKFNWYVDGVEVHN | YKTTPPVLDSDGSFFLYSKLTVDKSRWQ | DKTHSGSSSSGSSSS |
| | AKTKPREEQYSSTYRVVSV | QGNVFSCSVMHEALHNHYTQKSLSLSPG | GSSSSTKKTQLQLEH |
| | LTVLHQDWLNGKEYKCKVS | STGSQVQLVQSGAEVKKPGSSVKVSCKA | LLLDLQMILNGINNY |
| | NKALPAPIEKTISKAKGQP | SGYTFSDYVINWVRQAPGQGLEWMGEIY | KNPKLTAMLAKKFYM |
| | REPQVYTLPPSREEMTKNQ | PGSGTNYYNEKFKAKATITADKSTSTAY | PKKATELKHLQCLEE |
| | VSLTCLVKGFYPSDIAVEW | MELSSLRSEDTAVYYCARRGRYGLYAMD | ELKPLEEVLNLAQSK |
| | ESNGQPENNYKTTPPVLDS | YWGQGTTVTVSSRTVAAPSVFIFPPSDE | NFHLRPRDLISNINV |
| | DGSFFLYSKLTVDKSRWQQ | QLKSGTASVVCLLNNFYPREAKVQWKVD | IVLELKGSETTFMCE |
| | GNVFSCSVMHEALHNHYTQ | NALQSGNSQESVTEQDSKDSTYSLSSTL | YADETATIVEFLNRW |
| | KSLSLSPGK (SEQ ID | TLSKADYEKHKVYACEVTHQGLSSPVTK | ITFCQSIISTLT |
| | NO: 190) | SENRGEC (SEQ ID NO: 191) | (SEQ ID NO: 192) |
| GA101- | DIVMTQTPLSLPVTPGEPA | QVQLVQSGAEVKKPGSSVKVSCKASGYA | DIQMTQSPSSLSASV |
| T5- | SISCRSSKSLLHSNGITYL | FSYSWINWVRQAPGQGLEWMGRIFPGDG | GDRVTITCRASENIY |
| NKp46- | YWYLQKPGQSPQLLIYQMS | DTDYNGKFKGRVTITADKSTSTAYMELS | SNLAWFQQKPGKAPK |
| 4-IL2v | NLVSGVPDRFSGSGSGTDF | SLRSEDTAVYYCARNVFDGYWLVYWGQG | LLVYAATNLADGVPS |
| | TLKISRVEAEDVGVYYCAQ | TLVTVSSASTKGPSVFPLAPSSKSTSGG | RFSGSGSGTDYTLTI |
| | NLELPYTFGGGTKVEIKRT | TAALGCLVKDYFPEPVTVSWNSGALTSG | SSLQPEDFATYYCQH |
| | VAAPSVFIFPPSDEQLKSG | VHTFPAVLQSSGLYSLSSVVTVPSSSLG | FWGTPRTFGGGTKVE |
| | TASVVCLLNNFYPREAKVQ | TQTYICNVNHKPSNTKVDKRVEPKSCDK | IKASTKGPSVFPLAP |
| | WKVDNALQSGNSQESVTEQ | THTCPPCPAPELLGGPSVFLFPPKPKDT | SSKSTSGGTAALGCL |
| | DSKDSTYSLSSTLTLSKAD | LMISRTPEVTCVVVDVSHEDPEVKENWY | VKDYFPEPVTVSWNS |
| | YEKHKVYACEVTHQGLSSP | VDGVEVHNAKTKPREEQYNSTYRVVSVL | GALTSGVHTFPAVLQ |
| | VTKSFNRGECDKTHTCPPC | TVLHQDWLNGKEYKCKVSNKALPAPIEK | SSGLYSLSSVVTVPS |
| | PAPELLGGPSVFLFPPKPK | TISKAKGQPREPQVYTLPPSREEMTKNQ | SSLGTQTYICNVNHK |
| | DTLMISRTPEVTCVVVDVS | VSLTCLVKGFYPSDIAVEWESNGQPENN | PSNTKVDKRVEPKSC |
| | HEDPEVKFNWYVDGVEVHN | YKTTPPVLDSDGSFFLYSKLTVDKSRWQ | DKTHSGSSSSGSSSS |
| | AKTKPREEQYNSTYRVVSV | QGNVFSCSVMHEALHNHYTQKSLSLSPG | APASSSTKKTQLQLE |
| | LTVLHQDWLNGKEYKCKVS | STGSQVQLVQSGAEVKKPGASVKVSCKA | HLLLDLQMILNGINN |
| | NKALPAPIEKTISKAKGQP | SGYTFTSFTMHWVRQAPGQGLEWIGYIN | YKNPKLTRMLTAKFA |
| | REPQVYTLPPSREEMTKNQ | PSSGYTEYNQKFKDRVTITADKSTSTAY | MPKKATELKHLQCLE |
| | VSLTCLVKGFYPSDIAVEW | MELSSLRSEDTAVYYCVRGSSRGFDYWG | EELKPLEEVLNGAQS |
| | ESNGQPENNYKTTPPVLDS | QGTLVTVSSRTVAAPSVFIFPPSDEQLK | KNFHLRPRDLISNIN |
| | DGSFFLYSKLTVDKSRWQQ | SGTASVVCLLNNFYPREAKVQWKVDNAL | VIVLELKGSETTFMC |
| | GNVFSCSVMHEALHNHYTQ | QSGNSQESVTEQDSKDSTYSLSSTLTLS | EYADETATIVEFLNR |
| | KSLSLSPGK (SEQ ID | KADYEKHKVYACEVTHQGLSSPVTKSEN | WITFAQSIISTLT |
| | NO: 193) | RGEC (SEQ ID NO: 194) | (SEQ ID NO: 195) |
| GA101- | DIVMTQTPLSLPVTPGEPA | QVQLVQSGAEVKKPGSSVKVSCKASGYA | DIQMTQSPSSLSASV |
| T6- | SISCRSSKSLLHSNGITYL | FSYSWINWVRQAPGQGLEWMGRIFPGDG | GDRVTITCRASENIY |
| NKp46- | YWYLQKPGQSPQLLIYQMS | DTDYNGKFKGRVTITADKSTSTAYMELS | SNLAWFQQKPGKAPK |
| 4-IL2v | NLVSGVPDRFSGSGSGTDF | SLRSEDTAVYYCARNVFDGYWLVYWGQG | LLVYAATNLADGVPS |
| | TLKISRVEAEDVGVYYCAQ | TLVTVSSASTKGPSVFPLAPSSKSTSGG | RFSGSGSGTDYTLTI |
| | NLELPYTFGGGTKVEIKRT | TAALGCLVKDYFPEPVTVSWNSGALTSG | SSLQPEDFATYYCQH |
| | VAAPSVFIFPPSDEQLKSG | VHTFPAVLQSSGLYSLSSVVTVPSSSLG | FWGTPRTFGGGTKVE |
| | TASVVCLLNNFYPREAKVQ | TQTYICNVNHKPSNTKVDKRVEPKSCDK | IKASTKGPSVFPLAP |
| | WKVDNALQSGNSQESVTEQ | THTCPPCPAPELLGGPSVFLFPPKPKDT | SSKSTSGGTAALGCL |
| | DSKDSTYSLSSTLTLSKAD | LMISRTPEVTCVVVDVSHEDPEVKENWY | VKDYFPEPVTVSWNS |
| | YEKHKVYACEVTHQGLSSP | VDGVEVHNAKTKPREEQYSSTYRVVSVL | GALTSGVHTFPAVLQ |
| | VTKSFNRGECDKTHTCPPC | TVLHQDWLNGKEYKCKVSNKALPAPIEK | SSGLYSLSSVVTVPS |
| | PAPELLGGPSVFLFPPKPK | TISKAKGQPREPQVYTLPPSREEMTKNQ | SSLGTQTYICNVNHK |
| | DTLMISRTPEVTCVVVDVS | VSLTCLVKGFYPSDIAVEWESNGQPENN | PSNTKVDKRVEPKSC |
| | HEDPEVKFNWYVDGVEVHN | YKTTPPVLDSDGSFFLYSKLTVDKSRWQ | DKTHSGSSSSGSSSS |
| | AKTKPREEQYSSTYRVVSV | QGNVFSCSVMHEALHNHYTQKSLSLSPG | APASSSTKKTQLQLE |
| | LTVLHQDWLNGKEYKCKVS | STGSQVQLVQSGAEVKKPGASVKVSCKA | HLLLDLQMILNGINN |
| | NKALPAPIEKTISKAKGQP | SGYTFTSFTMHWVRQAPGQGLEWIGYIN | YKNPKLTRMLTAKFA |
| | REPQVYTLPPSREEMTKNQ | PSSGYTEYNQKFKDRVTITADKSTSTAY | MPKKATELKHLQCLE |
| | VSLTCLVKGFYPSDIAVEW | MELSSLRSEDTAVYYCVRGSSRGFDYWG | EELKPLEEVLNGAQS |
| | ESNGQPENNYKTTPPVLDS | QGTLVTVSSRTVAAPSVFIFPPSDEQLK | KNFHLRPRDLISNIN |
| | DGSFFLYSKLTVDKSRWQQ | SGTASVVCLLNNFYPREAKVQWKVDNAL | VIVLELKGSETTFMC |
| | GNVFSCSVMHEALHNHYTQ | QSGNSQESVTEQDSKDSTYSLSSTLTLS | EYADETATIVEFLNR |
| | KSLSLSPGK (SEQ ID | KADYEKHKVYACEVTHQGLSSPVTKSEN | WITFAQSIISTLT |
| | NO: 196) | RGEC (SEQ ID NO: 197) | (SEQ ID NO: 198) |
| GA101- | DIVMTQTPLSLPVTPGEPA | QVQLVQSGAEVKKPGSSVKVSCKASGYA | DIQMTQSPSSLSASV |
| T25- | SISCRSSKSLLHSNGITYL | FSYSWINWVRQAPGQGLEWMGRIFPGDG | GDRVTITCRASQDIS |
| NKp46- | YWYLQKPGQSPQLLIYQMS | DTDYNGKFKGRVTITADKSTSTAYMELS | NYLNWYQQKPGKAPK |
| 1-IL2v | NLVSGVPDRESGSGSGTDE | SLRSEDTAVYYCARNVFDGYWLVYWGQG | LLIYYTSRLHSGVPS |
| | TLKISRVEAEDVGVYYCAQ | TLVTVSSASTKGPSVFPLAPSSKSTSGG | RFSGSGSGTDFTFTI |
| | NLELPYTFGGGTKVEIKRT | TAALGCLVKDYFPEPVTVSWNSGALTSG | SSLQPEDIATYFCQQ |
| | VAAPSVFIFPPSDEQLKSG | VHTFPAVLQSSGLYSLSSVVTVPSSSLG | GNTRPWTFGGGTKVE |
| | TASVVCLLNNFYPREAKVQ | TQTYICNVNHKPSNTKVDKRVEPKSCDK | IKRTVAAPSVFIFPP |
| | WKVDNALQSGNSQESVTEQ | THTCPPCPAPELLGGPSVFLFPPKPKDT | SDEQLKSGTASVVCL |
| | DSKDSTYSLSSTLTLSKAD | LMISRTPEVTCVVVDVSHEDPEVKENWY | LNNFYPREAKVQWKV |
| | YEKHKVYACEVTHQGLSSP | VDGVEVHNAKTKPREEQYNSTYRVVSVL | DNALQSGNSQESVTE |
| | VTKSFNRGECDKTHTCPPC | TVLHQDWLNGKEYKCKVSNKALPAPIEK | QDSKDSTYSLSSTLT |
| | PAPELLGGPSVFLFPPKPK | TISKAKGQPREPQVYTLPPSREEMTKNQ | LSKADYEKHKVYACE |
| | DTLMISRTPEVTCVVVDVS | VSLTCLVKGFYPSDIAVEWESNGQPENN | VTHQGLSSPVTKSEN |
| | HEDPEVKFNWYVDGVEVHN | YKTTPPVLDSDGSFFLYSKLTVDKSRWQ | RGECGSSSSGSSSSA |
| | AKTKPREEQYNSTYRVVSV | QGNVFSCSVMHEALHNHYTQKSLSLSPG | PASSSTKKTQLQLEH |
| | LTVLHQDWLNGKEYKCKVS | STGSQVQLVQSGAEVKKPGSSVKVSCKA | LLLDLQMILNGINNY |
| | NKALPAPIEKTISKAKGQP | SGYTFSDYVINWVRQAPGQGLEWMGEIY | KNPKLTRMLTAKFAM |
| | REPQVYTLPPSREEMTKNQ | PGSGTNYYNEKFKAKATITADKSTSTAY | PKKATELKHLQCLEE |
| | VSLTCLVKGFYPSDIAVEW | MELSSLRSEDTAVYYCARRGRYGLYAMD | ELKPLEEVLNGAQSK |
| | ESNGQPENNYKTTPPVLDS | YWGQGTTVTVSSASTKGPSVFPLAPSSK | NFHLRPRDLISNINV |
| | DGSFFLYSKLTVDKSRWQQ | STSGGTAALGCLVKDYFPEPVTVSWNSG | IVLELKGSETTFMCE |
| | GNVFSCSVMHEALHNHYTQ | ALTSGVHTFPAVLQSSGLYSLSSVVTVP | YADETATIVEFLNRW |
| | KSLSLSPGK (SEQ ID | SSSLGTQTYICNVNHKPSNTKVDKRVEP | ITFAQSIISTLT |
| | NO: 199) | KSCDKTHS (SEQ ID NO: 200) | (SEQ ID NO: 201) |
| GA101- | DIVMTQTPLSLPVTPGEPA | QVQLVQSGAEVKKPGSSVKVSCKASGYA | DIQMTQSPSSLSASV |
| T26- | SISCRSSKSLLHSNGITYL | FSYSWINWVRQAPGQGLEWMGRIFPGDG | GDRVTITCRASQDIS |
| NKp46- | YWYLQKPGQSPQLLIYQMS | DTDYNGKFKGRVTITADKSTSTAYMELS | NYLNWYQQKPGKAPK |
| 1-IL2v | NLVSGVPDRFSGSGSGTDF | SLRSEDTAVYYCARNVFDGYWLVYWGQG | LLIYYTSRLHSGVPS |
| | TLKISRVEAEDVGVYYCAQ | TLVTVSSASTKGPSVFPLAPSSKSTSGG | RFSGSGSGTDFTFTI |
| | NLELPYTFGGGTKVEIKRT | TAALGCLVKDYFPEPVTVSWNSGALTSG | SSLQPEDIATYFCQQ |
| | VAAPSVFIFPPSDEQLKSG | VHTFPAVLQSSGLYSLSSVVTVPSSSLG | GNTRPWTFGGGTKVE |
| | TASVVCLLNNFYPREAKVQ | TQTYICNVNHKPSNTKVDKRVEPKSCDK | IKRTVAAPSVFIFPP |
| | WKVDNALQSGNSQESVTEQ | THTCPPCPAPELLGGPSVFLFPPKPKDT | SDEQLKSGTASVVCL |
| | DSKDSTYSLSSTLTLSKAD | LMISRTPEVTCVVVDVSHEDPEVKENWY | LNNFYPREAKVQWKV |
| | YEKHKVYACEVTHQGLSSP | VDGVEVHNAKTKPREEQYSSTYRVVSVL | DNALQSGNSQESVTE |
| | VTKSFNRGECDKTHTCPPC | TVLHQDWLNGKEYKCKVSNKALPAPIEK | QDSKDSTYSLSSTLT |
| | PAPELLGGPSVFLFPPKPK | TISKAKGQPREPQVYTLPPSREEMTKNQ | LSKADYEKHKVYACE |
| | DTLMISRTPEVTCVVVDVS | VSLTCLVKGFYPSDIAVEWESNGQPENN | VTHQGLSSPVTKSEN |
| | HEDPEVKFNWYVDGVEVHN | YKTTPPVLDSDGSFFLYSKLTVDKSRWQ | RGECGSSSSGSSSSA |
| | AKTKPREEQYSSTYRVVSV | QGNVFSCSVMHEALHNHYTQKSLSLSPG | PASSSTKKTQLQLEH |
| | LTVLHQDWLNGKEYKCKVS | STGSQVQLVQSGAEVKKPGSSVKVSCKA | LLLDLQMILNGINNY |
| | NKALPAPIEKTISKAKGQP | SGYTFSDYVINWVRQAPGQGLEWMGEIY | KNPKLTRMLTAKFAM |
| | REPQVYTLPPSREEMTKNQ | PGSGTNYYNEKFKAKATITADKSTSTAY | PKKATELKHLQCLEE |
| | VSLTCLVKGFYPSDIAVEW | MELSSLRSEDTAVYYCARRGRYGLYAMD | ELKPLEEVLNGAQSK |
| | ESNGQPENNYKTTPPVLDS | YWGQGTTVTVSSASTKGPSVFPLAPSSK | NFHLRPRDLISNINV |
| | DGSFFLYSKLTVDKSRWQQ | STSGGTAALGCLVKDYFPEPVTVSWNSG | IVLELKGSETTFMCE |
| | GNVFSCSVMHEALHNHYTQ | ALTSGVHTFPAVLQSSGLYSLSSVVTVP | YADETATIVEFLNRW |
| | KSLSLSPGK (SEQ ID | SSSLGTQTYICNVNHKPSNTKVDKRVEP | ITFAQSIISTLT |
| | NO: 202) | KSCDKTHS (SEQ ID NO: 203) | (SEQ ID NO: 204) |
| GA101- | DIVMTQTPLSLPVTPGEPA | QVQLVQSGAEVKKPGSSVKVSCKASGYA | |
| T13A- | SISCRSSKSLLHSNGITYL | FSYSWINWVRQAPGQGLEWMGRIFPGDG | |
| NKp46- | YWYLQKPGQSPQLLIYQMS | DTDYNGKFKGRVTITADKSTSTAYMELS | |
| 1-IL2v | NLVSGVPDRFSGSGSGTDF | SLRSEDTAVYYCARNVFDGYWLVYWGQG | |
| | TLKISRVEAEDVGVYYCAQ | TLVTVSSASTKGPSVFPLAPSSKSTSGG | |
| | NLELPYTFGGGTKVEIKRT | TAALGCLVKDYFPEPVTVSWNSGALTSG | |
| | VAAPSVFIFPPSDEQLKSG | VHTFPAVLQSSGLYSLSSVVTVPSSSLG | |
| | TASVVCLLNNFYPREAKVQ | TQTYICNVNHKPSNTKVDKRVEPKSCDK | |
| | WKVDNALQSGNSQESVTEQ | THTCPPCPAPELLGGPSVFLFPPKPKDT | |
| | DSKDSTYSLSSTLTLSKAD | LMISRTPEVTCVVVDVSHEDPEVKENWY | |
| | YEKHKVYACEVTHQGLSSP | VDGVEVHNAKTKPREEQYNSTYRVVSVL | |
| | VTKSFNRGECDKTHTCPPC | TVLHQDWLNGKEYKCKVSNKALPAPIEK | |
| | PAPELLGGPSVFLFPPKPK | TISKAKGQPREPQVYTLPPSREEMTKNQ | |
| | DTLMISRTPEVTCVVVDVS | VSLTCLVKGFYPSDIAVEWESNGQPENN | |
| | HEDPEVKFNWYVDGVEVHN | YKTTPPVLDSDGSFFLYSKLTVDKSRWQ | |
| | AKTKPREEQYNSTYRVVSV | QGNVFSCSVMHEALHNHYTQKSLSLSPG | |
| | LTVLHQDWLNGKEYKCKVS | STGSQVQLVQSGAEVKKPGSSVKVSCKA | |
| | NKALPAPIEKTISKAKGQP | SGYTFSDYVINWVRQAPGQGLEWMGEIY | |
| | REPQVYTLPPSREEMTKNQ | PGSGTNYYNEKFKAKATITADKSTSTAY | |
| | VSLTCLVKGFYPSDIAVEW | MELSSLRSEDTAVYYCARRGRYGLYAMD | |
| | ESNGQPENNYKTTPPVLDS | YWGQGTTVTVSSVEGGSGGSGGSGGSGG | |
| | DGSFFLYSKLTVDKSRWQQ | VDDIQMTQSPSSLSASVGDRVTITCRAS | |
| | GNVFSCSVMHEALHNRFTQ | QDISNYLNWYQQKPGKAPKLLIYYTSRL | |
| | KSLSLSPGK (SEQ ID | HSGVPSRFSGSGSGTDFTFTISSLQPED | |
| | NO: 205) | IATYFCQQGNTRPWTFGGGTKVEIKGSS | |
| | | SSGSSSSAPASSSTKKTQLQLEHLLLDL | |
| | | QMILNGINNYKNPKLTRMLTAKFAMPKK | |
| | | ATELKHLQCLEEELKPLEEVLNGAQSKN | |
| | | FHLRPRDLISNINVIVLELKGSETTFMC | |
| | | EYADETATIVEFLNRWITFAQSIISTLT | |
| | | (SEQ ID NO: 206) | |
| GA101- | DIVMTQTPLSLPVTPGEPA | QVQLVQSGAEVKKPGSSVKVSCKASGYA | |
| T14A- | SISCRSSKSLLHSNGITYL | FSYSWINWVRQAPGQGLEWMGRIFPGDG | |
| NKp46- | YWYLQKPGQSPQLLIYQMS | DTDYNGKFKGRVTITADKSTSTAYMELS | |
| 1-IL2v | NLVSGVPDRFSGSGSGTDF | SLRSEDTAVYYCARNVFDGYWLVYWGQG | |
| | TLKISRVEAEDVGVYYCAQ | TLVTVSSASTKGPSVFPLAPSSKSTSGG | |
| | NLELPYTFGGGTKVEIKRT | TAALGCLVKDYFPEPVTVSWNSGALTSG | |
| | VAAPSVFIFPPSDEQLKSG | VHTFPAVLQSSGLYSLSSVVTVPSSSLG | |
| | TASVVCLLNNFYPREAKVQ | TQTYICNVNHKPSNTKVDKRVEPKSCDK | |
| | WKVDNALQSGNSQESVTEQ | THTCPPCPAPELLGGPSVFLFPPKPKDT | |
| | DSKDSTYSLSSTLTLSKAD | LMISRTPEVTCVVVDVSHEDPEVKENWY | |
| | YEKHKVYACEVTHQGLSSP | VDGVEVHNAKTKPREEQYSSTYRVVSVL | |
| | VTKSFNRGECDKTHTCPPC | TVLHQDWLNGKEYKCKVSNKALPAPIEK | |
| | PAPELLGGPSVFLFPPKPK | TISKAKGQPREPQVYTLPPSREEMTKNQ | |
| | DTLMISRTPEVTCVVVDVS | VSLTCLVKGFYPSDIAVEWESNGQPENN | |
| | HEDPEVKFNWYVDGVEVHN | YKTTPPVLDSDGSFFLYSKLTVDKSRWQ | |
| | AKTKPREEQYSSTYRVVSV | QGNVFSCSVMHEALHNHYTQKSLSLSPG | |
| | LTVLHQDWLNGKEYKCKVS | STGSQVQLVQSGAEVKKPGSSVKVSCKA | |
| | NKALPAPIEKTISKAKGQP | SGYTFSDYVINWVRQAPGQGLEWMGEIY | |
| | REPQVYTLPPSREEMTKNQ | PGSGTNYYNEKFKAKATITADKSTSTAY | |
| | VSLTCLVKGFYPSDIAVEW | MELSSLRSEDTAVYYCARRGRYGLYAMD | |
| | ESNGQPENNYKTTPPVLDS | YWGQGTTVTVSSVEGGSGGSGGSGGSGG | |
| | DGSFFLYSKLTVDKSRWQQ | VDDIQMTQSPSSLSASVGDRVTITCRAS | |
| | GNVFSCSVMHEALHNRFTQ | QDISNYLNWYQQKPGKAPKLLIYYTSRL | |
| | KSLSLSPGK (SEQ ID | HSGVPSRFSGSGSGTDFTFTISSLQPED | |
| | NO: 207) | IATYFCQQGNTRPWTFGGGTKVEIKGSS | |
| | | SSGSSSSAPASSSTKKTQLQLEHLLLDL | |
| | | QMILNGINNYKNPKLTRMLTAKFAMPKK | |
| | | ATELKHLQCLEEELKPLEEVLNGAQSKN | |
| | | FHLRPRDLISNINVIVLELKGSETTFMC | |
| | | EYADETATIVEFLNRWITFAQSIISTLT | |
| | | (SEQ ID NO: 208) | |
| HER2 | DIQMTQSPSSLSASVGDRV | EVQLVESGGGLVQPGGSLRLSCAASGEN | DIQMTQSPSSLSASV |
| (trastu)- | TITCRASQDVNTAVAWYQQ | IKDTYIHWVRQAPGKGLEWVARIYPTNG | GDRVTITCRASQDIS |
| T5- | KPGKAPKLLIYSASFLYSG | YTRYADSVKGRFTISADTSKNTAYLQMN | NYLNWYQQKPGKAPK |
| NKp46- | VPSRFSGSRSGTDFTLTIS | SLRAEDTAVYYCSRWGGDGFYAMDYWGQ | LLIYYTSRLHSGVPS |
| 1-IL2v | SLQPEDFATYYCQQHYTTP | GTLVTVSSASTKGPSVFPLAPSSKSTSG | RFSGSGSGTDFTFTI |
| | PTFGQGTKVEIKRTVAAPS | GTAALGCLVKDYFPEPVTVSWNSGALTS | SSLQPEDIATYFCQQ |
| | VFIFPPSDEQLKSGTASVV | GVHTFPAVLQSSGLYSLSSVVTVPSSSL | GNTRPWTFGGGTKVE |
| | CLLNNFYPREAKVQWKVDN | GTQTYICNVNHKPSNTKVDKRVEPKSCD | IKASTKGPSVFPLAP |
| | ALQSGNSQESVTEQDSKDS | KTHTCPPCPAPELLGGPSVFLFPPKPKD | SSKSTSGGTAALGCL |
| | TYSLSSTLTLSKADYEKHK | TLMISRTPEVTCVVVDVSHEDPEVKENW | VKDYFPEPVTVSWNS |
| | VYACEVTHQGLSSPVTKSF | YVDGVEVHNAKTKPREEQYNSTYRVVSV | GALTSGVHTFPAVLQ |
| | NRGECDKTHTCPPCPAPEL | LTVLHQDWLNGKEYKCKVSNKALPAPIE | SSGLYSLSSVVTVPS |
| | LGGPSVFLFPPKPKDTLMI | KTISKAKGQPREPQVYTLPPSREEMTKN | SSLGTQTYICNVNHK |
| | SRTPEVTCVVVDVSHEDPE | QVSLTCLVKGFYPSDIAVEWESNGQPEN | PSNTKVDKRVEPKSC |
| | VKFNWYVDGVEVHNAKTKP | NYKTTPPVLDSDGSFFLYSKLTVDKSRW | DKTHSGSSSSGSSSS |
| | REEQYNSTYRVVSVLTVLH | QQGNVFSCSVMHEALHNHYTQKSLSLSP | APASSSTKKTQLQLE |
| | QDWLNGKEYKCKVSNKALP | GSTGSQVQLVQSGAEVKKPGSSVKVSCK | HLLLDLQMILNGINN |
| | APIEKTISKAKGQPREPQV | ASGYTFSDYVINWVRQAPGQGLEWMGEI | YKNPKLTRMLTAKFA |
| | YTLPPSREEMTKNQVSLTC | YPGSGTNYYNEKFKAKATITADKSTSTA | MPKKATELKHLQCLE |
| | LVKGFYPSDIAVEWESNGQ | YMELSSLRSEDTAVYYCARRGRYGLYAM | EELKPLEEVLNGAQS |
| | PENNYKTTPPVLDSDGSFF | DYWGQGTTVTVSSRTVAAPSVFIFPPSD | KNFHLRPRDLISNIN |
| | LYSKLTVDKSRWQQGNVFS | EQLKSGTASVVCLLNNFYPREAKVQWKV | VIVLELKGSETTFMC |
| | CSVMHEALHNHYTQKSLSL | DNALQSGNSQESVTEQDSKDSTYSLSST | EYADETATIVEFLNR |
| | SPGK | LTLSKADYEKHKVYACEVTHQGLSSPVT | WITFAQSIISTLT |
| | (SEQ ID NO: 209) | KSENRGEC (SEQ ID NO: 210) | (SEQ ID NO: 211) |
| HER2 | DIQMTQSPSSLSASVGDRV | EVQLVESGGGLVQPGGSLRLSCAASGEN | DIQMTQSPSSLSASV |
| (trastu)- | TITCRASQDVNTAVAWYQQ | IKDTYIHWVRQAPGKGLEWVARIYPTNG | GDRVTITCRASQDIS |
| T25- | KPGKAPKLLIYSASFLYSG | YTRYADSVKGRFTISADTSKNTAYLQMN | NYLNWYQQKPGKAPK |
| NKp46- | VPSRFSGSRSGTDFTLTIS | SLRAEDTAVYYCSRWGGDGFYAMDYWGQ | LLIYYTSRLHSGVPS |
| 1-IL2v | SLQPEDFATYYCQQHYTTP | GTLVTVSSASTKGPSVFPLAPSSKSTSG | RFSGSGSGTDFTFTI |
| | PTFGQGTKVEIKRTVAAPS | GTAALGCLVKDYFPEPVTVSWNSGALTS | SSLQPEDIATYFCQQ |
| | VFIFPPSDEQLKSGTASVV | GVHTFPAVLQSSGLYSLSSVVTVPSSSL | GNTRPWTFGGGTKVE |
| | CLLNNFYPREAKVQWKVDN | GTQTYICNVNHKPSNTKVDKRVEPKSCD | IKRTVAAPSVFIFPP |
| | ALQSGNSQESVTEQDSKDS | KTHTCPPCPAPELLGGPSVFLFPPKPKD | SDEQLKSGTASVVCL |
| | TYSLSSTLTLSKADYEKHK | TLMISRTPEVTCVVVDVSHEDPEVKENW | LNNFYPREAKVQWKV |
| | VYACEVTHQGLSSPVTKSF | YVDGVEVHNAKTKPREEQYNSTYRVVSV | DNALQSGNSQESVTE |
| | NRGECDKTHTCPPCPAPEL | LTVLHQDWLNGKEYKCKVSNKALPAPIE | QDSKDSTYSLSSTLT |
| | LGGPSVFLFPPKPKDTLMI | KTISKAKGQPREPQVYTLPPSREEMTKN | LSKADYEKHKVYACE |
| | SRTPEVTCVVVDVSHEDPE | QVSLTCLVKGFYPSDIAVEWESNGQPEN | VTHQGLSSPVTKSEN |
| | VKFNWYVDGVEVHNAKTKP | NYKTTPPVLDSDGSFFLYSKLTVDKSRW | RGECGSSSSGSSSSA |
| | REEQYNSTYRVVSVLTVLH | QQGNVFSCSVMHEALHNHYTQKSLSLSP | PASSSTKKTQLQLEH |
| | QDWLNGKEYKCKVSNKALP | GSTGSQVQLVQSGAEVKKPGSSVKVSCK | LLLDLQMILNGINNY |
| | APIEKTISKAKGQPREPQV | ASGYTFSDYVINWVRQAPGQGLEWMGEI | KNPKLTRMLTAKFAM |
| | YTLPPSREEMTKNQVSLTC | YPGSGTNYYNEKFKAKATITADKSTSTA | PKKATELKHLQCLEE |
| | LVKGFYPSDIAVEWESNGQ | YMELSSLRSEDTAVYYCARRGRYGLYAM | ELKPLEEVLNGAQSK |
| | PENNYKTTPPVLDSDGSFF | DYWGQGTTVTVSSASTKGPSVFPLAPSS | NFHLRPRDLISNINV |
| | LYSKLTVDKSRWQQGNVFS | KSTSGGTAALGCLVKDYFPEPVTVSWNS | IVLELKGSETTFMCE |
| | CSVMHEALHNHYTQKSLSL | GALTSGVHTFPAVLQSSGLYSLSSVVTV | YADETATIVEFLNRW |
| | SPGK | PSSSLGTQTYICNVNHKPSNTKVDKRVE | ITFAQSIISTLT |
| | (SEQ ID NO: 212) | PKSCDKTHS (SEQ ID NO: 213) | (SEQ ID NO: 214) |
| EGFR | DILLTQSPVILSVSPGERV | QVQLKQSGPGLVQPSQSLSITCTVSGES | DIQMTQSPSSLSASV |
| (Cetux)- | SFSCRASQSIGTNIHWYQQ | LTNYGVHWVRQSPGKGLEWLGVIWSGGN | GDRVTITCRASQDIS |
| T5- | RTNGSPRLLIKYASESISG | TDYNTPFTSRLSINKDNSKSQVFFKMNS | NYLNWYQQKPGKAPK |
| NKp46- | IPSRFSGSGSGTDFTLSIN | LQSNDTAIYYCARALTYYDYEFAYWGQG | LLIYYTSRLHSGVPS |
| 1-IL2v | SVESEDIADYYCQQNNNWP | TLVTVSAASTKGPSVFPLAPSSKSTSGG | RFSGSGSGTDFTFTI |
| | TTFGAGTKLELKRTVAAPS | TAALGCLVKDYFPEPVTVSWNSGALTSG | SSLQPEDIATYFCQQ |
| | VFIFPPSDEQLKSGTASVV | VHTFPAVLQSSGLYSLSSVVTVPSSSLG | GNTRPWTFGGGTKVE |
| | CLLNNFYPREAKVQWKVDN | TQTYICNVNHKPSNTKVDKRVEPKSCDK | IKASTKGPSVFPLAP |
| | ALQSGNSQESVTEQDSKDS | THTCPPCPAPELLGGPSVFLFPPKPKDT | SSKSTSGGTAALGCL |
| | TYSLSSTLTLSKADYEKHK | LMISRTPEVTCVVVDVSHEDPEVKENWY | VKDYFPEPVTVSWNS |
| | VYACEVTHQGLSSPVTKSF | VDGVEVHNAKTKPREEQYNSTYRVVSVL | GALTSGVHTFPAVLQ |
| | NRGECDKTHTCPPCPAPEL | TVLHQDWLNGKEYKCKVSNKALPAPIEK | SSGLYSLSSVVTVPS |
| | LGGPSVFLFPPKPKDTLMI | TISKAKGQPREPQVYTLPPSREEMTKNQ | SSLGTQTYICNVNHK |
| | SRTPEVTCVVVDVSHEDPE | VSLTCLVKGFYPSDIAVEWESNGQPENN | PSNTKVDKRVEPKSC |
| | VKFNWYVDGVEVHNAKTKP | YKTTPPVLDSDGSFFLYSKLTVDKSRWQ | DKTHSGSSSSGSSSS |
| | REEQYNSTYRVVSVLTVLH | QGNVFSCSVMHEALHNHYTQKSLSLSPG | APASSSTKKTQLQLE |
| | QDWLNGKEYKCKVSNKALP | STGSQVQLVQSGAEVKKPGSSVKVSCKA | HLLLDLQMILNGINN |
| | APIEKTISKAKGQPREPQV | SGYTFSDYVINWVRQAPGQGLEWMGEIY | YKNPKLTRMLTAKFA |
| | YTLPPSREEMTKNQVSLTC | PGSGTNYYNEKFKAKATITADKSTSTAY | MPKKATELKHLQCLE |
| | LVKGFYPSDIAVEWESNGQ | MELSSLRSEDTAVYYCARRGRYGLYAMD | EELKPLEEVLNGAQS |
| | PENNYKTTPPVLDSDGSFF | YWGQGTTVTVSSRTVAAPSVFIFPPSDE | KNFHLRPRDLISNIN |
| | LYSKLTVDKSRWQQGNVES | QLKSGTASVVCLLNNFYPREAKVQWKVD | VIVLELKGSETTFMC |
| | CSVMHEALHNHYTQKSLSL | NALQSGNSQESVTEQDSKDSTYSLSSTL | EYADETATIVEFLNR |
| | SPGK | TLSKADYEKHKVYACEVTHQGLSSPVTK | WITFAQSIISTLT |
| | (SEQ ID NO: 215) | SFNRGEC (SEQ ID NO: 216) | (SEQ ID NO: 217) |
| EGFR | DILLTQSPVILSVSPGERV | QVQLKQSGPGLVQPSQSLSITCTVSGFS | DIQMTQSPSSLSASV |
| (Cetux)- | SFSCRASQSIGTNIHWYQQ | LTNYGVHWVRQSPGKGLEWLGVIWSGGN | GDRVTITCRASQDIS |
| T25- | RTNGSPRLLIKYASESISG | TDYNTPFTSRLSINKDNSKSQVFFKMNS | NYLNWYQQKPGKAPK |
| NKp46- | IPSRFSGSGSGTDFTLSIN | LQSNDTAIYYCARALTYYDYEFAYWGQG | LLIYYTSRLHSGVPS |
| 1-IL2v | SVESEDIADYYCQQNNNWP | TLVTVSAASTKGPSVFPLAPSSKSTSGG | RFSGSGSGTDFTFTI |
| | TTFGAGTKLELKRTVAAPS | TAALGCLVKDYFPEPVTVSWNSGALTSG | SSLQPEDIATYFCQQ |
| | VFIFPPSDEQLKSGTASVV | VHTFPAVLQSSGLYSLSSVVTVPSSSLG | GNTRPWTFGGGTKVE |
| | CLLNNFYPREAKVQWKVDN | TQTYICNVNHKPSNTKVDKRVEPKSCDK | IKRTVAAPSVFIFPP |
| | ALQSGNSQESVTEQDSKDS | THTCPPCPAPELLGGPSVFLFPPKPKDT | SDEQLKSGTASVVCL |
| | TYSLSSTLTLSKADYEKHK | LMISRTPEVTCVVVDVSHEDPEVKENWY | LNNFYPREAKVQWKV |
| | VYACEVTHQGLSSPVTKSF | VDGVEVHNAKTKPREEQYNSTYRVVSVL | DNALQSGNSQESVTE |
| | NRGECDKTHTCPPCPAPEL | TVLHQDWLNGKEYKCKVSNKALPAPIEK | QDSKDSTYSLSSTLT |
| | LGGPSVFLFPPKPKDTLMI | TISKAKGQPREPQVYTLPPSREEMTKNQ | LSKADYEKHKVYACE |
| | SRTPEVTCVVVDVSHEDPE | VSLTCLVKGFYPSDIAVEWESNGQPENN | VTHQGLSSPVTKSEN |
| | VKFNWYVDGVEVHNAKTKP | YKTTPPVLDSDGSFFLYSKLTVDKSRWQ | RGECGSSSSGSSSSA |
| | REEQYNSTYRVVSVLTVLH | QGNVFSCSVMHEALHNHYTQKSLSLSPG | PASSSTKKTQLQLEH |
| | QDWLNGKEYKCKVSNKALP | STGSQVQLVQSGAEVKKPGSSVKVSCKA | LLLDLQMILNGINNY |
| | APIEKTISKAKGQPREPQV | SGYTFSDYVINWVRQAPGQGLEWMGEIY | KNPKLTRMLTAKFAM |
| | YTLPPSREEMTKNQVSLTC | PGSGTNYYNEKFKAKATITADKSTSTAY | PKKATELKHLQCLEE |
| | LVKGFYPSDIAVEWESNGQ | MELSSLRSEDTAVYYCARRGRYGLYAMD | ELKPLEEVLNGAQSK |
| | PENNYKTTPPVLDSDGSFF | YWGQGTTVTVSSASTKGPSVFPLAPSSK | NFHLRPRDLISNINV |
| | LYSKLTVDKSRWQQGNVFS | STSGGTAALGCLVKDYFPEPVTVSWNSG | IVLELKGSETTFMCE |
| | CSVMHEALHNHYTQKSLSL | ALTSGVHTFPAVLQSSGLYSLSSVVTVP | YADETATIVEFLNRW |
| | SPGK (SEQ ID NO: 218) | SSSLGTQTYICNVNHKPSNTKVDKRVEP | ITFAQSIISTLT |
| | | KSCDKTHS (SEQ ID NO: 219) | (SEQ ID NO: 220) |
| CD20- | DIVMTQTPLSLPVTPGEPA | QVQLVQSGAEVKKPGSSVKVSCKASGYA | DIQMTQSPSSLSASV |
| T5- | SISCRSSKSLLHSNGITYL | FSYSWINWVRQAPGQGLEWMGRIFPGDG | GDRVTITCRASQDIS |
| NKp46- | YWYLQKPGQSPQLLIYQMS | DTDYNGKFKGRVTITADKSTSTAYMELS | NYLNWYQQKPGKAPK |
| IL 15 | NLVSGVPDRFSGSGSGTDF | SLRSEDTAVYYCARNVEDGYWLVYWGQG | LLIYYTSRLHSGVPS |
| | TLKISRVEAEDVGVYYCAQ | TLVTVSSASTKGPSVFPLAPSSKSTSGG | RFSGSGSGTDFTFTI |
| | NLELPYTFGGGTKVEIKRT | TAALGCLVKDYFPEPVTVSWNSGALTSG | SSLQPEDIATYFCQQ |
| | VAAPSVFIFPPSDEQLKSG | VHTFPAVLQSSGLYSLSSVVTVPSSSLG | GNTRPWTFGGGTKVE |
| | TASVVCLLNNFYPREAKVQ | TQTYICNVNHKPSNTKVDKRVEPKSCDK | IKASTKGPSVFPLAP |
| | WKVDNALQSGNSQESVTEQ | THTCPPCPAPELLGGPSVFLFPPKPKDT | SSKSTSGGTAALGCL |
| | DSKDSTYSLSSTLTLSKAD | LMISRTPEVTCVVVDVSHEDPEVKENWY | VKDYFPEPVTVSWNS |
| | YEKHKVYACEVTHQGLSSP | VDGVEVHNAKTKPREEQYNSTYRVVSVL | GALTSGVHTFPAVLQ |
| | VTKSFNRGECDKTHTCPPC | TVLHQDWLNGKEYKCKVSNKALPAPIEK | SSGLYSLSSVVTVPS |
| | PAPELLGGPSVFLFPPKPK | TISKAKGQPREPQVYTLPPSREEMTKNQ | SSLGTQTYICNVNHK |
| | DTLMISRTPEVTCVVVDVS | VSLTCLVKGFYPSDIAVEWESNGQPENN | PSNTKVDKRVEPKSC |
| | HEDPEVKFNWYVDGVEVHN | YKTTPPVLDSDGSFFLYSKLTVDKSRWQ | DKTHSGGGGSGGGGS |
| | AKTKPREEQYNSTYRVVSV | QGNVFSCSVMHEALHNHYTQKSLSLSPG | GGGGSNWVNVISDLK |
| | LTVLHQDWLNGKEYKCKVS | STGSQVQLVQSGAEVKKPGSSVKVSCKA | KIEDLIQSMHIDATL |
| | NKALPAPIEKTISKAKGQP | SGYTFSDYVINWVRQAPGQGLEWMGEIY | YTESDVHPSCKVTAM |
| | REPQVYTLPPSREEMTKNQ | PGSGTNYYNEKFKAKATITADKSTSTAY | KCFLLELQVISLESG |
| | VSLTCLVKGFYPSDIAVEW | MELSSLRSEDTAVYYCARRGRYGLYAMD | DASIHDTVENLIILA |
| | ESNGQPENNYKTTPPVLDS | YWGQGTTVTVSSRTVAAPSVFIFPPSDE | NNSLSSNGNVTESGC |
| | DGSFFLYSKLTVDKSRWQQ | QLKSGTASVVCLLNNFYPREAKVQWKVD | KECEELEEKNIKEFL |
| | GNVFSCSVMHEALHNHYTQ | NALQSGNSQESVTEQDSKDSTYSLSSTL | QSFVHIVQMFINTS |
| | KSLSLSPGK | TLSKADYEKHKVYACEVTHQGLSSPVTK | (SEQ ID NO: 443) |
| | (SEQ ID NO: 441) | SFNRGEC (SEQ ID NO: 442) | |
| CD20- | DIVMTQTPLSLPVTPGEPA | QVQLVQSGAEVKKPGSSVKVSCKASGYA | DIQMTQSPSSLSASV |
| T5- | SISCRSSKSLLHSNGITYL | FSYSWINWVRQAPGQGLEWMGRIFPGDG | GDRVTITCRASQDIS |
| NKp46- | YWYLQKPGQSPQLLIYQMS | DTDYNGKFKGRVTITADKSTSTAYMELS | NYLNWYQQKPGKAPK |
| IL 18v | NLVSGVPDRFSGSGSGTDE | SLRSEDTAVYYCARNVFDGYWLVYWGQG | LLIYYTSRLHSGVPS |
| | TLKISRVEAEDVGVYYCAQ | TLVTVSSASTKGPSVFPLAPSSKSTSGG | RFSGSGSGTDFTFTI |
| | NLELPYTFGGGTKVEIKRT | TAALGCLVKDYFPEPVTVSWNSGALTSG | SSLQPEDIATYFCQQ |
| | VAAPSVFIFPPSDEQLKSG | VHTFPAVLQSSGLYSLSSVVTVPSSSLG | GNTRPWTFGGGTKVE |
| | TASVVCLLNNFYPREAKVQ | TQTYICNVNHKPSNTKVDKRVEPKSCDK | IKASTKGPSVFPLAP |
| | WKVDNALQSGNSQESVTEQ | THTCPPCPAPELLGGPSVFLFPPKPKDT | SSKSTSGGTAALGCL |
| | DSKDSTYSLSSTLTLSKAD | LMISRTPEVTCVVVDVSHEDPEVKENWY | VKDYFPEPVTVSWNS |
| | YEKHKVYACEVTHQGLSSP | VDGVEVHNAKTKPREEQYNSTYRVVSVL | GALTSGVHTFPAVLQ |
| | VTKSENRGECDKTHTCPPC | TVLHQDWLNGKEYKCKVSNKALPAPIEK | SSGLYSLSSVVTVPS |
| | PAPELLGGPSVFLFPPKPK | TISKAKGQPREPQVYTLPPSREEMTKNQ | SSLGTQTYICNVNHK |
| | DTLMISRTPEVTCVVVDVS | VSLTCLVKGFYPSDIAVEWESNGQPENN | PSNTKVDKRVEPKSC |
| | HEDPEVKENWYVDGVEVHN | YKTTPPVLDSDGSFFLYSKLTVDKSRWQ | DKTHSGGSGGGGSGG |
| | AKTKPREEQYNSTYRVVSV | QGNVFSCSVMHEALHNHYTQKSLSLSPG | GGSGGYFGKLESKLS |
| | LTVLHQDWLNGKEYKCKVS | STGSQVQLVQSGAEVKKPGSSVKVSCKA | VIRNLNDQVLFIDQG |
| | NKALPAPIEKTISKAKGQP | SGYTFSDYVINWVRQAPGQGLEWMGEIY | NRPLFEDMTDSDCRD |
| | REPQVYTLPPSREEMTKNQ | PGSGTNYYNEKFKAKATITADKSTSTAY | NAPRTIFIISKYGDS |
| | VSLTCLVKGFYPSDIAVEW | MELSSLRSEDTAVYYCARRGRYGLYAMD | GARGLAVTISVKCEK |
| | ESNGQPENNYKTTPPVLDS | YWGQGTTVTVSSRTVAAPSVFIFPPSDE | ISTLSCENKIISFKE |
| | DGSFFLYSKLTVDKSRWQQ | QLKSGTASVVCLLNNFYPREAKVQWKVD | MNPPDNIKDTKSDII |
| | GNVFSCSVMHEALHNHYTQ | NALQSGNSQESVTEQDSKDSTYSLSSTL | FFERDVPGHSGKVQF |
| | KSLSLSPGK | TLSKADYEKHKVYACEVTHQGLSSPVTK | ESSSYEGYFLACEKE |
| | (SEQ ID NO: 444) | SFNRGEC | RDLFKLILKKEDELG |
| | | (SEQ ID NO: 445) | DRSIMFTVQNED |
| | | | (SEQ ID NO: 446) |
| CD20- | DIVMTQTPLSLPVTPGEPA | QVQLVQSGAEVKKPGSSVKVSCKASGYA | DIQMTQSPSSLSASV |
| T5- | SISCRSSKSLLHSNGITYL | FSYSWINWVRQAPGQGLEWMGRIFPGDG | GDRVTITCRASQDIS |
| NKp46- | YWYLQKPGQSPQLLIYQMS | DTDYNGKFKGRVTITADKSTSTAYMELS | NYLNWYQQKPGKAPK |
| IFNαv | NLVSGVPDRFSGSGSGTDF | SLRSEDTAVYYCARNVFDGYWLVYWGQG | LLIYYTSRLHSGVPS |
| | TLKISRVEAEDVGVYYCAQ | TLVTVSSASTKGPSVFPLAPSSKSTSGG | RFSGSGSGTDFTFTI |
| | NLELPYTFGGGTKVEIKRT | TAALGCLVKDYFPEPVTVSWNSGALTSG | SSLQPEDIATYFCQQ |
| | VAAPSVFIFPPSDEQLKSG | VHTFPAVLQSSGLYSLSSVVTVPSSSLG | GNTRPWTFGGGTKVE |
| | TASVVCLLNNFYPREAKVQ | TQTYICNVNHKPSNTKVDKRVEPKSCDK | IKASTKGPSVFPLAP |
| | WKVDNALQSGNSQESVTEQ | THTCPPCPAPELLGGPSVFLFPPKPKDT | SSKSTSGGTAALGCL |
| | DSKDSTYSLSSTLTLSKAD | LMISRTPEVTCVVVDVSHEDPEVKENWY | VKDYFPEPVTVSWNS |
| | YEKHKVYACEVTHQGLSSP | VDGVEVHNAKTKPREEQYNSTYRVVSVL | GALTSGVHTFPAVLQ |
| | VTKSFNRGECDKTHTCPPC | TVLHQDWLNGKEYKCKVSNKALPAPIEK | SSGLYSLSSVVTVPS |
| | PAPELLGGPSVFLFPPKPK | TISKAKGQPREPQVYTLPPSREEMTKNQ | SSLGTQTYICNVNHK |
| | DTLMISRTPEVTCVVVDVS | VSLTCLVKGFYPSDIAVEWESNGQPENN | PSNTKVDKRVEPKSC |
| | HEDPEVKFNWYVDGVEVHN | YKTTPPVLDSDGSFFLYSKLTVDKSRWQ | DKTHSGGGGSGGGGS |
| | AKTKPREEQYNSTYRVVSV | QGNVFSCSVMHEALHNHYTQKSLSLSPG | GGGGSDLPQTHSLGS |
| | LTVLHQDWLNGKEYKCKVS | STGSQVQLVQSGAEVKKPGSSVKVSCKA | RRTLMLLAQMRRISL |
| | NKALPAPIEKTISKAKGQP | SGYTFSDYVINWVRQAPGQGLEWMGEIY | FSCAKDRHDFGFPQE |
| | REPQVYTLPPSREEMTKNQ | PGSGTNYYNEKFKAKATITADKSTSTAY | EFGNQFQKAETIPVL |
| | VSLTCLVKGFYPSDIAVEW | MELSSLRSEDTAVYYCARRGRYGLYAMD | HEMIQQIFNLFSTKD |
| | ESNGQPENNYKTTPPVLDS | YWGQGTTVTVSSRTVAAPSVFIFPPSDE | SSAAWDETLLDKFYT |
| | DGSFFLYSKLTVDKSRWQQ | QLKSGTASVVCLLNNFYPREAKVQWKVD | ELYQQLNDLEACVIQ |
| | GNVFSCSVMHEALHNHYTQ | NALQSGNSQESVTEQDSKDSTYSLSSTL | GVGVTETPLMKEDSI |
| | KSLSLSPGK | TLSKADYEKHKVYACEVTHQGLSSPVTK | LAVRKYFQRITLYLK |
| | (SEQ ID NO: 447) | SENRGEC | EKKYSPCAWEVVRAE |
| | | (SEQ ID NO: 448) | IMRSFSLSTNLQESL |
| | | | RSKE |
| | | | (SEQ ID NO: 449) |
| IC-T6- | EIVLTQSPATLSLSPGERA | EVQLVESGGGLVQPGRSLRLSCAASGFT | EIVLTQSPGTLSLSP |
| IC-IL2v | TLSCRASQSVRSYLAWYQQ | FDNYAMHWVRQAPGKGLEWVSGISRSSG | GERATLSCRASQSVS |
| | KPGQAPRLLFSDASNRATG | DIDYADSVKGRFTISRDNAKNSLYLQMN | SSYLAWYQQKPGQAP |
| | IPARFSGSGSGTDFTLTIS | SLRAEDTALYYCARGGVGSFDTWGQGTM | RLLIYGASSRATGIP |
| | SLEPEDFAVYYCQQYRYSP | VTVSSASTKGPSVFPLAPSSKSTSGGTA | DRFSGSGSGTDFTLT |
| | RTFGQGTKVEIKRTVAAPS | ALGCLVKDYFPEPVTVSWNSGALTSGVH | ISRLEPEDFAVYYCQ |
| | VFIFPPSDEQLKSGTASVV | TFPAVLQSSGLYSLSSVVTVPSSSLGTQ | QYGSSTWTFGQGTKV |
| | CLLNNFYPREAKVQWKVDN | TYICNVNHKPSNTKVDKRVEPKSCDKTH | EIKASTKGPSVFPLA |
| | ALQSGNSQESVTEQDSKDS | TCPPCPAPELLGGPSVFLFPPKPKDTLM | PSSKSTSGGTAALGC |
| | TYSLSSTLTLSKADYEKHK | ISRTPEVTCVVVDVSHEDPEVKENWYVD | LVKDYFPEPVTVSWN |
| | VYACEVTHQGLSSPVTKSF | GVEVHNAKTKPREEQYSSTYRVVSVLTV | SGALTSGVHTFPAVL |
| | NRGECDKTHTCPPCPAPEL | LHQDWLNGKEYKCKVSNKALPAPIEKTI | QSSGLYSLSSVVTVP |
| | LGGPSVFLFPPKPKDTLMI | SKAKGQPREPQVYTLPPSREEMTKNQVS | SSSLGTQTYICNVNH |
| | SRTPEVTCVVVDVSHEDPE | LTCLVKGFYPSDIAVEWESNGQPENNYK | KPSNTKVDKRVEPKS |
| | VKFNWYVDGVEVHNAKTKP | TTPPVLDSDGSFFLYSKLTVDKSRWQQG | CDKTHSGSSSSGSSS |
| | REEQYSSTYRVVSVLTVLH | NVFSCSVMHEALHNHYTQKSLSLSPGST | SAPASSSTKKTQLQL |
| | QDWLNGKEYKCKVSNKALP | GSEVQLVQSGAEVKKSGESLKISCKGSG | EHLLLDLQMILNGIN |
| | APIEKTISKAKGQPREPQV | YSFTSYWIGWVRQMPGKGLEWMGIFYPG | NYKNPKLTRMLTAKF |
| | YTLPPSREEMTKNQVSLTC | DSSTRYSPSFQGQVTISADKSVNTAYLQ | AMPKKATELKHLQCL |
| | LVKGFYPSDIAVEWESNGQ | WSSLKASDTAMYYCARRRNWGNAFDIWG | EEELKPLEEVLNGAQ |
| | PENNYKTTPPVLDSDGSFF | QGTMVTVSSRTVAAPSVFIFPPSDEQLK | SKNFHLRPRDLISNI |
| | LYSKLTVDKSRWQQGNVFS | SGTASVVCLLNNFYPREAKVQWKVDNAL | NVIVLELKGSETTFM |
| | CSVMHEALHNHYTQKSLSL | QSGNSQESVTEQDSKDSTYSLSSTLTLS | CEYADETATIVEFLN |
| | SPGK | KADYEKHKVYACEVTHQGLSSPVTKSEN | RWITFAQSIISTLT |
| | (SEQ ID NO: 450) | RGEC | (SEQ ID NO: 452) |
| | | (SEQ ID NO: 451) | |
| IC-T6- | EIVLTQSPATLSLSPGERA | EVQLVESGGGLVQPGRSLRLSCAASGFT | EIVLTQSPGTLSLSP |
| IC- | TLSCRASQSVRSYLAWYQQ | FDNYAMHWVRQAPGKGLEWVSGISRSSG | GERATLSCRASQSVS |
| IL2pWT | KPGQAPRLLFSDASNRATG | DIDYADSVKGRFTISRDNAKNSLYLQMN | SSYLAWYQQKPGQAP |
| | IPARFSGSGSGTDFTLTIS | SLRAEDTALYYCARGGVGSFDTWGQGTM | RLLIYGASSRATGIP |
| | SLEPEDFAVYYCQQYRYSP | VTVSSASTKGPSVFPLAPSSKSTSGGTA | DRFSGSGSGTDFTLT |
| | RTFGQGTKVEIKRTVAAPS | ALGCLVKDYFPEPVTVSWNSGALTSGVH | ISRLEPEDFAVYYCQ |
| | VFIFPPSDEQLKSGTASVV | TFPAVLQSSGLYSLSSVVTVPSSSLGTQ | QYGSSTWTFGQGTKV |
| | CLLNNFYPREAKVQWKVDN | TYICNVNHKPSNTKVDKRVEPKSCDKTH | EIKASTKGPSVFPLA |
| | ALQSGNSQESVTEQDSKDS | TCPPCPAPELLGGPSVFLFPPKPKDTLM | PSSKSTSGGTAALGC |
| | TYSLSSTLTLSKADYEKHK | ISRTPEVTCVVVDVSHEDPEVKFNWYVD | LVKDYFPEPVTVSWN |
| | VYACEVTHQGLSSPVTKSF | GVEVHNAKTKPREEQYSSTYRVVSVLTV | SGALTSGVHTFPAVL |
| | NRGECDKTHTCPPCPAPEL | LHQDWLNGKEYKCKVSNKALPAPIEKTI | QSSGLYSLSSVVTVP |
| | LGGPSVFLFPPKPKDTLMI | SKAKGQPREPQVYTLPPSREEMTKNQVS | SSSLGTQTYICNVNH |
| | SRTPEVTCVVVDVSHEDPE | LTCLVKGFYPSDIAVEWESNGQPENNYK | KPSNTKVDKRVEPKS |
| | VKFNWYVDGVEVHNAKTKP | TTPPVLDSDGSFFLYSKLTVDKSRWQQG | CDKTHSGSSSSGSSS |
| | REEQYSSTYRVVSVLTVLH | NVFSCSVMHEALHNHYTQKSLSLSPGST | SGSSSSTKKTQLQLE |
| | QDWLNGKEYKCKVSNKALP | GSEVQLVQSGAEVKKSGESLKISCKGSG | HLLLDLQMILNGINN |
| | APIEKTISKAKGQPREPQV | YSFTSYWIGWVRQMPGKGLEWMGIFYPG | YKNPKLTRMLTFKFY |
| | YTLPPSREEMTKNQVSLTC | DSSTRYSPSFQGQVTISADKSVNTAYLQ | MPKKATELKHLQCLE |
| | LVKGFYPSDIAVEWESNGQ | WSSLKASDTAMYYCARRRNWGNAFDIWG | EELKPLEEVLNLAQS |
| | PENNYKTTPPVLDSDGSFF | QGTMVTVSSRTVAAPSVFIFPPSDEQLK | KNFHLRPRDLISNIN |
| | LYSKLTVDKSRWQQGNVES | SGTASVVCLLNNFYPREAKVQWKVDNAL | VIVLELKGSETTFMC |
| | CSVMHEALHNHYTQKSLSL | QSGNSQESVTEQDSKDSTYSLSSTLTLS | EYADETATIVEFLNR |
| | SPGK | KADYEKHKVYACEVTHQGLSSPVTKSEN | WITFCQSIISTLT |
| | (SEQ ID NO: 453) | RGEC | (SEQ ID NO: 455) |
| | | (SEQ ID NO: 454) | |
| CD20- | DIVMTQTPLSLPVTPGEPA | QVQLVQSGAEVKKPGSSVKVSCKASGYA | EIVLTQSPGTLSLSP |
| T6-IC- | SISCRSSKSLLHSNGITYL | FSYSWINWVRQAPGQGLEWMGRIFPGDG | GERATLSCRASQSVS |
| IL2v | YWYLQKPGQSPQLLIYQMS | DTDYNGKFKGRVTITADKSTSTAYMELS | SSYLAWYQQKPGQAP |
| | NLVSGVPDRFSGSGSGTDF | SLRSEDTAVYYCARNVEDGYWLVYWGQG | RLLIYGASSRATGIP |
| | TLKISRVEAEDVGVYYCAQ | TLVTVSSASTKGPSVFPLAPSSKSTSGG | DRFSGSGSGTDFTLT |
| | NLELPYTFGGGTKVEIKRT | TAALGCLVKDYFPEPVTVSWNSGALTSG | ISRLEPEDFAVYYCQ |
| | VAAPSVFIFPPSDEQLKSG | VHTFPAVLQSSGLYSLSSVVTVPSSSLG | QYGSSTWTFGQGTKV |
| | TASVVCLLNNFYPREAKVQ | TQTYICNVNHKPSNTKVDKRVEPKSCDK | EIKASTKGPSVFPLA |
| | WKVDNALQSGNSQESVTEQ | THTCPPCPAPELLGGPSVFLFPPKPKDT | PSSKSTSGGTAALGC |
| | DSKDSTYSLSSTLTLSKAD | LMISRTPEVTCVVVDVSHEDPEVKENWY | LVKDYFPEPVTVSWN |
| | YEKHKVYACEVTHQGLSSP | VDGVEVHNAKTKPREEQYSSTYRVVSVL | SGALTSGVHTFPAVL |
| | VTKSFNRGECDKTHTCPPC | TVLHQDWLNGKEYKCKVSNKALPAPIEK | QSSGLYSLSSVVTVP |
| | PAPELLGGPSVFLFPPKPK | TISKAKGQPREPQVYTLPPSREEMTKNQ | SSSLGTQTYICNVNH |
| | DTLMISRTPEVTCVVVDVS | VSLTCLVKGFYPSDIAVEWESNGQPENN | KPSNTKVDKRVEPKS |
| | HEDPEVKFNWYVDGVEVHN | YKTTPPVLDSDGSFFLYSKLTVDKSRWQ | CDKTHSGSSSSGSSS |
| | AKTKPREEQYSSTYRVVSV | QGNVFSCSVMHEALHNHYTQKSLSLSPG | SAPASSSTKKTQLQL |
| | LTVLHQDWLNGKEYKCKVS | STGSEVQLVQSGAEVKKSGESLKISCKG | EHLLLDLQMILNGIN |
| | NKALPAPIEKTISKAKGQP | SGYSFTSYWIGWVRQMPGKGLEWMGIFY | NYKNPKLTRMLTAKF |
| | REPQVYTLPPSREEMTKNQ | PGDSSTRYSPSFQGQVTISADKSVNTAY | AMPKKATELKHLQCL |
| | VSLTCLVKGFYPSDIAVEW | LQWSSLKASDTAMYYCARRRNWGNAFDI | EEELKPLEEVLNGAQ |
| | ESNGQPENNYKTTPPVLDS | WGQGTMVTVSSRTVAAPSVFIFPPSDEQ | SKNFHLRPRDLISNI |
| | DGSFFLYSKLTVDKSRWQQ | LKSGTASVVCLLNNFYPREAKVQWKVDN | NVIVLELKGSETTEM |
| | GNVFSCSVMHEALHNHYTQ | ALQSGNSQESVTEQDSKDSTYSLSSTLT | CEYADETATIVEFLN |
| | KSLSLSPGK | LSKADYEKHKVYACEVTHQGLSSPVTKS | RWITFAQSIISTLT |
| | (SEQ ID NO: 456) | FNRGEC | (SEQ ID NO: 458) |
| | | (SEQ ID NO: 457) | |
| CD20- | DIVMTQTPLSLPVTPGEPA | QVQLVQSGAEVKKPGSSVKVSCKASGYA | EIVLTQSPGTLSLSP |
| T5-IC- | SISCRSSKSLLHSNGITYL | FSYSWINWVRQAPGQGLEWMGRIFPGDG | GERATLSCRASQSVS |
| IL2v | YWYLQKPGQSPQLLIYQMS | DTDYNGKFKGRVTITADKSTSTAYMELS | SSYLAWYQQKPGQAP |
| | NLVSGVPDRFSGSGSGTDF | SLRSEDTAVYYCARNVFDGYWLVYWGQG | RLLIYGASSRATGIP |
| | TLKISRVEAEDVGVYYCAQ | TLVTVSSASTKGPSVFPLAPSSKSTSGG | DRESGSGSGTDFTLT |
| | NLELPYTFGGGTKVEIKRT | TAALGCLVKDYFPEPVTVSWNSGALTSG | ISRLEPEDFAVYYCQ |
| | VAAPSVFIFPPSDEQLKSG | VHTFPAVLQSSGLYSLSSVVTVPSSSLG | QYGSSTWTFGQGTKV |
| | TASVVCLLNNFYPREAKVQ | TQTYICNVNHKPSNTKVDKRVEPKSCDK | EIKASTKGPSVFPLA |
| | WKVDNALQSGNSQESVTEQ | THTCPPCPAPELLGGPSVFLFPPKPKDT | PSSKSTSGGTAALGC |
| | DSKDSTYSLSSTLTLSKAD | LMISRTPEVTCVVVDVSHEDPEVKENWY | LVKDYFPEPVTVSWN |
| | YEKHKVYACEVTHQGLSSP | VDGVEVHNAKTKPREEQYNSTYRVVSVL | SGALTSGVHTFPAVL |
| | VTKSFNRGECDKTHTCPPC | TVLHQDWLNGKEYKCKVSNKALPAPIEK | QSSGLYSLSSVVTVP |
| | PAPELLGGPSVFLFPPKPK | TISKAKGQPREPQVYTLPPSREEMTKNQ | SSSLGTQTYICNVNH |
| | DTLMISRTPEVTCVVVDVS | VSLTCLVKGFYPSDIAVEWESNGQPENN | KPSNTKVDKRVEPKS |
| | HEDPEVKENWYVDGVEVHN | YKTTPPVLDSDGSFFLYSKLTVDKSRWQ | CDKTHSGSSSSGSSS |
| | AKTKPREEQYNSTYRVVSV | QGNVFSCSVMHEALHNHYTQKSLSLSPG | SAPASSSTKKTQLQL |
| | LTVLHQDWLNGKEYKCKVS | STGSEVQLVQSGAEVKKSGESLKISCKG | EHLLLDLQMILNGIN |
| | NKALPAPIEKTISKAKGQP | SGYSFTSYWIGWVRQMPGKGLEWMGIFY | NYKNPKLTRMLTAKF |
| | REPQVYTLPPSREEMTKNQ | PGDSSTRYSPSFQGQVTISADKSVNTAY | AMPKKATELKHLQCL |
| | VSLTCLVKGFYPSDIAVEW | LQWSSLKASDTAMYYCARRRNWGNAFDI | EEELKPLEEVLNGAQ |
| | ESNGQPENNYKTTPPVLDS | WGQGTMVTVSSRTVAAPSVFIFPPSDEQ | SKNFHLRPRDLISNI |
| | DGSFFLYSKLTVDKSRWQQ | LKSGTASVVCLLNNFYPREAKVQWKVDN | NVIVLELKGSETTFM |
| | GNVFSCSVMHEALHNHYTQ | ALQSGNSQESVTEQDSKDSTYSLSSTLT | CEYADETATIVEFLN |
| | KSLSLSPGK | LSKADYEKHKVYACEVTHQGLSSPVTKS | RWITFAQSIISTLT |
| | (SEQ ID NO: 459) | FNRGEC | (SEQ ID NO: 461) |
| | | (SEQ ID NO: 460) | |
| CD20- | DIVMTQTPLSLPVTPGEPA | QVQLVQSGAEVKKPGSSVKVSCKASGYA | DIQMTQSPSSLSASV |
| T6- | SISCRSSKSLLHSNGITYL | FSYSWINWVRQAPGQGLEWMGRIFPGDG | GDRVTITCRASQDIS |
| NKp46- | YWYLQKPGQSPQLLIYQMS | DTDYNGKFKGRVTITADKSTSTAYMELS | NYLNWYQQKPGKAPK |
| IL2v | NLVSGVPDRFSGSGSGTDF | SLRSEDTAVYYCARNVFDGYWLVYWGQG | LLIYYTSRLHSGVPS |
| | TLKISRVEAEDVGVYYCAQ | TLVTVSSASTKGPSVFPLAPSSKSTSGG | RFSGSGSGTDFTFTI |
| | NLELPYTFGGGTKVEIKRT | TAALGCLVKDYFPEPVTVSWNSGALTSG | SSLQPEDIATYFCQQ |
| | VAAPSVFIFPPSDEQLKSG | VHTFPAVLQSSGLYSLSSVVTVPSSSLG | GNTRPWTFGGGTKVE |
| | TASVVCLLNNFYPREAKVQ | TQTYICNVNHKPSNTKVDKRVEPKSCDK | IKASTKGPSVFPLAP |
| | WKVDNALQSGNSQESVTEQ | THTCPPCPAPELLGGPSVFLFPPKPKDT | SSKSTSGGTAALGCL |
| | DSKDSTYSLSSTLTLSKAD | LMISRTPEVTCVVVDVSHEDPEVKENWY | VKDYFPEPVTVSWNS |
| | YEKHKVYACEVTHQGLSSP | VDGVEVHNAKTKPREEQYSSTYRVVSVL | GALTSGVHTFPAVLQ |
| | VTKSFNRGECDKTHTCPPC | TVLHQDWLNGKEYKCKVSNKALPAPIEK | SSGLYSLSSVVTVPS |
| | PAPELLGGPSVFLFPPKPK | TISKAKGQPREPQVYTLPPSREEMTKNQ | SSLGTQTYICNVNHK |
| | DTLMISRTPEVTCVVVDVS | VSLTCLVKGFYPSDIAVEWESNGQPENN | PSNTKVDKRVEPKSC |
| | HEDPEVKFNWYVDGVEVHN | YKTTPPVLDSDGSFFLYSKLTVDKSRWQ | DKTHSGSSSSGSSSS |
| | AKTKPREEQYSSTYRVVSV | QGNVFSCSVMHEALHNHYTQKSLSLSPG | APASSSTKKTQLQLE |
| | LTVLHQDWLNGKEYKCKVS | STGSQVQLVQSGAEVKKPGSSVKVSCKA | HLLLDLQMILNGINN |
| | NKALPAPIEKTISKAKGQP | SGYTFSDYVINWVRQAPGQGLEWMGEIY | YKNPKLTRMLTAKFA |
| | REPQVYTLPPSREEMTKNQ | PGSGTNYYNEKFKAKATITADKSTSTAY | MPKKATELKHLQCLE |
| | VSLTCLVKGFYPSDIAVEW | MELSSLRSEDTAVYYCARRGRYGLYAMD | EELKPLEEVLNGAQS |
| | ESNGQPENNYKTTPPVLDS | YWGQGTTVTVSSRTVAAPSVFIFPPSDE | KNFHLRPRDLISNIN |
| | DGSFFLYSKLTVDKSRWQQ | QLKSGTASVVCLLNNFYPREAKVQWKVD | VIVLELKGSETTFMC |
| | GNVFSCSVMHEALHNHYTQ | NALQSGNSQESVTEQDSKDSTYSLSSTL | EYADETATIVEFLNR |
| | KSLSLSPGK | TLSKADYEKHKVYACEVTHQGLSSPVTK | WITFAQSIISTLT |
| | (SEQ ID NO: 462) | SFNRGEC | (SEQ ID NO: 464) |
| | | (SEQ ID NO: 463) | |
| CD20- | DIVMTQTPLSLPVTPGEPA | QVQLVQSGAEVKKPGSSVKVSCKASGYA | DIQMTQSPSSLSASV |
| F5- | SISCRSSKSLLHSNGITYL | FSYSWINWVRQAPGQGLEWMGRIFPGDG | GDRVTITCRASQDIS |
| NKp46 | YWYLQKPGQSPQLLIYQMS | DTDYNGKFKGRVTITADKSTSTAYMELS | NYLNWYQQKPGKAPK |
| | NLVSGVPDRESGSGSGTDF | SLRSEDTAVYYCARNVFDGYWLVYWGQG | LLIYYTSRLHSGVPS |
| | TLKISRVEAEDVGVYYCAQ | TLVTVSSASTKGPSVFPLAPSSKSTSGG | RFSGSGSGTDFTFTI |
| | NLELPYTFGGGTKVEIKRT | TAALGCLVKDYFPEPVTVSWNSGALTSG | SSLQPEDIATYFCQQ |
| | VAAPSVFIFPPSDEQLKSG | VHTFPAVLQSSGLYSLSSVVTVPSSSLG | GNTRPWTFGGGTKVE |
| | TASVVCLLNNFYPREAKVQ | TQTYICNVNHKPSNTKVDKRVEPKSCDK | IKASTKGPSVFPLAP |
| | WKVDNALQSGNSQESVTEQ | THTCPPCPAPELLGGPSVFLFPPKPKDT | SSKSTSGGTAALGCL |
| | DSKDSTYSLSSTLTLSKAD | LMISRTPEVTCVVVDVSHEDPEVKENWY | VKDYFPEPVTVSWNS |
| | YEKHKVYACEVTHQGLSSP | VDGVEVHNAKTKPREEQYNSTYRVVSVL | GALTSGVHTFPAVLQ |
| | VTKSFNRGECDKTHTCPPC | TVLHQDWLNGKEYKCKVSNKALPAPIEK | SSGLYSLSSVVTVPS |
| | PAPELLGGPSVFLFPPKPK | TISKAKGQPREPQVYTLPPSREEMTKNQ | SSLGTQTYICNVNHK |
| | DTLMISRTPEVTCVVVDVS | VSLTCLVKGFYPSDIAVEWESNGQPENN | PSNTKVDKRVEPKSC |
| | HEDPEVKFNWYVDGVEVHN | YKTTPPVLDSDGSFFLYSKLTVDKSRWQ | DKTHS |
| | AKTKPREEQYNSTYRVVSV | QGNVFSCSVMHEALHNHYTQKSLSLSPG | (SEQ ID NO: 467) |
| | LTVLHQDWLNGKEYKCKVS | STGSQVQLVQSGAEVKKPGSSVKVSCKA | |
| | NKALPAPIEKTISKAKGQP | SGYTFSDYVINWVRQAPGQGLEWMGEIY | |
| | REPQVYTLPPSREEMTKNQ | PGSGTNYYNEKFKAKATITADKSTSTAY | |
| | VSLTCLVKGFYPSDIAVEW | MELSSLRSEDTAVYYCARRGRYGLYAMD | |
| | ESNGQPENNYKTTPPVLDS | YWGQGTTVTVSSRTVAAPSVFIFPPSDE | |
| | DGSFFLYSKLTVDKSRWQQ | QLKSGTASVVCLLNNFYPREAKVQWKVD | |
| | GNVFSCSVMHEALHNHYTQ | NALQSGNSQESVTEQDSKDSTYSLSSTL | |
| | KSLSLSPGK | TLSKADYEKHKVYACEVTHQGLSSPVTK | |
| | (SEQ ID NO: 465) | SFNRGEC | |
| | | (SEQ ID NO: 466) | |
| CD20- | DIVMTQTPLSLPVTPGEPA | QVQLVQSGAEVKKPGSSVKVSCKASGYA | DIQMTQSPSSLSASV |
| F25- | SISCRSSKSLLHSNGITYL | FSYSWINWVRQAPGQGLEWMGRIFPGDG | GDRVTITCRASQDIS |
| NKp46 | YWYLQKPGQSPQLLIYQMS | DTDYNGKFKGRVTITADKSTSTAYMELS | NYLNWYQQKPGKAPK |
| | NLVSGVPDRFSGSGSGTDF | SLRSEDTAVYYCARNVFDGYWLVYWGQG | LLIYYTSRLHSGVPS |
| | TLKISRVEAEDVGVYYCAQ | TLVTVSSASTKGPSVFPLAPSSKSTSGG | RFSGSGSGTDFTFTI |
| | NLELPYTFGGGTKVEIKRT | TAALGCLVKDYFPEPVTVSWNSGALTSG | SSLQPEDIATYFCQQ |
| | VAAPSVFIFPPSDEQLKSG | VHTFPAVLQSSGLYSLSSVVTVPSSSLG | GNTRPWTFGGGTKVE |
| | TASVVCLLNNFYPREAKVQ | TQTYICNVNHKPSNTKVDKRVEPKSCDK | IKRTVAAPSVFIFPP |
| | WKVDNALQSGNSQESVTEQ | THTCPPCPAPELLGGPSVFLFPPKPKDT | SDEQLKSGTASVVCL |
| | DSKDSTYSLSSTLTLSKAD | LMISRTPEVTCVVVDVSHEDPEVKENWY | LNNFYPREAKVQWKV |
| | YEKHKVYACEVTHQGLSSP | VDGVEVHNAKTKPREEQYNSTYRVVSVL | DNALQSGNSQESVTE |
| | VTKSENRGECDKTHTCPPC | TVLHQDWLNGKEYKCKVSNKALPAPIEK | QDSKDSTYSLSSTLT |
| | PAPELLGGPSVFLFPPKPK | TISKAKGQPREPQVYTLPPSREEMTKNQ | LSKADYEKHKVYACE |
| | DTLMISRTPEVTCVVVDVS | VSLTCLVKGFYPSDIAVEWESNGQPENN | VTHQGLSSPVTKSEN |
| | HEDPEVKFNWYVDGVEVHN | YKTTPPVLDSDGSFFLYSKLTVDKSRWQ | RGEC |
| | AKTKPREEQYNSTYRVVSV | QGNVFSCSVMHEALHNHYTQKSLSLSPG | (SEQ ID NO: 470) |
| | LTVLHQDWLNGKEYKCKVS | STGSQVQLVQSGAEVKKPGSSVKVSCKA | |
| | NKALPAPIEKTISKAKGQP | SGYTFSDYVINWVRQAPGQGLEWMGEIY | |
| | REPQVYTLPPSREEMTKNQ | PGSGTNYYNEKFKAKATITADKSTSTAY | |
| | VSLTCLVKGFYPSDIAVEW | MELSSLRSEDTAVYYCARRGRYGLYAMD | |
| | ESNGQPENNYKTTPPVLDS | YWGQGTTVTVSSASTKGPSVFPLAPSSK | |
| | DGSFFLYSKLTVDKSRWQQ | STSGGTAALGCLVKDYFPEPVTVSWNSG | |
| | GNVFSCSVMHEALHNHYTQ | ALTSGVHTFPAVLQSSGLYSLSSVVTVP | |
| | KSLSLSPGK | SSSLGTQTYICNVNHKPSNTKVDKRVEP | |
| | (SEQ ID NO: 468) | KSCDKTHS | |
| | | (SEQ ID NO: 469) | |
| IC-T5- | EIVLTQSPATLSLSPGERA | EVQLVESGGGLVQPGRSLRLSCAASGFT | DIQMTQSPSSLSASV |
| NKp46- | TLSCRASQSVRSYLAWYQQ | FDNYAMHWVRQAPGKGLEWVSGISRSSG | GDRVTITCRASQDIS |
| IL2v | KPGQAPRLLFSDASNRATG | DIDYADSVKGRFTISRDNAKNSLYLQMN | NYLNWYQQKPGKAPK |
| | IPARFSGSGSGTDFTLTIS | SLRAEDTALYYCARGGVGSFDTWGQGTM | LLIYYTSRLHSGVPS |
| | SLEPEDFAVYYCQQYRYSP | VTVSSASTKGPSVFPLAPSSKSTSGGTA | RFSGSGSGTDFTFTI |
| | RTFGQGTKVEIKRTVAAPS | ALGCLVKDYFPEPVTVSWNSGALTSGVH | SSLQPEDIATYFCQQ |
| | VFIFPPSDEQLKSGTASVV | TFPAVLQSSGLYSLSSVVTVPSSSLGTQ | GNTRPWTFGGGTKVE |
| | CLLNNFYPREAKVQWKVDN | TYICNVNHKPSNTKVDKRVEPKSCDKTH | IKASTKGPSVFPLAP |
| | ALQSGNSQESVTEQDSKDS | TCPPCPAPELLGGPSVFLFPPKPKDTLM | SSKSTSGGTAALGCL |
| | TYSLSSTLTLSKADYEKHK | ISRTPEVTCVVVDVSHEDPEVKENWYVD | VKDYFPEPVTVSWNS |
| | VYACEVTHQGLSSPVTKSF | GVEVHNAKTKPREEQYNSTYRVVSVLTV | GALTSGVHTFPAVLQ |
| | NRGECDKTHTCPPCPAPEL | LHQDWLNGKEYKCKVSNKALPAPIEKTI | SSGLYSLSSVVTVPS |
| | LGGPSVFLFPPKPKDTLMI | SKAKGQPREPQVYTLPPSREEMTKNQVS | SSLGTQTYICNVNHK |
| | SRTPEVTCVVVDVSHEDPE | LTCLVKGFYPSDIAVEWESNGQPENNYK | PSNTKVDKRVEPKSC |
| | VKFNWYVDGVEVHNAKTKP | TTPPVLDSDGSFFLYSKLTVDKSRWQQG | DKTHSGSSSSGSSSS |
| | REEQYNSTYRVVSVLTVLH | NVFSCSVMHEALHNHYTQKSLSLSPGST | APASSSTKKTQLQLE |
| | QDWLNGKEYKCKVSNKALP | GSQVQLVQSGAEVKKPGSSVKVSCKASG | HLLLDLQMILNGINN |
| | APIEKTISKAKGQPREPQV | YTFSDYVINWVRQAPGQGLEWMGEIYPG | YKNPKLTRMLTAKFA |
| | YTLPPSREEMTKNQVSLTC | SGTNYYNEKFKAKATITADKSTSTAYME | MPKKATELKHLQCLE |
| | LVKGFYPSDIAVEWESNGQ | LSSLRSEDTAVYYCARRGRYGLYAMDYW | EELKPLEEVLNGAQS |
| | PENNYKTTPPVLDSDGSFF | GQGTTVTVSSRTVAAPSVFIFPPSDEQL | KNFHLRPRDLISNIN |
| | LYSKLTVDKSRWQQGNVES | KSGTASVVCLLNNFYPREAKVQWKVDNA | VIVLELKGSETTFMC |
| | CSVMHEALHNHYTQKSLSL | LQSGNSQESVTEQDSKDSTYSLSSTLTL | EYADETATIVEFLNR |
| | SPGK | SKADYEKHKVYACEVTHQGLSSPVTKSF | WITFAQSIISTLT |
| | (SEQ ID NO: 471) | NRGEC | (SEQ ID NO: 473) |
| | | (SEQ ID NO: 472) | |
| CD20- | DIVMTQTPLSLPVTPGEPA | QVQLVQSGAEVKKPGSSVKVSCKASGYA | EIVLTQSPGTLSLSP |
| T5-IC- | SISCRSSKSLLHSNGITYL | FSYSWINWVRQAPGQGLEWMGRIFPGDG | GERATLSCRASQSVS |
| IL2v3 | YWYLQKPGQSPQLLIYQMS | DTDYNGKFKGRVTITADKSTSTAYMELS | SSYLAWYQQKPGQAP |
| | NLVSGVPDRFSGSGSGTDF | SLRSEDTAVYYCARNVFDGYWLVYWGQG | RLLIYGASSRATGIP |
| | TLKISRVEAEDVGVYYCAQ | TLVTVSSASTKGPSVFPLAPSSKSTSGG | DRFSGSGSGTDFTLT |
| | NLELPYTFGGGTKVEIKRT | TAALGCLVKDYFPEPVTVSWNSGALTSG | ISRLEPEDFAVYYCQ |
| | VAAPSVFIFPPSDEQLKSG | VHTFPAVLQSSGLYSLSSVVTVPSSSLG | QYGSSTWTFGQGTKV |
| | TASVVCLLNNFYPREAKVQ | TQTYICNVNHKPSNTKVDKRVEPKSCDK | EIKASTKGPSVFPLA |
| | WKVDNALQSGNSQESVTEQ | THTCPPCPAPELLGGPSVFLFPPKPKDT | PSSKSTSGGTAALGC |
| | DSKDSTYSLSSTLTLSKAD | LMISRTPEVTCVVVDVSHEDPEVKENWY | LVKDYFPEPVTVSWN |
| | YEKHKVYACEVTHQGLSSP | VDGVEVHNAKTKPREEQYNSTYRVVSVL | SGALTSGVHTFPAVL |
| | VTKSFNRGECDKTHTCPPC | TVLHQDWLNGKEYKCKVSNKALPAPIEK | QSSGLYSLSSVVTVP |
| | PAPELLGGPSVFLFPPKPK | TISKAKGQPREPQVYTLPPSREEMTKNQ | SSSLGTQTYICNVNH |
| | DTLMISRTPEVTCVVVDVS | VSLTCLVKGFYPSDIAVEWESNGQPENN | KPSNTKVDKRVEPKS |
| | HEDPEVKFNWYVDGVEVHN | YKTTPPVLDSDGSFFLYSKLTVDKSRWQ | CDKTHSGSSSSGSSS |
| | AKTKPREEQYNSTYRVVSV | QGNVFSCSVMHEALHNHYTQKSLSLSPG | SGSSSSTKKTQLQLE |
| | LTVLHQDWLNGKEYKCKVS | STGSEVQLVQSGAEVKKSGESLKISCKG | HLLLDLQMILNGINN |
| | NKALPAPIEKTISKAKGQP | SGYSFTSYWIGWVRQMPGKGLEWMGIFY | YKNPKLTAMLAKKFY |
| | REPQVYTLPPSREEMTKNQ | PGDSSTRYSPSFQGQVTISADKSVNTAY | MPKKATELKHLQCLE |
| | VSLTCLVKGFYPSDIAVEW | LQWSSLKASDTAMYYCARRRNWGNAFDI | EELKPLEEVLNLAQS |
| | ESNGQPENNYKTTPPVLDS | WGQGTMVTVSSRTVAAPSVFIFPPSDEQ | KNFHLRPRDLISNIN |
| | DGSFFLYSKLTVDKSRWQQ | LKSGTASVVCLLNNFYPREAKVQWKVDN | VIVLELKGSETTFMC |
| | GNVFSCSVMHEALHNHYTQ | ALQSGNSQESVTEQDSKDSTYSLSSTLT | EYADETATIVEFLNR |
| | KSLSLSPGK | LSKADYEKHKVYACEVTHQGLSSPVTKS | WITFCQSIISTLT |
| | (SEQ ID NO: 474) | FNRGEC | (SEQ ID NO: 476) |
| | | (SEQ ID NO: 475) | |
| CD20- | DIVMTQTPLSLPVTPGEPA | QVQLVQSGAEVKKPGSSVKVSCKASGYA | DIQMTQSPSSLSASV |
| T5A- | SISCRSSKSLLHSNGITYL | FSYSWINWVRQAPGQGLEWMGRIFPGDG | GDRVTITCRASQDIS |
| NKp46- | YWYLQKPGQSPQLLIYQMS | DTDYNGKFKGRVTITADKSTSTAYMELS | NYLNWYQQKPGKAPK |
| IL 15 | NLVSGVPDRFSGSGSGTDF | SLRSEDTAVYYCARNVFDGYWLVYWGQG | LLIYYTSRLHSGVPS |
| | TLKISRVEAEDVGVYYCAQ | TLVTVSSASTKGPSVFPLAPSSKSTSGG | RFSGSGSGTDFTFTI |
| | NLELPYTFGGGTKVEIKRT | TAALGCLVKDYFPEPVTVSWNSGALTSG | SSLQPEDIATYFCQQ |
| | VAAPSVFIFPPSDEQLKSG | VHTFPAVLQSSGLYSLSSVVTVPSSSLG | GNTRPWTFGGGTKVE |
| | TASVVCLLNNFYPREAKVQ | TQTYICNVNHKPSNTKVDKRVEPKSCDK | IKASTKGPSVFPLAP |
| | WKVDNALQSGNSQESVTEQ | THTCPPCPAPELLGGPSVFLFPPKPKDT | SSKSTSGGTAALGCL |
| | DSKDSTYSLSSTLTLSKAD | LMISRTPEVTCVVVDVSHEDPEVKENWY | VKDYFPEPVTVSWNS |
| | YEKHKVYACEVTHQGLSSP | VDGVEVHNAKTKPREEQYNSTYRVVSVL | GALTSGVHTFPAVLQ |
| | VTKSFNRGECDKTHTCPPC | TVLHQDWLNGKEYKCKVSNKALPAPIEK | SSGLYSLSSVVTVPS |
| | PAPELLGGPSVFLFPPKPK | TISKAKGQPREPQVYTLPPSREEMTKNQ | SSLGTQTYICNVNHK |
| | DTLMISRTPEVTCVVVDVS | VSLTCLVKGFYPSDIAVEWESNGQPENN | PSNTKVDKRVEPKSC |
| | HEDPEVKFNWYVDGVEVHN | YKTTPPVLDSDGSFFLYSKLTVDKSRWQ | DKTHSGGGGSGGGGS |
| | AKTKPREEQYNSTYRVVSV | QGNVFSCSVMHEALHNHYTQKSLSLSPG | GGGGSNWVNVISDLK |
| | LTVLHQDWLNGKEYKCKVS | STGSQVQLVQSGAEVKKPGSSVKVSCKA | KIEDLIQSMHIDATL |
| | NKALPAPIEKTISKAKGQP | SGYTFSDYVINWVRQAPGQGLEWMGEIY | YTESDVHPSCKVTAM |
| | REPQVYTLPPSREEMTKNQ | PGSGTNYYNEKFKAKATITADKSTSTAY | KCFLLELQVISLESG |
| | VSLTCLVKGFYPSDIAVEW | MELSSLRSEDTAVYYCARRGRYGLYAMD | DASIHDTVENLIILA |
| | ESNGQPENNYKTTPPVLDS | YWGQGTTVTVSSRTVAAPSVFIFPPSDE | NNSLSSNGNVTESGC |
| | DGSFFLYSKLTVDKSRWQQ | QLKSGTASVVCLLNNFYPREAKVQWKVD | KECEELEEKNIKEFL |
| | GNVFSCSVMHEALHNRFTQ | NALQSGNSQESVTEQDSKDSTYSLSSTL | QSFVHIVQMFINTS |
| | KSLSLSPGK | TLSKADYEKHKVYACEVTHQGLSSPVTK | (SEQ ID NO: 479) |
| | (SEQ ID NO: 477) | SENRGEC | |
| | | (SEQ ID NO: 478) | |
| CD20- | DIVMTQTPLSLPVTPGEPA | QVQLVQSGAEVKKPGSSVKVSCKASGYA | EIVLTQSPGTLSLSP |
| T6AB3- | SISCRSSKSLLHSNGITYL | FSYSWINWVRQAPGQGLEWMGRIFPGDG | GERATLSCRASQSVS |
| IC-IL15 | YWYLQKPGQSPQLLIYQMS | DTDYNGKFKGRVTITADKSTSTAYMELS | SSYLAWYQQKPGQAP |
| | NLVSGVPDRFSGSGSGTDF | SLRSEDTAVYYCARNVFDGYWLVYWGQG | RLLIYGASSRATGIP |
| | TLKISRVEAEDVGVYYCAQ | TLVTVSSASTKGPSVFPLAPSSKSTSGG | DRFSGSGSGTDFTLT |
| | NLELPYTFGGGTKVEIKRT | TAALGCLVKDYFPEPVTVSWNSGALTSG | ISRLEPEDFAVYYCQ |
| | VAAPSVFIFPPSDEQLKSG | VHTFPAVLQSSGLYSLSSVVTVPSSSLG | QYGSSTWTFGQGTKV |
| | TASVVCLLNNFYPREAKVQ | TQTYICNVNHKPSNTKVDKRVEPKSCDK | EIKASTKGPSVFPLA |
| | WKVDNALQSGNSQESVTEQ | THTCPPCPAPEAEGAPSVELFPPKPKDT | PSSKSTSGGTAALGC |
| | DSKDSTYSLSSTLTLSKAD | LMISRTPEVTCVVVDVSHEDPEVKENWY | LVKDYFPEPVTVSWN |
| | YEKHKVYACEVTHQGLSSP | VDGVEVHNAKTKPREEQYNSTYRVVSVL | SGALTSGVHTFPAVL |
| | VTKSFNRGECDKTHTCPPC | TVLHQDWLNGKEYKCKVSNKALPSSIEK | QSSGLYSLSSVVTVP |
| | PAPEAEGAPSVFLFPPKPK | TISKAKGQPREPQVYTLPPSREEMTKNQ | SSSLGTQTYICNVNH |
| | DTLMISRTPEVTCVVVDVS | VSLTCLVKGFYPSDIAVEWESNGQPENN | KPSNTKVDKRVEPKS |
| | HEDPEVKFNWYVDGVEVHN | YKTTPPVLDSDGSFFLYSKLTVDKSRWQ | CDKTHSGGGGSGGGG |
| | AKTKPREEQYNSTYRVVSV | QGNVFSCSVMHEALHNHYTQKSLSLSPG | SGGGGSNWVNVISDL |
| | LTVLHQDWLNGKEYKCKVS | STGSEVQLVQSGAEVKKSGESLKISCKG | KKIEDLIQSMHIDAT |
| | NKALPSSIEKTISKAKGQP | SGYSFTSYWIGWVRQMPGKGLEWMGIFY | LYTESDVHPSCKVTA |
| | REPQVYTLPPSREEMTKNQ | PGDSSTRYSPSFQGQVTISADKSVNTAY | MKCFLLELQVISLES |
| | VSLTCLVKGFYPSDIAVEW | LQWSSLKASDTAMYYCARRRNWGNAFDI | GDASIHDTVENLIIL |
| | ESNGQPENNYKTTPPVLDS | WGQGTMVTVSSRTVAAPSVFIFPPSDEQ | ANNSLSSNGNVTESG |
| | DGSFFLYSKLTVDKSRWQQ | LKSGTASVVCLLNNFYPREAKVQWKVDN | CKECEELEEKNIKEF |
| | GNVFSCSVMHEALHNRFTQ | ALQSGNSQESVTEQDSKDSTYSLSSTLT | LQSFVHIVQMFINTS |
| | KSLSLSPGK | LSKADYEKHKVYACEVTHQGLSSPVTKS | (SEQ ID NO: 482) |
| | (SEQ ID NO: 480) | FNRGEC | |
| | | (SEQ ID NO: 481) | |
| IC-T5A- | EIVLTQSPATLSLSPGERA | EVQLVESGGGLVQPGRSLRLSCAASGFT | DIQMTQSPSSLSASV |
| NKp46- | TLSCRASQSVRSYLAWYQQ | FDNYAMHWVRQAPGKGLEWVSGISRSSG | GDRVTITCRASQDIS |
| IL 15 | KPGQAPRLLFSDASNRATG | DIDYADSVKGRFTISRDNAKNSLYLQMN | NYLNWYQQKPGKAPK |
| | IPARFSGSGSGTDFTLTIS | SLRAEDTALYYCARGGVGSFDTWGQGTM | LLIYYTSRLHSGVPS |
| | SLEPEDFAVYYCQQYRYSP | VTVSSASTKGPSVFPLAPSSKSTSGGTA | RFSGSGSGTDFTFTI |
| | RTFGQGTKVEIKRTVAAPS | ALGCLVKDYFPEPVTVSWNSGALTSGVH | SSLQPEDIATYFCQQ |
| | VFIFPPSDEQLKSGTASVV | TFPAVLQSSGLYSLSSVVTVPSSSLGTQ | GNTRPWTFGGGTKVE |
| | CLLNNFYPREAKVQWKVDN | TYICNVNHKPSNTKVDKRVEPKSCDKTH | IKASTKGPSVFPLAP |
| | ALQSGNSQESVTEQDSKDS | TCPPCPAPELLGGPSVFLFPPKPKDTLM | SSKSTSGGTAALGCL |
| | TYSLSSTLTLSKADYEKHK | ISRTPEVTCVVVDVSHEDPEVKFNWYVD | VKDYFPEPVTVSWNS |
| | VYACEVTHQGLSSPVTKSF | GVEVHNAKTKPREEQYNSTYRVVSVLTV | GALTSGVHTFPAVLQ |
| | NRGECDKTHTCPPCPAPEL | LHQDWLNGKEYKCKVSNKALPAPIEKTI | SSGLYSLSSVVTVPS |
| | LGGPSVFLFPPKPKDTLMI | SKAKGQPREPQVYTLPPSREEMTKNQVS | SSLGTQTYICNVNHK |
| | SRTPEVTCVVVDVSHEDPE | LTCLVKGFYPSDIAVEWESNGQPENNYK | PSNTKVDKRVEPKSC |
| | VKFNWYVDGVEVHNAKTKP | TTPPVLDSDGSFFLYSKLTVDKSRWQQG | DKTHSGGGGSGGGGS |
| | REEQYNSTYRVVSVLTVLH | NVFSCSVMHEALHNHYTQKSLSLSPGST | GGGGSNWVNVISDLK |
| | QDWLNGKEYKCKVSNKALP | GSQVQLVQSGAEVKKPGSSVKVSCKASG | KIEDLIQSMHIDATL |
| | APIEKTISKAKGQPREPQV | YTFSDYVINWVRQAPGQGLEWMGEIYPG | YTESDVHPSCKVTAM |
| | YTLPPSREEMTKNQVSLTC | SGTNYYNEKFKAKATITADKSTSTAYME | KCFLLELQVISLESG |
| | LVKGFYPSDIAVEWESNGQ | LSSLRSEDTAVYYCARRGRYGLYAMDYW | DASIHDTVENLIILA |
| | PENNYKTTPPVLDSDGSFF | GQGTTVTVSSRTVAAPSVFIFPPSDEQL | NNSLSSNGNVTESGC |
| | LYSKLTVDKSRWQQGNVFS | KSGTASVVCLLNNFYPREAKVQWKVDNA | KECEELEEKNIKEFL |
| | CSVMHEALHNRFTQKSLSL | LQSGNSQESVTEQDSKDSTYSLSSTLTL | QSFVHIVQMFINTS |
| | SPGK | SKADYEKHKVYACEVTHQGLSSPVTKSF | (SEQ ID NO: 485) |
| | (SEQ ID NO: 483) | NRGEC | |
| | | (SEQ ID NO: 484) | |
| CD20- | DIVMTQTPLSLPVTPGEPA | QVQLVQSGAEVKKPGSSVKVSCKASGYA | DIQMTQSPSSLSASV |
| T5A- | SISCRSSKSLLHSNGITYL | FSYSWINWVRQAPGQGLEWMGRIFPGDG | GDRVTITCRASQDIS |
| NKp46- | YWYLQKPGQSPQLLIYQMS | DTDYNGKFKGRVTITADKSTSTAYMELS | NYLNWYQQKPGKAPK |
| IL 18v | NLVSGVPDRFSGSGSGTDF | SLRSEDTAVYYCARNVEDGYWLVYWGQG | LLIYYTSRLHSGVPS |
| | TLKISRVEAEDVGVYYCAQ | TLVTVSSASTKGPSVFPLAPSSKSTSGG | RFSGSGSGTDFTFTI |
| | NLELPYTFGGGTKVEIKRT | TAALGCLVKDYFPEPVTVSWNSGALTSG | SSLQPEDIATYFCQQ |
| | VAAPSVFIFPPSDEQLKSG | VHTFPAVLQSSGLYSLSSVVTVPSSSLG | GNTRPWTFGGGTKVE |
| | TASVVCLLNNFYPREAKVQ | TQTYICNVNHKPSNTKVDKRVEPKSCDK | IKASTKGPSVFPLAP |
| | WKVDNALQSGNSQESVTEQ | THTCPPCPAPELLGGPSVFLFPPKPKDT | SSKSTSGGTAALGCL |
| | DSKDSTYSLSSTLTLSKAD | LMISRTPEVTCVVVDVSHEDPEVKENWY | VKDYFPEPVTVSWNS |
| | YEKHKVYACEVTHQGLSSP | VDGVEVHNAKTKPREEQYNSTYRVVSVL | GALTSGVHTFPAVLQ |
| | VTKSFNRGECDKTHTCPPC | TVLHQDWLNGKEYKCKVSNKALPAPIEK | SSGLYSLSSVVTVPS |
| | PAPELLGGPSVFLFPPKPK | TISKAKGQPREPQVYTLPPSREEMTKNQ | SSLGTQTYICNVNHK |
| | DTLMISRTPEVTCVVVDVS | VSLTCLVKGFYPSDIAVEWESNGQPENN | PSNTKVDKRVEPKSC |
| | HEDPEVKFNWYVDGVEVHN | YKTTPPVLDSDGSFFLYSKLTVDKSRWQ | DKTHSGGSGGGGSGG |
| | AKTKPREEQYNSTYRVVSV | QGNVFSCSVMHEALHNHYTQKSLSLSPG | GGSGGYFGKLESKLS |
| | LTVLHQDWLNGKEYKCKVS | STGSQVQLVQSGAEVKKPGSSVKVSCKA | VIRNLNDQVLFIDQG |
| | NKALPAPIEKTISKAKGQP | SGYTFSDYVINWVRQAPGQGLEWMGEIY | NRPLFEDMTDSDCRD |
| | REPQVYTLPPSREEMTKNQ | PGSGTNYYNEKFKAKATITADKSTSTAY | NAPRTIFIISKYGDS |
| | VSLTCLVKGFYPSDIAVEW | MELSSLRSEDTAVYYCARRGRYGLYAMD | GARGLAVTISVKCEK |
| | ESNGQPENNYKTTPPVLDS | YWGQGTTVTVSSRTVAAPSVFIFPPSDE | ISTLSCENKIISFKE |
| | DGSFFLYSKLTVDKSRWQQ | QLKSGTASVVCLLNNFYPREAKVQWKVD | MNPPDNIKDTKSDII |
| | GNVFSCSVMHEALHNRFTQ | NALQSGNSQESVTEQDSKDSTYSLSSTL | FFERDVPGHSGKVQF |
| | KSLSLSPGK | TLSKADYEKHKVYACEVTHQGLSSPVTK | ESSSYEGYFLACEKE |
| | (SEQ ID NO: 486) | SFNRGEC | RDLFKLILKKEDELG |
| | | (SEQ ID NO: 487) | DRSIMFTVQNED |
| | | | (SEQ ID NO: 488) |
| CD20- | DIVMTQTPLSLPVTPGEPA | QVQLVQSGAEVKKPGSSVKVSCKASGYA | EIVLTQSPGTLSLSP |
| T6AB3- | SISCRSSKSLLHSNGITYL | FSYSWINWVRQAPGQGLEWMGRIFPGDG | GERATLSCRASQSVS |
| IC-IL18v | YWYLQKPGQSPQLLIYQMS | DTDYNGKFKGRVTITADKSTSTAYMELS | SSYLAWYQQKPGQAP |
| | NLVSGVPDRFSGSGSGTDF | SLRSEDTAVYYCARNVFDGYWLVYWGQG | RLLIYGASSRATGIP |
| | TLKISRVEAEDVGVYYCAQ | TLVTVSSASTKGPSVFPLAPSSKSTSGG | DRFSGSGSGTDFTLT |
| | NLELPYTFGGGTKVEIKRT | TAALGCLVKDYFPEPVTVSWNSGALTSG | ISRLEPEDFAVYYCQ |
| | VAAPSVFIFPPSDEQLKSG | VHTFPAVLQSSGLYSLSSVVTVPSSSLG | QYGSSTWTFGQGTKV |
| | TASVVCLLNNFYPREAKVQ | TQTYICNVNHKPSNTKVDKRVEPKSCDK | EIKASTKGPSVFPLA |
| | WKVDNALQSGNSQESVTEQ | THTCPPCPAPEAEGAPSVELFPPKPKDT | PSSKSTSGGTAALGC |
| | DSKDSTYSLSSTLTLSKAD | LMISRTPEVTCVVVDVSHEDPEVKENWY | LVKDYFPEPVTVSWN |
| | YEKHKVYACEVTHQGLSSP | VDGVEVHNAKTKPREEQYNSTYRVVSVL | SGALTSGVHTFPAVL |
| | VTKSFNRGECDKTHTCPPC | TVLHQDWLNGKEYKCKVSNKALPSSIEK | QSSGLYSLSSVVTVP |
| | PAPEAEGAPSVFLFPPKPK | TISKAKGQPREPQVYTLPPSREEMTKNQ | SSSLGTQTYICNVNH |
| | DTLMISRTPEVTCVVVDVS | VSLTCLVKGFYPSDIAVEWESNGQPENN | KPSNTKVDKRVEPKS |
| | HEDPEVKFNWYVDGVEVHN | YKTTPPVLDSDGSFFLYSKLTVDKSRWQ | CDKTHSGGSGGGGSG |
| | AKTKPREEQYNSTYRVVSV | QGNVFSCSVMHEALHNHYTQKSLSLSPG | GGGSGGYFGKLESKL |
| | LTVLHQDWLNGKEYKCKVS | STGSEVQLVQSGAEVKKSGESLKISCKG | SVIRNLNDQVLFIDQ |
| | NKALPSSIEKTISKAKGQP | SGYSFTSYWIGWVRQMPGKGLEWMGIFY | GNRPLFEDMTDSDCR |
| | REPQVYTLPPSREEMTKNQ | PGDSSTRYSPSFQGQVTISADKSVNTAY | DNAPRTIFIISKYGD |
| | VSLTCLVKGFYPSDIAVEW | LQWSSLKASDTAMYYCARRRNWGNAFDI | SGARGLAVTISVKCE |
| | ESNGQPENNYKTTPPVLDS | WGQGTMVTVSSRTVAAPSVFIFPPSDEQ | KISTLSCENKIISFK |
| | DGSFFLYSKLTVDKSRWQQ | LKSGTASVVCLLNNFYPREAKVQWKVDN | EMNPPDNIKDTKSDI |
| | GNVFSCSVMHEALHNRFTQ | ALQSGNSQESVTEQDSKDSTYSLSSTLT | IFFERDVPGHSGKVQ |
| | KSLSLSPGK | LSKADYEKHKVYACEVTHQGLSSPVTKS | FESSSYEGYFLACEK |
| | (SEQ ID NO: 489) | FNRGEC | ERDLFKLILKKEDEL |
| | | (SEQ ID NO: 490) | GDRSIMFTVQNED |
| | | | (SEQ ID NO: 491) |
| IC-T5A- | EIVLTQSPATLSLSPGERA | EVQLVESGGGLVQPGRSLRLSCAASGFT | DIQMTQSPSSLSASV |
| NKp46- | TLSCRASQSVRSYLAWYQQ | FDNYAMHWVRQAPGKGLEWVSGISRSSG | GDRVTITCRASQDIS |
| IL 18v | KPGQAPRLLFSDASNRATG | DIDYADSVKGRFTISRDNAKNSLYLQMN | NYLNWYQQKPGKAPK |
| | IPARFSGSGSGTDFTLTIS | SLRAEDTALYYCARGGVGSFDTWGQGTM | LLIYYTSRLHSGVPS |
| | SLEPEDFAVYYCQQYRYSP | VTVSSASTKGPSVFPLAPSSKSTSGGTA | RFSGSGSGTDFTFTI |
| | RTFGQGTKVEIKRTVAAPS | ALGCLVKDYFPEPVTVSWNSGALTSGVH | SSLQPEDIATYFCQQ |
| | VFIFPPSDEQLKSGTASVV | TFPAVLQSSGLYSLSSVVTVPSSSLGTQ | GNTRPWTFGGGTKVE |
| | CLLNNFYPREAKVQWKVDN | TYICNVNHKPSNTKVDKRVEPKSCDKTH | IKASTKGPSVFPLAP |
| | ALQSGNSQESVTEQDSKDS | TCPPCPAPELLGGPSVFLFPPKPKDTLM | SSKSTSGGTAALGCL |
| | TYSLSSTLTLSKADYEKHK | ISRTPEVTCVVVDVSHEDPEVKFNWYVD | VKDYFPEPVTVSWNS |
| | VYACEVTHQGLSSPVTKSF | GVEVHNAKTKPREEQYNSTYRVVSVLTV | GALTSGVHTFPAVLQ |
| | NRGECDKTHTCPPCPAPEL | LHQDWLNGKEYKCKVSNKALPAPIEKTI | SSGLYSLSSVVTVPS |
| | LGGPSVFLFPPKPKDTLMI | SKAKGQPREPQVYTLPPSREEMTKNQVS | SSLGTQTYICNVNHK |
| | SRTPEVTCVVVDVSHEDPE | LTCLVKGFYPSDIAVEWESNGQPENNYK | PSNTKVDKRVEPKSC |
| | VKFNWYVDGVEVHNAKTKP | TTPPVLDSDGSFFLYSKLTVDKSRWQQG | DKTHSGGSGGGGSGG |
| | REEQYNSTYRVVSVLTVLH | NVFSCSVMHEALHNHYTQKSLSLSPGST | GGSGGYFGKLESKLS |
| | QDWLNGKEYKCKVSNKALP | GSQVQLVQSGAEVKKPGSSVKVSCKASG | VIRNLNDQVLFIDQG |
| | APIEKTISKAKGQPREPQV | YTFSDYVINWVRQAPGQGLEWMGEIYPG | NRPLFEDMTDSDCRD |
| | YTLPPSREEMTKNQVSLTC | SGTNYYNEKFKAKATITADKSTSTAYME | NAPRTIFIISKYGDS |
| | LVKGFYPSDIAVEWESNGQ | LSSLRSEDTAVYYCARRGRYGLYAMDYW | GARGLAVTISVKCEK |
| | PENNYKTTPPVLDSDGSFF | GQGTTVTVSSRTVAAPSVFIFPPSDEQL | ISTLSCENKIISFKE |
| | LYSKLTVDKSRWQQGNVFS | KSGTASVVCLLNNFYPREAKVQWKVDNA | MNPPDNIKDTKSDII |
| | CSVMHEALHNRFTQKSLSL | LQSGNSQESVTEQDSKDSTYSLSSTLTL | FFERDVPGHSGKVQF |
| | SPGK | SKADYEKHKVYACEVTHQGLSSPVTKSF | ESSSYEGYFLACEKE |
| | (SEQ ID NO: 492) | NRGEC | RDLFKLILKKEDELG |
| | | (SEQ ID NO: 493) | DRSIMFTVQNED |
| | | | (SEQ ID NO: 494) |
| CD20- | DIVMTQTPLSLPVTPGEPA | QVQLVQSGAEVKKPGSSVKVSCKASGYA | DIQMTQSPSSLSASV |
| T5A- | SISCRSSKSLLHSNGITYL | FSYSWINWVRQAPGQGLEWMGRIFPGDG | GDRVTITCRASQDIS |
| NKp46- | YWYLQKPGQSPQLLIYQMS | DTDYNGKFKGRVTITADKSTSTAYMELS | NYLNWYQQKPGKAPK |
| IFNαv | NLVSGVPDRFSGSGSGTDF | SLRSEDTAVYYCARNVEDGYWLVYWGQG | LLIYYTSRLHSGVPS |
| | TLKISRVEAEDVGVYYCAQ | TLVTVSSASTKGPSVFPLAPSSKSTSGG | RFSGSGSGTDFTFTI |
| | NLELPYTFGGGTKVEIKRT | TAALGCLVKDYFPEPVTVSWNSGALTSG | SSLQPEDIATYFCQQ |
| | VAAPSVFIFPPSDEQLKSG | VHTFPAVLQSSGLYSLSSVVTVPSSSLG | GNTRPWTFGGGTKVE |
| | TASVVCLLNNFYPREAKVQ | TQTYICNVNHKPSNTKVDKRVEPKSCDK | IKASTKGPSVFPLAP |
| | WKVDNALQSGNSQESVTEQ | THTCPPCPAPELLGGPSVFLFPPKPKDT | SSKSTSGGTAALGCL |
| | DSKDSTYSLSSTLTLSKAD | LMISRTPEVTCVVVDVSHEDPEVKENWY | VKDYFPEPVTVSWNS |
| | YEKHKVYACEVTHQGLSSP | VDGVEVHNAKTKPREEQYNSTYRVVSVL | GALTSGVHTFPAVLQ |
| | VTKSFNRGECDKTHTCPPC | TVLHQDWLNGKEYKCKVSNKALPAPIEK | SSGLYSLSSVVTVPS |
| | PAPELLGGPSVFLFPPKPK | TISKAKGQPREPQVYTLPPSREEMTKNQ | SSLGTQTYICNVNHK |
| | DTLMISRTPEVTCVVVDVS | VSLTCLVKGFYPSDIAVEWESNGQPENN | PSNTKVDKRVEPKSC |
| | HEDPEVKFNWYVDGVEVHN | YKTTPPVLDSDGSFFLYSKLTVDKSRWQ | DKTHSGGGGSGGGGS |
| | AKTKPREEQYNSTYRVVSV | QGNVFSCSVMHEALHNHYTQKSLSLSPG | GGGGSDLPQTHSLGS |
| | LTVLHQDWLNGKEYKCKVS | STGSQVQLVQSGAEVKKPGSSVKVSCKA | RRTLMLLAQMRRISL |
| | NKALPAPIEKTISKAKGQP | SGYTFSDYVINWVRQAPGQGLEWMGEIY | FSCAKDRHDFGFPQE |
| | REPQVYTLPPSREEMTKNQ | PGSGTNYYNEKFKAKATITADKSTSTAY | EFGNQFQKAETIPVL |
| | VSLTCLVKGFYPSDIAVEW | MELSSLRSEDTAVYYCARRGRYGLYAMD | HEMIQQIFNLFSTKD |
| | ESNGQPENNYKTTPPVLDS | YWGQGTTVTVSSRTVAAPSVFIFPPSDE | SSAAWDETLLDKFYT |
| | DGSFFLYSKLTVDKSRWQQ | QLKSGTASVVCLLNNFYPREAKVQWKVD | ELYQQLNDLEACVIQ |
| | GNVFSCSVMHEALHNRFTQ | NALQSGNSQESVTEQDSKDSTYSLSSTL | GVGVTETPLMKEDSI |
| | KSLSLSPGK | TLSKADYEKHKVYACEVTHQGLSSPVTK | LAVRKYFQRITLYLK |
| | (SEQ ID NO: 495) | SFNRGEC | EKKYSPCAWEVVRAE |
| | | (SEQ ID NO: 496) | IMRSFSLSTNLQESL |
| | | | RSKE |
| | | | (SEQ ID NO: 497) |
| CD20- | DIVMTQTPLSLPVTPGEPA | QVQLVQSGAEVKKPGSSVKVSCKASGYA | EIVLTQSPGTLSLSP |
| T6AB3- | SISCRSSKSLLHSNGITYL | FSYSWINWVRQAPGQGLEWMGRIFPGDG | GERATLSCRASQSVS |
| IC- | YWYLQKPGQSPQLLIYQMS | DTDYNGKFKGRVTITADKSTSTAYMELS | SSYLAWYQQKPGQAP |
| IFNαv | NLVSGVPDRFSGSGSGTDF | SLRSEDTAVYYCARNVFDGYWLVYWGQG | RLLIYGASSRATGIP |
| | TLKISRVEAEDVGVYYCAQ | TLVTVSSASTKGPSVFPLAPSSKSTSGG | DRFSGSGSGTDFTLT |
| | NLELPYTFGGGTKVEIKRT | TAALGCLVKDYFPEPVTVSWNSGALTSG | ISRLEPEDFAVYYCQ |
| | VAAPSVFIFPPSDEQLKSG | VHTFPAVLQSSGLYSLSSVVTVPSSSLG | QYGSSTWTFGQGTKV |
| | TASVVCLLNNFYPREAKVQ | TQTYICNVNHKPSNTKVDKRVEPKSCDK | EIKASTKGPSVFPLA |
| | WKVDNALQSGNSQESVTEQ | THTCPPCPAPEAEGAPSVELFPPKPKDT | PSSKSTSGGTAALGC |
| | DSKDSTYSLSSTLTLSKAD | LMISRTPEVTCVVVDVSHEDPEVKENWY | LVKDYFPEPVTVSWN |
| | YEKHKVYACEVTHQGLSSP | VDGVEVHNAKTKPREEQYNSTYRVVSVL | SGALTSGVHTFPAVL |
| | VTKSFNRGECDKTHTCPPC | TVLHQDWLNGKEYKCKVSNKALPSSIEK | QSSGLYSLSSVVTVP |
| | PAPEAEGAPSVFLFPPKPK | TISKAKGQPREPQVYTLPPSREEMTKNQ | SSSLGTQTYICNVNH |
| | DTLMISRTPEVTCVVVDVS | VSLTCLVKGFYPSDIAVEWESNGQPENN | KPSNTKVDKRVEPKS |
| | HEDPEVKFNWYVDGVEVHN | YKTTPPVLDSDGSFFLYSKLTVDKSRWQ | CDKTHSGGGGSGGGG |
| | AKTKPREEQYNSTYRVVSV | QGNVFSCSVMHEALHNHYTQKSLSLSPG | SGGGGSDLPQTHSLG |
| | LTVLHQDWLNGKEYKCKVS | STGSEVQLVQSGAEVKKSGESLKISCKG | SRRTLMLLAQMRRIS |
| | NKALPSSIEKTISKAKGQP | SGYSFTSYWIGWVRQMPGKGLEWMGIFY | LFSCAKDRHDFGFPQ |
| | REPQVYTLPPSREEMTKNQ | PGDSSTRYSPSFQGQVTISADKSVNTAY | EEFGNQFQKAETIPV |
| | VSLTCLVKGFYPSDIAVEW | LQWSSLKASDTAMYYCARRRNWGNAFDI | LHEMIQQIFNLFSTK |
| | ESNGQPENNYKTTPPVLDS | WGQGTMVTVSSRTVAAPSVFIFPPSDEQ | DSSAAWDETLLDKFY |
| | DGSFFLYSKLTVDKSRWQQ | LKSGTASVVCLLNNFYPREAKVQWKVDN | TELYQQLNDLEACVI |
| | GNVFSCSVMHEALHNRFTQ | ALQSGNSQESVTEQDSKDSTYSLSSTLT | QGVGVTETPLMKEDS |
| | KSLSLSPGK | LSKADYEKHKVYACEVTHQGLSSPVTKS | ILAVRKYFQRITLYL |
| | (SEQ ID NO: 498) | FNRGEC | KEKKYSPCAWEVVRA |
| | | (SEQ ID NO: 499) | EIMRSFSLSTNLQES |
| | | | LRSKE |
| | | | (SEQ ID NO: 500) |
| IC-T5A- | EIVLTQSPATLSLSPGERA | EVQLVESGGGLVQPGRSLRLSCAASGFT | DIQMTQSPSSLSASV |
| NKp46- | TLSCRASQSVRSYLAWYQQ | FDNYAMHWVRQAPGKGLEWVSGISRSSG | GDRVTITCRASQDIS |
| IFNαv | KPGQAPRLLFSDASNRATG | DIDYADSVKGRFTISRDNAKNSLYLQMN | NYLNWYQQKPGKAPK |
| | IPARFSGSGSGTDFTLTIS | SLRAEDTALYYCARGGVGSFDTWGQGTM | LLIYYTSRLHSGVPS |
| | SLEPEDFAVYYCQQYRYSP | VTVSSASTKGPSVFPLAPSSKSTSGGTA | RFSGSGSGTDFTFTI |
| | RTFGQGTKVEIKRTVAAPS | ALGCLVKDYFPEPVTVSWNSGALTSGVH | SSLQPEDIATYFCQQ |
| | VFIFPPSDEQLKSGTASVV | TFPAVLQSSGLYSLSSVVTVPSSSLGTQ | GNTRPWTFGGGTKVE |
| | CLLNNFYPREAKVQWKVDN | TYICNVNHKPSNTKVDKRVEPKSCDKTH | IKASTKGPSVFPLAP |
| | ALQSGNSQESVTEQDSKDS | TCPPCPAPELLGGPSVFLFPPKPKDTLM | SSKSTSGGTAALGCL |
| | TYSLSSTLTLSKADYEKHK | ISRTPEVTCVVVDVSHEDPEVKFNWYVD | VKDYFPEPVTVSWNS |
| | VYACEVTHQGLSSPVTKSF | GVEVHNAKTKPREEQYNSTYRVVSVLTV | GALTSGVHTFPAVLQ |
| | NRGECDKTHTCPPCPAPEL | LHQDWLNGKEYKCKVSNKALPAPIEKTI | SSGLYSLSSVVTVPS |
| | LGGPSVFLFPPKPKDTLMI | SKAKGQPREPQVYTLPPSREEMTKNQVS | SSLGTQTYICNVNHK |
| | SRTPEVTCVVVDVSHEDPE | LTCLVKGFYPSDIAVEWESNGQPENNYK | PSNTKVDKRVEPKSC |
| | VKFNWYVDGVEVHNAKTKP | TTPPVLDSDGSFFLYSKLTVDKSRWQQG | DKTHSGGGGSGGGGS |
| | REEQYNSTYRVVSVLTVLH | NVFSCSVMHEALHNHYTQKSLSLSPGST | GGGGSDLPQTHSLGS |
| | QDWLNGKEYKCKVSNKALP | GSQVQLVQSGAEVKKPGSSVKVSCKASG | RRTLMLLAQMRRISL |
| | APIEKTISKAKGQPREPQV | YTFSDYVINWVRQAPGQGLEWMGEIYPG | FSCAKDRHDFGFPQE |
| | YTLPPSREEMTKNQVSLTC | SGTNYYNEKFKAKATITADKSTSTAYME | EFGNQFQKAETIPVL |
| | LVKGFYPSDIAVEWESNGQ | LSSLRSEDTAVYYCARRGRYGLYAMDYW | HEMIQQIFNLFSTKD |
| | PENNYKTTPPVLDSDGSFF | GQGTTVTVSSRTVAAPSVFIFPPSDEQL | SSAAWDETLLDKFYT |
| | LYSKLTVDKSRWQQGNVFS | KSGTASVVCLLNNFYPREAKVQWKVDNA | ELYQQLNDLEACVIQ |
| | CSVMHEALHNRFTQKSLSL | LQSGNSQESVTEQDSKDSTYSLSSTLTL | GVGVTETPLMKEDSI |
| | SPGK | SKADYEKHKVYACEVTHQGLSSPVTKSF | LAVRKYFQRITLYLK |
| | (SEQ ID NO: 501) | NRGEC | EKKYSPCAWEVVRAE |
| | | (SEQ ID NO: 502) | IMRSFSLSTNLQESL |
| | | | RSKE |
| | | | (SEQ ID NO: 503) |

### Example 1: IL2v Limits IL2R Activation on Treg

The heterotrimeric Fc-domain-containing protein IC-T6-IC-IL2v containing one C-terminal moiety of mutant IL-2 was assessed for its ability to activate Treg cells. The IC-T6-IC-IL2v incorporates a variant IL-2 polypeptide (IL-2v), a human IL-2 polypeptide comprising the mutations T3A, C125A, F42A, Y45A and L72G, conferring decreased binding affinity for CD25 compared to wild-type human IL-2.

The heterotrimeric IC-T6-IC-IL2v protein is a T6 format protein (see FIG. 21 for the general structure) having an IL2v fused to the C-terminus of one of the chains of an isotype control (IC) Fab, which in turn was fused to the C-terminus of an Fc domain mutated to substantially eliminate CD16A binding, in turn fused to another IC Fab. The IC Fabs have VH/VL which do not bind to any protein in the test system. The heterotrimeric IC-T6-IC-IL2v was compared to an identical heterotrimeric protein in which IL2v was replaced by a wild-type human IL-2 polypeptide (IC-T6-IC-IL2pWT), and to recombinantly produced full-length wild-type IL-2 (rec IL-2).

Briefly, 1M/well of purified PBMC were seeded in 96-well plates and treated with increasing doses of recombinant huIL-2, IC-T6-IC-IL2 or IC-T6-IC-IL2v (dose from 133 nM to 0,0000013 nM) for 20 min at 37° C., 5.5% CO2 in incubator. STAT5 phosphorylation was then analysed by flow cytometry on Tregs (gated on CD3+ CD4+ CD25+ FoxP3+).

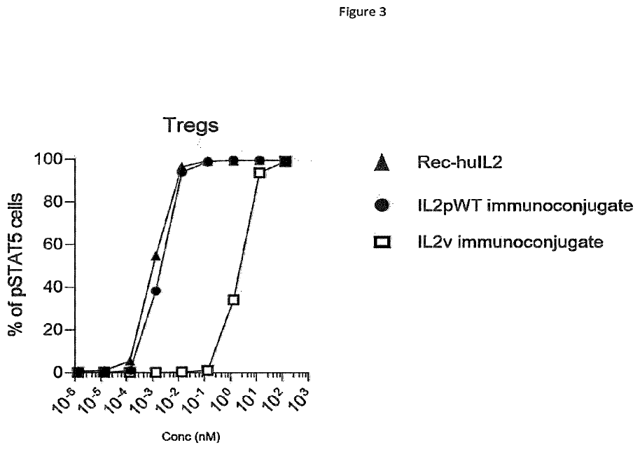

Results are shown in FIG. 3. IC-T6-IC-IL2v (“IL2v immunoconjugate”) resulted in an approximately 3-log increase in the EC₅₀ for STAT5 phosphorylation among the Treg, compared to IC-T6-IC-IL2WT (“IL2pWT immunoconjugate”) and wild-type IL-2 (“Rec-huIL2”). The IC-T6-IC-IL2v protein incorporating a mutated human IL-2 therefore displays a strongly decreased ability to activate cytokine receptor signalling in Treg cells compared to wild-type IL-2.

### Example 2: NKCE-IL2v Promotes IL2R Activation Selectively in NK Cells

We assessed whether adding a NKp46-binding function to the Fc-domain-containing and IL2v-containing heterotrimeric protein of Example 1 would promote IL-2R signalling in NK cells. We therefore evaluated different multispecific proteins (also referred to as NK cell engager (NKCE) proteins that bound NKp46 and contained an IL2v moiety (NKCE-IL2v). In order to provide a comparison to well-known anti-tumor antibodies, the NKCE were made to bind CD20 by incorporating anti-CD20 VH/VL pair from the FDA-approved humanized antibody GA101 (Obinutuzumab, Roche).

The heterotrimeric Fc-domain-containing protein CD20-T5-NKp46-IL2v was assessed for its ability to activate cytokine receptor signalling on Treg cells, NK cells, CD4 T cells and CD8 T cells. The CD20-T5-NKp46-IL2v protein format, shown in FIGS. 2A and 2G, has an antigen binding domain that binds CD20 positioned N-terminally, and incorporates a C-terminal positioned IL-2v comprising the mutations T3A, C125A, F42A, Y45A and L72G, a wild-type Fc domain (i.e. that binds human CD16A), and an antigen binding domain that binds NKp46 placed between the Fc domain and the IL2v.

Briefly, 1M/well of purified PBMC were seeded in 96-well plate and treated with increasing doses of NKCE-IL2v or IL2v immunoconjugate (dose from 133 nM to 0.0000013 nM) for 20 min at 37° C., 5.5% CO₂ in incubator. STAT5 phosphorylation was then analysed by flow cytometry on NK cells (CD3−CD56+), CD8 T cells (CD3+CD8+), CD4 T cells (CD3+CD4+FoxP3−) and Tregs (gated on CD3+CD4+CD25+FoxP3+).

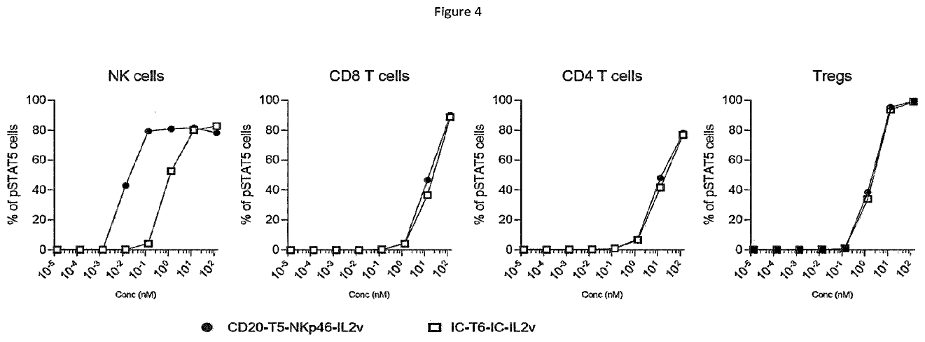

Results are shown in FIG. 4 showing % of pSTAT5 cells among targeted cells on the y-axis and concentration of test protein on the x-axis. CD20-T5-NKp46-IL2v and IC-T6-IC-IL2v displayed comparable activation of Treg cells, CD4 T cells and CD8 T cells. However, the CD20-T5-NKp46-IL2v induced a decrease of about two orders of magnitude in the EC₅₀ for STAT5 phosphorylation among the NK cells, compared to IC-T6-IC-IL2 that did not bind NKp46 or CD16A. The CD20-T5-NKp46-IL2v protein therefore permitted selective cytokine receptor signalling in NK cells over Treg cells, CD4 T cells and CD8 T cells.

### Example 3: Both CD16 and NKp46 are Important to Promote IL2R Signaling on NK Cells Induced by NKCE-IL2v

We sought to investigate the effect on IL-2R signaling in NK cells by a multispecific protein that binds to CD16 and/or NKp46 in addition to IL-2R.

A series of heterotrimeric IL2v- and Fc-domain-containing proteins were constructed that shared the same overall structure, but in which the NKp46 and tumor-antigen binding domains were individually replaced with isotype control (IC) domains that maintained the domain structure but that lacked binding to any protein in the assay system, or where the Fc domain was an Fc domain that bound CD16A (in “T5” proteins) or a mutated Fc domain (N297S) that lacked CD16A binding (in “T6” proteins). The test proteins included in this experiment:
IC-T6-IC-IL2v: contains topologically from N- to C-terminus, IC VH/VL, Fc domain dimer mutated to abolish CD16 binding, IC VH/VL pair, IL2v.
CD20-T6-IC-IL2v: contains from topological N- to C-terminus, anti-CD20 VH/VL pair, Fc domain dimer mutated to abolish CD16 binding, IC VH/VL pair, IL2v.
CD20-T5-IC-IL2v: contains from topological N- to C-terminus, anti-CD20 VH/VL pair, wild-type Fc domain dimer that binds CD16, IC VH/VL pair, IL2v.
CD20-T6-NKp46-IL2v: contains from topological N- to C-terminus, anti-CD20 VH/VL pair, Fc domain dimer mutated to abolish CD16 binding, anti-NKp46-1 VH/VL pair, IL2v.
CD20-T5-NKp46-IL2v: contains from topological N- to C-terminus, anti-CD20 VH/VL pair, wild-type Fc domain dimer that binds CD16, anti-NKp46-1 VH/VL pair, IL2v.

Briefly, 1M/well of purified PBMC were seeded in 96-well plate and treated with increasing doses of various NKCE-IL2v (dose from 133 nM to 0.0000013 nM) for 20 min at 37° C., 5.5% CO₂ in incubator. STAT5 phosphorylation was then analyzed by flow cytometry on NK cells (gated on CD3-CD56+).

Results are shown in FIG. 5 showing % of pSTAT5 cells among NK cells on the y-axis and concentration of test protein on the x-axis. IC-T6-IC-IL2v and CD20-T6-IC-IL2v displayed comparable promoting IL2R signaling in NK cells, showing that the CD20 binding domain did not have an effect in this model in which tumor target cells are absent. The CD20-T5-IC-IL2v which additionally has CD16 binding ability via its wild-type Fc domain dimer, resulted in increased IL2R signaling in the NK cells. The CD20-T6-NKp46-IL2v which can bind NKp46 but not CD16 was more potent in promoting IL2R signaling in the NK cells compared to the CD20-T5-IC-IL2v which bound CD16 but not NKp46. However, the CD20-T5-NKp46-IL2v which bound both CD16 and NKp46 (in addition to the IL2v moiety) resulted in strong increase in potency (an approximately 1-log decrease in EC₅₀) in percent of pSTAT5+ cells among the NK cells, compared to CD20-T6-NKp46-IL2v. IL-2R signaling in NK cells was therefore enhanced by each of NKp46 and CD16, and with a particularly strong enhancement when both NKp46 and CD16 were bound in addition to IL-2R.

### Example 4: NKCE-IL2v Induces NK Cell Activation in the Presence and Absence of Target Cells

In this experiment, NK cell engager proteins were assessed for their ability to induce activation of NK cells, either in the presence or absence of tumor target cells.

A series of heterotrimeric IL2v- and Fc-domain-containing proteins were tested to evaluate the effect of binding to different NK and target cell receptors, individually and in combination. The test proteins included in this experiment:
CD20-T5-NKp46-IL2v: contains topologically from N- to C-terminus, anti-CD20 VH/VL pair, Fc domain dimer that binds CD16, anti-NKp46-1 VH/VL pair, IL-2v.
CD20-F5-NKp46: contains from topological N- to C-terminus, anti-CD20 VH/VL pair, Fc domain dimer that binds CD16, anti-NKp46-1 VH/VL pair. The F5 protein shares the same essential domain arrangement with the T5 protein, except that the IL2v is absent.
CD20-T5-IC-IL2v: contains from topological N- to C-terminus, anti-CD20 VH/VL pair, Fc domain dimer that binds CD16, IC VH/VL pair, IL2v.
CD20-T6-NKp46-IL2v: contains from topological N- to C-terminus, anti-CD20 VH/VL pair, Fc domain dimer mutated to abolish CD16 binding, anti-NKp46-1 VH/VL pair, IL2v.
IC-T5-NKp46-IL2v: contains from topological N- to C-terminus, IC VH/VL pair, Fc domain dimer that binds CD16, anti-NKp46-1 VH/VL pair, IL2v.
IC-T6-IC-IL2v: contains from topological N- to C-terminus, IC VH/VL pair, Fc domain dimer mutated to abolish CD16 binding, IC VH/VL pair, IL2v.

Briefly, purified NK cells were cultured for 5 days in presence of the test proteins. NK cell activation was evaluated by quantification of the CD69 activation marker expression on NK cells monitored by flow cytometry.

Results are shown in FIG. 6 showing % of CD69-expressing NK cells on the y-axis and concentration of test protein on the x-axis, in the absence of tumor cells. The CD20-F5-NKp46 that bound CD20, NKp46 and CD16 but lacked the IL2v moiety did not activate NK cells in the absence of tumor cells, while all the proteins containing the IL2v moiety resulted in strong NK cell activation, with an additional benefit seen for the proteins that had a NKp46 binding domain and a wild-type Fc domain compared to the IC-T6-IC-IL2v protein that lacked CD16 and NKp46 binding.

### Example 5: NKCE-IL2v Promotes NK Cell Proliferation

In this experiment, NK cell engager proteins were assessed for their ability to induce proliferation of NK cells upon 5 days of incubation.

The test proteins included in this experiment:
CD20-T5-NKp46-IL2v: contains topologically from N- to C-terminus, anti-CD20 VH/VL pair, Fc domain dimer that binds CD16, anti-NKp46-1 VH/VL pair, IL-2v.
CD20-F5-NKp46: contains from topological N- to C-terminus, anti-CD20 VH/VL pair, Fc domain dimer that binds CD16, anti-NKp46-1 VH/VL pair.
CD20-T5-IC-IL2v: contains from topological N- to C-terminus, anti-CD20 VH/VL pair, Fc domain dimer that binds CD16, IC VH/VL pair (isotype control VH/VL pair that does not bind to any protein in the assay system), IL2v.
CD20-T6-NKp46-IL2v: contains from topological N- to C-terminus, anti-CD20 VH/VL pair, Fc domain dimer mutated to abolish CD16 binding, anti-NKp46-1 VH/VL pair, IL2v.
IC-T5-NKp46-IL2v: contains from topological N- to C-terminus, IC VH/VL pair, Fc domain dimer that binds CD16, anti-NKp46-1 VH/VL pair, IL2v.
IC-T6-IC-IL2v: contains from topological N- to C-terminus, IC VH/VL pair, Fc domain dimer mutated to abolish CD16 binding, IC VH/VL pair, IL2v.

Briefly, CellTraceViolet™ (CTV)-labelled-purified NK cells were cultured with a dose range of NKCE-IL2v from 133 nM to 0.0001 nM for 5 days. NK cell proliferation was evaluated by quantification of the percentage of NK cells showing a diluted CTV signal monitored by flow cytometry.

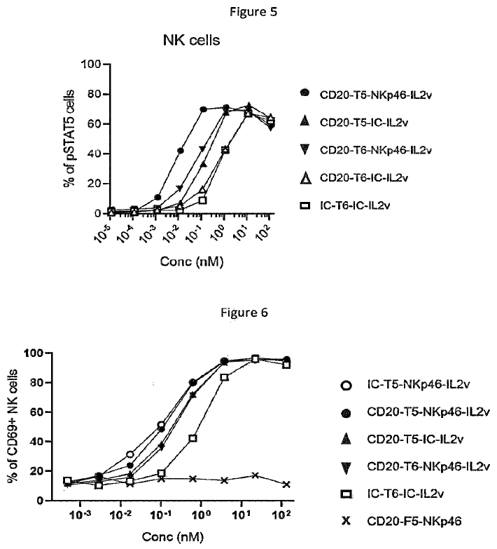

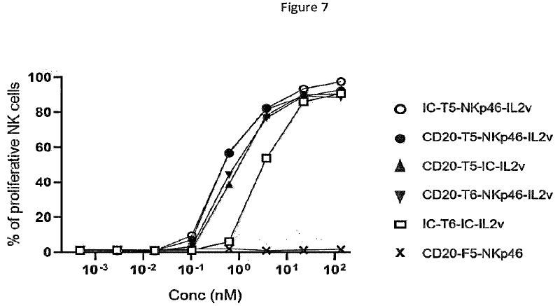

Results are shown in FIG. 7 showing % of proliferating NK cells in the absence of tumor cells on the y-axis and concentration of test protein on the x-axis. The CD20-F5-NKp46 that bound CD20, NKp46 and CD16 but lacked the IL2v moiety did not induce proliferation of NK cells, while all the proteins containing the IL2v moiety resulted in strong NK cell proliferation, although with differences in potency. All NK cell engager proteins with NKp46 binding domain and/or wild-type Fc domain (in addition to IL2v) were more potent in inducing NK cell proliferation compared to the IC-T6-IC-IL2v protein that lacked CD16 and NKp46 binding.

### Example 6: NKCE-IL2v Promotes Tumor Cell Killing in a Standard Vitro Cytotoxicity Assay at ET Ratio 10:1

In this experiment, NK cell engager proteins were assessed for their ability to induce killing of RAJI tumor cells by NK cells from two human donors at effector:target ratio of 10:1 in a standard 4-hour cytotoxicity assay using calcein release as readout.

The test proteins included in this experiment:
CD20-T5-NKp46-IL2v3: contains topologically from topological N- to C-terminus, anti-CD20 VH/VL pair, Fc domain dimer that binds CD16, anti-NKp46-1 VH/VL pair, IL-2v3.
CD20-T5-NKp46-IL2pWT: contains from topological N- to C-terminus, anti-CD20 VH/VL pair, Fc domain dimer that binds CD16, anti-NKp46-1 VH/VL pair, IL-2pWT which is wild-type human IL-2 polypeptide.
CD20-F5-NKp46: contains from topological N- to C-terminus, anti-CD20 VH/VL pair, Fc domain dimer that binds CD16, anti-NKp46-1 VH/VL pair.
CD20-T5-IC-IL2v3: contains from topological N- to C-terminus, anti-CD20 VH/VL pair, Fc domain dimer that binds CD16, IC VH/VL pair, IL2v3.
CD20-T6-NKp46-IL2v: contains from topological N- to C-terminus, anti-CD20 VH/VL pair, Fc domain dimer mutated to abolish CD16 binding, anti-NKp46-1 VH/VL pair.
IC-T5-NKp46-IL2v: contains from topological N- to C-terminus, IC VH/VL pair, Fc domain dimer that binds CD16, anti-NKp46-1 VH/VL pair, IL2v.
CD20-T5-NKp46-IL2v: contains from topological N- to C-terminus, anti-CD20 VH/VL pair, Fc domain dimer that binds CD16, anti-NKp46-1 VH/VL pair, IL2v.

Briefly, purified NK cells were rested overnight in complete medium. Purified NK Cells were then cocultured with Raji tumor cells previously loaded with calcein, in a 10 to 1 ratio. Cells were incubated with test proteins described above (doses from 66 nM to 0.0000006 nM) for 4 h at 37° C., 5.5% CO₂ in incubator.

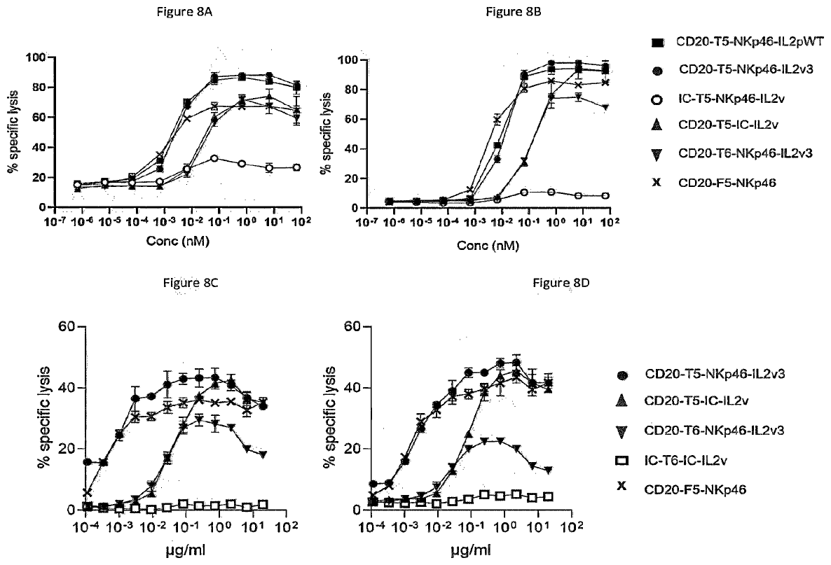

Results are shown in FIGS. 8A and 8B, each panel representing one human NK cell donor, showing % specific lysis induced by NK cells on the y-axis and concentration of test protein on the x-axis. The overall results were consistent across the two human donors. The IC-T5-NKp46-IL2v that lacked binding to CD20 on targeted cells did not induce significant cytotoxicity. All of the NK cell engagers that retained the ability to bind both CD16 and NKp46 (in addition to CD20) displayed similarly high potency in terms of EC50 values in induction of NK cell cytotoxicity toward the tumor cells. In contrast, the NK cell engagers that lacked either CD16 or NKp46 binding displayed lower potency. The nature of the IL-2 polypeptide (either wild-type of the mutated IL2v) did not appear to differentially affect NK cell cytotoxicity, and furthermore the presence of IL2, whether as wild-type or IL2v, did not result in improved EC50 values in induction of cytotoxicity compared to the CD20-F5-NKp46 NK cell engager that did not have any IL-2 moiety. However the presence of the IL-2 moiety increased the maximum level of lysis.

### Example 7: NKCE-IL2v Promotes Tumor Cell Killing in a Standard Vitro Cytotoxicity Assay at ET Ratio 2:1

In this experiment, NK cell engager proteins were assessed for their ability to induce killing of RAJI tumor cells by NK cells from two human donors at effector:target ratio of 2:1 in a standard 4-hour cytotoxicity assay suing Cr⁵¹ as readout.

The test proteins included in this experiment:
CD20-T5-NKp46-IL2v: contains from topological N- to C-terminus, anti-CD20 VH/VL pair, Fc domain dimer that binds CD16, anti-NKp46-1 VH/VL pair, IL-2v.
CD20-F5-NKp46: contains from topological N- to C-terminus, anti-CD20 VH/VL pair, Fc domain dimer that binds CD16, anti-NKp46-1 VH/VL pair.
CD20-T5-IC-IL2v: contains from topological N- to C-terminus, anti-CD20 VH/VL pair, Fc domain dimer that binds CD16, IC VH/VL pair, IL2v.
CD20-T6-NKp46-IL2v: contains from topological N- to C-terminus, anti-CD20 VH/VL pair, Fc domain dimer mutated to abolish CD16 binding, anti-NKp46-1 VH/VL pair.
IC-T6-IC-IL2v: contains from topological N- to C-terminus, IC VH/VL pair, Fc domain dimer mutated to abolish CD16 binding, IC VH/VL pair, IL2v.

Briefly, purified NK cells were rested overnight in complete medium. Resting NK cells were then cocultured with Raji tumor cells previously loaded with ⁵¹Cr, in a 2 to 1 ratio. Cells were incubated with test proteins described above (doses from 20 to 0.0001 ug/ml) for 4 h at 37° C., 5.5% CO² in incubator.

Results are shown in FIGS. 8C and 8D each representing one human NK cell donor, showing % specific lysis induced by NK cells on the y-axis and concentration of test protein on the x-axis. The overall results were consistent across the two human donors. The IC-T6-NKp46-IL2v that lacked binding to CD20, CD16 and NKp46 did not induce significant cytotoxicity. All of the NK cell engagers that retained the ability to bind both CD16 and NKp46 (in addition to CD20) displayed strong ability to potentiate NK cell cytotoxicity toward the tumor cells. The NK cell engagers that lacked either CD16 or NKp46 binding displayed lower potency in terms of EC50 values. The CD20-F5-NKp46 NK cell engager which did not have any IL2v moiety was of comparable potency as the CD20-T5-NKp46-IL2v3 NK cell engager, although the latter showed a higher plateau of lysis.

### Example 8: NKCE-IL2v Induce Cytokines when Engaged on Tumor Cells

In this experiment, NK cell engager proteins were assessed for their ability to induce cytokine production by NK cells when they were co-cultured with RAJI tumor cells at effector:target ratio of 0.5:1. The test was a standard 4-hour cytokine assay in presence of Golgi stop using intracellular flow cytometry as readout.

The test proteins included in this experiment:
CD20-T5-NKp46-IL2v: contains from topological N- to C-terminus, anti-CD20 VH/VL pair, Fc domain dimer that binds CD16, anti-NKp46-1 VH/VL pair, IL-2v.
CD20-F25-NKp46: contains from topological N- to C-terminus, anti-CD20 VH/VL pair, Fc domain dimer that binds CD16, anti-NKp46-1 VH/VL pair.
CD20-T5-IC-IL2v: contains from topological N- to C-terminus, anti-CD20 VH/VL pair, Fc domain dimer that binds CD16, IC VH/VL pair, IL2v.
CD20-T6-NKp46-IL2v: contains from topological N- to C-terminus, anti-CD20 VH/VL pair, Fc domain dimer mutated to abolish CD16 binding, anti-NKp46-1 VH/VL pair, IL2v.
IC-T5-NKp46-IL2v: contains from topological N- to C-terminus, IC VH/VL pair, Fc domain dimer that binds CD16, anti-NKp46-1 VH/VL pair, IL2v.

Briefly, freshly purified NK cells were cocultured with Raji tumor cells in a 0.5 to 1 ratio. Cells were incubated in presence of Golgi Stop with the test proteins described above (doses from 13 nM to 0.00001 nM) at 37° C., 5.5% CO² in incubator. After 4 h, IFNγ and MIPβ were analysed by intracellular flow cytometry gated on NK cells.

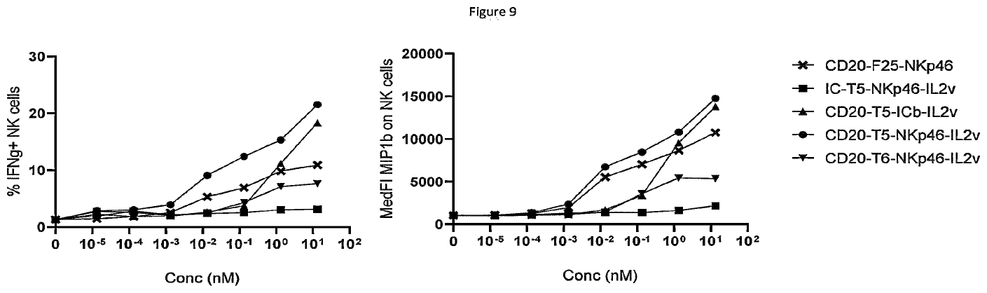

Results, shown in FIG. 9, show the % of IFN-γ positive NK cells (left) and Medfi of MIP1β on NK cells (right) on the y-axis and the test molecule concentration on the x-axis. The NK cell engager lacking the binding to CD20 on targeted cells did not induce IFN-γ nor MIP1β production. All of the NK cell engagers that retained the ability to bind both CD16 and NKp46 (in addition to CD20) induced the highest levels of IFN-γ and MIP1β at the lower doses of test molecules. In contrast, the NK cell engagers that lacked either CD16 or NKp46 binding displayed lower potency of cytokine production at the lower doses of test molecules. The CD20-T6-NKp46-IL2v which can bind NKp46 but not CD16 was the less efficient at inducing cytokines while the CD20-T5-IC-IL2v which bind CD16 but not NKp46 via its wild-type Fc domain dimer, resulted in high cytokine production at the highest doses of test molecules. The presence of IL2v in the CD20-T5-NKp46-IL2v molecule compared to the CD20-F25-NKp46 NK cell engager that did not have any IL-2 moiety seems to slightly increase the ability of NK cells to produce these cytokines.

### Example 9: NKCE-IL2v has Strong Anti-Tumor Efficacy In Vivo

In this experiment, a single injection of NK cell engager protein CD20-T5-NKp46-IL2v (containing from N- to C-terminus, anti-CD20 VH/VL pair, wild-type Fc domain dimer that binds CD16 (not comprising mutations to decrease CD16 binding), anti-NKp46 VH/VL pair that binds the Mus musculus NKp46, IL2v) was assessed for its in vivo anti-tumor activity in a murine model of human cancer.

The NK cell engager protein was compared to the commercial gold standard Fc-enhanced anti-CD20 antibody obinutuzumab. Briefly, CB17-SCID mice were engrafted subcutaneously by 5×10⁶ RAJI cells in matrigel. 9 days after engraftment, mice were treated with a single intravenous injection of different doses of CD20-T5-NKp46-IL2v (70 ug, 10 ug, 2 ug and 0.4 ug), different doses of obinutuzumab (1500 ug, 150 ug, 50 ug and 15 ug) and PBS as vehicle. Tumor growth was followed over time.

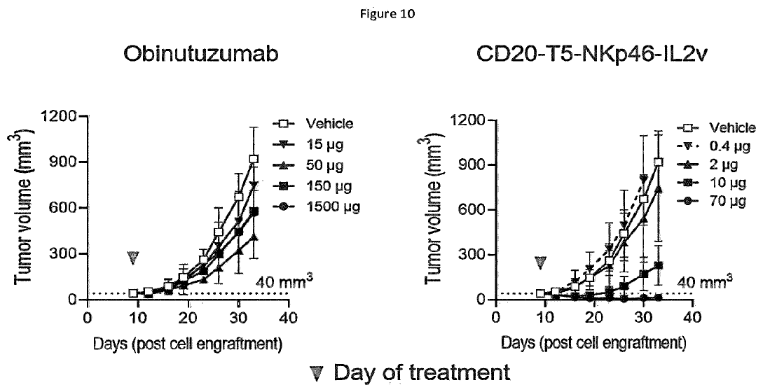

Results are shown in FIG. 10. The CD20-T5-NKp46-IL2v NK cell engager protein that bound CD20, NKp46, CD16A and CD122 (right panel) showed strong efficacy as a single injection, compared to obinutuzumab (left panel) which displayed limited activity in this model. The 10 μg dose of CD20-T5-NKp46-IL2v resulted in anti-tumor activity which prevented tumor growth past 40 mm³ in volume for about 20 days. The 70 μg dose of CD20-T5-NKp46-IL2v resulted in very strong anti-tumor activity, in which tumors did not grow past 40 mm³ in volume for the duration of the study.

### Example 10: NKCE-IL2v has Strong Anti-Tumor Efficacy In Vivo Against Large Volume Tumors

In this experiment, weekly treatment with NK cell engager protein CD20-T5-NKp46-IL2v (containing from N- to C-terminus, anti-CD20 VH/VL pair, Fc domain dimer that binds CD16, anti-NKp46 VH/VL pair, IL2v) was assessed for its in vivo anti-tumor activity in a murine model of human cancer in which tumors were allowed to grow to a large volume of 300 m³ before treatment.

Briefly, CB17-SCID mice were engrafted subcutaneously by 5×10⁶ RAJI cells in matrigel. 11 days after engraftment, when tumors reach a mean volume of 280 mm³, mice were treated with weekly repeated intravenous injections of 25 ug of CD20-T5-NKp46-IL2v or PBS as control vehicle. Tumor growth was followed over time.

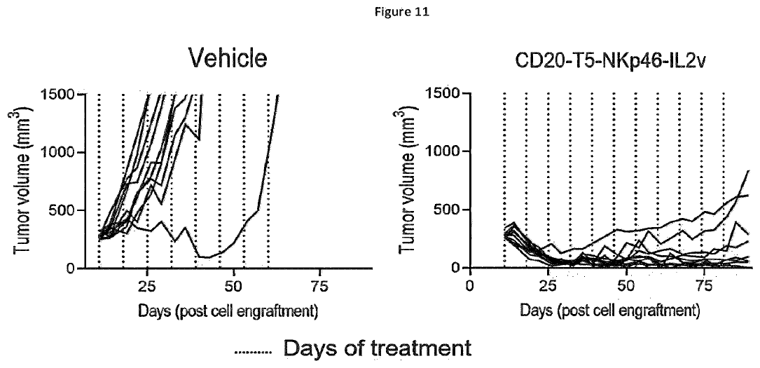

Results are shown in FIG. 11. The CD20-T5-NKp46-IL2v NK cell engager protein that bound CD20, NKp46, CD16A and CD122 (right panel) showed strong efficacy as a weekly injection starting at day 11 post tumor engraftment. The 25 μg dose of CD20-T5-NKp46-IL2v resulted in very strong and long anti-tumor activity, in which tumors first decreased in volume and then generally stayed below 300 mm³ in volume for the duration of the study. In comparison, in the vehicle control group, tumor rapidly grew past 300 mm³ in volume in the days following tumor engraftment. The experiment showed that the CD20-T5-NKp46-IL2v molecule was efficient to control large volume tumor on a long period of time.

### Example 11: Both NKp46 and IL2v are Required for Strong Efficacy In Vivo

In this experiment, treatment with two injections of different NK cell engager proteins was assessed for their in vivo anti-tumor activity in a murine model of human cancer. The proteins tested were:
CD20-T5-NKp46-IL2v, containing from N- to C-terminus, anti-CD20 VH/VL pair, Fc domain dimer that binds CD16, anti-NKp46 VH/VL pair that bind to NKp46 from Mus musculus, IL-2v.
CD20-T5-IC-IL2v which lacks NKp46 binding,
CD20-F5-NKp46 which lacks CD122 binding.
PBS was injected as vehicle and negative control.

Briefly, CB17-SCID mice were engrafted subcutaneously by 5×10⁶ RAJI cells in matrigel. Mice were treated by 2 intravenous injections of 25 ug of CD20-T5-NKp46-IL2v, 25 ug of CD20-T5-IC-IL2v, 25 ug of CD20-F5-NKp46 or PBS as control vehicle at day 9 and day 16 after tumor cell engraftment. Tumor growth was followed over time.

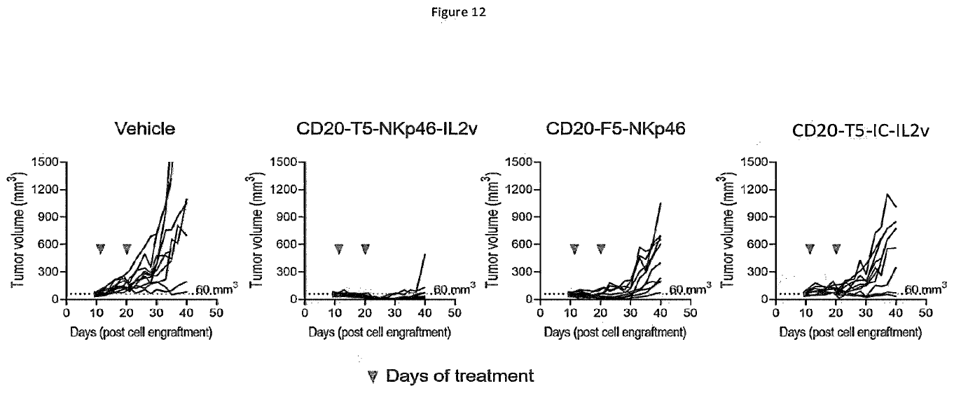

Results are shown in FIG. 12. The CD20-T5-NKp46-IL2v NK cell engager protein that binds CD20, NKp46, CD16A and CD122 showed strong efficacy as two injections separated by a one week, starting at day 9 post tumor engraftment when tumors had grown to 60 mm³ in volume. The 25 μg dose of CD20-T5-NKp46-IL2v resulted in very strong anti-tumor activity, in which tumors generally stayed in the range of 60 mm³ in volume for the duration of the study. In comparison, in the CD20-T5-IC-IL2v group and in the CD20-F5-NKp46, tumors were somewhat initially controlled during week after treatment but then rapidly grew past 300 mm³ in volume thereafter. The experiments show that both NKp46 and CD122 binding are important to efficient control tumor growth and their simultaneous targeting drives the strong antitumor efficacy of the CD20-T5-NKp46-IL2v molecule.

### Example 12: NKCE-IL2v Induces NK Cell Accumulation and an Inflammatory Microenvironment in Solid Tumor

In this experiment, we tested the ability of the NK cell engagers CD20-F5-NKp46 and CD20-T5-NKp46-IL2v to induce NK cell accumulation in tumors, in an in vivo murine model of human cancer. The molecules tested were:
CD20-T5-NKp46-IL2v: contains from N- to C-terminus, anti-CD20 VH/VL pair, Fc domain dimer that binds CD16, anti-mouse NKp46-1 VH/VL pair, IL-2v.
CD20-F5-NKp46: contains from N- to C-terminus, anti-CD20 VH/VL pair, Fc domain dimer that binds CD16, anti-mouse NKp46-1 VH/VL pair.
Obinutuzumab.

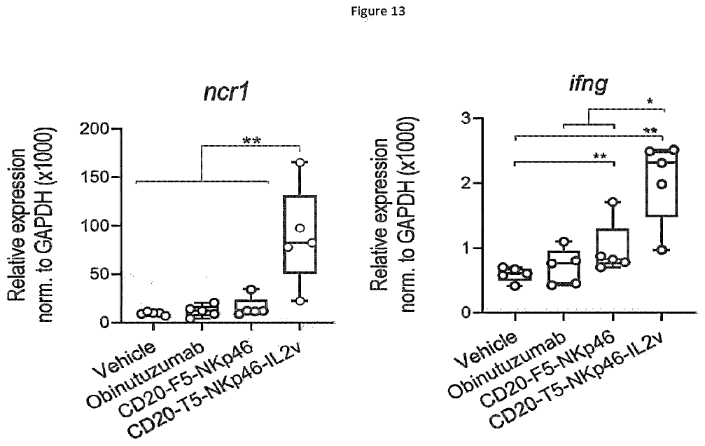

Briefly, CB17-SCID mice were engrafted subcutaneously by 5×10⁶ RAJI cells in matrigel. 20 days after Raji cell engraftment, mice were treated by a single intravenous injections of 70 ug of CD20-T5-NKp46-IL2v, 125 ug of CD20-F5-NKp46, 600 ug of obinutuzumab and PBS as control vehicle. After 3 days, tumors were harvested for RNA extraction. cDNA were prepared from whole tumor mRNA extract and qPCR performed to quantify ncr1 and ifn-γ (ifng) transcript expression to evaluate NK cells infiltrate and activation in the tumor. Results are shown in FIG. 13. Tumors harvested from mice treated by the CD20-T5-NKp46-IL2v NK cell engager protein that bound CD20, NKp46, CD16A and CD122 showed high expression of the ncr1 transcript (encoding for NKp46 protein and highly specific for NK cells), demonstrating an increase of NK cell infiltration in tumor. In comparison, tumors harvested in mice treated by the CD20-F5-NKp46 protein or obinutuzumab showed only minor increase of ncr1 transcripts revealing a much lower NK cell infiltrate in tumors. Furthermore, tumors harvested from mice treated by the CD20-T5-NKp46-IL2v NK cell engager protein showed higher expression of the ifng transcript compare to other treated conditions. Tumors harvested in mice treated by the CD20-F5-NKp46 which lacks CD122 binding also showed a lower but significant increase of ifng transcripts compare to tumors from obinutuzumab or PBS injected mice. The results show that CD20-T5-NKp46-IL2v proteins promote NK cell recruitment and activation in the targeted tumors.

### Example 13: NK Cells Drive NKCE-IL2v Efficacy to Control Solid Tumor Growth

In this experiment, we assessed the effect of in vivo depletion of NK cells on the ability of the NK cell engager CD20-T5-NKp46-IL2v to control tumor cell growth in an in vivo murine model of human cancer.

Briefly, CB17-SCID mice were engrafted subcutaneously by 5×10⁶ RAJI cells in matrigel. Mice were injected with 25 ug CD20-T5-NKp46-IL2v at day 10 when tumors had reached 70 mm³, followed by a second injection one week later. NK depletion was carried out by injection of anti-Asialo GM1 antibody, administered once a week, starting at day 9 (before treatment) and repeated for a total of six administrations. Tumor growth was followed over time.

Results are shown in FIG. 14. The upper right hand panel shows that treatment with the CD20-T5-NKp46-IL2v NK cell engager controlled tumor growth. However, NK cell depletion resulted in a loss of control of tumor growth by about day 30 (lower right hand panel) while tumor are still controlled in mice not depleted for NK cells. These data show that NK cells are important for the antitumor activity induced by the CD20-T5-NKp46-IL2v NK cell engager.

### Example 14: NKCE-IL2v Molecules with Different Linker Length

In view of the potential effect of domain positioning within the NKCE on the ability to simultaneously bind each of NKp46, CD16 and CD122 on NK cells, we sought to investigate the effect of different structural modifications in the NKCE-IL2v proteins on their ability to induce IL-2R signaling.

A series of different heterotrimeric proteins were compared that all bound tumor antigen (CD20, indicated interchangeable also as “GA101” referring to the anti-CD20 VH/VL pair), CD16 (by inclusion of a dimeric wild-type Fc domain), NKp46 and CD122 (by inclusion of an IL2v molecule). In one series of proteins, exemplified by GA101-T5-NKp46-IL2v, the NKp46-binding domain (as a Fab) was placed at the C-terminus of the Fc domain dimer, and the IL2v was placed at the C-terminus of the NKp46-binding Fab (domain arrangement of the NKp46, Fc and IL2v moieties, from N- to C-terminus: dimeric Fc-anti-NKp46 Fab-IL2v). The length of the flexible domain linker separating IL2v from the NKp46-binding domain was varied, including a short linker of 5 amino acid residues, the 10 amino acid residue linker of GA101-T5-NKp46-IL2v, and a long linker of 15 amino acid residues.

The proteins tested were:
Recombinant human IL-2
CD20-T5-NKp46-IL2v
CD20-T5-NKp46-IL2v Short Linker
CD20-T5-NKp46-IL2v Long Linker

Briefly, 1M/well of purified PBMC were seeded in 96-well plate and treated with increasing doses of various NKCE-IL2v (dose from 133 nM to 0.0000013 nM) for 20 min at 37° C., 5.5% CO² in incubator. STAT5 phosphorylation was then analyzed by flow cytometry on NK cells (gated on CD3− CD56+), CD4 T cells (CD3+ CD4+ FoxP3−), CD8 T cells (CD3+ CD8+) and Treg cells (CD3+ CD4+ CD25hi FoxP3+).

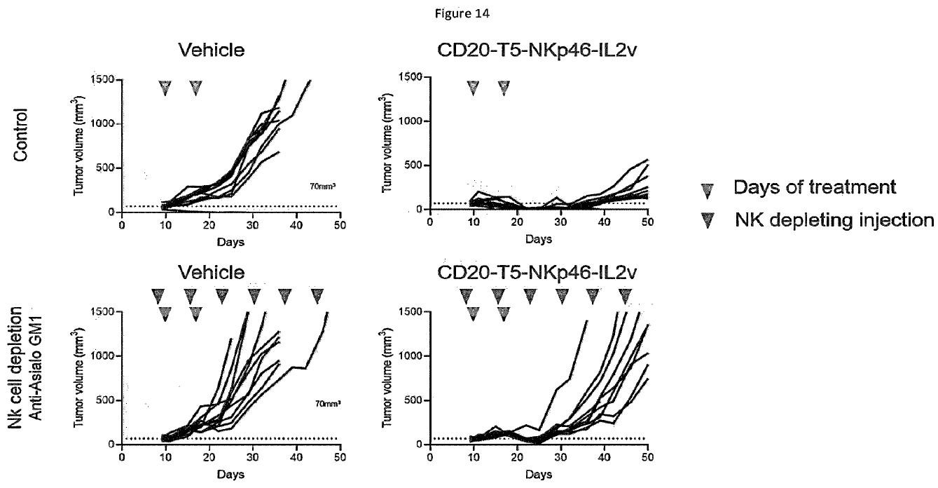

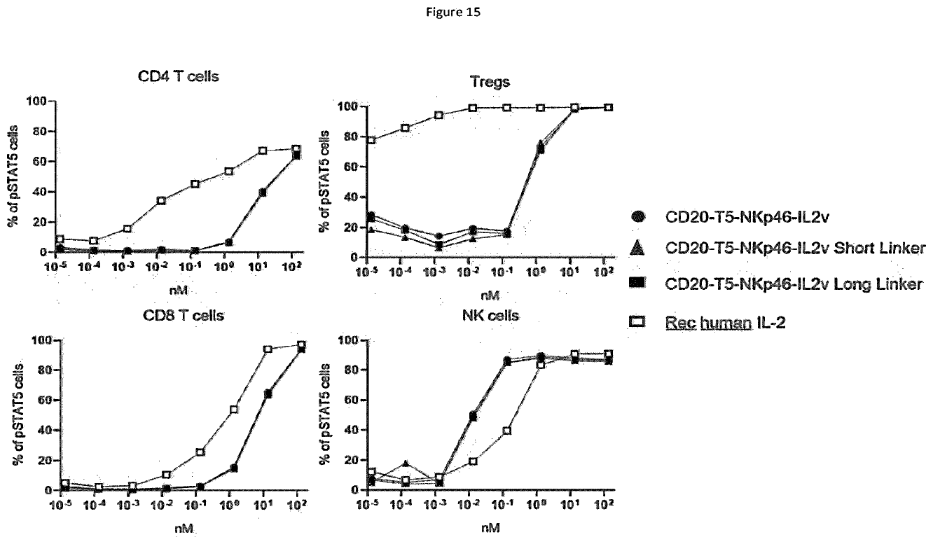

Results are shown in FIG. 15 showing % of pSTAT5 cells among PBMC on the y-axis and concentration of test protein on the x-axis. All the GA101-T5-NKp46-IL2v NK cell engager proteins induced potent IL2R signaling preferentially in NK cells over TReg cells, CD8 T cells and CD4 T cells whatever the length of the linker used.

### Example 15: NKCE-IL2v Binding to Distant Epitopes on NKp46 Retain Strong Ability to Induce Cytotoxicity

This experiment was to investigate the effect of different structural modifications in the NKCE-IL2v proteins on their ability to potentiate the cytotoxicity of NK cells toward target cells. In this experiment, NK cell engager proteins were assessed for their ability to induce killing of RAJI tumor cells by resting purified NK cells at effector:target ratio of 10:1 in a standard 4-hour cytotoxicity assay suing Cr⁵¹ as readout. In this series of experiments, we tested different domain positioning within the multispecific protein, different lengths of domain linkers separating the IL2v moiety from the rest of the multispecific protein, and different NKp46 binding domains that bind to distant sites on the NKp46 molecule.

In addition to the multispecific proteins described in Examples 2-14 which all made use of NKCE proteins which incorporated the NKp46-1 VH/VL pair, additional CD20-binding NKCE proteins in the CD20-T5-NKp46-4-IL2v were tested that incorporated instead the NKp46-4 VH/VL pair. As shown in epitope mapping experiments and in Gauthier et al. 2019 (Cell 177:1701-1713), while the NKp46-1 antibody binds to an epitope on NKp46 located at the transition between domains 1 and 2, the NKp46-4 antibody binds to an epitope on the N-terminal part of the D1 domain of NKp46.

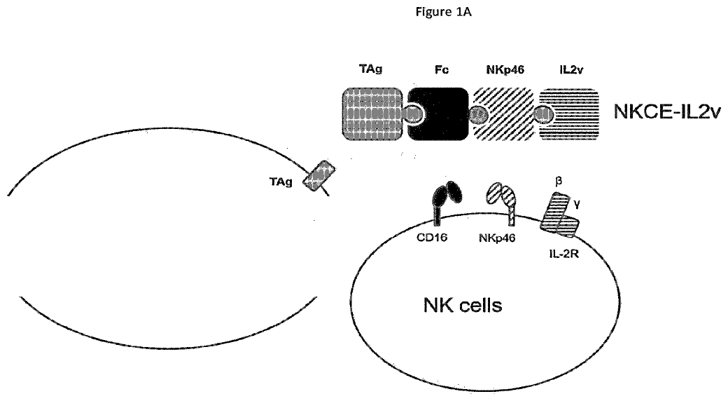

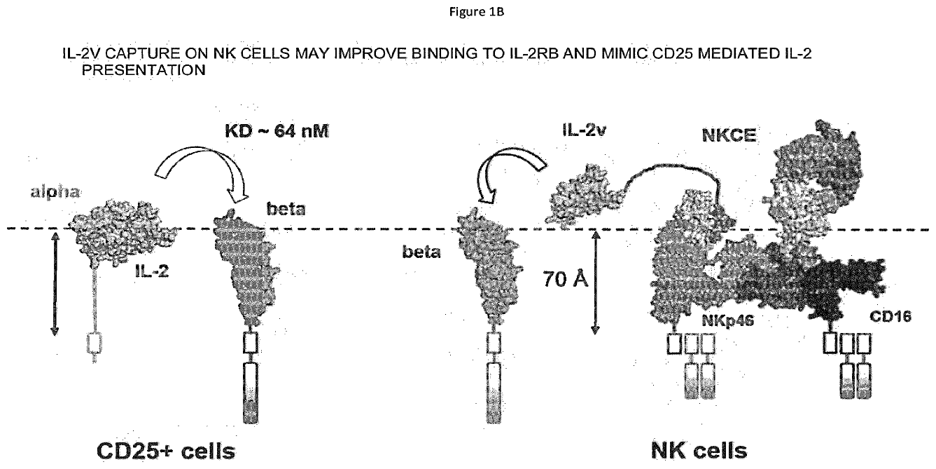

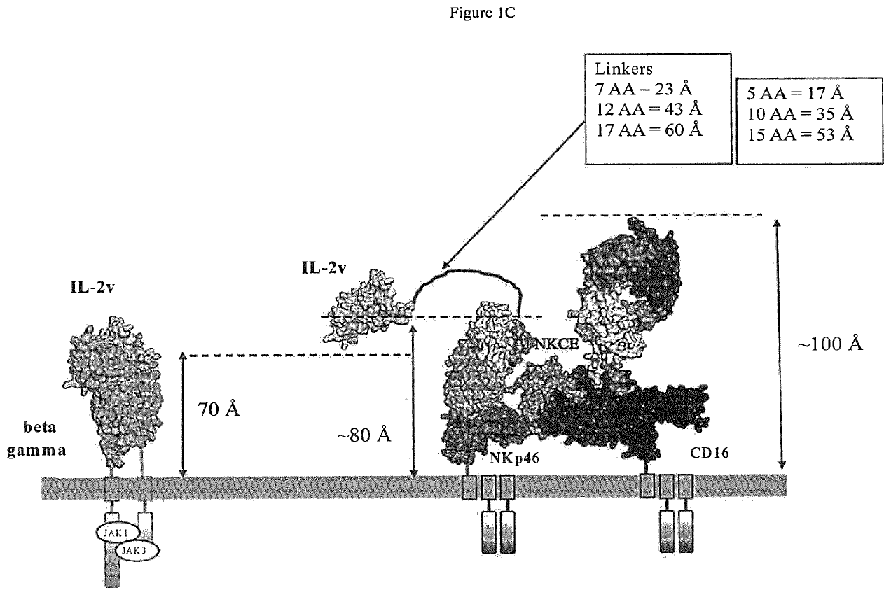

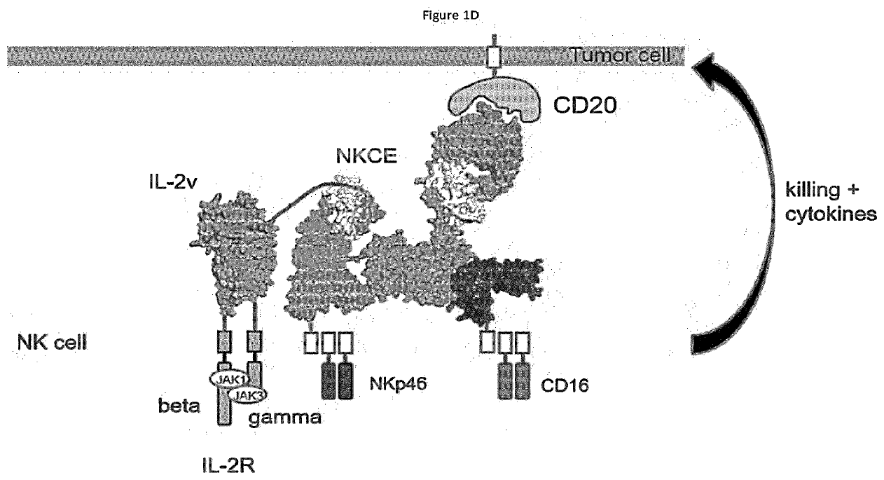

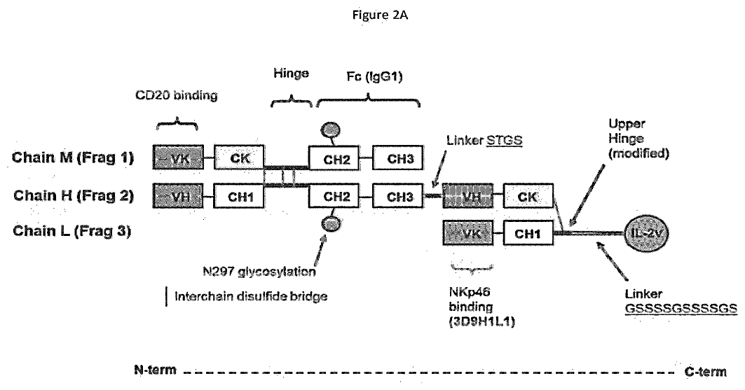

Three-dimensional modelling of the binding to the N-terminal part of NKp46 suggested that in such a setting the anti-NKp46-4 VH/VL pair would be positioned closer to the cell membrane compared to the NKp46-1 VH/VL pair, and that such binding might impair the ability of the IL2v moiety to interact with CD122 whose IL-2 binding site is positioned about 70 Angstroms from the cell surface (see FIG. 1C). Consequently, the CD20-T5-NKp46-4-IL2v was produced with three different lengths for the flexible domain linker separating IL2v from the NKp46-binding domain, including a short linker of 5 amino acid residues, a standard 10 amino acid residue linker, and a long linker of 15 amino acid residues.

The proteins tested were:
CD20-T5-NKp46-1-IL2v
CD20-T5-NKp46-1-IL2v Short Linker
CD20-T5-NKp46-1-IL2v Long Linker
CD20-T6-NKp46-1-IL2v
IC-T6-NKp46-1-IL2v
IC-T5-NKp46-1-IL2v
CD20-T5-NKp46-4-IL2v
CD20-T5-NKp46-4-IL2v Short Linker
CD20-T5-NKp46-4-IL2v Long Linker

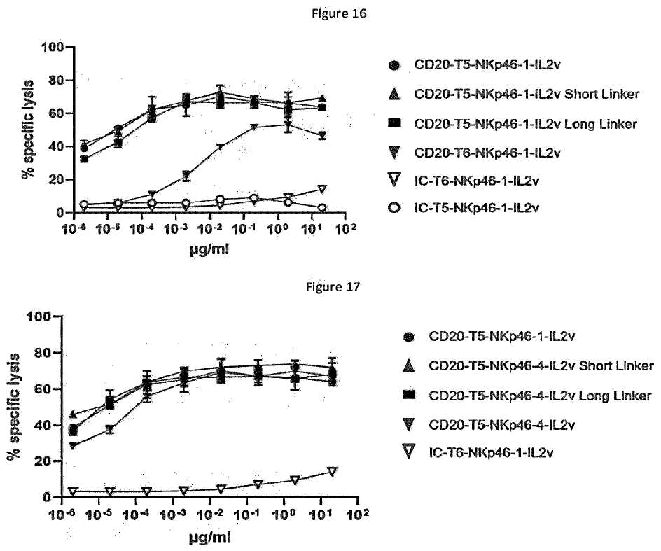

Results are shown in FIGS. 16, and 17 showing % specific lysis on the y-axis and concentration of test protein on the x-axis. FIG. 16 shows cytotoxicity potentiated by the NKCE proteins in the “T5” and “T6” formats in which the NKp46-binding domain based on the NKp46-1 VH/VL pair is positioned between the wild-type Fc domain dimer and the C-terminal IL2v, with 10 amino linker, short (5 aa) linker or long (15 aa) linker; the proteins were all comparable in their ability to potentiate NK cell cytotoxicity towards tumors cells, without negative impact on NK cytotoxicity by the shorter or longer linkers.

FIG. 17 shows cytotoxicity potentiated by the NKCE proteins in the “T5” and “T6” formats in which the NKp46-binding domain based on the NKp46-4 VH/VL pair is positioned between the wild-type Fc domain dimer and the C-terminal IL2v, with 10 amino linker, short (5 aa) linker or long (15 aa) linker; the proteins were all comparable in their ability to potentiate NK cell cytotoxicity towards tumors cells. The NKp46 binding site targeted by the NKCE protein therefore does not impact ability cytotoxicity.

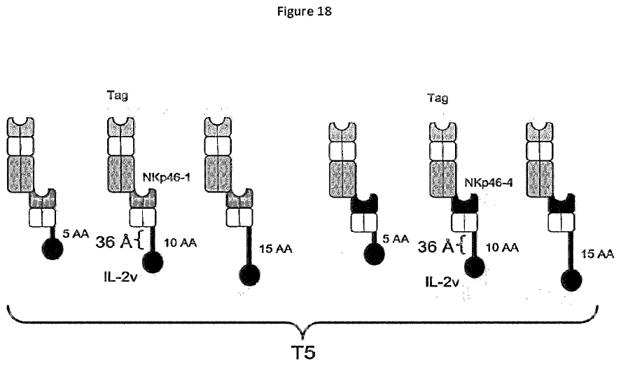

FIG. 18 shows the structure of the proteins tested in FIGS. 16, 17 and 18.

### Example 16: Different Cytokine Variants do not Negatively Affect IL2R Signaling Induced by NKCE-IL2v

The IL2v moiety used in NKCE proteins of Examples 2-16, together with a new IL2v moiety referred to as IL2v3, was chosen in order to confer preference for binding to IL-2Rβ on NK cells over IL-2Rα on T cells, including Tregs. This experiment was to investigate the effect of different structural modifications in the cytokine proteins on their ability to affect IL2R signaling induced by NKCE-IL2v.

Different NKCE-IL2v proteins were prepared in which the IL2v amino acid sequence was varied, however while maintaining a preferential binding to the NK-expressed receptor chain (IL-2Rβ) over the Treg cell expressed chain (IL-2Rα). The NKCE proteins were then tested for the ability to differentially induce IL2R in NK cells over T cells, to assess whether different cytokine variants will affect the selective targeting of IL-2Rβ over the high affinity IL-2Rαβγ complex.

Three IL-2 mutant polypeptides were produced and placed C-terminally on the NKCE-IL2v protein, as follows:
CD20-T5-NKp46-IL2v: contains from N- to C-terminus, anti-CD20 VH/VL pair, Fc domain dimer that binds CD16, anti-NKp46-1 VH/VL pair, IL2v (mutations T3A, C125A, F42A, Y45A and L72G with reference to the mature wild-type human IL-2).
CD20-T5-NKp46-IL2v2: contains from N- to C-terminus, anti-CD20 VH/VL pair, Fc domain dimer that binds CD16, anti-NKp46-1 VH/VL pair, IL2v2 (mutations R38A/F42K with reference to the mature wild-type human IL-2).
CD20-T5-NKp46-IL2v3: contains from N- to C-terminus, anti-CD20 VH/VL pair, Fc domain dimer that binds CD16, anti-NKp46-1 VH/VL pair, IL2v3 (mutations R38A/T41A/F42K with reference to the mature wild-type human IL-2).

Briefly, 1M/well of purified PBMC were seeded in 96-well plate and treated with increasing doses of various NKCE-IL2v (dose from 133 nM to 0,0000013 nM) for 20 min at 37° C., 5.5% CO2 in incubator. STAT5 phosphorylation was then analyzed by flow cytometry on PBMC and gated on NK cells (CD3−CD56+), CD8 T cells (CD3+ CD8+), CD4 T cells (CD3+ CD4+ FoxP3−) and Tregs (gated on CD3+ CD4+ CD25+FoxP3+).

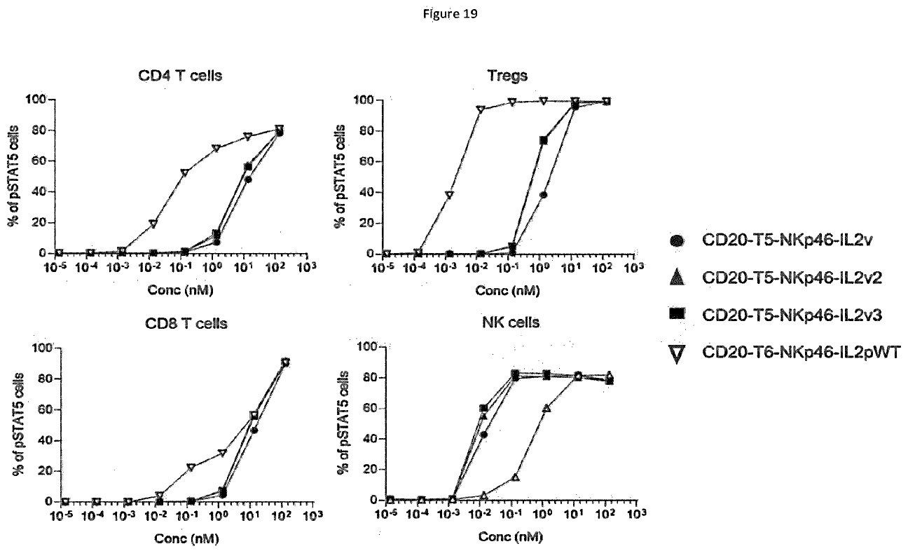

Results are shown in FIG. 19 showing % of pSTAT5 cells among PBMC cells on the y-axis and concentration of test protein on the x-axis. The CD20-T5-NKp46-IL2v, CD20-T5-NKp46-IL2v2 and CD20-T5-NKp46-IL2v3 were comparable and each resulted in an approximately 2-log increase in percent of pSTAT5+ cells among the NK cells, compared to IC-T6-IC-IL2 (IL2pWT) that contained wild-type IL-2 and did not bind NKp46 or CD16A. The CD20-T5-NKp46-IL2v protein therefore permitted a selective activation of NK cells over Treg cells, CD4 T cells and CD8 T cells. Substitution of different “not-alpha” cytokine variants does not impact ability of the NKCE-IL2v protein to selectively activate NK cells over Treg cells, CD4 T cells and CD8 T cells. EC50 values for activation of NK cells and Tregs are shown below.

| % pSTAT5 nM | EC50 NK cells | EC50 Tregs |
|---|---|---|
| IL2pWT | 0.6 | 0.002 |
| IL2v | 0.013 | 1.8 |
| IL2v2 | 0.011 | 0.73 |
| IL2v3 | 0.010 | 0.7 |

### Example 17: NKCE-IL2v Induces Minimal Systemic Cytokine Release In Vivo in Mice

In this experiment, we assessed the effect on systemic cytokine release of treatment with the NK cell engager CD20-T5-NKp46-IL2v in mice. CB17 SCID mice were injected with a single injection of either 25 μg or 70 μg of CD20-T5-NKp46-IL2v, with four mice per dose level. Murine IL-6 and TNFα production in plasma was monitored over time for 14 days.

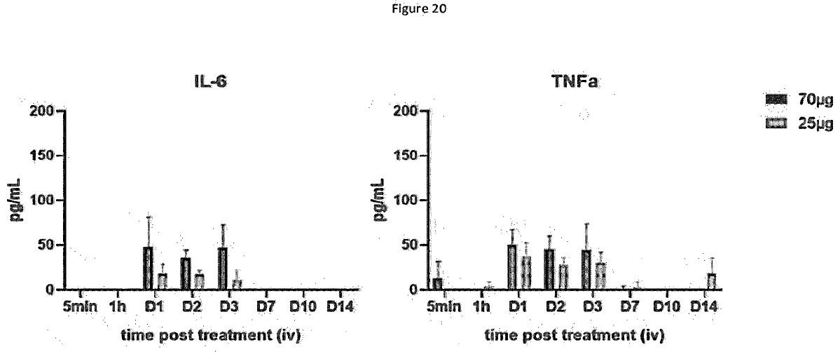

Results are shown in FIG. 20, with the left panel showing IL-6 production and the right panel showing TNFα production. For each time point post treatment (D indicates days), the left bar shows plasma concentration of cytokine for the 70 μg dose and the right bar shows plasma concentration of cytokine for the 25 μg dose. FIG. 10 shows that treatment with the CD20-T5-NKp46-IL2v NK cell engager controlled tumor growth using single injection of doses over 10 ug. These data show that the CD20-T5-NKp46-IL2v induced only minimal systemic cytokine production during the 3 days following treatment, and without systemic cytokine production thereafter, suggesting that the CD20-T5-NKp46-IL2v does not involve immune toxicity, despite the treatment being at doses where it displays potent anti-tumor and NK cell cytotoxicity activity.

### Example 18: Mechanism of Action of Tumor Antigen, NKp46 and CD16 Binding Components of NKCE Proteins

These experiments are shown in order to illustrate the role of NKp46 and CD16A on NK cells in the mechanism of action of the NKCE proteins, in the absence of inclusion of the cytokine moiety. Different NKCE protein formats were produced that either had low or substantially lack of binding to FcγR (including CD16) or which had binding to FcγRs (including CD16), e.g. the binding affinity to human CD16 was within 1-log of that of wild-type human IgG1 antibodies, as assessed by SPR.

Different constructs were made for use in the preparation of a multispecific protein using the variable domains DNA and amino acid sequences derived from the scFv specific for tumor antigen CD19 described and different variable regions from antibodies specific for NKp46. Proteins were cloned, produced and purified as in PCT publication no. WO2016/207273. Domains structures are shown in FIGS. 21A and 21B for the different formats.

Format 2 (F2): CD19-F2-NKp46-3

The domain structure of F2 polypeptides having a monomeric Fc domain and therefore not binding CD16 is shown in FIG. 21A. The DNA and amino acid sequences for the monomeric CH2-CH3 Fc portion contains CH3 domain mutations (EU numbering) L351K, T366S, P395V, F405R, T407A and K409Y to prevent CH3 heterodimerization. The heterodimer is made up of:
(1) a first (central) polypeptide chain having domains arranged as follows (N- to C-termini):

(Vκ-VH)^{anti-CD19}-CH2-CH3-V_{H}^{anti-NKp46}-CH1

and
(2) a second polypeptide chain having domains arranged as follows (N- to C-termini): VK^{anti-NKp46}-CK.

The (VK-VH) unit was made up of a VH domain, a linker and a VK unit (i.e. an scFv). As with other formats of the bispecific polypeptides, the DNA sequence coded for a CH3/VH linker peptide having the amino acid sequence STGS designed in order to insert a specific SalI restriction site at the CH3-VH junction. Proteins were cloned, produced and purified as in Example 2-1. The amino acid sequences for the CD19-F2-NKp46-3 Polypeptide chain 1 is shown in SEQ ID NO: 221 and CD19-F2-NKp46-3 Polypeptide chain 2 in SEQ ID NO: 222.

Format 5 (F5): CD19-F5-NKp46-3

The domain structure of the trimeric F5 polypeptide is shown in FIG. 21A, wherein the interchain bonds between hinge domains (indicated in the figures between CH1/Cκ and CH2 domains on a chain) and interchain bonds between the CH1 and Cκ domains are interchain disulfide bonds. The heterotrimer is made up of:
(1) a first (central) polypeptide chain having domains arranged as follows (N- to C-termini):

V_{H}^{anti-CD19}-CH1-CH2-CH3-V_{H}^{anti-NKp46}-Cκ

and
(2) a second polypeptide chain having domains arranged as follows (N- to C-termini):

Vκ^{anti-CD19}-CK-CH2-CH3

and
(3) a third polypeptide chain having domains arranged as follows (N- to C-termini):

Vκ^{anti-NKp46}-CH1.

The amino acid sequences of the three chains are shown in SEQ ID NOS: 223 (chain 2), 224 (chain 1) and 225 (chain 3).

Format 6 (F6): CD19-F6-NKp46-3

The F6 protein is the same as F5, but contains a N297S substitution to avoid N-linked glycosylation. The amino acid sequences of the three chains of the F6 protein are shown in SEQ ID NOS: 226 (chain 2), 227 (chain 1) and 228 (chain 3).

Format 7 (F7): CD19-F7-NKp46-3

The domain structure of heterotrimeric F7 polypeptides is shown in FIG. 21A. The F7 protein is the same as F6, except for cysteine to serine substitutions in the CH1 and Cκ domains that are linked at their C-termini to Fc domains, in order to prevent formation of a minor population of dimeric species of the central chain with the Vκ^{anti-NKp46}-CH1 chain. The amino acid sequences of the three chains of the 76 protein are shown in SEQ ID NOS: 229 (chain 2), 230 (chain 1) and 231 (chain 3).

Format 13 (F13): CD19-F13-NKp46-3

The domain structure of the dimeric F13 polypeptide is shown in FIG. 21A, wherein the interchain bonds between hinge domains (indicated between CH1/Cκ and CH2 domains on a chain) and interchain bonds between the CH1 and Cκ domains are interchain disulfide bonds. The heterodimer is made up of:
(1) a first (central) polypeptide chain having domains arranged as follows (N- to C-termini):

V_{H}^{anti-CD19}-CH1-CH2-CH3-(V_{H}-Vκ)^{anti-NKp46}

and
(2) a second polypeptide chain having domains arranged as follows (N- to C-termini):

Vκ^{anti-CD19}-Cκ-CH2-CH3.

The (V_{H}-Vκ) unit was made up of a V_{H} domain, a linker and a Vκ unit (scFv).

The amino acid sequences of the two chains of the F13 protein are shown in SEQ ID NOS: 232 and 233.

Format 14 (F14): CD19-F14-NKp46-3

The domain structure of the dimeric F14 polypeptide is shown in FIG. 21B. The F14 polypeptide is a dimeric polypeptide which shares the structure of the F13 format, but instead of a wild-type Fc domain (CH2-CH3), the F14 bispecific format has CH2 domain mutations N297S to abolish N-linked glycosylation. The amino acid sequences of the two chains of the F14 protein are shown in SEQ ID NOS: 234 and 235.

### NKp46 Binding Affinity Determination by Surface Plasmon Resonance (SPR)

### Biacore T100 General Procedure and Reagents

SPR measurements were performed on a Biacore T100 apparatus (Biacore GE Healthcare) at 25° C. In all Biacore experiments HBS-EP+ (Biacore GE Healthcare) and NaOH 10 mM served as running buffer and regeneration buffer respectively. Sensorgrams were analyzed with Biacore T100 Evaluation software. Protein-A was purchased from (GE Healthcare). Human NKp46 recombinant proteins were cloned, produced and purified at Innate Pharma.

### Immobilization of Protein-A

Protein-A proteins were immobilized covalently to carboxyl groups in the dextran layer on a Sensor Chip CM5. The chip surface was activated with EDC/NHS (N-ethyl-N′-(3-dimethylaminopropyl) carbodiimidehydrochloride and N-hydroxysuccinimide (Biacore GE Healthcare)). Protein-A was diluted to 10 μg/ml in coupling buffer (10 mM acetate, pH 5.6) and injected until the appropriate immobilization level was reached (i.e. 2000 RU). Deactivation of the remaining activated groups was performed using 100 mM ethanolamine pH 8 (Biacore GE Healthcare).

### Binding Study

Antibodies were next tested as different formats having the anti-NKp46 variable regions from the NKp46-3 antibody, and were compared to the NKp46-3 antibody as a full-length human IgG1. Bispecific proteins at 1 μg/mL were captured onto Protein-A chip and recombinant human NKp46 proteins were injected at 5 μg/mL over captured bispecific antibodies. For blank subtraction, cycles were performed again replacing NKp46 proteins with running buffer. For FACS screening, the presence of reactive antibodies in the supernatants was detected using Goat anti-mouse polyclonal antibody (pAb) labeled with PE.

### Affinity Study

Monovalent affinity study was conducted following a regular Capture-Kinetic protocol as recommended by the manufacturer (Biacore GE Healthcare kinetic wizard). Seven serial dilutions of human NKp46 recombinant proteins, ranging from 6.25 to 400 nM were sequentially injected over the captured Bi-Specific antibodies and allowed to dissociate for 10 min before regeneration. The entire sensorgram sets were fitted using the 1:1 kinetic binding model.

### Results

All of the formats tested retained binding to NKp46 when using the NKp46-3 variable regions. Monovalent affinities and kinetic association and dissociation rate constants are shown below in Table 7 below.

TABLE 7

| Bispecific mAb | ka (1/Ms) | kd (1/s) | KD (M) |
|---|---|---|---|
| CD19-F5-NKp46-3 | 7.555E+4 | 0.00510 | 67E−09 |
| CD19-F6-NKp46-3 | 7.934E+4 | 0.00503 | 63E−09 |
| CD19-F13-NKp46-3 | 8.300E+4 | 0.00565 | 68E−09 |
| CD19-F14-NKp46-3 | 8.826E+4 | 0.00546 | 62E−09 |
| NKp46-3 IgG1 | 2.224E+5 | 0.00433 | 20E−09 |

Comparative Efficacy with Depleting Anti-Tumor mAbs: NKp46×CD19 Bispecific Proteins at Low ET Ratio

These studies aimed to investigate whether NKCE proteins can mediate NKp46-mediated NK cell activation toward cancer target cells at low effector:target ratios. The ET ratio used in this Example was 1:1 which is believed to be closer to the setting that would be encountered in vivo. NKp46×CD19 bispecific proteins having an arrangement according to the F2 format (lacking CD16A binding) with anti-NKp46 variable domains from NKp46-1, NKp46-2, NKp46-3, NKp46-4 or NKp46-9 were compared to:
(a) full-length monospecific anti-NKp46 antibodies (NKp46-3 as human IgG1), and
(b) the anti-CD19 antibody as a full-length human IgG1 as ADCC inducing antibody control comparator.

The experiments further included as controls: rituximab (an anti-CD20 ADCC inducing antibody control for a target antigen with high expression levels); anti-CD52 antibody alemtuzumab (a human IgG1, binds CD52 target present on both targets and NK cells), and negative control isotype control therapeutic antibody (a human IgG1 that does not bind a target present on the target cells (HUG1-IC). The different proteins were tested for functional effect on NK cell activation as assessed by CD69 or CD107 expression in the presence of CD19-positive tumor target cells (Daudi cells), in the presence of CD19-negative, CD16-positive target cells (HUT78 T-lymphoma cells), and in the absence of target cells.

NK activation was tested by assessing CD69 and CD107 expression on NK cells by flow cytometry. The assay was carried out in 96 U well plates in completed RPMI, 150 μL final/well. Effector cells were fresh NK cells purified from donors. Target cells were Daudi (CD19-positive), HUT78 (CD19-negative) or K562 (NK activation control cell line). In addition to K562 positive control, three conditions were tested, as follows:
NK cell alone
NK cells vs Daudi (CD19+)
NK cells vs HUT78 (CD19-)

Effector:Target (E:T) ratio was 1:1, with an antibody dilution range starting to 10 μg/mL with 1/4 dilution (n=8 concentrations). Antibodies, target cells and effector cells were mixed; spun 1 min at 300 g; incubated 4 h at 37° C.; spun 3 min at 500 g; washed twice with Staining Buffer (SB); added 50 μL of staining Ab mix; incubated 30 min at 300 g; washed twice with SB resuspended pellet with CellFix; stored overnight at 4° C.; and fluorescence detected with Canto II (HTS).

Results

The results of the above experiments are shown in FIG. 22 (22A: CD107 and 22B: CD69). In the presence of target-antigen expressing cells, each of the bispecific anti-NKp46×anti-CD19 antibodies (respectively including NKp46-1, NKp46-2, NKp46-3, NKp46-4 or NKp46-9 variable regions) activated NK cells in the presence of Daudi cells.

The activation induced by bispecific anti-NKp46×anti-CD19 antibody in the presence of Daudi cells was far more potent than that elicited by the full-length human IgG1 anti-CD19 antibody. This ADCC inducing antibody had low activity in this setting. Furthermore, in this low E:T ratio setting the activation induced by the NKCE was as potent as the anti-CD20 antibody rituximab, with a difference being observed only at the highest concentrations that were 10 fold higher than concentrations in which differences were observed at the 2.5:1 ET ratio.

In the absence of target cells or in the presence of target antigen-negative HUT78 cells, full-length anti-NKp46 antibodies and alemtuzumab showed a similar level of baseline activation as was observed in the presence of Daudi cells. Anti-NKp46×anti-CD19 antibody did not activate NK cells in presence of HUT78 cells.

Combining NKp46 and CD16 triggering

NKp46×CD19 NKCE proteins that bind human CD16 having an arrangement according to the F5 format with anti-NKp46 variable domains from NKp46-3 were compared to the same bispecific antibody in a F6 format (which lacks CD16 binding), and to a human IgG1 isotype anti-CD19 antibody, as well as to a human IgG1 isotype control antibody for functional ability to direct purified NK cells to lyse CD19-positive Daudi tumor target cells.

Briefly, the cytolytic activity of fresh human purified NK cells from EFS Buffy Coat was assessed in a classical 4-h ⁵¹Cr-release assay in U-bottom 96 well plates. Daudi or HUT78 cells (negative control cells that do not express CD19) were labelled with ⁵¹Cr and then mixed with NK cells at an effector/target ratio equal to 10:1, in the presence of test antibodies at dilution range starting from 10 μg/ml with 1/10 dilution (n=8 concentrations).

After brief centrifugation and 4 hours of incubation at 37° C., 50 μL of supernatant were removed and transferred into a LumaPlate (Perkin Elmer Life Sciences, Boston, MA), and ⁵¹Cr release was measured with a TopCount NXT beta detector (PerkinElmer Life Sciences, Boston, MA). All experimental conditions were analyzed in triplicate, and the percentage of specific lysis was determined as follows: 100×(mean cpm experimental release−mean cpm spontaneous release)/(mean cpm total release−mean cpm spontaneous release). Percentage of total release is obtained by lysis of target cells with 2% Triton X100 (Sigma) and spontaneous release corresponds to target cells in medium (without effectors or Abs).

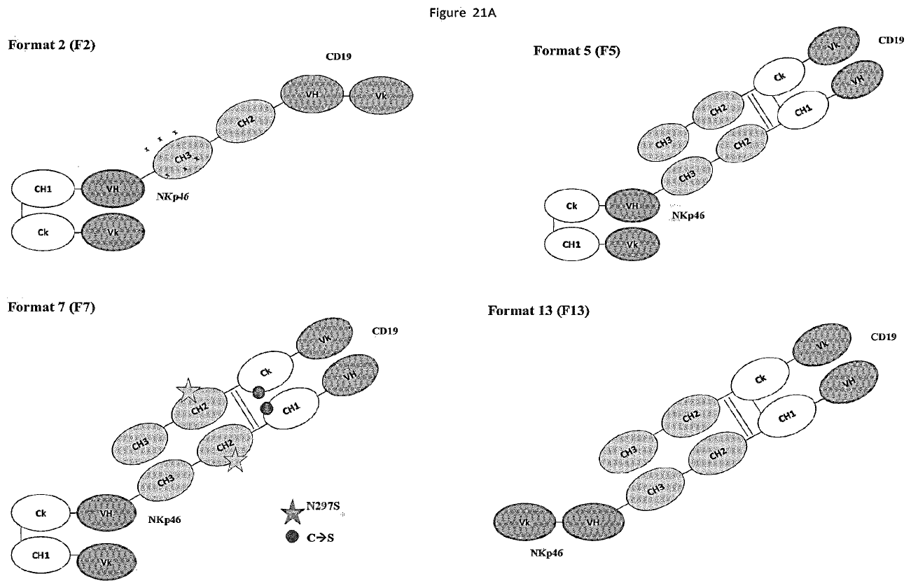

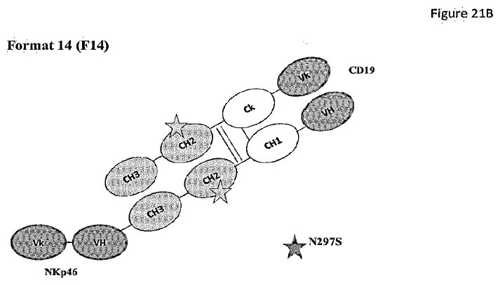

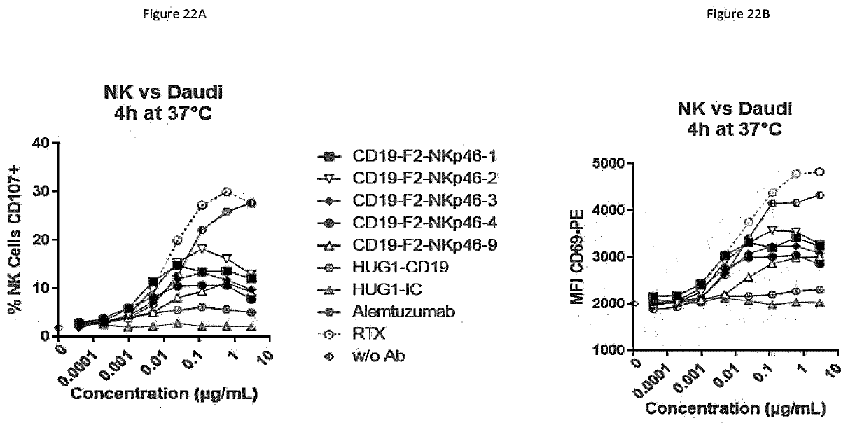

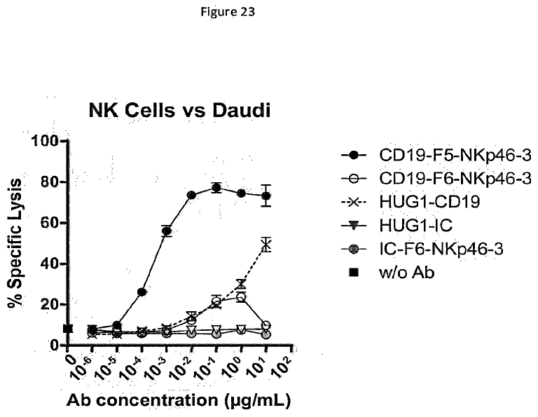

The results of these experiments are shown in FIG. 23. The CD19-F6-NKp46 (bispecific protein in F6 format) whose Fc domain does not bind CD16 due to a N297 substitution was as potent in mediating NK cell lysis of Daudi target cells as the full-length IgG1 anti-CD19 antibody. This result is remarkable especially considering that the control IgG1 anti-CD19 antibody binds CD19 bivalently and further since the anti-CD19 antibody is bound by CD16. The F6 protein was also compared to a protein CD19-F5-NKp46 that was identical to the CD19-F6-NKp46 protein with the exception of an asparagine at Kabat residue 297. Despite the strong NK activation mediated by CD16 triggering by the CD19-F5-NKp46 (F5 format protein) whose Fc domain binds CD16, the F5 format was far more potent in mediating Daudi target cell lysis that the full-length IgG1 anti-CD19 antibody or the F6 format bispecific protein. This suggests that NKp46 can enhance target cell lysis even when CD16 is triggered. In fact, at comparable levels of target cell lysis, the CD19-F5-NKp46 was at least 1000 times more potent than the full-length anti-CD19 IgG1.

### Binding of Different Bispecific Formats to FcRn

The affinity of different antibody formats for human FcRn was studied by Surface Plasmon Resonance (SPR) by immobilizing recombinant FcRn proteins covalently to carboxyl groups in the dextran layer on a Sensor Chip CM5, as described in PCT publication no. WO2016/207273. A chimeric full length anti-CD19 antibody having intact human IgG1 constant regions and NKp46×CD19 NKCE with anti-NKp46 variable domains from NKp46-3 were tested; for each analyte, the entire sensorgram was fitted using the steady state or 1:1 SCκ binding model.

The results of these experiments are shown in Table 8 below. The NKCE proteins having Fc domain dimers (formats F5, F6, F13, F14) bound to FcRn with affinity similar to that of the full-length IgG1 antibody. Other NKCE proteins with monomeric Fc domains (F3, F4, F9, F10, F11, not shown below) also displayed binding affinity to FcRn, however with lower affinity that the bispecific proteins having Fc domain dimers.

TABLE 8

| Antibody/Bispecific | SPR method | KD nM |
|---|---|---|
| Human IgG1/K Anti- | SCK/Two state | 7.8 |
| CD19 | reaction | |
| CD19-F5-NKp46-3 | SCK/Two state | 2.6 |
| | reaction | |
| CD19-F6- NKp46-3 | SCK/Two state | 6.0 |
| | reaction | |
| CD19-F13- NKp46-3 | SCK/Two state | 15.2 |
| | reaction | |
| CD19-F14- NKp46-3 | SCK/Two state | 14.0 |
| | reaction | |

### Binding to FcγR

Different multimeric Fc proteins were evaluated to assess whether such bispecific monomeric Fc proteins could retain binding to Fcγ receptors.

SPR measurements were performed on a Biacore T100 apparatus (Biacore GE Healthcare) at 25° C. In all Biacore experiments HBS-EP+ (Biacore GE Healthcare) and 10 mM NaOH, 500 mM NaCl served as running buffer and regeneration buffer respectively. Sensorgrams were analyzed with Biacore T100 Evaluation software. Recombinant human FcR's (CD64, CD32a, CD32b, CD16a and CD16b) were cloned, produced and purified.

F5 and F6 NKCE proteins CD19-F5-NKp46-3 or CD19-F6-NKp46-3 were immobilized covalently to carboxyl groups in the dextran layer on a Sensor Chip CM5. The chip surface was activated with EDC/NHS (N-ethyl-N′-(3-dimethylaminopropyl) carbodiimidehydrochloride and N-hydroxysuccinimide (Biacore GE Healthcare)). Bispecific antibodies were diluted to 10 μg/ml in coupling buffer (10 mM acetate, pH 5.6) and injected until the appropriate immobilization level was reached (i.e. 800 to 900 RU). Deactivation of the remaining activated groups was performed using 100 mM ethanolamine pH 8 (Biacore GE Healthcare).

Monovalent affinity study was assessed following a classical kinetic wizard (as recommended by the manufacturer). Serial dilutions of soluble analytes (FcRs) ranging from 0.7 to 60 nM for CD64 and from 60 to 5000 nM for all the other FcRs were injected over the immobilized bispecific antibodies and allowed to dissociate for 10 min before regeneration. The entire sensorgram sets were fitted using the 1:1 kinetic binding model for CD64 and with the Steady State Affinity model for all the other FcRs.

The results showed that while full length wild type human IgG1 bound to all cynomolgus and human Fcγ receptors, the CD19-F6-NKp46-3 bi-specific antibodies did not bind to any of the receptors. The CD19-F5-NKp46-3, on the other hand, bound to each of the human receptors CD64 (KD=0.7 nM), CD32a (KD=846 nM), CD32b (KD=1850 nM), CD16a (KD=1098 nM) and CD16b (KD=2426 nM). Conventional human anti-IgG1 antibodies have comparable binding to these Fc receptors (KD shown in the table below).

| | | Full length human |
|---|---|---|
| | CD19-F5-NKp46-3 | IgG1 antibody |
| Human Fcγ receptor | KD (nM) | KD (nM) |
| CD64 | 0.7 | 0.24 |
| CD32a | 846 | 379 |
| CD32b | 1850 | 1180 |
| CD16a | 1098 | 630 |
| CD16b | 2426 | 2410 |

### NK Cell Engager Proteins are Potent Across Different Tumor Antigens

NKp46-binding NKCE proteins with Fc domain dimers that bind human CD16 having an arrangement according to the F5 format were constructed in which the tumor antigen-binding VH/VL pairs were varied.

EGFR:

Epidermal growth factor receptor (EGFR) is a transmembrane protein that is activated by binding of its specific ligands, including EGFR and TGFα. Mutations that lead to EGFR overexpression or overactivity have been associated with a number of cancers, including squamous-cell carcinoma of the lung, anal cancer, glioblastoma, and head and neck cancer. An NKp46×EGFR NKCE protein having the F5 format was prepared, incorporating an EGFR ABD comprising the VH and VL domains from the FDA-approved antibody cetuximab (Erbitux™), an NKp46-1 VH/VL pair, and an Fc domain that binds human CD16. The protein was compared to the full-length anti-EGFR IgG1 antibody having the same VH and VL domains.

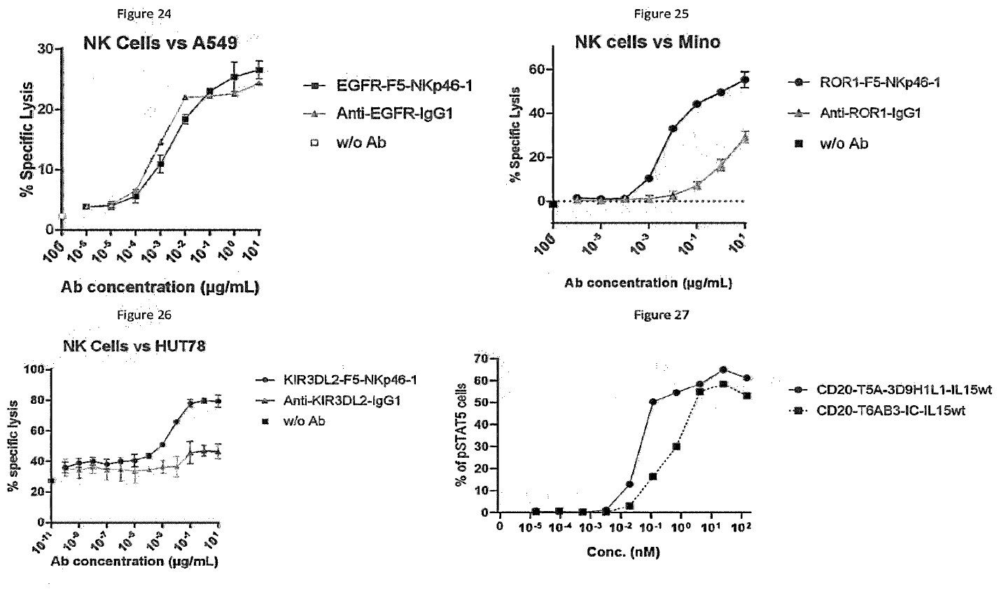

FIG. 24 shows that the NKp46×EGFR NKCE protein, whose Fc domain binds CD16A, is highly potent in mediating A549 target cell lysis, as potent as the full-length IgG1 anti-EGFR antibody, despite the ability of the full-length IgG1 to bind EGFR bivalently.

ROR1:

ROR1 (receptor tyrosine kinase-like orphan receptor 1) is a glycosylated type I membrane protein that belongs to the ROR subfamily of cell surface receptors. It is a pseudokinase that lacks catalytic activity and may interact with the non-canonical Wnt signalling pathway. ROR1 is expressed at very low levels in adult tissues but was found to be expressed at higher levels initially in B-cell chronic lymphocytic leukemia. ROR1 was subsequently found to be expressed moderately or strongly in human cancers derived from a diverse array of tissues, including, e.g., breast, lung, ovarian and pancreatic cancers. An NKp46×ROR1 NKCE protein having the F5 format was prepared, incorporating an NKp46-1 ABD and an Fc domain that binds human CD16. The protein was compared to a full-length IgG1 anti-ROR1 antibody having the same VH and VL domains.

FIG. 25 shows that the NKp46×ROR1 NKCE protein, whose Fc domain binds CD16A, is highly potent in mediating Mino tumor target cell lysis, and moreover, with greater than ten-fold the potency of the full-length IgG1 anti-ROR1 antibody, despite the ability of the full-length IgG1 to bind ROR1 bivalently.

KIR3DL2

KIR3DL2 (CD158k) is a cell surface receptor expressed on a subset of healthy circulating NK and CD8+ T lymphocytes. KIR3DL2 has also been found on the surface of malignant tumor cells in cutaneous T cell lymphomas and in a variety of types of peripheral T cell lymphomas. An NKp46×KIR3DL2 NKCE having the structure of Format 5 was prepared, incorporating an NKp46-1 ABD and an Fc domain that binds human CD16. The protein was compared to a full-length IgG1 anti-KIR3DL2 antibody having the same VH and VL domains.

FIG. 26 shows that the NKp46×KIR3DL2 NKCE protein, whose Fc domain binds CD16A, is highly potent in mediating HUT78 tumor target cell lysis, and moreover, it is significantly more potent than the full-length IgG1 anti-KIR3DL2 antibody despite the ability of the full-length IgG1 to bind KIR3DL2 bivalently.

### Example 19: NKCE-IL15 Promotes Activation of NK Cells

Heterotrimeric NKp46-binding NKCE proteins in the “T5” format of FIG. 2 were prepared as for the IL-2v-containing NKCE molecules, but comprising a wild-type IL-15 moiety in place of the IL-2v moiety, and assessed for its ability to promote IL-2R activation of NK cells. The NKCE were made to bind CD20 by incorporating anti-CD20 VH/VL pair from the FDA-approved humanized antibody GA101.

The heterotrimeric Fc-domain-containing protein CD20-T5A-NKp46-IL15 was assessed for its ability to activate cytokine receptor signalling in NK cells. The CD20-T5A-NKp46-IL15 protein has the domain structure shown in FIG. 2G (structure T5A), and the amino acid sequence shown in Table 6 and polypeptide chains of SEQ ID NOS: 447-479, and has an antigen binding domain that binds CD20 positioned N-terminally, and incorporates a C-terminal positioned IL15, a wild-type Fc domain (i.e. that binds human CD16A), and an antigen binding domain that binds NKp46 derived from the VH/VL pair NKp46-4 placed between the Fc domain and the IL15 moiety. Also tested was the CD20-T6AB3-IC-IL15 protein which has the same amino acid sequence as the CD20-T5A-NKp46-IL15 except that the NKp46 binding VH/VL pair is replaced by a VH/VL pair that does not bind to any antigen in the experimental system, and the Fc domain comprises the mutation N297S (Kabat EU numbering) to abolish binding to CD16A.

Briefly, 1M/well of purified PBMC were seeded in 96-well plates and treated with increasing doses of NKCE-IL15 for 20 min at 37° C. STAT5 phosphorylation was then analysed by flow cytometry on NK cells (CD3-CD56+).

Results are shown in FIG. 27 showing % of pSTAT5 cells among NK cells on the y-axis and concentration of test protein on the x-axis. CD20-T5A-NKp46-IL15 induced a decrease of at least one order of magnitude in the EC₅₀ for STAT5 phosphorylation among the NK cells, compared to CD20-T6AB3-IC-IL15 that did not bind NKp46 or CD16A.

### Example 20: NKCE-IL15 Promotes NK Cell Proliferation

In this experiment, wild-type IL15-containing NK cell engager proteins CD20-T5A-NKp46-IL15 and CD20-T6AB3-ICb-IL15 were assessed for their ability to induce proliferation of NK cells, CD4 T cells or CD8 T cells, upon 6 days of incubation.

Briefly, CellTraceViolet labelled-purified PBMC were cultured with a dose range of NKCE-IL15 for 6 days. Cell proliferation was evaluated by quantification of the percentage of cells showing a diluted CellTraceViolet (CTV) signal monitored by flow cytometry, on NK cells (CD3− CD56+), CD4 T cells (CD3+ CD4+) and CD8 T cells (CD3+ CD8+).

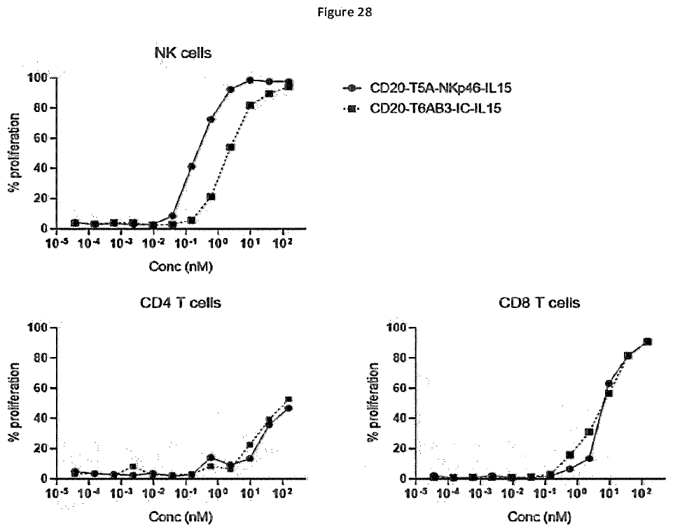

Results are shown in FIG. 28 showing % of proliferating NK, CD4 T or CD8 T cells on the y-axis and concentration of test protein on the x-axis. The CD20-T5A-NKp46-IL15 resulted in strong NK cell proliferation, with an increase in potency of at least 1-log for NK cell proliferation compared to the CD20-T6AB3-IC-IL15 protein that did not bind NKp46 or CD16A. The increase in potency was selective for NK cells, as there was no increase potency by CD20-T5A-NKp46-IL15 for induction of CD4 and CD8 T cell proliferation.

### Example 21: NKCE-IL15 Promotes Tumor Cell Killing in a Standard Vitro Cytotoxicity Assay

In this experiment, wild-type IL15-containing NK cell engager proteins CD20-T5A-NKp46-IL15 and IC-T5A-NKp46-IL15 were assessed for their ability to induce killing of RAJI tumor cells by purified human NK cells. IC-T5A-NKp46-IL15 protein is identical to CD20-T5A-NKp46-IL15 except that the CD20-binding VH/VL pair is replaced by a VH/VL pair (IC) that does not bind to any antigen present in the experimental system. Briefly, purified NK Cells were cocultured with Raji tumor cells previously loaded with calcein, in a 10 to 1 ratio. Cells were incubated with test proteins described above NKCE-IL15 for 4 h at 37° C., 5.5% CO₂ in incubator.

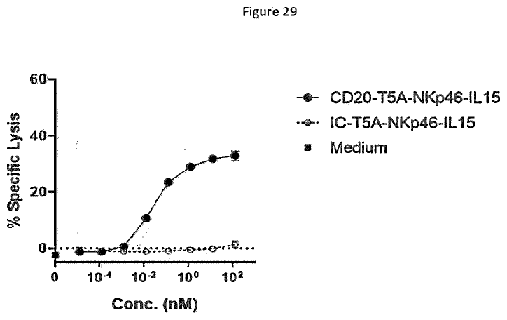

Results are shown in FIG. 29, showing % specific lysis induced by NK cells on the y-axis and concentration of test protein on the x-axis. The IC-T5A-NKp46-IL15 that lacked binding to CD20 on targeted cells did not induce significant cytotoxicity. CD20-T5A-NKp46-IL15 on the other hand displayed high potency in terms of EC50 values in induction of NK cell cytotoxicity toward the tumor cells.

### Example 22: NKCE-IL18v Redirects IL18v Activity to Promote Activation of NK Cells

Heterotrimeric NKp46-binding NKCE proteins in the “T5A” format of FIG. 2G were prepared as for the IL-2v-containing NKCE molecules, but having a variant of IL-18 (IL-18v) moiety in place of the IL-2v moiety, and assessed for its ability to promote IL-18R activation of NK cells. A control protein was prepared having the “T6AB3” structure of FIG. 21. The NKCE proteins were made to bind CD20 by incorporating anti-CD20 VH/VL pair from the FDA-approved humanized antibody GA101.

The heterotrimeric Fc-domain-containing protein CD20-T5A-NKp46-IL18v was assessed for its ability to activate NK cells. The CD20-T5A-NKp46-IL18v protein has the domain structure shown in FIG. 2G, and the amino acid sequence shown in Table 6 and the polypeptide chains of SEQ ID NOS: 486-488, with an antigen binding domain that binds CD20 positioned N-terminally, and incorporates a C-terminal positioned IL18v, a wild-type Fc domain (i.e. that binds human CD16A), and an antigen binding domain that binds NKp46 derived from the VH/VL pair NKp46-4 placed between the Fc domain and the IL18v moiety. The CD20-T6AB3-IC-IL18v protein that did not bind NKp46 or CD16A was used as control molecule that did not bind NK cells.

Briefly, 0.1M/well of purified NK cells were seeded in 96-well plate and treated overnight with increasing doses of each NKCE-IL18v protein, in presence of 10 ng/mL IL-12. Cells were analyzed by flow cytometry for % of CD69 expressing cells and medFI of CD69 expression.

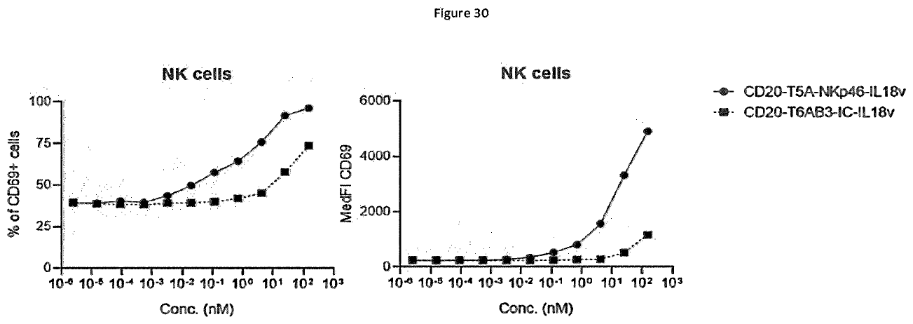

Results are shown in FIG. 30, the left hand panel showing % of CD69 expressing NK cells on the y-axis and concentration of test protein on the x-axis, and right hand panel showing median fluorescence intensity (medFI) of CD69 expression in NK cells. CD20-T5A-NKp46-IL18v induced a decrease in the range of two orders of magnitude in the EC₅₀ for activation of NK cells, compared to CD20-T6AB3-IC-IL18v that did not bind CD16A or NKp46 on NK cells.

### Example 23: NKCE-IL18v Redirects IL18v Activity to Promote IFNγ Production by NK Cells

In this experiment, IL18v-containing NK cell engager proteins CD20-T5A-NKp46-IL18v and CD20-T6AB3-IC-IL18v were assessed for their ability to promote interferon-gamma (IFN-γ) production by NK cells. Purified NK cells were treated overnight with CD20-T5A-NKp46-IL18v or CD20-T6AB3-IC-IL18v, in presence of 10 ng/mL of IL-12 in 96-well plates. IFN-γ was analysed by flow cytometry.

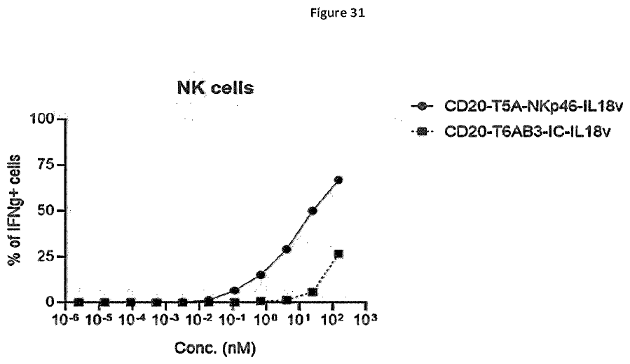

Results are shown in FIG. 31, with % of IFN-γ expressing NK cells on the y-axis and concentration of test protein on the x-axis. CD20-T5A-NKp46-IL18v induced a decrease in the range of two orders of magnitude in the EC₅₀ for activation of NK cells, compared to CD20-T6AB3-IC-IL18v that did not bind CD16A or NKp46 on NK cells.

### Example 24: NKCE-IL18v Promotes NK Cell Proliferation

In this experiment, IL18v-containing NK cell engager proteins CD20-T5A-NKp46-IL18v and CD20-T6AB3-IC-IL18v were assessed for their ability to induce proliferation of NK cells, CD4 T cells or CD8 T cells, upon 6 days of incubation. The CD20-T6AB3-IC-IL18v protein has the same amino acid sequence as the CD20-T5A-NKp46-IL18v except that the NKp46 binding VH/VL pair is replaced by a VH/VL pair that does not bind to any antigen in the experimental system, and the Fc domain comprises the mutations N297S to abolish binding to CD16A.

Briefly, CellTraceViolet labelled-purified PBMC were cultured with a dose range of NKCE-IL18v for 6 days days. Cell proliferation was evaluated by quantification of the percentage of cells showing a diluted CellTraceViolet (CTV) signal monitored by flow cytometry, on NK cells (CD3-CD56+), CD4 T cells (CD3⁺CD4+) and CD8 T cells (CD3+CD8+).

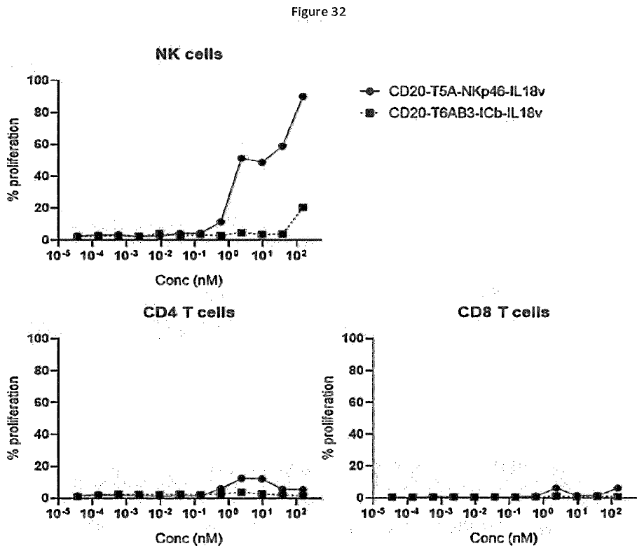

Results are shown in FIG. 32 showing % of proliferating NK, CD4 T or CD8 T cells on the y-axis and concentration of test protein on the x-axis. The CD20-T5A-NKp46-IL18v resulted in strong NK cell proliferation. The CD20-T6AB3-IC-IL18v protein that did not bind CD16A or NKp46 did not show significant activation of NK cells. The strong NK cell proliferation by CD20A-T5-NKp46-IL18v was selective for NK cells, as there was no significant proliferation induced in CD4 or CD8 T cells. CD20-T6AB3-IC-IL18v also showed no significant induction of CD4 or CD8 T cell proliferation.

### Example 25: NKCE-IL18v Promotes Tumor Cell Killing in a Standard Vitro Cytotoxicity Assay

In this experiment, the IL18v-containing NK cell engager proteins CD20-T5A-NKp46-IL18v and IC-T5A-NKp46-IL18v were assessed for their ability to induce killing of RAJI tumor cells by NK cells from human donors. IC-T5A-NKp46-IL18v protein is identical to CD20-T5A-NKp46-IL18v except that the CD20-binding VH/VL pair is replaced by a VH/VL pair (IC) that does not bind to any antigen present in the experimental system.

Briefly, human purified NK Cells were cocultured with Raji tumor cells previously loaded with calcein, in a 10 to 1 ratio. Cells were incubated with test NKCE-IL18v proteins for 4 h at 37° C., 5.5% CO₂ in an incubator.

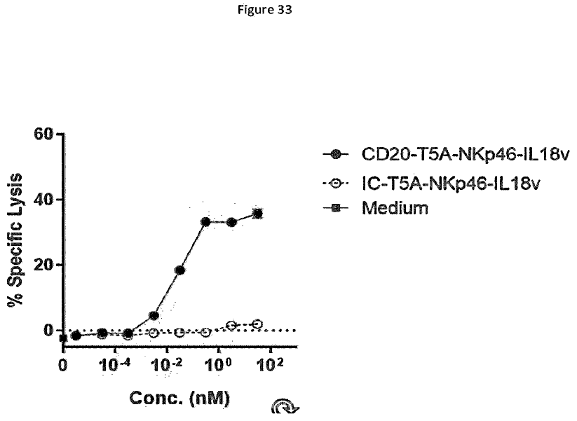

Results are shown in FIG. 33, showing % specific lysis induced by NK cells on the y-axis and concentration of test protein on the x-axis. The IC-T5A-NKp46-IL18v that lacked binding to CD20 on targeted cells did not induce significant cytotoxicity. CD20-T5A-NKp46-IL18v on the other hand displayed high potency in terms of EC₅₀ values in induction of NK cell cytotoxicity toward the tumor cells.

### Example 26: NK Cell Targeting Redirects IFNαv Activity to NK Cells to Promote Activation

Heterotrimeric NKp46-binding NKCE proteins in the “T5A” format of FIG. 2 were prepared as for the IL-2v-containing NKCE molecules, but comprising a variant IFNα (IFNαv) moiety in place of the IL-2v moiety, and assessed for its ability to promote IFNAR-mediated activation of NK cells. The NKCE were made to bind CD20 by incorporating anti-CD20 VH/VL pair from the FDA-approved humanized antibody GA101.

The heterotrimeric Fc-domain-containing protein CD20-T5A-NKp46-IFNαv was assessed for its ability to activate NK cells. The CD20-T5A-NKp46-IFNαv protein has the domain structure shown in FIG. 2G, and the amino acid sequence shown in Table 6 and the polypeptide chains of SEQ ID NOS: 495-497, and has an antigen binding domain that binds CD20 positioned N-terminally, and incorporates a C-terminal positioned IFNαv, a wild-type Fc domain (i.e. that binds human CD16A), and an antigen binding domain that binds NKp46 derived from the VH/VL pair NKp46-4 placed between the Fc domain and the IFNαv moiety. Also tested as control was the CD20-T6AB3-IC-IFNαv protein which has the same amino acid sequence as the CD20-T5A-NKp46-IFNαv except that the NKp46 binding VH/VL pair is replaced by a VH/VL pair that does not bind to any antigen in the experimental system, and the Fc domain comprises the mutation N297S to abolish binding to CD16A.

Briefly, 1M/well of purified PBMC were seeded in 96-well plates and treated with increasing doses of NKCE-IFNαv for 20 minutes at 37° C. STAT3 phosphorylation was then analysed by flow cytometry on NK cells (CD3− CD56+), CD4 T cells (CD3+ CD4+) and CD8 T cells (CD3+ CD8+).

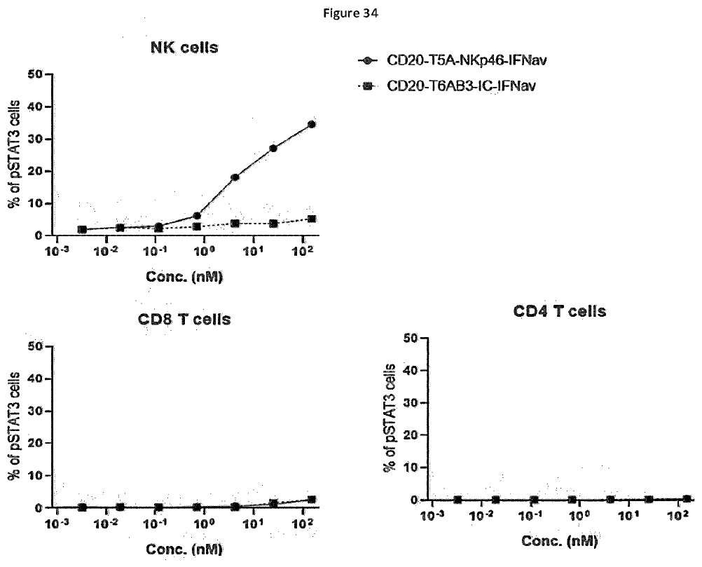

Results are shown in FIG. 34 showing % of pSTAT3 cells among NK cells, CD4 T or CD8 T cells on the y-axis and concentration of test protein on the x-axis. CD20-T5A-NKp46-IFNαv induced potent STAT3 phosphorylation among the NK cells, compared to CD20-T6AB3-IC-IFNαv that did not induce any significant NK cell activation. The potent NK cell activating activity of CD20-T5A-NKp46-IFNαv was selective for NK cells, as there was no significant activation by of CD4 or CD8 T cells.

### Example 27: NKCE-IFNαv Promotes Tumor Cell Killing in a Standard Vitro Cytotoxicity Assay

In this experiment, the IFNαv-containing NK cell engager proteins CD20-T5A-NKp46-IFNαv and IC-T5A-NKp46-IFNαv were assessed for their ability to induce killing of RAJI tumor cells by NK cells. IC-T5A-NKp46-IFNαv protein is identical to CD20-T5A-NKp46-IFNαv except that the CD20-binding VH/VL pair is replaced by a VH/VL pair (IC) that does not bind to any antigen present in the experimental system.

Briefly, human purified NK Cells were cocultured with Raji tumor cells previously loaded with calcein, in a 10 to 1 ratio. Cells were incubated with test NKCE-IFNαv proteins for 4 h at 37° C., 5.5% CO₂ in an incubator.

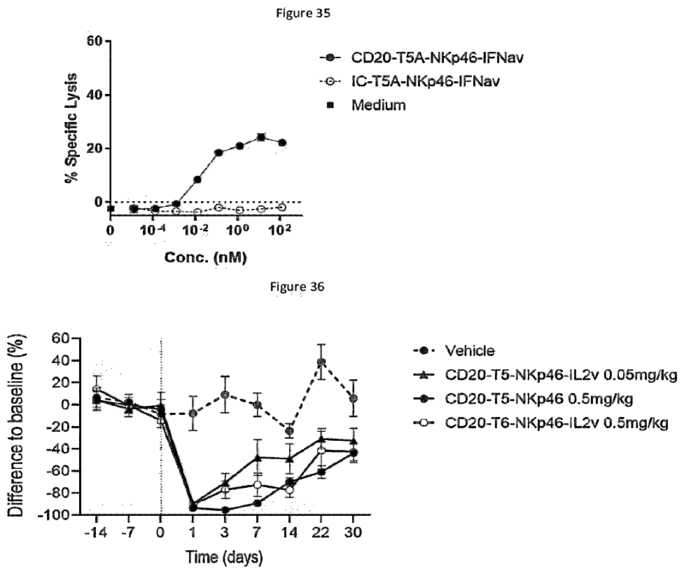

Results are shown in FIG. 35, showing % specific lysis induced by NK cells on the y-axis and concentration of test protein on the x-axis. The IC-T5A-NKp46-IFNαv that lacked binding to CD20 on targeted cells did not induce significant cytotoxicity. CD20-T5A-NKp46-IFNαv on the other hand displayed high potency in terms of EC₅₀ values in induction of NK cell cytotoxicity toward the tumor cells.

### Example 28: NKCE-IL2v Induces Minimal Systemic Cytokine Release In Vivo in Non-Human Primates

Following the favorable safety profile observed in mice following CD20-T5-NKp46-IL2v treatment (see Example 18), we assessed the effect of treatment with the NK cell engager GA101-T5-NKp46-IL2v (SEQ ID NOS: 175-177) in non-human primates. M. fascicularis were injected with a single intravenous injection of either 0.05 mg/kg body weight (the low dose) or 0.5 mg/kg body weight (the high dose) of CD20-T5-NKp46-IL2v (with CD16A binding), or 0.5 mg/kg of GA101-T6-NKp46-IL2v (SEQ ID NOS: 184-186; N297S mutation conferring lack of binding to CD16A), with four animals per dose level.

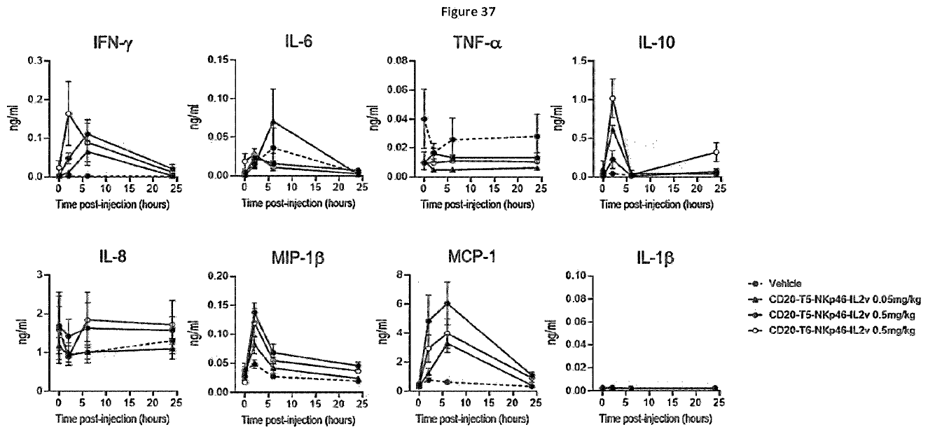

FIG. 36 shows the difference from baseline of the B cell count (cells/μL) over the 14 days before and 30 days following treatment with the NKCE proteins (treatment is Day 0), showing that the NKCE proteins induced B cell depletion, while control (vehicle) did not.

FIG. 37 shows production of different cytokines over the course of 24 hours following administration of the NKCE proteins. For each time point post treatment, the plasma concentration of cytokine is shown in ng/mL sera. These data show that the CD20-T5-NKp46-IL2v induced only minimal systemic cytokine production during the 24 hours following treatment, suggesting that the CD20-T5-NKp46-IL2v does not involve immune toxicity, despite its potent cell depleting activity. The safety profile is particularly remarkable considering the cytokine concentration values observed in comparable experiments with T cell engager protein, for example Engelberts et al., (Ebiomedicine 2020 vol. 52, 102625) report the following values upon administration of 1 mg/kg DuoBody®-CD3×CD20, a full-length human IgG1 bispecific antibody (bsAb) recognizing CD3 and CD20, generated by controlled Fab-arm exchange, with an Fc domain silenced by introduction of mutations: IFN-γ (greater than 1 ng/mL), IL-6 (greater than 8 ng/mL), TNFα (greater than 1.7 ng/mL), IL-10 (greater than 10 ng/mL), IL-8 (greater than 5 ng/mL), MCP (greater than 500 ng/mL).

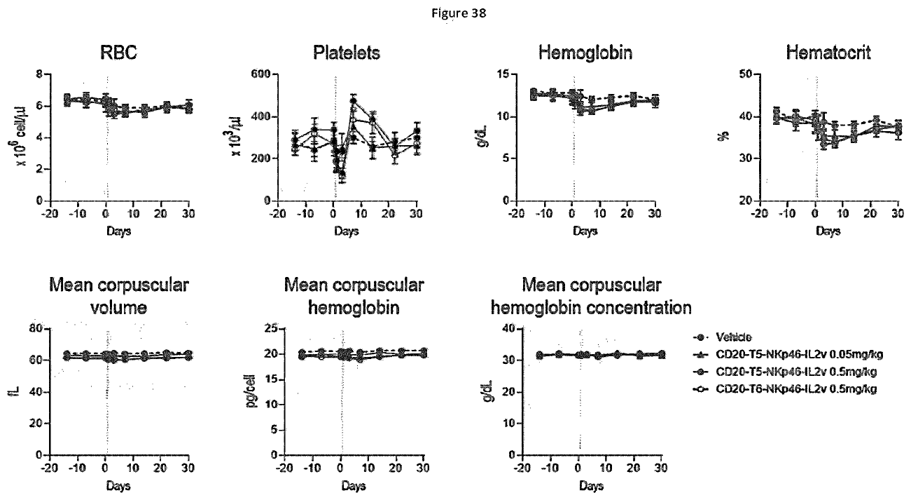

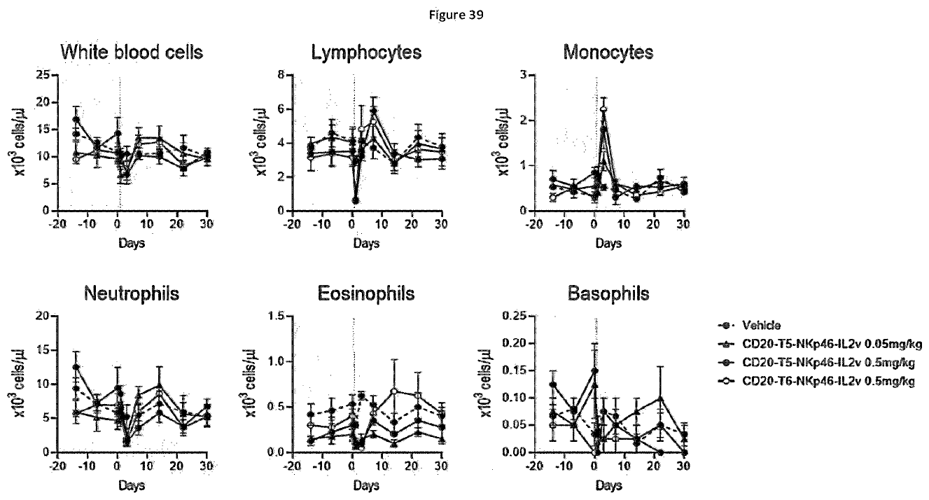

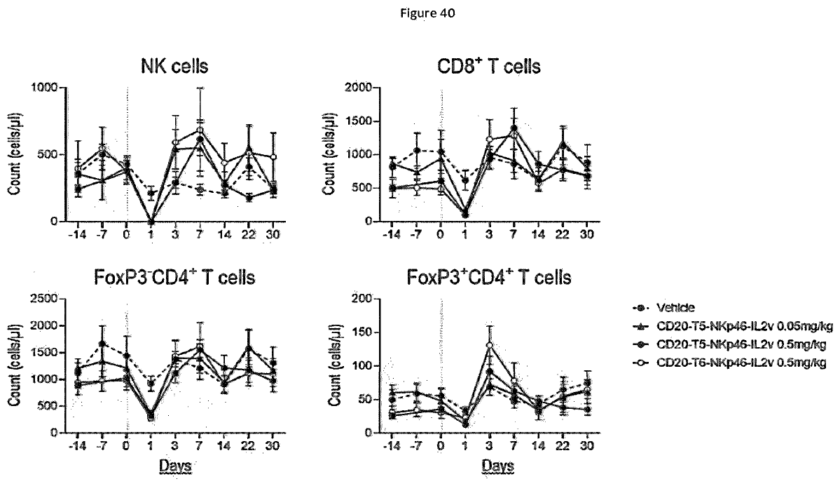

No reactions were observed at the injection site. Behavioral scoring was carried out on a scale from 1 to 30, where 20-30 indicates increasing severe distress, 12-20 indicates increasing signs of suffering, 12 indicates strong alteration of behavior, 7 indicates moderate behavior alteration and 1 indicates healthy individuals. NKCEs did not cause any suffering and yet further were well below the level associated with even moderate behavioral alteration. Upon clinical examination, all parameters observed remained within the normal range of values. Upon blood analysis, all parameters observed for animals treated with the NKCEs remained generally similar to vehicle and within the normal range of values, including blood levels of sodium, potassium, chlorine, bicarbonates, phosphorus, ferritin and fructosamine, albumin, globulin, creatinine, urea, total protein, cholesterol, triglycerides, ALAT, ASAT an alkaline phosphatase. FIG. 38 shows mean values for red blood cells, platelets, hemoglobin, hematocrit, mean corpuscular volume, mean corpuscular hemoglobin and mean corpuscular hemoglobin concentration for animals treated with the NKCEs or vehicle over a period from 14 days before treatment through 30 days following treatment. FIG. 39 shows mean levels of white blood cells, lymphocytes, monocytes, neutrophils, eosinophils and basophils cells over a period from 14 days before treatment through 30 days following treatment. FIG. 40 shows mean levels of NK cells, CD8⁺ T cells, FoxP3⁻CD4⁺ T cells and FoxP3⁺CD4⁺ T cells over a period from 14 days before treatment through 30 days following treatment. All of the NKCE proteins induced an expansion of NK cells at day 3 and day 7. An increase of CD8+ T cells was observed with both CD20-T5-NKp46-IL2v and CD20-T6-NKp46-IL2v at the high dose of proteins but not lower dose of CD20-T5-NKp46-IL2v. An increase of FoxP3⁺CD4⁺ T cells was observed with CD20-T6-NKp46-IL2v but not with either low or high dose of CD20-T5-NKp46-IL2v.

### Example 29: NKCE-IL2v Therapy Increases NK Cells in the Tumor Bed in Immunocompetent Mice

NKCE-IL2v proteins were assessed for their effect on intratumoral accumulation of NK cells in an immunocompetent mouse solid tumor model.

Briefly huCD20 (human CD20) expressing B16F10 melanoma cells were engrafted in C57BL6 mice. At day 1 and day 8, mice were treated with 25 μg of CD20-T5-NKp46-IL2v. Tumors were analysed at day 13 for NK cell infiltration by flow cytometry. NK cells were identified as NK1.1+CD3−.

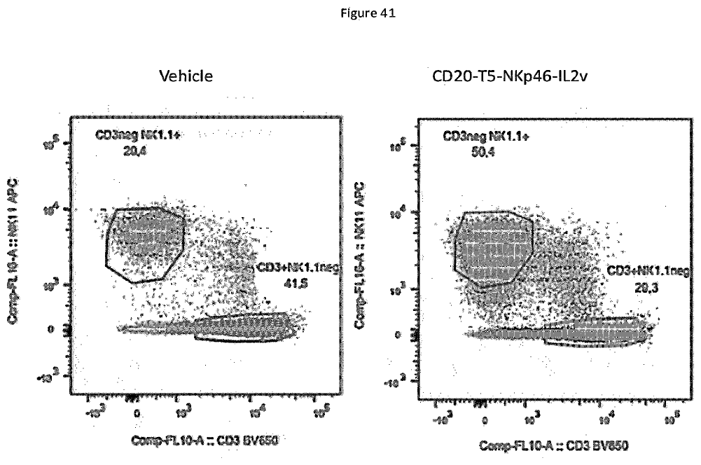

Results are shown in FIG. 41. NK cells (NK1.1+CD3−) represented 20.4% of CD45+ cells in the tumor in the absence of treatment with CD20-T5-NKp46-IL2v. However, when mice were treated with CD20-T5-NKp46-IL2v, the NK cells (NK1.1+CD3−) represented 50.4% of total CD45+ cells in the tumor demonstrating the strong capacity of NKCE-IL2v to stimulate NK cell accumulation at the tumor site.

### Example 30: NKCE-IL2v Treatment Induces NK Cell Expansion and Activation in Spleen of Immunocompromised Mice

NKCE-IL2v proteins were assessed for their effect on NK number and activation cells outside the tumor compartment in an immunocompromised mice model in which NK cells remain functional.

Briefly, CB17 SCID (immunocompromised) bearing RAJI sc tumor were treated with a single i.v injection of 70 μg CD20-T5-NKp46-IL2v, 125 μg CD20-F5-NKp46 (shares the same structure as CD20-T5-NKp46-IL2v but lacks IL2v moiety) or 600 μg obinutuzumab. 3 days after treatment, spleens were analysed by flow cytometry for NK cell number, CD69 and Ki67 expression.

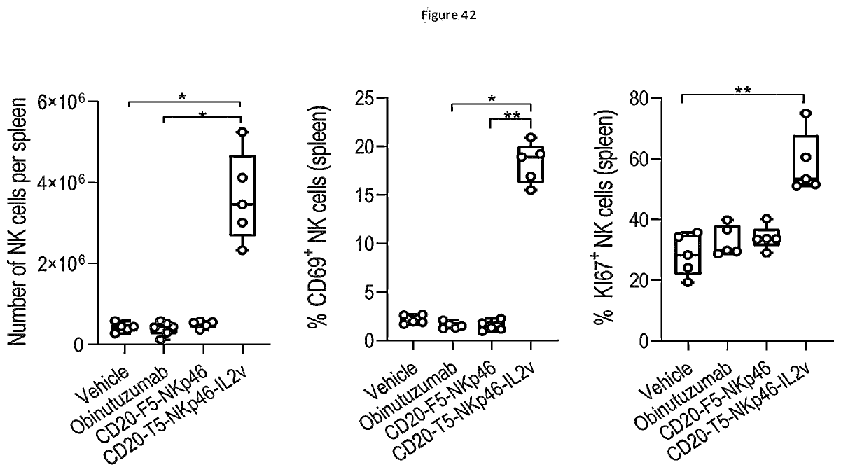

Results are shown in FIG. 42. While treatment with CD20-F5-NKp46 and obinutuzumab did not increase the number of NK cells, treatment with CD20-T5-NKp46-IL2v caused a strong increase in NK cells in the spleen. Additionally, while treatment with CD20-F5-NKp46 or obinutuzumab did not increase the proportion of activated or proliferating NK cells among total NK cells, treatment with CD20-T5-NKp46-IL2v caused a strong increase in activated or proliferating NK cells among total NK cells. These data indicate that NKCE-IL2v can expand the population of NK cells outside of the tumor microenvironment, generating a pool of NK cells that could potentially contribute to antitumor activity in immunocompromised mice.

### Example 31: NKCE-IL2v Treatment Induces NK Cell Expansion and Activation in Blood and Spleen of Immunocompetent Tumor-Bearing Mice

NKCE-IL2v proteins were assessed for their effect on NK cells number and activation in blood and spleen in immunocompenent mice bearing a solid tumor.

Briefly, C57BL6 mice (immunocompetent) bearing huCD20B16F10 (human CD20-expressing mouse B16F10 cells) subcutaneous tumors were treated with 25 μg of CD20-T5-NKp46-IL2v in a single i.v injection. NK cell number and CD69 expression in spleen and blood were analyzed by flow cytometry at day +4 after treatment.

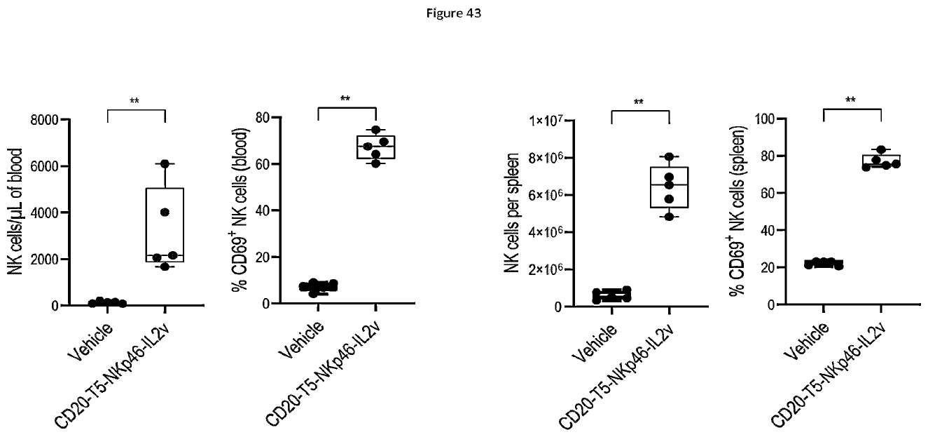

Results are shown in FIG. 43. Treatment with CD20-T5-NKp46-IL2v caused a strong increase in NK cell number per μl of blood and also in the spleen. Additionally, treatment with CD20-T5-NKp46-IL2v caused a strong increase in activated (CD69-expressing) NK cells among NK cells in blood and spleen. The majority of cells in both spleen and blood were activated (in the range of 80% of NK cells were CD69-positive, compared to 20% in the control (vehicle) treated mice). These data indicate that NKCE-IL2v can expand the population of NK cells in blood and spleen, generating a pool of NK cells that could potentially contribute to antitumor activity in immunocompetent mice.

### Example 32: Epitope Mapping of Anti-NKp46 Antibodies

A. Competition Assays

Competition assays were conducted by Surface Plasmon Resonance (SPR according to the methods described below.

SPR measurements were performed on a Biacore T100 apparatus (Biacore GE Healthcare) at 25° C. In all Biacore experiments HBS-EP+(Biacore GE Healthcare) and NaOH 10 mM NaCl 500 mM served as running buffer and regeneration buffer respectively. Sensorgrams were analyzed with Biacore T100 Evaluation software. Anti-6×His tag antibody was purchased from QIAGEN. Human 6×His tagged NKp46 recombinant proteins (NKp46-His) were cloned, produced and purified at Innate Pharma.

Anti-His antibodies were immobilized covalently to carboxyl groups in the dextran layer on a Sensor Chip CM5. The chip surface was activated with EDC/NHS (N-ethyl-N′-(3-dimethylaminopropyl) carbodiimidehydrochloride and N-hydroxysuccinimide (Biacore GE Healthcare)). Protein-A and anti-His antibodies were diluted to 10 μg/ml in coupling buffer (10 mM acetate, pH 5.6) and injected until the appropriate immobilization level was reached (i.e. 2000 to 2500 RU). Deactivation of the remaining activated groups was performed using 100 mM ethanolamine pH 8 (Biacore GE Healthcare).

Parental regular human IgG1 chimeric antibodies having NKp46 binding region corresponding to NKp46-1, NKp46-2, NKp46-3 or NKp46-4 were used for the competition study which has been performed using an Anti-6×His tag antibody chip. Bispecific antibodies having NKp46 binding region based on NKp46-1, NKp46-2, NKp46-3 or NKp46-4 at 1 μg/mL were captured onto Protein-A chip and recombinant human NKp46 proteins were injected at 5 μg/mL together with a second test bispecific antibody of the NKp46-1, NKp46-2, NKp46-3 or NKp46-4 group.

The results demonstrated that none of NKp46-1, NKp46-2, NKp46-3 or NKp46-4 competed with one another for binding to NKp46. Accordingly these antibodies each bind or interact with a different NKp46 epitope.

B. Binding to NKp46 Mutants

In order to define the epitopes of these anti-NKp46 antibodies, we designed NKp46 mutants defined by one, two or three substitutions of amino acids exposed at the molecular surface over the 2 domains of NKp46. This approach led to the generation of 42 mutants which were transfected in Hek-293T cells, as shown in the table below. The targeted amino acid mutations in Table 9 below are shown both according to the numbering of SEQ ID NO: 1 (also corresponding to the numbering used in Jaron-Mendelson et al. (2012) J. Immunol. 88(12):6165-74.

TABLE 9

| | |
|---|---|
| | Substitution (Numbering according to: |
| Mutant | Jaron-Mendelson and SEQ ID NO 1) |

| | | | |
|---|---|---|---|
| 1 | P40A | K43S | Q44A |
| 2 | K41S | E42A | E119A |
| 3 | P86A | D87A | |
| 4 | N89A | R91A | |
| 5 | K80A | K82A | |
| 5bis | E34A | T46A | |
| 6 | R101A | V102A | |
| 7 | N52A | Y53A | |
| 8 | V56A | P75A | E76A |
| 9 | R77A | I78A | |
| 10 | S97A | I99A | |
| 10bis | Q59A | H61A | |
| 11 | L66A | V69A | |
| 12 | E108A | | |
| 13 | N111A | L112A | |
| 14 | D114A | | |
| 15 | T125A | R145S | D147A |
| 16 | S127A | Y143A | |
| 17 | H129A | K139A | |
| 18 | K170A | V172A | |
| 19 | I135A | S136A | |
| 19bis | T182A | R185A | |
| 20 | R160A | | |
| 21 | K207A | | |
| 22 | M152A | R166A | |
| 23 | N195A | N196A | |
| Stalk1 | D213A | I214A | T217A |
| Stalk2 | F226A | T233A | |
| Stalk3 | L236A | T240A | |
| Supp1 | F30A | W32A | |
| Supp2 | F62A | F67A | |
| Supp3 | E63A | Q95A | |
| Supp4 | R71A | K73A | |
| Supp5 | Y84A | | |
| Supp6 | E104A | L105A | |
| Supp7 | Y121A | Y194A | |
| Supp8 | P132A | E133A | |
| Supp9 | S151A | Y168A | |
| Supp10 | S162A | H163A | |
| Supp11 | E174A | P176A | |
| Supp12 | P179A | H184A | |
| Supp13 | R189A | E204A | P205A |

C. Generation of Mutants

NKp46 mutants were generated by PCR. The sequences amplified were run on agarose gel and purified using the Macherey Nagel PCR Clean-Up Gel Extraction kit. Two or three purified PCR products generated for each mutant were then ligated into an expression vector, with the ClonTech InFusion system. The vectors containing the mutated sequences were prepared as Miniprep and sequenced. After sequencing, the vectors containing the mutated sequences were prepared as Midiprep using the Promega PureYield™ Plasmid Midiprep System. HEK293T cells were grown in DMEM medium (Invitrogen), transfected with vectors using Invitrogen's Lipofectamine 2000 and incubated at 37° C. in a CO₂ incubator for 24 hours prior to testing for transgene expression.

D. Flow Cytometry Analysis of Anti-NKp46 Binding to the HEK293T Transfected Cells

All the anti-NKp46 antibodies were tested for their binding to each mutant by flow cytometry. A first experiment was performed to identify antibodies that lose their binding to one or several mutants at a particular concentration (10 μg/ml). To confirm the loss of binding, titration of antibodies was done using antibodies for which binding seemed to be affected by the NKp46 mutations (1-0.1-0.01-0.001 μg/ml).

E. Results

Antibody NKp46-1 had decreased binding to the mutant 2 (having a mutation at residues K41, E42 and E119) (numbering in NKp46 wild-type) compared to wild-type NK46. Similarly, NKp46-1 also had decreased binding to the supplementary mutant Supp7 (having a mutation at residues Y121 and Y194).

Antibody NKp46-3 had decreased binding to the mutant 19 (having a mutation at residues 1135, and S136). Similarly, NKp46-3 also had decreased binding to the supplementary mutant Supp8 (having a mutation at residues P132 and E133). Antibody NKp46-4 had decreased binding to the mutant 6 (having a mutation at residues R101, and V102). Similarly, NKp46-4 also had decreased binding to the supplementary mutant Supp6 having a mutation at residues E104 and L105.

Using these methods we identified the epitopes for anti-NKp46 antibodies NKp46-1, NKp46-3 and NKp46-4. We determined that the epitopes of NKp46-4, NKp46-3 and NKp46-1 are on NKp46 D1 domain, D2 domain and D1/D2 junction, respectively. R101, V102, E104 and L105 are essential residues for NKp46-4 binding and defined a part of NKp46-4 epitope. The epitope of NKp46-1 epitope includes K41, E42, E119, Y121 and Y194 residues. The epitope of NKp46-3 includes P132, E133, 1135, and S136 residues.

All headings and sub-headings are used herein for convenience only and should not be construed as limiting the invention in any way. Any combination of the above-described elements in all possible variations thereof is encompassed by the invention unless otherwise indicated herein or otherwise clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e. g., all exact exemplary values provided with respect to a particular factor or measurement can be considered to also provide a corresponding approximate measurement, modified by “about,” where appropriate). All methods described herein can be performed in any suitable order unless otherwise indicated herein or otherwise clearly contradicted by context.

The use of any and all examples, or exemplary language (e.g., “such as”) provided herein is intended merely to better illuminate the invention and does not pose a limitation on the scope of the invention unless otherwise indicated. No language in the specification should be construed as indicating any element is essential to the practice of the invention unless as much is explicitly stated.

The description herein of any aspect or embodiment of the invention using terms such as reference to an element or elements is intended to provide support for a similar aspect or embodiment of the invention that “consists of’,” “consists essentially of” or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

This invention includes all modifications and equivalents of the subject matter recited in the aspects or claims presented herein to the maximum extent permitted by applicable law.

All publications and patent applications cited in this specification are herein incorporated by reference in their entireties as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

1. A multispecific protein which is capable of binding to an antigen of interest expressed by a target cell, and which is further capable of binding to NKp46, a cytokine receptor and optionally CD16A at the surface of an NK cell, and which multispecific protein is capable of potentiating NK cell cytotoxicity toward a target cell expressing an antigen of interest, the multispecific protein comprising:
(a) an antigen binding domain (ABD) that binds to an antigen of interest;
(b) an ABD that binds to a human NKp46 polypeptide;
(c) an Fc domain, or a portion an Fc domain capable of binding FcRn; and
(d) an ABD that binds a human cytokine receptor present on NK cells,
wherein:
the ABD that binds to a human NKp46 polypeptide is connected to the Fc domain, optionally via an Ig-derived or non-Ig-derived polypeptide linker, and wherein the the ABD that binds a human cytokine receptor is connected, via a polypeptide linker, to the ABD that binds to a human NKp46 polypeptide or to the Fc domain.

2. The protein of claim 1, wherein the protein has only one ABD that binds to an antigen of interest, such that the protein binds to the antigen of interest monovalently, optionally further wherein the protein has only one ABD that binds to a human NKp46 polypeptide, only one ABD that binds a cytokine receptor, and only one Fc domain dimer.

3. The protein of claim 1 or 2, wherein the ABD that binds the cytokine receptor is connected via a polypeptide linker to the ABD that binds NKp46.

4. The protein of claims 1, 2 or 3, wherein the ABD that binds to a human NKp46 polypeptide and the ABD that binds a human cytokine receptor, and optionally further the Fc domain, are positioned within the multispecific protein in series with respect to the N- and C-termini of the multispecific protein.

5. The protein of any one of the above claims, wherein the ABD that binds to a human NKp46 polypeptide and the ABD that binds a human cytokine receptor, and optionally the Fc domain, are configured to be capable of adopting a membrane-planar binding conformation such that the protein is capable of binding to NKp46 and the cytokine receptor, and optionally further CD16A, on the surface of an NK cell.

6. The protein of any one of the above claims, wherein the ABD that binds a human cytokine is connected to the ABD that binds to a human NKp46 polypeptide or the Fc domain by a linker peptide having 20 or less than 20 amino acid residues, optionally less than 15 amino acid residues, optionally less than 10 amino acid residues, optionally between 5 and 15 residues, optionally between 5 and 10 residues, optionally between 3 and 5 residues.

7. The protein of any one of the above claims, wherein the ABD that binds to a human NKp46 polypeptide is connected to the Fc domain by a linker peptide having 15 or less than 15 amino acid residues, optionally less than 10 amino acid residues, optionally between 4 and 15 residues, optionally between 3 and 10 residues, optionally between 4 and 5 residues.

8. The protein of any one of the above claims, wherein the ABD that binds to a human NKp46 polypeptide is connected to the Fc domain by an immunoglulin-derived linker peptide having 20 or less than 20 amino acid residues, optionally less than 15 amino acid residues, optionally between 4 and 15 residues, optionally between 4 and 10 residues, optionally between 3 and 5 residues, optionally wherein the linker comprises the amino acid sequence RTVA.

9. The protein of any one of the foregoing claims, wherein the ABD that binds NKp46 is interposed between the Fc domain and the ABD that binds the cytokine receptor.

10. The protein of any one of the foregoing claims, wherein each ABD comprises a V_{H} and/or V_{L} domain or fragment thereof, optionally wherein a VH and/or VL comprises three complementary determining regions (CDR1, CDR2 and CDR-3).

11. A protein comprising an ABD that binds to a human NKp46 polypeptide fused, optionally via a domain linker having 15 or less than 15 amino acid residues, to a cytokine that binds a receptor expressed at the surface of an NK cell.

12. The protein of claim of any one of the above claims, wherein the ABD that binds to a human NKp46 polypeptide comprises a V_{H} and/or V_{L} domain that binds to the D1/D2 junction of the human NKp46 polypeptide, and wherein the cytokine is a modified IL-2 polypeptide.

13. The protein of claim 11 or 12, wherein the ABD that binds to a human NKp46 polypeptide is fused at its C-terminus, via a domain linker, to the N-terminus of the cytokine, optionally wherein the ABD that binds to a human NKp46 polypeptide has an scFv or a Fab structure.

14. The protein of any one of the foregoing claims, wherein the protein is a heterodimer comprising a first and second polypeptide chain, or a heterotimer comprising a first, second and third polypeptide chain.

15. The protein of any one of the foregoing claims, comprising:
(i) a first polypeptide chain comprising, from N- to C-terminal, a variable domain that binds NKp46, a human CH1 or CL constant domain, optionally a domain linker, and a wild-type or variant IL-2, IL-15, IL-21, IL-7, IL-27, IL-12, IL-18, IFN-α or IFN-β polypeptide, and
(ii) a second polypeptide chain comprising, from N- to C-terminal, a variable domain that associates with the variable domain of (i) to form a NKp46-binding domain, and a human CH1 or CL constant domain;
wherein one of the constant domains of (i) and (ii) is a CH1 and the other is a CL such that the constants domains of (i) and (ii) associate by CH1-CL dimerization.

16. The protein of any one of the foregoing claims, wherein the protein comprises a NKp46 ABD-cytokine unit having a domain arrangement: 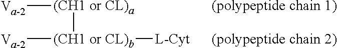
wherein:
Vₐ₋₂ and V_{b-2} are each a V_{H} domain or a V_{L} domain, wherein one of Vₐ₋₂ and V_{b-2} is a V_{H} and the other is a V_{L} and wherein Vₐ₋₂ and V_{b-2} form a second ABD that binds NKp46;
CH1 is a human immunoglobulin CH1 domain and CL is a human light chain constant domain;
one of (CH1 or CL)ₐ and (CH1 or CL)_{b} is a CH1 and the other is a CL such that a (CH1/CL) pair is formed;
L is a domain linker; and
Cyt is a cytokine polypeptide or portion thereof that binds to a cytokine receptor present on NK cells, optionally wherein Cyt is a wild-type or variant human IL-2, IL-15, IL-21, IL-7, IL-27, IL-12, IL-18, IFN-α or IFN-β polypeptide.

17. The protein of any one of the foregoing claims, comprising:
(a) (i) a first polypeptide chain comprising, from N- to C-terminal, an NKp46-binding domain or portion thereof comprising a variable domain comprising an amino acid sequence at least 80%, 90%, 95%, 98% or 99% identical to an amino acid sequence of any of SEQ ID NOS: 3, 5, 7, 9, 11, 13, 112, 113, 115, 116, 117, 119, 120, 121, 123, 124, 125, 127, 128, 129 or 236-313, a human CH1 or CL constant domain, a domain linker, and a wild-type or variant IL-2, IL-15, IL-21, IL-7, IL-27, IL-12, IL-18, IFN-α or IFN-β polypeptide comprising an amino acid sequence at least 80%, 90%, 95%, 98% or 99% identical to an amino acid sequence of any of SEQ ID NOS: 404-439 or to a fragment thereof of at least 40, 50, 60, 80 or 100 contiguous amino acids thereof, and (ii) a second polypeptide chain comprising, from N- to C-terminal, a variable domain that associates with the variable domain of (i) to form a NKp46-binding domain wherein the variable domain comprises an amino acid sequence at least 80%, 90%, 95%, 98% or 99% identical to an amino acid sequence of any of SEQ ID NOS: 4, 6, 8, 10, 12, 14, 114, 118, 122, 126, 130 or 314-403, and a human CH1 or CL constant domain; or
(b) (i) a first polypeptide chain comprising, from N- to C-terminal, an NKp46-binding domain or portion thereof comprising a variable domain comprising an amino acid sequence at least 80%, 90%, 95%, 98% or 99% identical to an amino acid sequence of any of SEQ ID NOS: 4, 6, 8, 10, 12, 14, 114, 118, 122, 126, 130 or 314-403, a human CH1 or CL constant domain, a domain linker, and a wild-type or variant IL-2, IL-15, IL-21, IL-7, IL-27, IL-12, IL-18, IFN-α or IFN-β polypeptide comprising an amino acid sequence at least 80%, 90%, 95%, 98% or 99% identical to an amino acid sequence of any of SEQ ID NOS: 404-439 or to a fragment thereof of at least 40, 50, 60, 80 or 100 contiguous amino acids thereof, and
(ii) a second polypeptide chain comprising, from N- to C-terminal, a variable domain that associates with the variable domain of (i) to form a NKp46-binding domain wherein the variable domain comprises an amino acid sequence at least 80%, 90%, 95%, 98% or 99% identical to an amino acid sequence of any of SEQ ID NOS: 3, 5, 7, 9, 11, 13, 112, 113, 115, 116, 117, 119, 120, 121, 123, 124, 125, 127, 128, 129 or 236-313, and a human CH1 or CL constant domain.

18. The protein of any one of claims 1-13, wherein the protein comprises a polypeptide comprising a NKp46 ABD-cytokine unit having a domain arrangement
L1-Vₐ₋₂-L2-V_{b-2}-L3-Cyt
wherein:
Vₐ₋₂ and V_{b-2} are each a V_{H} domain or a V_{L} domain, wherein one of Vₐ₋₂ and V_{b-2} is a V_{H} and the other is a V_{L} and wherein Vₐ₋₂ and V_{b-2} form a second ABD that binds NKp46; L1, L2 and L3 are each a domain linker, wherein L1, L2 and L3 can be different or the same, wherein L1 is a domain linker connecting the NKp46 ABD-cytokine unit to the rest of the protein; and Cyt is a cytokine polypeptide or portion thereof that binds to a cytokine receptor present on NK cells, optionally wherein Cyt is a wild-type or variant human IL-2, IL-15, IL-21, IL-7, IL-27, IL-12, IL-18, IFN-α or IFN-β polypeptide.

19. The protein of any one of the foregoing claims, wherein the ABD that binds a cytokine receptor comprises an IL-2 polypeptide displaying reduced binding to CD25 compared to a wild-type human IL-2 polypeptide.

20. The protein of any one of the foregoing claims, wherein the protein or the ABD that binds to a human NKp46 polypeptide binds human NKp46 polypeptide with a KD of between 1 and 100 nM, as determined by SPR and/or wherein the protein or the cytokine binds the human cytokine receptor with a KD of between 10 nM and 1 μM, as determined by SPR.

21. The protein of any one of the above claims, wherein the multispecific polypeptide binds to a human Fcγ receptor, optionally CD16A, with an affinity for monovalent binding, as assessed by surface plasmon resonance, that is substantially equivalent to that of a full-length wild type human IgG1 antibody.

22. The protein of any one of the above claims, wherein the protein, immobilized on a surface, binds a soluble human CD16A polypeptide with a KD for monovalent binding that is no more than 2000 nM, optionally 1000-2000 nM, optionally 1100 or 1300 nM, as determined by surface plasmon resonance using Biacore.

23. The protein of any one of the foregoing claims, wherein the cytokine receptor is a receptor present at the surface of NK cells and that associates with the common γ-chain (γC; CD132), optionally further wherein wherein the cytokine receptor is the mid-affinity IL-2/IL-15Rβ receptor (CD122) or IL-21R (CD360), and optionally wherein the cytokine polypeptide is IL-2, IL-15 or IL-21.

24. The protein of any one of claims 1-17 or 19-23, wherein the protein is an isolated heterotrimeric polypeptide comprising:
(a) a first polypeptide chain comprising, from N- to C-terminus, a first variable domain (V), a first CH1 of CK constant region, a Fc domain or portion thereof, a second variable domain (V) and a second CH1 of CK constant region;
(b) a second polypeptide chain comprising, from N- to C-terminus, a first variable domain (V), a CH1 or CK constant region, and a Fc domain or portion thereof, wherein the V domain and CH1 or Cκ constant region are selected to be complementary to the first V domain and first CH1 or Cκ constant region of the first polypeptide chain such that the first and second polypeptides form a CH1-Cκ heterodimer in which the first variable domain of the first polypeptide chain and the first variable domain of the second polypeptide form a first antigen binding domain that binds the antigen of interest; and
(c) a third polypeptide chain comprising, from N- to C-terminus, a variable domain (V) and a CH1 or CK constant region, wherein the V domain and CH1 or Cκ constant region are selected to be complementary to the second V domain and second CH1 or Cκ constant region of the first polypeptide chain such that the first and third polypeptides form a CH1-Cκ heterodimer in which the second variable domain of the first polypeptide chain and the variable domain of the third polypeptide form a second antigen binding domain that binds NKp46, and
wherein one of the first or third polypeptide chains further comprises a cytokine, optionally an IL-2, IL-15, IL-21, IL-7, IL-27, IL-12, IL-18, IFN-α or IFN-β polypeptide, optionally an IL-2v or IL15v, covalently attached, optionally via a domain linker, to its C-terminus.

25. The multispecific protein of claims 1-17 or 19-24, wherein the protein is a heterotrimer having a polypeptide chain 1, 2 and 3: 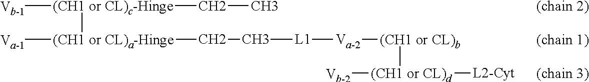
wherein:
Vₐ₋₁, V_{b-1}, Vₐ₋₂ and V_{b-2} are each a V_{H} domain or a V_{L} domain, wherein one of Vₐ₋₁ and V_{b-1} is a V_{H} and the other is a V_{L} and wherein Vₐ₋₁ and V_{b-1} form a first antigen binding domain (ABD) that binds an antigen of interest, wherein one of Vₐ₋₂ and V_{b-2} is a V_{H} and the other is a V_{L} and wherein Vₐ₋₂ and V_{b-2} form a second ABD that binds NKp46;
CH1 is a human heavy chain constant domain 1 and CL is a human light chain constant domain;
one of (CH1 or CL)ₐ and (CH1 or CL)_{c} is a CH1 and the other is a CL such that a (CH1/CL) pair is formed;
one of (CH1 or CL)_{b} and (CH1 or CL)_{d} is a CH1 and the other is a CL such that a (CH1/CL) pair is formed;
Hinge is an immunoglobulin hinge region or portion thereof;
L1 and L2 are each a domain linker, wherein L1 and L2 can be different or the same;
CH2 and CH3 are human immunoglobulin CH2 and CH3 domains, respectively; and
Cyt is a cytokine polypeptide or portion thereof that binds to a cytokine receptor present on NK cells, optionally wherein Cyt is a wild-type or variant human IL-2, IL-15, IL-21, IL-7, IL-27, IL-12, IL-18, IFN-α or IFN-β polypeptide.

26. The protein of any one of claims 1-17 or 19-25, having the domain arrangement:

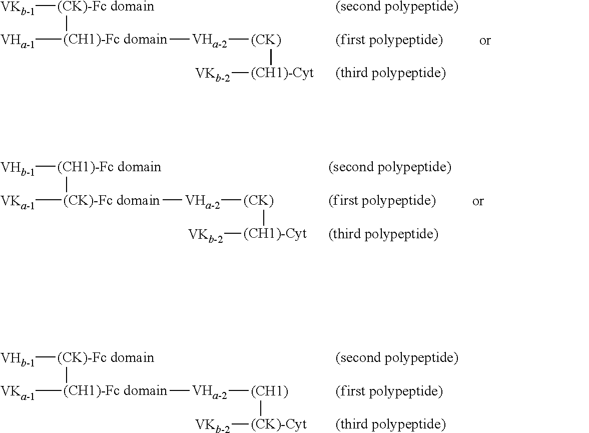

27. The protein of any one of claims 1-17 or 19-24, wherein the protein is a heterotrimer having a polypeptide chain 1, 2 and 3: 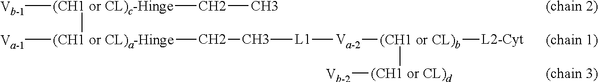
wherein:
Vₐ₋₁, V_{b-1}, Vₐ₋₂ and V_{b-2} are each a V_{H} domain or a V_{L} domain, wherein one of Vₐ₋₁ and V_{b-1} is a V_{H} and the other is a V_{L} and wherein Vₐ₋₁ and V_{b-1} form a first antigen binding domain (ABD) that binds an antigen of interest, wherein one of Vₐ₋₂ and V_{b-2} is a V_{H} and the other is a V_{L} and wherein Vₐ₋₂ and V_{b-2} form a second ABD that binds NKp46;
CH1 is a human heavy chain constant domain 1 and CL is a human light chain constant domain;
one of (CH1 or CL)ₐ and (CH1 or CL)_{c} is a CH1 and the other is a CL such that a (CH1/CL) pair is formed;
one of (CH1 or CL)_{b} and (CH1 or CL)_{d} is a CH1 and the other is a CL such that a (CH1/CL) pair is formed;
Hinge is an immunoglobulin hinge region or portion thereof;
L1 and L2 are each a domain linker, wherein L1 and L2 can be different or the same;
CH2 and CH3 are human immunoglobulin CH2 and CH3 domains, respectively; and
Cyt is a cytokine polypeptide or portion thereof that binds to a cytokine receptor present on NK cells, optionally wherein Cyt is a wild-type or variant human IL-2, IL-15, IL-21, IL-7, IL-27, IL-12, IL-18, IFN-α or IFN-β polypeptide.

28. The protein of any one of claims 1-17, 19-24 or 27, having the domain arrangement:

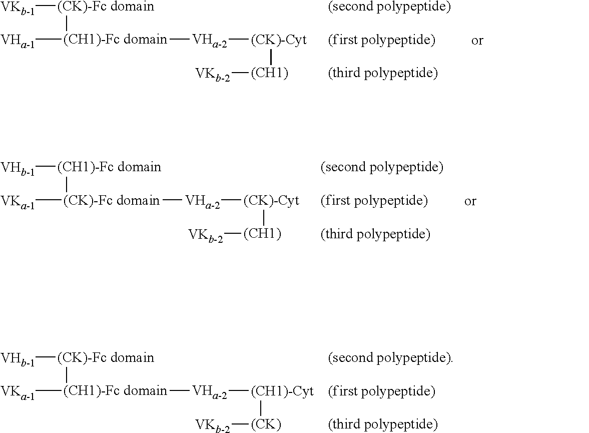

29. The protein of any one of claims 1-14 or 18-23, wherein the protein is an isolated heterodimeric polypeptide comprising:
(a) a first polypeptide chain comprising, from N- to C-terminus, a first variable domain (V), a CH1 of Cκ constant region, a Fc domain or portion thereof, a second variable domain and a third variable domain, and a wild-type or variant cytokine that binds to a cytokine receptor present on NK cells; and
(b) a second polypeptide chain comprising, from N- to C-terminus, a first variable domain (V), a CH1 or CK constant region, and a Fc domain or portion thereof, wherein the CH1 or Cκ constant region is selected to be complementary to the CH1 or Cκ constant region of the first polypeptide chain such that the first and second polypeptides form a CH1-Cκ heterodimer in which the first variable domain of the first polypeptide chain and the first variable domain of the second polypeptide form a first antigen binding domain; and wherein the second variable domain and third variable domain forms a second antigen binding domain.

30. The protein of any one of claims 1-14, 18-23 or 29, wherein the protein is a heterodimer having a polypeptide chain 1 and 2: 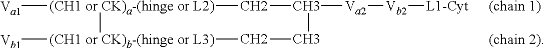
wherein:
Vₐ₋₁, V_{b-1}, Vₐ₋₂ and V_{b-2} are each a V_{H} domain or a V_{L} domain, wherein one of Vₐ₋₁ and V_{b-1} is a V_{H} and the other is a V_{L} and wherein Vₐ₋₁ and V_{b-1} form a first antigen binding domain (ABD) that binds an antigen of interest, wherein one of Vₐ₋₂ and V_{b-2} is a V_{H} and the other is a V_{L} and wherein Vₐ₋₂ and V_{b-2} form a second ABD that binds NKp46;
CH1 is a heavy chain constant domain 1 and CL is a light chain constant domain;
one of (CH1 or CL)ₐ and (CH1 or CL)_{b} is a CH1 and the other is a CL such that a (CH1/CL) pair is formed;
Hinge is an immunoglobulin hinge region or portion thereof;
L1, L2 and L3 are each a domain linker, wherein L1, L2 and L3 can be different or the same;
CH2 and CH3 are human immunoglobulin CH2 and CH3 domains, respectively; and
Cyt is a cytokine polypeptide or portion thereof that binds to a cytokine receptor present on NK cells, optionally wherein Cyt is a wild-type or variant human IL-2, IL-15, IL-21, IL-7, IL-27, IL-12, IL-18, IFN-α or IFN-β polypeptide.

31. The protein of any one of the above claims, wherein the ABD that binds to an antigen of interest comprises an immunoglobulin heavy chain variable domain (VH) and an immunoglobulin light chain variable domain (VL), wherein the VH region comprises an amino acid sequence having at least about 80%, 85%, 90%, 95%, 97%, 98% or 99% identity, to the amino acid sequence of SEQ ID NOS: 132, 134, 136, 138, 140, 142, 144, 146, 148, 150, 152, 154 or any of 236-313, and wherein the VL comprises an amino acid sequence having at least about 80%, 85%, 90%, 95%, 97%, 98% or 99% identity, to the amino acid sequence of SEQ ID NOS: 133, 135, 137, 139, 141, 143, 145, 147, 149, 151, 153, 155 or any of 314-403.

32. The protein of any one of the above claims, wherein the ABD that binds to NKp46 comprises an immunoglobulin heavy chain variable domain (VH) and an immunoglobulin light chain variable domain (VL), wherein the VH region comprises an amino acid sequence having at least about 80%, 85%, 90%, 95%, 97%, 98% or 99% identity, to the amino acid sequence of SEQ ID NOS: 3, 5, 7, 9, 11, 13, 112, 113, 115, 116, 117, 119, 120, 121, 123, 124, 125, 127, 128, 129 or any of 236-313, and wherein the VL comprises an amino acid sequence having at least about 80%, 85%, 90%, 95%, 97%, 98% or 99% identity, to the amino acid sequence of SEQ ID NOS: 4, 6, 8, 10, 12, 14, 114, 118, 122, 126, 130 or any of 314-403.

33. The protein of any one of the above claims, wherein an Fc domain comprises an amino acid sequence having at least about 80%, 85%, 90%, 95%, 97%, 98% or 99% identity to the Fc polypeptide of any of SEQ ID NOS: 160-165.

34. The protein of any one of the above claims, wherein a CH1 domain comprises an amino acid sequence having at least about 80%, 85%, 90%, 95%, 97%, 98% or 99% identity to the CH1 polypeptide of any of SEQ ID NOS: 156.

35. The protein of any one of the above claims, wherein a CK or CL domain comprises an amino acid sequence having at least about 80%, 85%, 90%, 95%, 97%, 98% or 99% identity to the CK polypeptide of any of SEQ ID NOS: 156.

36. The protein of any one of the above claims, wherein the multispecific protein comprises:
(a) an ABD that binds to the antigen of interest, wherein the ABD comprises an scFv or Fab,
a. wherein the scFv comprises a VH comprising an amino acid sequence at least 90% identical to a sequence selected from SEQ ID NOS: 132, 134, 136, 138, 140, 142, 144, 146, 148, 150, 152, 154 and any of 236-313, a domain linker, and a VL comprising an amino acid sequence at least 90% identical to a sequence selected from SEQ ID NOS: 133, 135, 137, 139, 141, 143, 145, 147, 149, 151, 153, 155 and any of 314-403; and
b. wherein the Fab comprises one VH comprising an amino acid sequence at least 90% identical to a selected from SEQ ID NOS: 132, 134, 136, 138, 140, 142, 144, 146, 148, 150, 152, 154 and any of 236-313, one VL comprising an amino acid sequence at least 90% identical to a sequence selected from SEQ ID NOS: 133, 135, 137, 139, 141, 143, 145, 147, 149, 151, 153, 155 and any of 314-403, one human CH1 domain comprising an amino acid sequence at least 90% identical to SEQ ID NO: 156 and one human CL domain comprising an amino acid sequence at least 90% identical to SEQ ID NOS: 159, wherein the VH is fused to one of the CH1 or CL domains, and the VL is fused to the other of the CH1 or CL domains,
(b) an ABD that binds to a human NKp46 polypeptide, wherein the ABD comprises an scFv or Fab,
a. wherein the scFv comprises a VH comprising an amino acid sequence at least 90% identical to a sequence selected from SEQ ID NOS: 3, 5, 7, 9, 11, 13, 112, 113, 115, 116, 117, 119, 120, 121, 123, 124, 125, 127, 128, 129 and any of 236-313, a domain linker, and a VL comprising an amino acid sequence at least 90% identical to a sequence selected from SEQ ID NOS: 4, 6, 8, 10, 12, 14, 114, 118, 122, 126, 130 and any of 314-403; and
b. wherein the Fab comprises one VH comprising an amino acid sequence at least 90% identical to a selected from SEQ ID NOS: 3, 5, 7, 9, 11, 13, 112, 113, 115, 116, 117, 119, 120, 121, 123, 124, 125, 127, 128, 129 and any of 236-313, one VL comprising an amino acid sequence at least 90% identical to a sequence selected from SEQ ID NOS: 4, 6, 8, 10, 12, 14, 114, 118, 122, 126, 130 and any of 314-403, one human CH1 domain comprising an amino acid sequence at least 90% identical to SEQ ID NO: 156 and one human CL domain comprising an amino acid sequence at least 90% identical to SEQ ID NOS: 159, wherein the VH is fused to one of the CH1 or CL domains, and the VL is fused to the other of the CH1 or CL domains,
(c) a Fc domain dimer comprising a first and second Fc monomer wherein each Fc monomer comprises an amino acid sequence at least 80% or 90% identical to a sequence selected from SEQ ID NOS: 160-165; and
(d) a cytokine polypeptide comprising an amino acid sequence at least 80% or 90% identical to a sequence selected from SEQ ID NOS: 404-439, or to a contiguous sequence of at least 40, 50, 60, 70, 80 or 100 amino acid residues thereof, fused, via a domain linker, to the C-terminus of one of the polypeptide chains of the multispecific protein.

37. The protein of any one of the above claims, wherein the target cell is a tumor cell.

38. The protein of any one of the above claims, wherein the antigen of interest is a cancer antigen.

39. The protein of any one of claims 1-37, wherein the antigen of interest is expressed by an infectious agent or infected cell.

40. The protein of any one of the above claims, wherein the multispecific protein competes for binding to a NKp46 polypeptide with a monoclonal antibody comprising the NKp46-1, NKp46-2, NKp46-3, NKp46-4, NKp46-6 or NKp46-9 VH and VL domains.

41. The protein of any one of the above claims, wherein the multispecific protein has decreased binding to a mutant NKp46 polypeptide selected from the group consisting of:
(a) a mutant NKp46 polypeptide having a mutation at residues R101, V102, E104 and/or L105 compared to binding to the wild-type NKp46;
(b) a mutant NKp46 polypeptide having a mutation any one or more of the residues K41, E42, E119, Y121 and/or Y194 compared to binding to the wild-type NKp46; and
(c) a mutant NKp46 polypeptide having a mutation any one or more of the residues P132, E133, 1135, and/or S136 compared to binding to the wild-type NKp46.

42. The protein of any one of the above claims, wherein the antigen binding domain that binds NKp46 comprises:
(a) a heavy chain comprising CDR 1, 2 and 3 of the heavy chain variable region of SEQ ID NO: 3 and a light chain comprising CDR 1, 2 and 3 of the light chain variable region of SEQ ID NO: 4;
(b) a heavy chain comprising CDR 1, 2 and 3 of the heavy chain variable region of SEQ ID NO: 5 and a light chain comprising CDR 1, 2 and 3 of the light chain variable region of SEQ ID NO: 6;
(c) a heavy chain comprising CDR 1, 2 and 3 of the heavy chain variable region of SEQ ID NO: 7 and a light chain comprising CDR 1, 2 and 3 of the light chain variable region of SEQ ID NO: 8;
(d) a heavy chain comprising CDR 1, 2 and 3 of the heavy chain variable region of SEQ ID NO: 9 and a light chain comprising CDR 1, 2 and 3 of the light chain variable region of SEQ ID NO: 10;
(e) a heavy chain comprising CDR 1, 2 and 3 of the heavy chain variable region of SEQ ID NO: 11 and a light chain comprising CDR 1, 2 and 3 of the light chain variable region of SEQ ID NO: 12; or
(f) a heavy chain comprising CDR 1, 2 and 3 of the heavy chain variable region of SEQ ID NO: 13 and a light chain comprising CDR 1, 2 and 3 of the light chain variable region of SEQ ID NO: 14.

43. A pharmaceutical composition comprising a multispecific protein of any one of the above claims, and a pharmaceutically acceptable carrier or adjuvant.

44. A recombinant cell which expresses at one, two, three of four of (or all of) the polypeptide chains of a multispecific protein according to any one of claims 1-42.

45. A method of preparing an NK cell composition, the method comprising incubating or contacting NK cells, optionally isolated NK cells, in vitro, with a multispecific protein according to any one of claims 1-42.

46. A composition of NK cells obtained according to the method of claim 45.

47. Use of a protein or composition of any one of claims 1-42, 43 or 46 as a medicament for the treatment of disease and/or in the manufacture of a medicament for the treatment of disease.

48. A method of potentiating NK cell activation, cytotoxicity and/or proliferation of NKp46⁺CD16⁺ NK cells and/or NKp46⁺CD16⁻ NK cells in a subject having a disease, the method comprising administering to the subject a multispecific protein according to any of claims 1-42.

49. A method delivering or bringing a highly potent cytokine to NK cells and/or to a tumor in a subject having a disease, optionally further with reduced toxicity, the method comprising administering to the subject a multispecific protein according to any of claims 1-42.

50. The method or use of claims 45-49, wherein the cytokine is a variant or wild-type cytokine or cytokine fragment thereof that retains at least 70%, 80% or 90% of the affinity for its cytokine receptor present on NK cells, compared to the wild-type cytokine counterpart.

51. The method or use of claims 45-50, wherein the multispecific protein (or the cytokine when included in the multispecific protein) exhibits an EC50 for cytokine pathway signaling in NK cells that is lower than that observed with the cytokine alone or in a protein in which the NKp46 ABD and/or a CD16 ABD are replaced by a control ABD, optionally wherein the EC50 for cytokine pathway signaling in NK cells is at least 10-fold or 100-fold lower, optionally wherein cytokine pathway signaling is assessed by bringing the cytokine into contact with an NK cell and measuring STAT phosphorylation in the NK cells.

52. The use of claims 47-51, wherein the disease is a cancer or infectious disease.

53. A method of making a heteromultimeric protein, comprising:
(a) providing a first nucleic acid encoding at least a first polypeptide chain according to any of claims 14-23 or 29-42;
(b) providing a second nucleic acid encoding at least a second polypeptide chain according to any of claims 14-23 or 29-42; and
(c) expressing said first and second nucleic acids in a host cell to produce a protein comprising said first and second polypeptide chains, respectively; loading the protein produced onto an affinity purification support, optionally a Protein-A support, and recovering the heteromultimeric protein.

54. A method of making a heteromultimeric protein, comprising:
(a) providing a first nucleic acid encoding a first polypeptide chain according to any of claims 14-28 or 31-42;
(b) providing a second nucleic acid encoding a second polypeptide chain according to any of claims 14-28 or 31-42;
(c) providing a third nucleic acid comprising a third polypeptide chain according to any of claims 14-28 or 31-42; and
(d) expressing said first, second and third nucleic acids in a host cell to produce a protein comprising said first, second and third polypeptide chains, respectively; loading the protein produced onto an affinity purification support, optionally a Protein-A support, and recovering the heteromultimeric protein.

55. A method for identifying or evaluating a polypeptide, comprising the steps of:
(a) providing nucleic acid(s) encoding one of more polypeptides of a protein of any of claims 1-42;
(b) expressing said nucleic acids in a host cell to produce said proteins, respectively; and recovering said protein; and
(c) evaluating the protein produced for a biological activity of interest.
